(19) United States
(12) Patent Application Publication
(10) Pub. No.: US 2008/0207970 A1
(43) Pub. Date: Aug. 28, 2008

(54) Heating an organic-rich rock formation in situ to produce products with improved properties

(76) Inventors: William P. Meurer, Pearland, TX (US); Robert D. Kaminsky, Houston, TX (US); Glenn A. Otten, The Woodlands, TX (US); William A. Symington, Houston, TX (US); Jesse D. Yeakel, Missouri City, TX (US); Ana L. Braun, Houston, TX (US); Lloyd M. Wenger, Houston, TX (US)

Correspondence Address: Exxon Mobil Upstream;Research Company, P.O. Box 2189, (CORP-URC-SW 359), Houston, TX, 77252-2189, US

(21) Appl. No.: 11/973,898

(22) Filed: Oct. 10, 2007

Related U.S. Application Data
(60) Provisional application No. 60/997,654, filed on Oct. 4, 2007.
(60) Provisional application No. 60/997,650, filed on Oct. 4, 2007.
(60) Provisional application No. 60/997,646, filed on Oct. 4, 2007.
(60) Provisional application No. 60/997,645, filed on Oct. 4, 2007.
(60) Provisional application No. 60/997,648, filed on Oct. 4, 2007.
(60) Provisional application No. 60/997,653, filed on Oct. 4, 2007.
(60) Provisional application No. 60/997,649, filed on Oct. 4, 2007.
(60) Provisional application No. 60/851,432, filed on Oct. 13, 2006.
(60) Provisional application No. 60/851,534, filed on Oct. 13, 2006.
(60) Provisional application No. 60/851,535, filed on Oct. 13, 2006.
(60) Provisional application No. 60/851,819, filed on Oct. 13, 2006.
(60) Provisional application No. 60/851,786, filed on Oct. 13, 2006.
(60) Provisional application No. 60/851,820, filed on Oct. 13, 2006.

(51) Int. Cl. C07C 13/16; C07C 5/00

(52) U.S. Cl. 585/24; 585/430

(57) ABSTRACT

A method of producing hydrocarbon fluids with improved hydrocarbon compound properties from a subsurface organic-rich rock formation, such as an oil shale formation, is provided. The method may include the step of heating the organic-rich rock formation in situ. In accordance with the method, the heating of the organic-rich rock formation may pyrolyze at least a portion of the formation hydrocarbons, for example kerogen, to create hydrocarbon fluids. Thereafter, the hydrocarbon fluids may be produced from the formation. Hydrocarbon fluids with improved hydrocarbon compound properties are also provided.

### CROSS-REFERENCE TO RELATED APPLICATIONS

This application claims the benefit of U.S. Provisional application 60/______, titled “Products with Improved Aromatic Hydrocarbon Properties Produced by, In Situ Heating of an Organic-Rich Rock Formation”, docket No. 2007EM267, which was filed on Oct. 4, 2007, U.S. Provisional application 60/______, titled “Heating an Organic-Rich Rock Formation In Situ to Produce Products with Improved Aromatic Hydrocarbon Properties”, docket No. 2007EM266, which was filed on Oct. 4, 2007, U.S. Provisional application 60/______, titled “Products with Improved Branched Hydrocarbon Properties Produced by In Situ Heating of an Organic-Rich Rock Formation”, docket No. 2007EM269, which was filed on Oct. 4, 2007, U.S. Provisional application 60/______, titled “Heating an Organic-Rich Rock Formation In Situ to Produce Products with Improved Branched Hydrocarbon Properties”, docket No. 2007EM268, which was filed on Oct. 4, 2007, U.S. Provisional application 60/______, titled “Products with Improved Cyclic Hydrocarbon Properties Produced by In Situ Heating of an Organic-Rich Rock Formation”, docket No. 2007EM271, which was filed on Oct. 4, 2007, U.S. Provisional application 60/______, titled “Heating an Organic-Rich Rock Formation In Situ to Produce Products with Improved Cyclic Hydrocarbon Properties”, docket No. 2007EM270, which was filed on Oct. 4, 2007, U.S. Provisional application 60/______, titled “Products with Identifying Compound Marker Properties Produced by In Situ Heating of an Organic-Rich Rock Formation”, docket No. 2007EM272, which was filed on Oct. 4, 2007, U.S. Provisional application 60/851,432 which was filed on Oct. 13, 2006, U.S. Provisional application 60/851,534 which was filed on Oct. 13, 2006, U.S. Provisional application 60/851,535 which was filed on Oct. 13, 2006, U.S. Provisional application 60/851,819 which was filed on Oct. 13, 2006, U.S. Provisional application 60/851,786 which was filed on Oct. 13, 2006, and U.S. Provisional application 60/851,820 which was filed on Oct. 13, 2006. The above-referenced provisional applications are incorporated herein in their entirety by reference.

### BACKGROUND OF THE INVENTION

1. Field of the Invention

The present invention relates to the field of hydrocarbon recovery from subsurface formations. More specifically, the present invention relates to in situ recovery of hydrocarbon fluids from organic-rich rock formations, including, for example, oil shale formations, coal formations and tar sands formations.

2. Background of the Invention

Certain geological formations are known to contain an organic matter known as “kerogen.” Kerogen is a solid, carbonaceous material. When kerogen is imbedded in rock formations, the mixture is referred to as oil shale. This is true whether or not the mineral is, in fact, technically shale, that is, a rock formed from compacted clay.

Kerogen is subject to decomposing upon exposure to heat over a period of time. Upon heating, kerogen molecularly decomposes to produce oil, gas, and carbonaceous coke. Small amounts of water may also be generated. The oil, gas and water fluids are mobile within the rock matrix, while the carbonaceous coke remains essentially immobile.

Oil shale formations are found in various areas world-wide, including the United States. Oil shale formations tend to reside at relatively shallow depths. In the United States, oil shale is most notably found in Wyoming, Colorado, and Utah. These formations are often characterized by limited permeability. Some consider oil shale formations to be hydrocarbon deposits which have not yet experienced the years of heat and pressure thought to be required to create conventional oil and gas reserves.

The decomposition rate of kerogen to produce mobile hydrocarbons is temperature dependent. Temperatures generally in excess of 270° C. (518° F.) over the course of many months may be required for substantial conversion. At higher temperatures substantial conversion may occur within shorter times. When kerogen is heated, chemical reactions break the larger molecules forming the solid kerogen into smaller molecules of oil and gas. The thermal conversion process is referred to as pyrolysis or retorting.

Attempts have been made for many years to extract oil from oil shale formations. Near-surface oil shales have been mined and retorted at the surface for over a century. In 1862, James Young began processing Scottish oil shales. The industry lasted for about 100 years. Commercial oil shale retorting through surface mining has been conducted in other countries as well such as Australia, Brazil, China, Estonia, France, Russia, South Africa, Spain, and Sweden. However, the practice has been mostly discontinued in recent years because it proved to be uneconomical or because of environmental constraints on spent shale disposal. (See T. F. Yen, and G. V. Chilingarian, “Oil Shale,” Amsterdam, Elsevier, p. 292, the entire disclosure of which is incorporated herein by reference.) Further, surface retorting requires mining of the oil shale, which limits application to very shallow formations.

In the United States, the existence of oil shale deposits in northwestern Colorado has been known since the early 1900's. While research projects have been conducted in this area from time to time, no serious commercial development has been undertaken. Most research on oil shale production has been carried out in the latter half of the 1900's. The majority of this research was on shale oil geology, geochemistry, and retorting in surface facilities.

In 1947, U.S. Pat. No. 2,732,195 issued to Ljungstrom. That patent, entitled “Method of Treating Oil Shale and Recovery of Oil and Other Mineral Products Therefrom,” proposed the application of heat at high temperatures to the oil shale formation in situ to distill and produce hydrocarbons. The '195 Ljungstrom patent is incorporated herein by reference.

Ljungstrom coined the phrase “heat supply channels” to describe bore holes drilled into the formation. The bore holes received an electrical heat conductor which transferred heat to the surrounding oil shale. Thus, the heat supply channels served as heat injection wells. The electrical heating elements in the heat injection wells were placed within sand or cement or other heat-conductive material to permit the heat injection well to transmit heat into the surrounding oil shale while preventing the inflow of fluid. According to Ljungstrom, the “aggregate” was heated to between 500° and 1,000° C. in some applications.

Along with the heat injection wells, fluid producing wells were also completed in near proximity to the heat injection wells. As kerogen was pyrolyzed upon heat conduction into the rock matrix, the resulting oil and gas would be recovered through the adjacent production wells.

Ljungstrom applied his approach of thermal conduction from heated wellbores through the Swedish Shale Oil Company. A full scale plant was developed that operated from 1944 into the 1950's. (See G. Salamonsson, “The Ljungstrom In Situ Method for Shale-Oil Recovery,” 2^{nd} Oil Shale and Cannel Coal Conference, v. 2, Glasgow, Scotland, Institute of Petroleum, London, p. 260-280 (1951), the entire disclosure of which is incorporated herein by reference.)

Additional in situ methods have been proposed. These methods generally involve the injection of heat and/or solvent into a subsurface oil shale. Heat may be in the form of heated methane (see U.S. Pat. No. 3,241,611 to J. L. Dougan), flue gas, or superheated steam (see U.S. Pat. No. 3,400,762 to D. W. Peacock). Heat may also be in the form of electric resistive heating, dielectric heating, radio frequency (RF) heating (U.S. Pat. No. 4,140,180, assigned to the ITT Research Institute in Chicago, Ill.) or oxidant injection to support in situ combustion. In some instances, artificial permeability has been created in the matrix to aid the movement of pyrolyzed fluids. Permeability generation methods include mining, rubblization, hydraulic fracturing (see U.S. Pat. No. 3,468,376 to M. L. Slusser and U.S. Pat. No. 3,513,914 to J. V. Vogel), explosive fracturing (see U.S. Pat. No. 1,422,204 to W. W. Hoover, et al.), heat fracturing (see U.S. Pat. No. 3,284,281 to R. W. Thomas), and steam fracturing (see U.S. Pat. No. 2,952,450 to H. Purre).

In 1989, U.S. Pat. No. 4,886,118 issued to Shell Oil Company, the entire disclosure of which is incorporated herein by reference. That patent, entitled “Conductively Heating a Subterranean Oil Shale to Create Permeability and Subsequently Produce Oil,” declared that “[c]ontrary to the implications of . . . prior teachings and beliefs . . . the presently described conductive heating process is economically feasible for use even in a substantially impermeable subterranean oil shale.” (col. 6, ln. 50-54). Despite this declaration, it is noted that few, if any, commercial in situ shale oil operations have occurred other than Ljungstrom's application. The '118 patent proposed controlling the rate of heat conduction within the rock surrounding each heat injection well to provide a uniform heat front.

Additional history behind oil shale retorting and shale oil recovery can be found in co-owned patent publication WO 2005/010320 entitled “Methods of Treating a Subterranean Formation to Convert Organic Matter into Producible Hydrocarbons,” and in patent publication WO 2005/045192 entitled “Hydrocarbon Recovery from Impermeable Oil Shales.” The Background and technical disclosures of these two patent publications are incorporated herein by reference.

A need exists for improved processes for the production of shale oil. In addition, a need exists for improved methods of producing shale oil with improved properties.

### SUMMARY OF THE INVENTION

In one embodiment, the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C6 to benzene weight ratio less than 35.0, a n-C7 to toluene weight ratio less than 7.0, a n-C8 to ethylbenzene weight ratio less than 16.0, a n-C8 to ortho-xylene weight ratio less than 7.0, a n-C8 to meta-xylene weight ratio less than 1.9, a n-C9 to 1-ethyl-3-methylbenzene weight ratio less than 8.2, a n-C9 to 1-ethyl-4-methylbenzene weight ratio less than 4.4, a n-C9 to 1,2,4-trimethylbenzene weight ratio less than 2.7, a n-C10 to 1-ethyl-2,3-dimethylbenzene weight ratio less than 13.5, a n-C10 to tetralin weight ratio less than 25.0, a n-C12 to 2-methylnaphthalene weight ratio less than 4.9, and a n-C12 to 1-methylnaphthalene weight ratio less than 6.9.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C6 to benzene weight ratio less than 24.0, a n-C7 to toluene weight ratio less than 6.6, a n-C8 to ethylbenzene weight ratio less than 15.0, a n-C8 to ortho-xylene weight ratio less than 6.6, a n-C8 to meta-xylene weight ratio less than 1.8, a n-C9 to 1-ethyl-3-methylbenzene weight ratio less than 7.9, a n-C9 to 1-ethyl-4-methylbenzene weight ratio less than 4.3, a n-C9 to 1,2,4-trimethylbenzene weight ratio less than 2.6, a n-C10 to 1-ethyl-2,3-dimethylbenzene weight ratio less than 13.1, a n-C10 to tetralin weight ratio less than 23.7, a n-C12 to 2-methylnaphthalene weight ratio less than 5.0, and a n-C12 to 1-methylnaphthalene weight ratio less than 6.8.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C6 to benzene weight ratio less than 13.4, a n-C7 to toluene weight ratio less than 5.1, a n-C8 to ethylbenzene weight ratio less than 12.3, a n-C8 to ortho-xylene weight ratio less than 5.3, a n-C8 to meta-xylene weight ratio less than 1.5, a n-C9 to 1-ethyl-3-methylbenzene weight ratio less than 5.9, a n-C9 to 1-ethyl-4-methylbenzene weight ratio less than 3.8, a n-C9 to 1,2,4-trimethylbenzene weight ratio less than 2.2, a n-C10 to 1-ethyl-2,3-dimethylbenzene weight ratio less than 12.2, a n-C10 to tetralin weight ratio less than 23.4, a n-C12 to 2-methylnaphthalene weight ratio less than 4.0, and a n-C12 to 1-methylnaphthalene weight ratio less than 6.1.

In one embodiment, the invention includes an in situ method of producing hydrocarbon fluids from an organic-rich rock formation. The method may include heating in situ a section of an organic-rich rock formation containing formation hydrocarbons, where the section of an organic-rich rock formation has a lithostatic stress greater than 200 psi, pyrolyzing at least a portion of the formation hydrocarbons thereby forming a hydrocarbon fluid, and producing the hydrocarbon fluid from the organic-rich rock formation. The produced hydrocarbon fluid may include a condensable hydrocarbon portion, where the condensable hydrocarbon portion has one or more of a n-C6 to benzene weight ratio less than 35.0, a n-C7 to toluene weight ratio less than 7.0, a n-C8 to ethylbenzene weight ratio less than 16.0, a n-C8 to ortho-xylene weight ratio less than 7.0, a n-C8 to meta-xylene weight ratio less than 1.9, a n-C9 to 1-ethyl-3-methylbenzene weight ratio less than 8.2, a n-C9 to 1-ethyl-4-methylbenzene weight ratio less than 4.4, a n-C9 to 1,2,4-trimethylbenzene weight ratio less than 2.7, a n-C10 to 1-ethyl-2,3-dimethylbenzene weight ratio less than 13.5, a n-C10 to tetralin weight ratio less than 25.0, a n-C12 to 2-methylnaphthalene weight ratio less than 4.9, and a n-C12 to 1-methylnaphthalene weight ratio less than 6.9.

In one embodiment, the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C9 to IP-9 weight ratio greater than 2.4, a n-C10 to IP-10 weight ratio greater than 1.4, a n-C11 to IP-11 weight ratio greater than 1.0, a n-C13 to IP-13 weight ratio greater than 1.1, a n-C14 to IP-14 weight ratio greater than 1.1, a n-C15 to IP-15 weight ratio greater than 1.0, a n-C16 to IP-16 weight ratio greater than 0.8, a n-C18 to IP-18 weight ratio greater than 1.0, and a n-C19 to pristane weight ratio greater than 1.6.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C9 to IP-9 weight ratio greater than 2.5, a n-C10 to IP-10 weight ratio greater than 1.5, a n-C11 to IP-11 weight ratio greater than 1.1, a n-C13 to IP-13 weight ratio greater than 1.2, a n-C14 to IP-14 weight ratio greater than 1.2, a n-C15 to IP-15 weight ratio greater than 1.1, a n-C16 to IP-16 weight ratio greater than 0.9, a n-C18 to IP-18 weight ratio greater than 1.1, and a n-C19 to pristane weight ratio greater than 1.8.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C9 to IP-9 weight ratio greater than 2.6, a n-C10 to IP-10 weight ratio greater than 1.6, a n-C11 to IP-11 weight ratio greater than 1.2, a n-C13 to IP-13 weight ratio greater than 1.3, a n-C14 to IP-14 weight ratio greater than 1.4, a n-C15 to IP-15 weight ratio greater than 1.4, a n-C16 to IP-16 weight ratio greater than 1.2, a n-C18 to IP-18 weight ratio greater than 1.5, and a n-C19 to pristane weight ratio greater than 2.4.

In one embodiment, the invention includes an in situ method of producing hydrocarbon fluids from an organic-rich rock formation. The method may include heating in situ a section of an organic-rich rock formation containing formation hydrocarbons, where the section of an organic-rich rock formation has a lithostatic stress greater than 200 psi, pyrolyzing at least a portion of the formation hydrocarbons thereby forming a hydrocarbon fluid, and producing the hydrocarbon fluid from the organic-rich rock formation. The produced hydrocarbon fluid may include a condensable hydrocarbon portion, where the condensable hydrocarbon portion has one or more of a n-C9 to IP-9 weight ratio greater than 2.4, a n-C10 to IP-10 weight ratio greater than 1.4, a n-C11 to IP-11 weight ratio greater than 1.0, a n-C13 to IP-13 weight ratio greater than 1.1, a n-C14 to IP-14 weight ratio greater than 1.1, a n-C15 to IP-15 weight ratio greater than 1.0, a n-C16 to IP-16 weight ratio greater than 0.8, a n-C18 to IP-18 weight ratio greater than 1.0, and a n-C19 to pristane weight ratio greater than 1.6.

In one embodiment, the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C7 to cis 1,3-dimethyl cyclopentane weight ratio less than 13.1, a n-C7 to trans 1,3-dimethyl cyclopentane weight ratio less than 14.9, a n-C7 to trans 1,2-dimethyl cyclopentane weight ratio less than 7.0, a n-C7 to methyl cyclohexane weight ratio less than 5.2, a n-C7 to ethyl cyclopentane weight ratio less than 11.3, a n-C8 to 1,1-dimethyl cyclohexane weight ratio less than 16.0, a n-C8 to trans 1,2-dimethyl cyclohexane weight ratio less than 17.5, and a n-C8 to ethyl cyclohexane weight ratio less than 12.3.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C7 to cis 1,3-dimethyl cyclopentane weight ratio less than 12.7, a n-C7 to trans 1,3-dimethyl cyclopentane weight ratio less than 14.7, a n-C7 to trans 1,2-dimethyl cyclopentane weight ratio less than 6.6, a n-C7 to methyl cyclohexane weight ratio less than 5.0, a n-C7 to ethyl cyclopentane weight ratio less than 10.9, a n-C8 to 1,1-dimethyl cyclohexane weight ratio less than 15.4, a n-C8 to trans 1,2-dimethyl cyclohexane weight ratio less than 16.5, and a n-C8 to ethyl cyclohexane weight ratio less than 12.0.

Another embodiment of the invention includes a hydrocarbon fluid produced through in situ pyrolysis of oil shale within an oil shale formation where the hydrocarbon fluid comprises a condensable hydrocarbon portion and the condensable hydrocarbon portion has one or more of a n-C7 to cis 1,3-dimethyl cyclopentane weight ratio less than 10.3, a n-C7 to trans 1,3-dimethyl cyclopentane weight ratio less than 11.6, a n-C7 to trans 1,2-dimethyl cyclopentane weight ratio less than 5.9, a n-C7 to methyl cyclohexane weight ratio less than 4.1, a n-C7 to ethyl cyclopentane weight ratio less than 9.5, a n-C8 to 1,1-dimethyl cyclohexane weight ratio less than 13.9, a n-C8 to trans 1,2-dimethyl cyclohexane weight ratio less than 12.3, and a n-C8 to ethyl cyclohexane weight ratio less than 10.3.

In one embodiment, the invention includes an in situ method of producing hydrocarbon fluids from an organic-rich rock formation. The method may include heating in situ a section of an organic-rich rock formation containing formation hydrocarbons, where the section of an organic-rich rock formation has a lithostatic stress greater than 200 psi, pyrolyzing at least a portion of the formation hydrocarbons thereby forming a hydrocarbon fluid, and producing the hydrocarbon fluid from the organic-rich rock formation. The produced hydrocarbon fluid may include a condensable hydrocarbon portion, where the condensable hydrocarbon portion has one or more of a n-C7 to cis 1,3-dimethyl cyclopentane weight ratio less than 13.1, a n-C7 to trans 1,3-dimethyl cyclopentane weight ratio less than 14.9, a n-C7 to trans 1,2-dimethyl cyclopentane weight ratio less than 7.0, a n-C7 to methyl cyclohexane weight ratio less than 5.2, a n-C7 to ethyl cyclopentane weight ratio less than 11.3, a n-C8 to 1,1-dimethyl cyclohexane weight ratio less than 16.0, a n-C8 to trans 1,2-dimethyl cyclohexane weight ratio less than 17.5, and a n-C8 to ethyl cyclohexane weight ratio less than 12.3.

In one embodiment, the invention includes a hydrocarbon fluid produced through in situ pyrolysis of oil shale within an oil shale formation. The hydrocarbon fluid including a condensable hydrocarbon portion. The condensable hydrocarbon portion having one or more of a total C9 to total C20 weight ratio between 2.5 and 6.0, a total C10 to total C20 weight ratio between 2.8 and 7.3, a total C11 to total C20 weight ratio between 2.6 and 6.5, a total C12 to total C20 weight ratio between 2.6 and 6.4 and a total C13 to total C20 weight ratio between 3.2 and 8.0.

Another embodiment of the invention includes a hydrocarbon fluid produced through in situ pyrolysis of oil shale within an oil shale formation. The hydrocarbon fluid including a condensable hydrocarbon portion. The condensable hydrocarbon portion having one or more of a total C10 to total C25 weight ratio between 7.1 and 24.5, a total C11 to total C25 weight ratio between 6.5 and 22.0, a total C12 to total C25 weight ratio between 6.5 and 22.0, and a total C13 to total C25 weight ratio between 8.0 and 27.0.

Another embodiment of the invention includes a hydrocarbon fluid produced through in situ pyrolysis of oil shale within an oil shale formation. The hydrocarbon fluid including a condensable hydrocarbon portion. The condensable hydrocarbon portion having one or more of a total C10 to total C29 weight ratio between 15.0 and 60.0, a total C11 to total C29 weight ratio between 13.0 and 54.0, a total C12 to total C29 weight ratio between 12.5 and 53.0, and a total C13 to total C29 weight ratio between 16.0 and 65.0.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a total C7 to total C20 weight ratio greater than 0.8, a total C8 to total C20 weight ratio greater than 1.7, a total C9 to total C20 weight ratio greater than 2.5, a total C10 to total C20 weight ratio greater than 2.8, a total C11 to total C20 weight ratio greater than 2.3, a total C12 to total C20 weight ratio greater than 2.3, a total C13 to total C20 weight ratio greater than 2.9, a total C14 to total C20 weight ratio greater than 2.2, a total C15 to total C20 weight ratio greater than 2.2, and a total C16 to total C20 weight ratio greater than 1.6.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a total C7 to total C25 weight ratio greater than 2.0, a total C8 to total C25 weight ratio greater than 4.5, a total C9 to total C25 weight ratio greater than 6.5, a total C10 to total C25 weight ratio greater than 7.5, a total C11 to total C25 weight ratio greater than 6.5, a total C12 to total C25 weight ratio greater than 6.5, a total C13 to total C25 weight ratio greater than 8.0, a total C14 to total C25 weight ratio greater than 6.0, a total C15 to total C25 weight ratio greater than 6.0, a total C16 to total C25 weight ratio greater than 4.5, a total C17 to total C25 weight ratio greater than 4.8, and a total C18 to total C25 weight ratio greater than 4.5.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a total C7 to total C29 weight ratio greater than 3.5, a total C8 to total C29 weight ratio greater than 9.0, a total C9 to total C29 weight ratio greater than 12.0, a total C10 to total C29 weight ratio greater than 15.0, a total C11 to total C29 weight ratio greater than 13.0, a total C12 to total C29 weight ratio greater than 12.5, a total C13 to total C29 weight ratio greater than 16.0, a total C14 to total C29 weight ratio greater than 12.0, a total C15 to total C29 weight ratio greater than 12.0, a total C16 to total C29 weight ratio greater than 9.0, a total C17 to total C29 weight ratio greater than 10.0, a total C18 to total C29 weight ratio greater than 8.8, a total C19 to total C29 weight ratio greater than 7.0, a total C20 to total C29 weight ratio greater than 6.0, a total C21 to total C29 weight ratio greater than 5.5, and a total C22 to total C29 weight ratio greater than 4.2.

In one embodiment, the invention includes a hydrocarbon fluid produced through in situ pyrolysis of oil shale within an oil shale formation. The hydrocarbon fluid including a condensable hydrocarbon portion. The condensable hydrocarbon portion having one or more of a normal-C7 to normal-C20 weight ratio greater than 0.9, a normal-C8 to normal-C20 weight ratio greater than 2.0, a normal-C9 to normal-C20 weight ratio greater than 1.9, a normal-C10 to normal-C20 weight ratio greater than 2.2, a normal-C11 to normal-C20 weight ratio greater than 1.9, a normal-C12 to normal-C20 weight ratio greater than 1.9, a normal-C13 to normal-C20 weight ratio greater than 2.3, a normal-C14 to normal-C20 weight ratio greater than 1.8, a normal-C15 to normal-C20 weight ratio greater than 1.8, and normal-C16 to normal-C20 weight ratio greater than 1.3.

Another embodiment of the invention includes a hydrocarbon fluid produced through in situ pyrolysis of oil shale within an oil shale formation. The hydrocarbon fluid including a condensable hydrocarbon portion. The condensable hydrocarbon portion having one or more of a normal-C7 to normal-C25 weight ratio greater than 1.9, a normal-C8 to normal-C25 weight ratio greater than 3.9, a normal-C9 to normal-C25 weight ratio greater than 3.7, a normal-C10 to normal-C25 weight ratio greater than 4.4, a normal-C11 to normal-C25 weight ratio greater than 3.8, a normal-C12 to normal-C25 weight ratio greater than 3.7, a normal-C13 to normal-C25 weight ratio greater than 4.7, a normal-C14 to normal-C25 weight ratio greater than 3.7, a normal-C15 to normal-C25 weight ratio greater than 3.7, a normal-C16 to normal-C25 weight ratio greater than 2.5, a normal-C17 to normal-C25 weight ratio greater than 3.0, and a normal-C18 to normal-C25 weight ratio greater than 3.4.

Another embodiment of the invention includes a hydrocarbon fluid produced through in situ pyrolysis of oil shale within an oil shale formation. The hydrocarbon fluid including a condensable hydrocarbon portion. The condensable hydrocarbon portion having one or more of a normal-C7 to normal-C29 weight ratio greater than 18.0, a normal-C8 to normal-C29 weight ratio greater than 16.0, a normal-C9 to normal-C29 weight ratio greater than 14.0, a normal-C10 to normal-C29 weight ratio greater than 14.0, a normal-C11 to normal-C29 weight ratio greater than 13.0, a normal-C12 to normal-C29 weight ratio greater than 11.0, a normal-C13 to normal-C29 weight ratio greater than 10.0, a normal-C14 to normal-C29 weight ratio greater than 9.0, a normal-C15 to normal-C29 weight ratio greater than 8.0, a normal-C16 to normal-C29 weight ratio greater than 8.0, a normal-C17 to normal-C29 weight ratio greater than 6.0, a normal-C18 to normal-C29 weight ratio greater than 6.0, a normal-C19 to normal-C29 weight ratio greater than 5.0, a normal-C20 to normal-C29 weight ratio greater than 4.0, a normal-C21 to normal-C29 weight ratio greater than 3.6, and a normal-C22 to normal-C29 weight ratio greater than 2.8.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a normal-C10 to normal-C20 weight ratio greater than 2.2, a normal-C11 to normal-C20 weight ratio greater than 1.9, a normal-C12 to normal-C20 weight ratio greater than 1.9, a normal-C13 to normal-C20 weight ratio greater than 2.3, a normal-C14 to normal-C20 weight ratio greater than 1.8, a normal-C15 to normal-C20 weight ratio greater than 1.8, and normal-C16 to normal-C20 weight ratio greater than 1.3.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a normal-C10 to normal-C25 weight ratio greater than 4.4, a normal-C11 to normal-C25 weight ratio greater than 3.8, a normal-C12 to normal-C25 weight ratio greater than 3.7, a normal-C13 to normal-C25 weight ratio greater than 4.7, a normal-C14 to normal-C25 weight ratio greater than 3.7, a normal-C15 to normal-C25 weight ratio greater than 3.7, a normal-C16 to normal-C25 weight ratio greater than 2.5, a normal-C17 to normal-C25 weight ratio greater than 3.0, and a normal-C18 to normal-C25 weight ratio greater than 3.4.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a normal-C7 to normal-C29 weight ratio greater than 18.0, a normal-C8 to normal-C29 weight ratio greater than 16.0, a normal-C9 to normal-C29 weight ratio greater than 14.0, a normal-C10 to normal-C29 weight ratio greater than 14.0, a normal-C11 to normal-C29 weight ratio greater than 13.0, a normal-C12 to normal-C29 weight ratio greater than 11.0, a normal-C13 to normal-C29 weight ratio greater than 10.0, a normal-C14 to normal-C29 weight ratio greater than 9.0, a normal-C15 to normal-C29 weight ratio greater than 8.0, a normal-C16 to normal-C29 weight ratio greater than 8.0, a normal-C17 to normal-C29 weight ratio greater than 6.0, a normal-C18 to normal-C29 weight ratio greater than 6.0, a normal-C19 to normal-C29 weight ratio greater than 5.0, a normal-C20 to normal-C29 weight ratio greater than 4.0, a normal-C21 to normal-C29 weight ratio greater than 3.6, and a normal-C22 to normal-C29 weight ratio greater than 2.8.

In one embodiment, the invention includes a hydrocarbon fluid produced through in situ pyrolysis of oil shale within an oil shale formation. The hydrocarbon fluid including a condensable hydrocarbon portion. The condensable hydrocarbon portion having one or more of a normal-C10 to total C10 weight ratio less than 0.31, a normal-C11 to total C11 weight ratio less than 0.32, a normal-C12 to total C12 weight ratio less than 0.29, a normal-C13 to total C13 weight ratio less than 0.28, a normal-C14 to total C14 weight ratio less than 0.31, a normal-C15 to total C15 weight ratio less than 0.27, a normal-C16 to total C16 weight ratio less than 0.31, a normal-C17 to total C17 weight ratio less than 0.31, a normal-C18 to total C18 weight ratio less than 0.37, normal-C19 to total C19 weight ratio less than 0.37, a normal-C20 to total C20 weight ratio less than 0.37, a normal-C21 to total C21 weight ratio less than 0.37, a normal-C22 to total C22 weight ratio less than 0.38, normal-C23 to total C23 weight ratio less than 0.43, a normal-C24 to total C24 weight ratio less than 0.48, and a normal-C25 to total C25 weight ratio less than 0.53.

In one embodiment, the invention includes an in situ method of producing hydrocarbon fluid from an organic-rich rock formation. The method includes heating in situ a section of an organic-rich rock formation having a lithostatic stress greater than 200 psi. The method further includes producing a hydrocarbon fluid from the organic-rich rock formation. The produced hydrocarbon fluid including a condensable hydrocarbon portion. The condensable hydrocarbon portion having one or more of a normal-C10 to total C10 weight ratio less than 0.31, a normal-C11 to total C11 weight ratio less than 0.32, a normal-C12 to total C12 weight ratio less than 0.29, a normal-C13 to total C13 weight ratio less than 0.28, a normal-C14 to total C14 weight ratio less than 0.31, a normal-C15 to total C15 weight ratio less than 0.27, a normal-C16 to total C16 weight ratio less than 0.31, a normal-C17 to total C17 weight ratio less than 0.31, a normal-C18 to total C18 weight ratio less than 0.37, normal-C19 to total C19 weight ratio less than 0.37, a normal-C20 to total C20 weight ratio less than 0.37, a normal-C21 to total C21 weight ratio less than 0.37, a normal-C22 to total C22 weight ratio less than 0.38, normal-C23 to total C23 weight ratio less than 0.43, a normal-C24 to total C24 weight ratio less than 0.48, and a normal-C25 to total C25 weight ratio less than 0.53.

### BRIEF DESCRIPTION OF THE DRAWINGS

So that the manner in which the features of the present invention can be better understood, certain drawings, graphs and flow charts are appended hereto. It is to be noted, however, that the drawings illustrate only selected embodiments of the inventions and are therefore not to be considered limiting of scope, for the inventions may admit to other equally effective embodiments and applications.

FIG. 1 is a cross-sectional view of an illustrative subsurface area. The subsurface area includes an organic-rich rock matrix that defines a subsurface formation.

FIG. 2 is a flow chart demonstrating a general method of in situ thermal recovery of oil and gas from an organic-rich rock formation, in one embodiment.

FIG. 3 is a cross-sectional view of an illustrative oil shale formation that is within or connected to groundwater aquifers and a formation leaching operation.

FIG. 4 is a plan view of an illustrative heater well pattern, around a production well. Two layers of heater wells are shown.

FIG. 5 is a bar chart comparing one ton of Green River oil shale before and after a simulated in situ, retorting process.

FIG. 6 is a process flow diagram of exemplary surface processing facilities for a subsurface formation development.

FIG. 7 is a graph of the weight percent of each carbon number pseudo component occurring from C6 to C38 for laboratory experiments conducted at three different stress levels.

FIG. 8 is a graph of the weight percent ratios of each carbon number pseudo component occurring from C6 to C38 as compared to the C20 pseudo component for laboratory experiments conducted at three different stress levels.

FIG. 9 is a graph of the weight percent ratios of each carbon number pseudo component occurring from C6 to C38 as compared to the C25 pseudo component for laboratory experiments conducted at three different stress levels.

FIG. 10 is a graph of the weight percent ratios of each carbon number pseudo component occurring from C6 to C38 as compared to the C29 pseudo component for laboratory experiments conducted at three different stress levels.

FIG. 11 is a graph of the weight percent of normal alkane hydrocarbon compounds occurring from normal-C6 to normal-C38 for laboratory experiments conducted at three different stress levels.

FIG. 12 is a graph of the weight percent of normal alkane hydrocarbon compounds occurring from normal-C6 to normal-C38 as compared to the normal-C20 hydrocarbon compound for laboratory experiments conducted at three different stress levels.

FIG. 13 is a graph of the weight percent of normal alkane hydrocarbon compounds occurring from normal-C6 to normal-C38 as compared to the normal-C25 hydrocarbon compound for laboratory experiments conducted at three different stress levels.

FIG. 14 is a graph of the weight percent of normal alkane hydrocarbon compounds occurring from normal-C6 to normal-C38 as compared to the normal-C29 hydrocarbon compound for laboratory experiments conducted at three different stress levels.

FIG. 15 is a graph of the weight ratio of normal alkane hydrocarbon compounds to pseudo components for each carbon number from C6 to C38 for laboratory experiments conducted at three different stress levels.

FIG. 16 is a bar graph showing the concentration, in molar percentage, of the hydrocarbon species present in the gas samples taken from duplicate laboratory experiments conducted at three different stress levels.

FIG. 17 is an exemplary view of the gold tube apparatus used in the unstressed Parr heating test described in Example 1.

FIG. 18 is a cross-sectional view of the Parr vessel used in Examples 1-5.

FIG. 19 is gas chromatogram of gas sampled from Example 1.

FIG. 20 is a whole oil gas chromatogram of liquid sampled from Example 1.

FIG. 21 is an exemplary view of a Berea cylinder, Berea plugs, and an oil shale core specimen as used in Examples 2-5.

FIG. 22 is an exemplary view of the mini load frame and sample assembly used in Examples 2-5.

FIG. 23 is gas chromatogram of gas sampled from Example 2.

FIG. 24 is gas chromatogram of gas sampled from Example 3.

FIG. 25 is a whole oil gas chromatogram of liquid sampled from Example 3.

FIG. 26 is gas chromatogram of gas sampled from Example 4.

FIG. 27 is a whole oil gas chromatogram of liquid sampled from Example 4.

FIG. 28 is gas chromatogram of gas sampled from Example 5.

FIG. 29 is a graph of the weight ratio of each identified compound occurring from n-C3 to n-C19 for each of the six 393° C. experiments (Examples 13-19) compared to the weight ratio of each identified compound occurring from n-C3 to n-C19 for Example 13 conducted at 393° C., 500 psig initial argon pressure and 0 psi stress.

FIG. 30 is a bar graph of the weight ratio of several normal hydrocarbon compounds to like carbon number aromatic hydrocarbon compounds for each of the seven 393° C. experiments (Examples 13-19) discussed in the Experimental section herein.

FIG. 31 is a bar graph of the weight ratio of several normal hydrocarbon compounds to like carbon number aromatic hydrocarbon compounds for each of the seven 375° C. experiments (Examples 6-12) discussed in the Experimental section herein.

FIG. 32 is a bar graph of the weight ratio of several normal hydrocarbon compounds to like carbon number aromatic hydrocarbon compounds for each of the seven 375° C. and seven 393° C. experiments (Examples 6-19) discussed in the Experimental section herein.

FIG. 33 is a bar graph of the weight ratio of several normal hydrocarbon compounds to like carbon number aromatic hydrocarbon compounds for each of the seven 375° C. and seven 393° C. experiments (Examples 6-19) discussed in the Experimental section herein.

FIG. 34 is a bar graph of the weight ratio of several normal hydrocarbon compounds to like carbon number cyclic hydrocarbon compounds for each of the seven 393° C. experiments (Examples 13-19) discussed in the Experimental section herein.

FIG. 35 is a bar graph of the weight ratio of several normal hydrocarbon compounds to like carbon number cyclic hydrocarbon compounds for each of the seven 375° C. and seven 393° C. experiments (Examples 6-19) discussed in the Experimental section herein.

FIG. 36 is a bar graph of the weight ratio of several normal hydrocarbon compounds to like carbon number isoprenoid hydrocarbon compounds for each of the seven 393° C. experiments (Examples 13-19) discussed in the Experimental section herein.

FIG. 37 is a bar graph of the weight ratio of several normal hydrocarbon compounds to like carbon number isoprenoid hydrocarbon compounds for each of the seven 375° C. and seven 393° C. experiments (6-19) discussed in the Experimental section herein.

FIG. 38 is a bar graph of the weight ratio of the certain hydrocarbon compounds to similar carbon number isoprenoid hydrocarbon compounds for each of the seven 375° C. and seven 393° C. experiments (Examples 6-19) discussed in the Experimental section herein.

FIG. 39 is a C4-C19 GC chromatogram for hydrocarbon liquid sampled in Example 6.

FIG. 40 is a C4-C19 GC chromatogram for hydrocarbon liquid sampled in Example 7.

FIG. 41 is a C4-C19 GC chromatogram for hydrocarbon liquid sampled in Example 8.

FIG. 42 is a C4-C19 GC chromatogram for hydrocarbon liquid sampled in Example 9.

FIG. 43 is a C4-C19 GC chromatogram for hydrocarbon liquid sampled in Example 10.

FIG. 44 is a C4-C19 GC chromatogram for hydrocarbon liquid sampled in Example 11.

FIG. 45 is a C4-C19 GC chromatogram for hydrocarbon liquid sampled in Example 12.

FIG. 46 is a C4-C19 GC chromatogram for hydrocarbon liquid sampled in Example 13.

FIG. 47 is a C4-C19 GC chromatogram for hydrocarbon liquid sampled in Example 14.

FIG. 48 is a C4-C19 GC chromatogram for hydrocarbon liquid sampled in Example 15.

FIG. 49 is a C4-C19 GC chromatogram for hydrocarbon liquid sampled in Example 16.

FIG. 50 is a C4-C19 GC chromatogram for hydrocarbon liquid sampled in Example 17.

FIG. 51 is a C4-C19 GC chromatogram for hydrocarbon liquid sampled in Example 18.

FIG. 52 is a C4-C19 GC chromatogram for hydrocarbon liquid sampled in Example 19.

FIG. 53 is a plot of the weight ratio of (trisnorhopane maturable) to (trisnorhopane maturable+trisnorhopane stable) for Examples 6-20.

FIG. 54 is a plot of the weight ratio of [C-29 17α (H), 21β (H) hopane] to [C-29 17α (H), 21β (H) hopane+C-29 17β (H), 21β (H) hopane] for Examples 6-20.

FIG. 55 is a plot of the weight ratio of [C-30 17α (H), 21β (H) hopane] to [C-30 17α (H), 21β (H) hopane+C-30 17β (H), 21β (H) hopane] for Examples 6-20.

FIG. 56 is a plot of the weight ratio of [C-31 17α (H), 21β (H), 22S homohopane] to [C-31 17α (H), 21β (H), 22S homohopane+C-31 17α (H), 21β (H), 22R homohopane] for Examples 6-20.

FIG. 57 is a plot of the weight ratio of [C-29 5α, 14α, 17α (H) 20R steranes] to [C-29 5α, 14α, 17α (H) 20R steranes+C-29 5α, 14α, 17α (H) 20S steranes] for examples 6-20.

FIG. 58 is a plot of the weight ratio of [C-29 5α, 14β, 17β (H) 20S+C-29 5α, 14β, 17β (H) 20R steranes] to [C-29 5α, 14β, 17β (H) 20S+C-29 5α, 14 β, 17β (H) 20R steranes+C-29 5α, 14α, 17α (H) 20S+C-29 5α, 14α, 17α (H) 20R steranes] for Examples 6-20.

FIG. 59 is a plot of the weight ratio of 3-methyl phenanthrene (3-MP)+2-methyl phenanthrene (2-MP) to 1-methyl phenanthrene (1-MP)+9-methyl phenanthrene (9-MP) for Examples 6-20.

FIG. 60 is a graph of the weight ratio of each identified compound occurring from i-C4 to n-C35 for each of the six 393° C. experiments (Examples 13-19) compared to the weight ratio of each identified compound occurring from i-C4 to n-C35 for Example 13 conducted at 393° C., 500 psig initial argon pressure and 0 psi stress.

FIG. 61 is a graph of the weight ratio of each identified compound occurring from i-C4 to n-C35 for each of the six 375° C. experiments (Examples 7-12) compared to the weight ratio of each identified compound occurring from i-C4 to n-C35 for Example 6 conducted at 375° C., 500 psig initial argon pressure and 0 psi stress.

FIG. 62 is a photograph of an unheated oil shale core plug used in experiments described herein.

FIG. 63 is a photograph of a thin section detail of the unheated oil shale core plug depicted in FIG. 62.

FIG. 64 is a photograph of an oil shale core plug that has been heated under no stress as used in experiments described herein.

FIG. 65 is a photograph of a thin section detail of the unstressed and heated oil shale core plug depicted in FIG. 64.

FIG. 66 is a photograph of an oil shale core plug that has been heated under stress as used in experiments described herein.

FIG. 67 is a photograph of a thin section detail of the stressed and heated oil shale core plug depicted in FIG. 66.

### DETAILED DESCRIPTION OF CERTAIN EMBODIMENTS

### Definitions

As used herein, the term “hydrocarbon(s)” refers to organic material with molecular structures containing carbon bonded to hydrogen. Hydrocarbons may also include other elements, such as, but not limited to, halogens, metallic elements, nitrogen, oxygen, and/or sulfur.

As used herein, the term “hydrocarbon fluids” refers to a hydrocarbon or mixtures of hydrocarbons that are gases or liquids. For example, hydrocarbon fluids may include a hydrocarbon or mixtures of hydrocarbons that are gases or liquids at formation conditions, at processing conditions or at ambient conditions (15° C. and 1 atm pressure). Hydrocarbon fluids may include, for example, oil, natural gas, coal bed methane, shale oil, pyrolysis oil, pyrolysis gas, a pyrolysis product of coal, and other hydrocarbons that are in a gaseous or liquid state.

As used herein, the terms “produced fluids” and “production fluids” refer to liquids and/or gases removed from a subsurface formation, including, for example, an organic-rich rock formation. Produced fluids may include both hydrocarbon fluids and non-hydrocarbon fluids. Production fluids may include, but are not limited to, pyrolyzed shale oil, synthesis gas, a pyrolysis product of coal, carbon dioxide, hydrogen sulfide and water (including steam). Produced fluids may include both hydrocarbon fluids and non-hydrocarbon fluids.

As used herein, the term “condensable hydrocarbons” means those hydrocarbons that condense at 25° C. and one atmosphere absolute pressure. Condensable hydrocarbons may include a mixture of hydrocarbons having carbon numbers greater than 4.

As used herein, the term “non-condensable hydrocarbons” means those hydrocarbons that do not condense at 25° C. and one atmosphere absolute pressure. Non-condensable hydrocarbons may include hydrocarbons having carbon numbers less than 5.

As used herein, the term “heavy hydrocarbons” refers to hydrocarbon fluids that are highly viscous at ambient conditions (15° C. and 1 atm pressure). Heavy hydrocarbons may include highly viscous hydrocarbon fluids such as heavy oil, tar, and/or asphalt. Heavy hydrocarbons may include carbon and hydrogen, as well as smaller concentrations of sulfur, oxygen, and nitrogen. Additional elements may also be present in heavy hydrocarbons in trace amounts. Heavy hydrocarbons may be classified by API gravity. Heavy hydrocarbons generally have an API gravity below about 20 degrees. Heavy oil, for example, generally has an API gravity of about 10-20 degrees, whereas tar generally has an API gravity below about 10 degrees. The viscosity of heavy hydrocarbons is generally greater than about 100 centipoise at 15° C.

As used herein, the term “solid hydrocarbons” refers to any hydrocarbon material that is found naturally in substantially solid form at formation conditions. Non-limiting examples include kerogen, coal, shungites, asphaltites, and natural mineral waxes.

As used herein, the term “formation hydrocarbons” refers to both heavy hydrocarbons and solid hydrocarbons that are contained in an organic-rich rock formation. Formation hydrocarbons may be, but are not limited to, kerogen, oil shale, coal, bitumen, tar, natural mineral waxes, and asphaltites.

As used herein, the term “tar” refers to a viscous hydrocarbon that generally has a viscosity greater than about 10,000 centipoise at 15° C. The specific gravity of tar generally is greater than 1.000. Tar may have an API gravity less than 10 degrees.

As used herein, the term “kerogen” refers to a solid, insoluble hydrocarbon that principally contains carbon, hydrogen, nitrogen, oxygen, and sulfur. Oil shale contains kerogen.

As used herein, the term “bitumen” refers to a non-crystalline solid or viscous hydrocarbon material that is substantially soluble in carbon disulfide.

As used herein, the term “oil” refers to a hydrocarbon fluid containing a mixture of condensable hydrocarbons.

As used herein, the term “subsurface” refers to geologic strata occurring below the earth's surface.

As used herein, the term “hydrocarbon-rich formation” refers to any formation that contains more than trace amounts of hydrocarbons. For example, a hydrocarbon-rich formation may include portions that contain hydrocarbons at a level of greater than 5 volume percent. The hydrocarbons located in a hydrocarbon-rich formation may include, for example, oil, natural gas, heavy hydrocarbons, and solid hydrocarbons.

As used herein, the term “organic-rich rock” refers to any rock matrix holding solid hydrocarbons and/or heavy hydrocarbons. Rock matrices may include, but are not limited to, sedimentary rocks, shales, siltstones, sands, silicilytes, carbonates, and diatomites.

As used herein, the term “formation” refers to any finite subsurface region. The formation may contain one or more hydrocarbon-containing layers, one or more non-hydrocarbon containing layers, an overburden, and/or an underburden of any subsurface geologic formation. An “overburden” and/or an “underburden” is geological material above or below the formation of interest. An overburden or underburden may include one or more different types of substantially impermeable materials. For example, overburden and/or underburden may include rock, shale, mudstone, or wet/tight carbonate (i.e., an impermeable carbonate without hydrocarbons). An overburden and/or an underburden may include a hydrocarbon-containing layer that is relatively impermeable. In some cases, the overburden and/or underburden may be permeable.

As used herein, the term “organic-rich rock formation” refers to any formation containing organic-rich rock. Organic-rich rock formations include, for example, oil shale formations, coal formations, and tar sands formations.

As used herein, the term “pyrolysis” refers to the breaking of chemical bonds through the application of heat. For example, pyrolysis may include transforming a compound into one or more other substances by heat alone or by heat in combination with an oxidant. Pyrolysis may include modifying the nature of the compound by addition of hydrogen atoms which may be obtained from molecular hydrogen, water, carbon dioxide, or carbon monoxide. Heat may be transferred to a section of the formation to cause pyrolysis.

As used herein, the term “water-soluble minerals” refers to minerals that are soluble in water. Water-soluble minerals include, for example, nahcolite (sodium bicarbonate), soda ash (sodium carbonate), dawsonite (NaAl(CO₃)(OH)₂), or combinations thereof. Substantial solubility may require heated water and/or a non-neutral pH solution.

As used herein, the term “formation water-soluble minerals” refers to water-soluble minerals that are found naturally in a formation.

As used herein, the term “migratory contaminant species” refers to species that are both soluble or moveable in water or an aqueous fluid, and are considered to be potentially harmful or of concern to human health or the environment. Migratory contaminant species may include inorganic and organic contaminants. Organic contaminants may include saturated hydrocarbons, aromatic hydrocarbons, and oxygenated hydrocarbons. Inorganic contaminants may include metal contaminants, and ionic contaminants of various types that may significantly alter pH or the formation fluid chemistry. Aromatic hydrocarbons may include, for example, benzene, toluene, xylene, ethylbenzene, and tri-methylbenzene, and various types of polyaromatic hydrocarbons such as anthracenes, naphthalenes, chrysenes and pyrenes. Oxygenated hydrocarbons may include, for example, alcohols, ketones, phenols, and organic acids such as carboxylic acid. Metal contaminants may include, for example, arsenic, boron, chromium, cobalt, molybdenum, mercury, selenium, lead, vanadium, nickel or zinc. Ionic contaminants include, for example, sulfides, sulfates, chlorides, fluorides, ammonia, nitrates, calcium, iron, magnesium, potassium, lithium, boron, and strontium.

As used herein, the term “cracking” refers to a process involving decomposition and molecular recombination of organic compounds to produce a greater number of molecules than were initially present. In cracking, a series of reactions take place accompanied by a transfer of hydrogen atoms between molecules. For example, naphtha may undergo a thermal cracking reaction to form ethene and H₂ among other molecules.

As used herein, the term “sequestration” refers to the storing of a fluid that is a by-product of a process rather than discharging the fluid to the atmosphere or open environment.

As used herein, the term “subsidence” refers to a downward movement of a surface relative to an initial elevation of the surface.

As used herein, the term “thickness” of a layer refers to the distance between the upper and lower boundaries of a cross section of a layer, wherein the distance is measured normal to the average tilt of the cross section.

As used herein, the term “thermal fracture” refers to fractures created in a formation caused directly or indirectly by expansion or contraction of a portion of the formation and/or fluids within the formation, which in turn is caused by increasing/decreasing the temperature of the formation and/or fluids within the formation, and/or by increasing/decreasing a pressure of fluids within the formation due to heating. Thermal fractures may propagate into or form in neighboring regions significantly cooler than the heated zone.

As used herein, the term “hydraulic fracture” refers to a fracture at least partially propagated into a formation, wherein the fracture is created through injection of pressurized fluids into the formation. The fracture may be artificially held open by injection of a proppant material. Hydraulic fractures may be substantially horizontal in orientation, substantially vertical in orientation, or oriented along any other plane.

As used herein, the term “wellbore” refers to a hole in the subsurface made by drilling or insertion of a conduit into the subsurface. A wellbore may have a substantially circular cross section, or other cross-sectional shapes (e.g., circles, ovals, squares, rectangles, triangles, slits, or other regular or irregular shapes). As used herein, the term “well”, when referring to an opening in the formation, may be used interchangeably with the term “wellbore.”

### DESCRIPTION OF SPECIFIC EMBODIMENTS

The inventions are described herein in connection with certain specific embodiments. However, to the extent that the following detailed description is specific to a particular embodiment or a particular use, such is intended to be illustrative only and is not to be construed as limiting the scope of the invention.

As discussed herein, some embodiments of the invention include or have application related to an in situ method of recovering natural resources. The natural resources may be recovered from an organic-rich rock formation, including, for example, an oil shale formation. The organic-rich rock formation may include formation hydrocarbons, including, for example, kerogen, coal, and heavy hydrocarbons. In some embodiments of the invention the natural resources may include hydrocarbon fluids, including, for example, products of the pyrolysis of formation hydrocarbons such as shale oil. In some embodiments of the invention the natural resources may also include water-soluble minerals, including, for example, nahcolite (sodium bicarbonate, or 2NaHCO₃), soda ash (sodium carbonate, or Na₂CO₃) and dawsonite (NaAl(CO₃)(OH)₂).

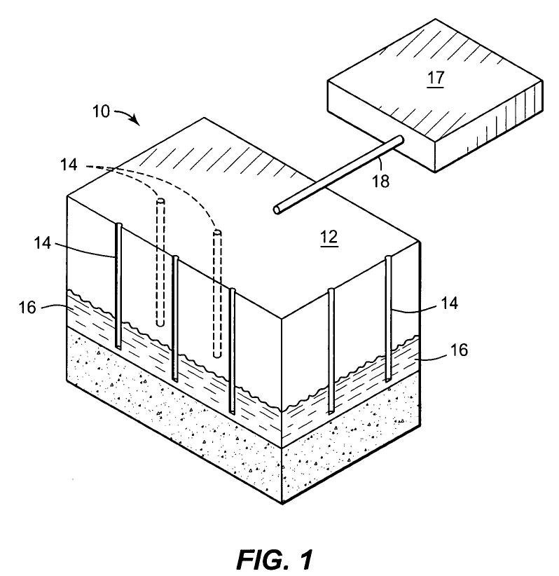

FIG. 1 presents a perspective view of an illustrative oil shale development area 10. A surface 12 of the development area 10 is indicated. Below the surface is an organic-rich rock formation 16. The illustrative subsurface formation 16 contains formation hydrocarbons (such as, for example, kerogen) and possibly valuable water-soluble minerals (such as, for example, nahcolite). It is understood that the representative formation 16 may be any organic-rich rock formation, including a rock matrix containing coal or tar sands, for example. In addition, the rock matrix making up the formation 16 may be permeable, semi-permeable or non-permeable. The present inventions are particularly advantageous in oil shale development areas initially having very limited or effectively no fluid permeability.

In order to access formation 16 and recover natural resources therefrom, a plurality of wellbores is formed. Wellbores are shown at 14 in FIG. 1. The representative wellbores 14 are essentially vertical in orientation relative to the surface 12. However, it is understood that some or all of the wellbores 14 could deviate into an obtuse or even horizontal orientation. In the arrangement of FIG. 1, each of the wellbores 14 is completed in the oil shale formation 16. The completions may be either open or cased hole. The well completions may also include propped or unpropped hydraulic fractures emanating therefrom.

In the view of FIG. 1, only seven wellbores 14 are shown. However, it is understood that in an oil shale development project, numerous additional wellbores 14 will most likely be drilled. The wellbores 14 may be located in relatively close proximity, being from 10 feet to up to 300 feet in separation. In some embodiments, a well spacing of 15 to 25 feet is provided. Typically, the wellbores 14 are also completed at shallow depths, being from 200 to 5,000 feet at total depth. In some embodiments the oil shale formation targeted for in situ retorting is at a depth greater than 200 feet below the surface or alternatively 400 feet below the surface. Alternatively, conversion and production occur at depths between 500 and 2,500 feet.

The wellbores 14 will be selected for certain functions and may be designated as heat injection wells, water injection wells, oil production wells and/or water-soluble mineral solution production wells. In one aspect, the wellbores 14 are dimensioned to serve two, three, or all four of these purposes. Suitable tools and equipment may be sequentially run into and removed from the wellbores 14 to serve the various purposes.

A fluid processing facility 17 is also shown schematically. The fluid processing facility 17 is equipped to receive fluids produced from the organic-rich rock formation 16 through one or more pipelines or flow lines 18. The fluid processing facility 17 may include equipment suitable for receiving and separating oil, gas, and water produced from the heated formation. The fluid processing facility 17 may further include equipment for separating out dissolved water-soluble minerals and/or migratory contaminant species, including, for example, dissolved organic contaminants, metal contaminants, or ionic contaminants in the produced water recovered from the organic-rich rock formation 16. The contaminants may include, for example, aromatic hydrocarbons such as benzene, toluene, xylene, and tri-methylbenzene. The contaminants may also include polyaromatic hydrocarbons such as anthracene, naphthalene, chrysene and pyrene. Metal contaminants may include species containing arsenic, boron, chromium, mercury, selenium, lead, vanadium, nickel, cobalt, molybdenum, or zinc. Ionic contaminant species may include, for example, sulfates, chlorides, fluorides, lithium, potassium, aluminum, ammonia, and nitrates.

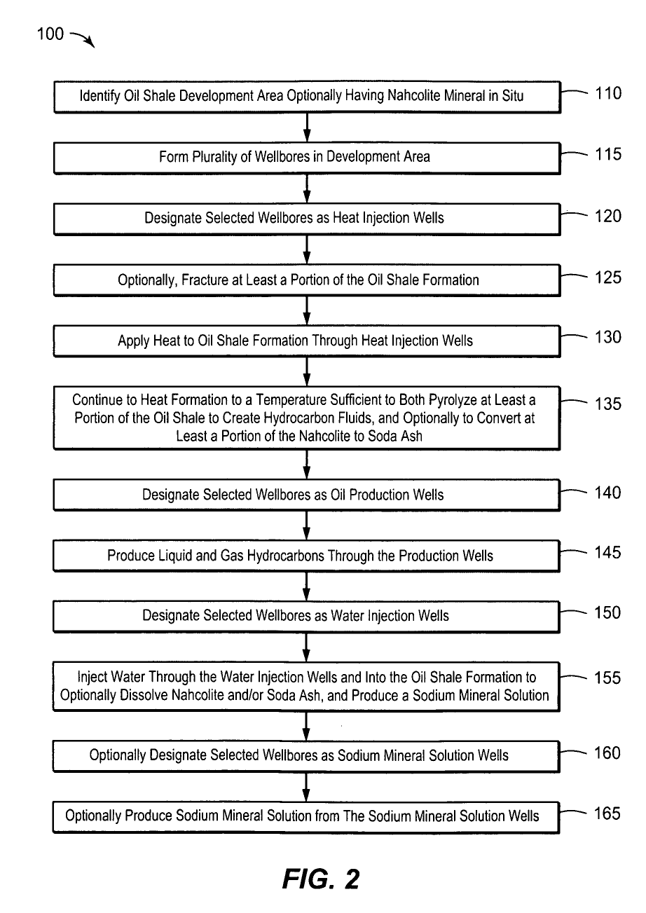

In order to recover oil, gas, and sodium (or other) water-soluble minerals, a series of steps may be undertaken. FIG. 2 presents a flow chart demonstrating a method of in situ thermal recovery of oil and gas from an organic-rich rock formation 100, in one embodiment. It is understood that the order of some of the steps from FIG. 2 may be changed, and that the sequence of steps is merely for illustration.

First, the oil shale (or other organic-rich rock) formation 16 is identified within the development area 10. This step is shown in box 110. Optionally, the oil shale formation may contain nahcolite or other sodium minerals. The targeted development area within the oil shale formation may be identified by measuring or modeling the depth, thickness and organic richness of the oil shale as well as evaluating the position of the organic-rich rock formation relative to other rock types, structural features (e.g. faults, anticlines or synclines), or hydrogeological units (i.e. aquifers). This is accomplished by creating and interpreting maps and/or models of depth, thickness, organic richness and other data from available tests and sources. This may involve performing geological surface surveys, studying outcrops, performing seismic surveys, and/or drilling boreholes to obtain core samples from subsurface rock. Rock samples may be analyzed to assess kerogen content and hydrocarbon fluid generating capability.

The kerogen content of the organic-rich rock formation may be ascertained from outcrop or core samples using a variety of data. Such data may include organic carbon content, hydrogen index, and modified Fischer assay analyses. Subsurface permeability may also be assessed via rock samples, outcrops, or studies of ground water flow. Furthermore the connectivity of the development area to ground water sources may be assessed.

Next, a plurality of wellbores 14 is formed across the targeted development area 10. This step is shown schematically in box 115. The purposes of the wellbores 14 are set forth above and need not be repeated. However, it is noted that for purposes of the wellbore formation step of box 115, only a portion of the wells need be completed initially. For instance, at the beginning of the project heat injection wells are needed, while a majority of the hydrocarbon production wells are not yet needed. Production wells may be brought in once conversion begins, such as after 4 to 12 months of heating.

It is understood that petroleum engineers will develop a strategy for the best depth and arrangement for the wellbores 14, depending upon anticipated reservoir characteristics, economic constraints, and work scheduling constraints. In addition, engineering staff will determine what wellbores 14 shall be used for initial formation 16 heating. This selection step is represented by box 120.

Concerning heat injection wells, there are various methods for applying heat to the organic-rich rock formation 16. The present methods are not limited to the heating technique employed unless specifically so stated in the claims. The heating step is represented generally by box 130. Preferably, for in situ processes the heating of a production zone takes place over a period of months, or even four or more years.

The formation 16 is heated to a temperature sufficient to pyrolyze at least a portion of the oil shale in order to convert the kerogen to hydrocarbon fluids. The bulk of the target zone of the formation may be heated to between 270° C. to 800° C. Alternatively, the targeted volume of the organic-rich formation is heated to at least 350° C. to create production fluids. The conversion step is represented in FIG. 2 by box 135. The resulting liquids and hydrocarbon gases may be refined into products which resemble common commercial petroleum products. Such liquid products include transportation fuels such as diesel, jet fuel and naptha. Generated gases include light alkanes, light alkenes, H₂, CO₂, CO, and NH₃.

Conversion of the oil shale will create permeability in the oil shale section in rocks that were originally impermeable. Preferably, the heating and conversion processes of boxes 130 and 135, occur over a lengthy period of time. In one aspect, the heating period is from three months to four or more years. Also as an optional part of box 135, the formation 16 may be heated to a temperature sufficient to convert at least a portion of nahcolite, if present, to soda ash. Heat applied to mature the oil shale and recover oil and gas will also convert nahcolite to sodium carbonate (soda ash), a related sodium mineral. The process of converting nahcolite (sodium bicarbonate) to soda ash (sodium carbonate) is described herein.

In connection with the heating step 130, the rock formation 16 may optionally be fractured to aid heat transfer or later hydrocarbon fluid production. The optional fracturing step is shown in box 125. Fracturing may be accomplished by creating thermal fractures within the formation through application of heat. By heating the organic-rich rock and transforming the kerogen to oil and gas, the permeability of portions of the formation are increased via thermal fracture formation and subsequent production of a portion of the hydrocarbon fluids generated from the kerogen. Alternatively, a process known as hydraulic fracturing may be used. Hydraulic fracturing is a process known in the art of oil and gas recovery where a fracture fluid is pressurized within the wellbore above the fracture pressure of the formation, thus developing fracture planes within the formation to relieve the pressure generated within the wellbore. Hydraulic fractures may be used to create additional permeability in portions of the formation and/or be used to provide a planar source for heating.

As part of the hydrocarbon fluid production process 100, certain wells 14 may be designated as oil and gas production wells. This step is depicted by box 140. Oil and gas production might not be initiated until it is determined that the kerogen has been sufficiently retorted to allow maximum recovery of oil and gas from the formation 16. In some instances, dedicated production wells are not drilled until after heat injection wells (box 130) have been in operation for a period of several weeks or months. Thus, box 140 may include the formation of additional wellbores 14. In other instances, selected heater wells are converted to production wells.

After certain wellbores 14 have been designated as oil and gas production wells, oil and/or gas is produced from the wellbores 14. The oil and/or gas production process is shown at box 145. At this stage (box 145), any water-soluble minerals, such as nahcolite and converted soda ash may remain substantially trapped in the rock formation 16 as finely disseminated crystals or nodules within the oil shale beds, and are not produced. However, some nahcolite and/or soda ash may be dissolved in the water created during heat conversion (box 135) within the formation.

Box 150 presents an optional next step in the oil and gas recovery method 100. Here, certain wellbores 14 are designated as water or aqueous fluid injection wells. Aqueous fluids are solutions of water with other species. The water may constitute “brine,” and may include dissolved inorganic salts of chloride, sulfates and carbonates of Group I and II elements of The Periodic Table of Elements. Organic salts can also be present in the aqueous fluid. The water may alternatively be fresh water containing other species. The other species may be present to alter the pH. Alternatively, the other species may reflect the availability of brackish water not saturated in the species wished to be leached from the subsurface. Preferably, the water injection wells are selected from some or all of the wellbores used for heat injection or for oil and/or gas production. However, the scope of the step of box 150 may include the drilling of yet additional wellbores 14 for use as dedicated water injection wells. In this respect, it may be desirable to complete water injection wells along a periphery of the development area 10 in order to create a boundary of high pressure.

Next, optionally water or an aqueous fluid is injected through the water injection wells and into the oil shale formation 16. This step is shown at box 155. The water may be in the form of steam or pressurized hot water. Alternatively the injected water may be cool and becomes heated as it contacts the previously heated formation. The injection process may further induce fracturing. This process may create fingered caverns and brecciated zones in the nahcolite-bearing intervals some distance, for example up to 200 feet out, from the water injection wellbores. In one aspect, a gas cap, such as nitrogen, may be maintained at the top of each “cavern” to prevent vertical growth.

Along with the designation of certain wellbores 14 as water injection wells, the design engineers may also designate certain wellbores 14 as water or water-soluble mineral solution production wells. This step is shown in box 160. These wells may be the same as wells used to previously produce hydrocarbons or inject heat. These recovery wells may be used to produce an aqueous solution of dissolved water-soluble minerals and other species, including, for example, migratory contaminant species. For example, the solution may be one primarily of dissolved soda ash. This step is shown in box 165. Alternatively, single wellbores may be used to both inject water and then to recover a sodium mineral solution. Thus, box 165 includes the option of using the same wellbores 14 for both water injection and solution production (Box 165).

Temporary control of the migration of the migratory contaminant species, especially during the pyrolysis process, can be obtained via placement of the injection and production wells 14 such that fluid flow out of the heated zone is minimized. Typically, this involves placing injection wells at the periphery of the heated zone so as to cause pressure gradients which prevent flow inside the heated zone from leaving the zone.

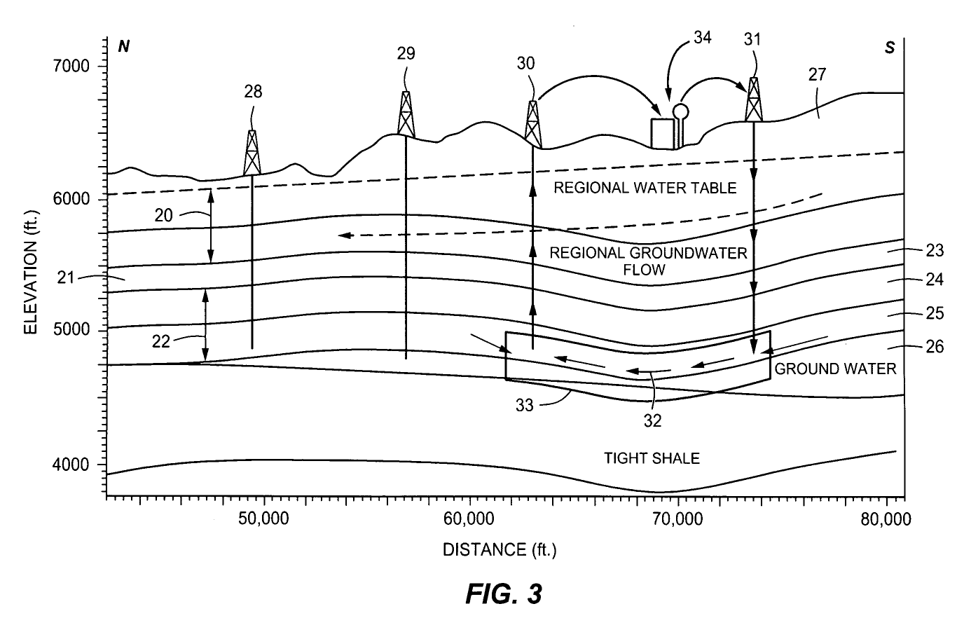

FIG. 3 is a cross-sectional view of an illustrative oil shale formation that is within or connected to ground water aquifers and a formation leaching operation. Four separate oil shale formation zones are depicted (23, 24, 25 and 26) within the oil shale formation. The water aquifers are below the ground surface 27, and are categorized as an upper aquifer 20 and a lower aquifer 22. Intermediate the upper and lower aquifers is an aquitard 21. It can be seen that certain zones of the formation are both aquifers or aquitards and oil shale zones. A plurality of wells (28, 29, 30 and 31) is shown traversing vertically downward through the aquifers. One of the wells is serving as a water injection well 31, while another is serving as a water production well 30. In this way, water is circulated 32 through at least the lower aquifer 22.

FIG. 3 shows diagrammatically the water circulation 32 through an oil shale volume that was heated 33, that resides within or is connected to an aquifer 22, and from which hydrocarbon fluids were previously recovered. Introduction of water via the water injection well 31 forces water into the previously heated oil shale 33 and water-soluble minerals and migratory contaminants species are swept to the water production well 30. The water may then be processed in a facility 34 wherein the water-soluble minerals (e.g. nahcolite or soda ash) and the migratory contaminants may be substantially removed from the water stream. Water is then reinjected into the oil shale volume 33 and the formation leaching is repeated. This leaching with water is intended to continue until levels of migratory contaminant species are at environmentally acceptable levels within the previously heated oil shale zone 33. This may require 1 cycle, 2 cycles, 5 cycles 10 cycles or more cycles of formation leaching, where a single cycle indicates injection and production of approximately one pore volume of water. It is understood that there may be numerous water injection and water production wells in an actual oil shale development. Moreover, the system may include monitoring wells (28 and 29) which can be utilized during the oil shale heating phase, the shale oil production phase, the leaching phase, or during any combination of these phases to monitor for migratory contaminant species and/or water-soluble minerals.

In order to expand upon various features and methods for shale oil development, certain sections are specifically entitled below.

In some fields, formation hydrocarbons, such as oil shale, may exist in more than one subsurface formation. In some instances, the organic-rich rock formations may be separated by rock layers that are hydrocarbon-free or that otherwise have little or no commercial value. Therefore, it may be desirable for the operator of a field under hydrocarbon development to undertake an analysis as to which of the subsurface, organic-rich rock formations to target or in which order they should be developed.

The organic-rich rock formation may be selected for development based on various factors. One such factor is the thickness of the hydrocarbon containing layer within the formation. Greater pay zone thickness may indicate a greater potential volumetric production of hydrocarbon fluids. Each of the hydrocarbon containing layers may have a thickness that varies depending on, for example, conditions under which the formation hydrocarbon containing layer was formed. Therefore, an organic-rich rock formation will typically be selected for treatment if that formation includes at least one formation hydrocarbon-containing layer having a thickness sufficient for economical production of produced fluids.

An organic-rich rock formation may also be chosen if the thickness of several layers that are closely spaced together is sufficient for economical production of produced fluids. For example, an in situ conversion process for formation hydrocarbons may include selecting and treating a layer within an organic-rich rock formation having a thickness of greater than about 5 meters, 10 meters, 50 m, or even 100 meters. In this manner, heat losses (as a fraction of total injected heat) to layers formed above and below an organic-rich rock formation may be less than such heat losses from a thin layer of formation hydrocarbons. A process as described herein, however, may also include selecting and treating layers that may include layers substantially free of formation hydrocarbons or thin layers of formation hydrocarbons.

The richness of one or more organic-rich rock formations may also be considered. Richness may depend on many factors including the conditions under which the formation hydrocarbon containing layer was formed, an amount of formation hydrocarbons in the layer, and/or a composition of formation hydrocarbons in the layer. A thin and rich formation hydrocarbon layer may be able to produce significantly more valuable hydrocarbons than a much thicker, less rich formation hydrocarbon layer. Of course, producing hydrocarbons from a formation that is both thick and rich is desirable.

The kerogen content of an organic-rich rock formation may be ascertained from outcrop or core samples using a variety of data. Such data may include organic carbon content, hydrogen index, and modified Fischer assay analyses. The Fischer Assay is a standard method which involves heating a sample of a formation hydrocarbon containing layer to approximately 500° C. in one hour, collecting fluids produced from the heated sample, and quantifying the amount of fluids produced.

Subsurface formation permeability may also be assessed via rock samples, outcrops, or studies of ground water flow. Furthermore the connectivity of the development area to ground water sources may be assessed. Thus, an organic-rich rock formation may be chosen for development based on the permeability or porosity of the formation matrix even if the thickness of the formation is relatively thin.

Other factors known to petroleum engineers may be taken into consideration when selecting a formation for development. Such factors include depth of the perceived pay zone, stratigraphic proximity of fresh ground water to kerogen-containing zones, continuity of thickness, and other factors. For instance, the assessed fluid production content within a formation will also effect eventual volumetric production.

In producing hydrocarbon fluids from an oil shale field, it may be desirable to control the migration of pyrolyzed fluids. In some instances, this includes the use of injection wells, particularly around the periphery of the field. Such wells may inject water, steam, CO₂, heated methane, or other fluids to drive cracked kerogen fluids inwardly towards production wells. In some embodiments, physical barriers may be placed around the area of the organic-rich rock formation under development. One example of a physical barrier involves the creation of freeze walls. Freeze walls are formed by circulating refrigerant through peripheral wells to substantially reduce the temperature of the rock formation. This, in turn, prevents the pyrolyzation of kerogen present at the periphery of the field and the outward migration of oil and gas. Freeze walls will also cause native water in the formation along the periphery to freeze.

The use of subsurface freezing to stabilize poorly consolidated soils or to provide a barrier to fluid flow is known in the art. Shell Exploration and Production Company has discussed the use of freeze walls for oil shale production in several patents, including U.S. Pat. No. 6,880,633 and U.S. Pat. No. 7,032,660. Shell's '660 patent uses subsurface freezing to protect against groundwater flow and groundwater contamination during in situ shale oil production. Additional patents that disclose the use of so-called freeze walls are U.S. Pat. No. 3,528,252, U.S. Pat. No. 3,943,722, U.S. Pat. No. 3,729,965, U.S. Pat. No. 4,358,222, U.S. Pat. No. 4,607,488, and WO Pat. No. 98996480.

Another example of a physical barrier that may be used to limit fluid flow into or out of an oil shale field is the creation of grout walls. Grout walls are formed by injecting cement into the formation to fill permeable pathways. In the context of an oil shale field, cement would be injected along the periphery of the field. This prevents the movement of pyrolyzed fluids out of the field under development, and the movement of water from adjacent aquifers into the field.

As noted above, several different types of wells may be used in the development of an organic-rich rock formation, including, for example, an oil shale field. For example, the heating of the organic-rich rock formation may be accomplished through the use of heater wells. The heater wells may include, for example, electrical resistance heating elements. The production of hydrocarbon fluids from the formation may be accomplished through the use of wells completed for the production of fluids. The injection of an aqueous fluid may be accomplished through the use of injection wells. Finally, the production of an aqueous solution may be accomplished through use of solution production wells.

The different wells listed above may be used for more than one purpose. Stated another way, wells initially completed for one purpose may later be used for another purpose, thereby lowering project costs and/or decreasing the time required to perform certain tasks. For example, one or more of the production wells may also be used as injection wells for later injecting water into the organic-rich rock formation. Alternatively, one or more of the production wells may also be used as solution production wells for later producing an aqueous solution from the organic-rich rock formation.

In other aspects, production wells (and in some circumstances heater wells) may initially be used as dewatering wells (e.g., before heating is begun and/or when heating is initially started). In addition, in some circumstances dewatering wells can later be used as production wells (and in some circumstances heater wells). As such, the dewatering wells may be placed and/or designed so that such wells can be later used as production wells and/or heater wells. The heater wells may be placed and/or designed so that such wells can be later used as production wells and/or dewatering wells. The production wells may be placed and/or designed so that such wells can be later used as dewatering wells and/or heater wells. Similarly, injection wells may be wells that initially were used for other purposes (e.g., heating, production, dewatering, monitoring, etc.), and injection wells may later be used for other purposes. Similarly, monitoring wells may be wells that initially were used for other purposes (e.g., heating, production, dewatering, injection, etc.). Finally, monitoring wells may later be used for other purposes such as water production.

The wellbores for the various wells may be located in relatively close proximity, being from 10 feet to up to 300 feet in separation. Alternatively, the wellbores may be spaced from 30 to 200 feet or 50 to 100 feet. Typically, the wellbores are also completed at shallow depths, being from 200 to 5,000 feet at total depth. Alternatively, the wellbores may be completed at depths from 1,000 to 4,000 feet, or 1,500 to 3,500 feet. In some embodiments, the oil shale formation targeted for in situ retorting is at a depth greater than 200 feet below the surface. In alternative embodiments, the oil shale formation targeted for in situ retorting is at a depth greater than 500, 1,000, or 1,500 feet below the surface. In alternative embodiments, the oil shale formation targeted for in situ retorting is at a depth between 200 and 5,000 feet, alternatively between 1,000 and 4,000 ft, 1,200 and 3,700 feet, or 1,500 and 3,500 feet below the surface.

It is desirable to arrange the various wells for an oil shale field in a pre-planned pattern. For instance, heater wells may be arranged in a variety of patterns including, but not limited to triangles, squares, hexagons, and other polygons. The pattern may include a regular polygon to promote uniform heating through at least the portion of the formation in which the heater wells are placed. The pattern may also be a line drive pattern. A line drive pattern generally includes a first linear array of heater wells, a second linear array of heater wells, and a production well or a linear array of production wells between the first and second linear array of heater wells. Interspersed among the heater wells are typically one or more production wells. The injection wells may likewise be disposed within a repetitive pattern of units, which may be similar to or different from that used for the heater wells.

One method to reduce the number of wells is to use a single well as both a heater well and a production well. Reduction of the number of wells by using single wells for sequential purposes can reduce project costs. One or more monitoring wells may be disposed at selected points in the field. The monitoring wells may be configured with one or more devices that measure a temperature, a pressure, and/or a property of a fluid in the wellbore. In some instances, a heater well may also serve as a monitoring well, or otherwise be instrumented.

Another method for reducing the number of heater wells is to use well patterns. Regular patterns of heater wells equidistantly spaced from a production well may be used. The patterns may form equilateral triangular arrays, hexagonal arrays, or other array patterns. The arrays of heater wells may be disposed such that a distance between each heater well is less than about 70 feet (21 m). A portion of the formation may be heated with heater wells disposed substantially parallel to a boundary of the hydrocarbon formation.

In alternative embodiments, the array of heater wells may be disposed such that a distance between each heater well may be less than about 100 feet, or 50 feet, or 30 feet. Regardless of the arrangement of or distance between the heater wells, in certain embodiments, a ratio of heater wells to production wells disposed within a organic-rich rock formation may be greater than about 5, 8, 10, 20, or more.

In one embodiment, individual production wells are surrounded by at most one layer of heater wells. This may include arrangements such as 5-spot, 7-spot, or 9-spot arrays, with alternating rows of production and heater wells. In another embodiment, two layers of heater wells may surround a production well, but with the heater wells staggered so that a clear pathway exists for the majority of flow away from the further heater wells. Flow and reservoir simulations may be employed to assess the pathways and temperature history of hydrocarbon fluids generated in situ as they migrate from their points of origin to production wells.

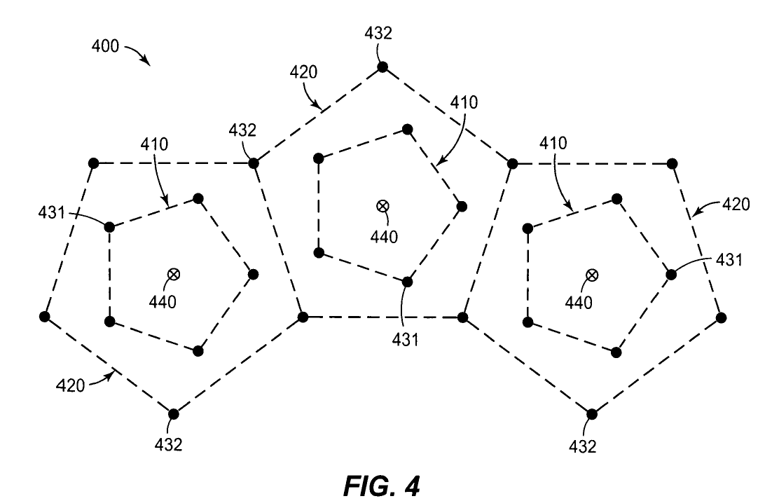

FIG. 4 provides a plan view of an illustrative heater well arrangement using more than one layer of heater wells. The heater well arrangement is used in connection with the production of hydrocarbons from a shale oil development area 400. In FIG. 4, the heater well arrangement employs a first layer of heater wells 410, surrounded by a second layer of heater wells 420. The heater wells in the first layer 410 are referenced at 431, while the heater wells in the second layer 420 are referenced at 432.

A production well 440 is shown central to the well layers 410 and 420. It is noted that the heater wells 432 in the second layer 420 of wells are offset from the heater wells 431 in the first layer 410 of wells, relative to the production well 440. The purpose is to provide a flowpath for converted hydrocarbons that minimizes travel near a heater well in the first layer 410 of heater wells. This, in turn, minimizes secondary cracking of hydrocarbons converted from kerogen as hydrocarbons flow from the second layer of wells 420 to the production wells 440.

In the illustrative arrangement of FIG. 4, the first layer 410 and the second layer 420 each defines a 5-spot pattern. However, it is understood that other patterns may be employed, such as 3-spot or 6-spot patterns. In any instance, a plurality of heater wells 431 comprising a first layer of heater wells 410 is placed around a production well 440, with a second plurality of heater wells 432 comprising a second layer of heater wells 420 placed around the first layer 410.

The heater wells in the two layers also may be arranged such that the majority of hydrocarbons generated by heat from each heater well 432 in the second layer 420 are able to migrate to a production well 440 without passing substantially near a heater well 431 in the first layer 410. The heater wells 431, 432 in the two layers 410, 420 further may be arranged such that the majority of hydrocarbons generated by heat from each heater well 432 in the second layer 420 are able to migrate to the production well 440 without passing through a zone of substantially increasing formation temperature.

One method to reduce the number of heater wells is to use well patterns that are elongated in a particular direction, particularly in the direction of most efficient thermal conductivity. Heat convection may be affected by various factors such as bedding planes and stresses within the formation. For instance, heat convection may be more efficient in the direction perpendicular to the least horizontal principal stress on the formation. In some instanced, heat convection may be more efficient in the direction parallel to the least horizontal principal stress.

In connection with the development of an oil shale field, it may be desirable that the progression of heat through the subsurface in accordance with steps 130 and 135 be uniform. However, for various reasons the heating and maturation of formation hydrocarbons in a subsurface formation may not proceed uniformly despite a regular arrangement of heater and production wells. Heterogeneities in the oil shale properties and formation structure may cause certain local areas to be more or less productive. Moreover, formation fracturing which occurs due to the heating and maturation of the oil shale can lead to an uneven distribution of preferred pathways and, thus, increase flow to certain production wells and reduce flow to others. Uneven fluid maturation may be an undesirable condition since certain subsurface regions may receive more heat energy than necessary where other regions receive less than desired. This, in turn, leads to the uneven flow and recovery of production fluids. Produced oil quality, overall production rate, and/or ultimate recoveries may be reduced.

To detect uneven flow conditions, production and heater wells may be instrumented with sensors. Sensors may include equipment to measure temperature, pressure, flow rates, and/or compositional information. Data from these sensors can be processed via simple rules or input to detailed simulations to reach decisions on how to adjust heater and production wells to improve subsurface performance. Production well performance may be adjusted by controlling backpressure or throttling on the well. Heater well performance may also be adjusted by controlling energy input. Sensor readings may also sometimes imply mechanical problems with a well or downhole equipment which requires repair, replacement, or abandonment.

In one embodiment, flow rate, compositional, temperature and/or pressure data are utilized from two or more wells as inputs to a computer algorithm to control heating rate and/or production rates. Unmeasured conditions at or in the neighborhood of the well are then estimated and used to control the well. For example, in situ fracturing behavior and kerogen maturation are estimated based on thermal, flow, and compositional data from a set of wells. In another example, well integrity is evaluated based on pressure data, well temperature data, and estimated in situ stresses. In a related embodiment the number of sensors is reduced by equipping only a subset of the wells with instruments, and using the results to interpolate, calculate, or estimate conditions at uninstrumented wells. Certain wells may have only a limited set of sensors (e.g., wellhead temperature and pressure only) where others have a much larger set of sensors (e.g., wellhead temperature and pressure, bottomhole temperature and pressure, production composition, flow rate, electrical signature, casing strain, etc.).

As noted above, there are various methods for applying heat to an organic-rich rock formation. For example, one method may include electrical resistance heaters disposed in a wellbore or outside of a wellbore. One such method involves the use of electrical resistive heating elements in a cased or uncased wellbore. Electrical resistance heating involves directly passing electricity through a conductive material such that resistive losses cause it to heat the conductive material. Other heating methods include the use of downhole combustors, in situ combustion, radio-frequency (RF) electrical energy, or microwave energy. Still others include injecting a hot fluid into the oil shale formation to directly heat it. The hot fluid may or may not be circulated. One method may include generating heat by burning a fuel external to or within a subsurface formation. For example, heat may be supplied by surface burners or downhole burners or by circulating hot fluids (such as methane gas or naphtha) into the formation through, for example, wellbores via, for example, natural or artificial fractures. Some burners may be configured to perform flameless combustion. Alternatively, some methods may include combusting fuel within the formation such as via a natural distributed combustor, which generally refers to a heater that uses an oxidant to oxidize at least a portion of the carbon in the formation to generate heat, and wherein the oxidation takes place in a vicinity proximate to a wellbore. The present methods are not limited to the heating technique employed unless so stated in the claims.

One method for formation heating involves the use of electrical resistors in which an electrical current is passed through a resistive material which dissipates the electrical energy as heat. This method is distinguished from dielectric heating in which a high-frequency oscillating electric current induces electrical currents in nearby materials and causes them to heat. The electric heater may include an insulated conductor, an elongated member disposed in the opening, and/or a conductor disposed in a conduit. An early patent disclosing the use of electrical resistance heaters to produce oil shale in situ is U.S. Pat. No. 1,666,488. The '488 patent issued to Crawshaw in 1928. Since 1928, various designs for downhole electrical heaters have been proposed. Illustrative designs are presented in U.S. Pat. No. 1,701,884, U.S. Pat. No. 3,376,403, U.S. Pat. No. 4,626,665, U.S. Pat. No. 4,704,514, and U.S. Pat. No. 6,023,554).

A review of application of electrical heating methods for heavy oil reservoirs is given by R. Sierra and S. M. Farouq Ali, “Promising Progress in Field Application of Reservoir Electrical Heating Methods”, Society of Petroleum Engineers Paper 69709, 2001. The entire disclosure of this reference is hereby incorporated by reference.

Certain previous designs for in situ electrical resistance heaters utilized solid, continuous heating elements (e.g., metal wires or strips). However, such elements may lack the necessary robustness for long-term, high temperature applications such as oil shale maturation. As the formation heats and the oil shale matures, significant expansion of the rock occurs. This leads to high stresses on wells intersecting the formation. These stresses can lead to bending and stretching of the wellbore pipe and internal components. Cementing (e.g., U.S. Pat. No. 4,886,118) or packing (e.g., U.S. Pat. No. 2,732,195) a heating element in place may provide some protection against stresses, but some stresses may still be transmitted to the heating element.

As an alternative, international patent publication WO 2005/010320 teaches the use of electrically conductive fractures to heat the oil shale. A heating element is constructed by forming wellbores and then hydraulically fracturing the oil shale formation around the wellbores. The fractures are filled with an electrically conductive material which forms the heating element. Calcined petroleum coke is an exemplary suitable conductant material. Preferably, the fractures are created in a vertical orientation along longitudinal, horizontal planes formed by horizontal wellbores. Electricity may be conducted through the conductive fractures from the heel to the toe of each well. The electrical circuit may be completed by an additional horizontal well that intersects one or more of the vertical fractures near the toe to supply the opposite electrical polarity. The WO 2005/010320 process creates an “in situ toaster” that artificially matures oil shale through the application of electric heat. Thermal conduction heats the oil shale to conversion temperatures in excess of 300° C. causing artificial maturation.

International patent publication WO 2005/045192 teaches an alternative heating means that employs the circulation of a heated fluid within an oil shale formation. In the process of WO 2005/045192 supercritical heated naphtha may be circulated through fractures in the formation. This means that the oil shale is heated by circulating a dense, hot hydrocarbon vapor through sets of closely-spaced hydraulic fractures. In one aspect, the fractures are horizontally formed and conventionally propped. Fracture temperatures of 320°-400° C. are maintained for up to five to ten years. Vaporized naptha may be the preferred heating medium due to its high volumetric heat capacity, ready availability and relatively low degradation rate at the heating temperature. In the WO 2005/045192 process, as the kerogen matures, fluid pressure will drive the generated oil to the heated fractures, where it will be produced with the cycling hydrocarbon vapor.

The purpose for heating the organic-rich rock formation is to pyrolyze at least a portion of the solid formation hydrocarbons to create hydrocarbon fluids. The solid formation hydrocarbons may be pyrolyzed in situ by raising the organic-rich rock formation, (or zones within the formation), to a pyrolyzation temperature. In certain embodiments, the temperature of the formation may be slowly raised through the pyrolysis temperature range. For example, an in situ conversion process may include heating at least a portion of the organic-rich rock formation to raise the average temperature of the zone above about 270° C. at a rate less than a selected amount (e.g., about 10° C., 5° C.; 3° C., 1° C., 0.5° C., or 0.1° C.) per day. In a further embodiment, the portion may be heated such that an average temperature of the selected zone may be less than about 375° C. or, in some embodiments, less than about 400° C. The formation may be heated such that a temperature within the formation reaches (at least) an initial pyrolyzation temperature (e.g., a temperature at the lower end of the temperature range where pyrolyzation begins to occur.

The pyrolysis temperature range may vary depending on the types of formation hydrocarbons within the formation, the heating methodology, and the distribution of heating sources. For example, a pyrolysis temperature range may include temperatures between about 270° C. and about 900° C. Alternatively, the bulk of the target zone of the formation may be heated to between 300° to 600° C. In an alternative embodiment, a pyrolysis temperature range may include temperatures between about 270° C. to about 500° C.

Preferably, for in situ processes the heating of a production zone takes place over a period of months, or even four or more years. Alternatively, the formation may be heated for one to fifteen years, alternatively, 3 to 10 years, 1.5 to 7 years, or 2 to 5 years. The bulk of the target zone of the formation may be heated to between 270° to 800° C. Preferably, the bulk of the target zone of the formation is heated to between 300° to 600° C. Alternatively, the bulk of the target zone is ultimately heated to a temperature below 400° C. (752° F.).

In certain embodiments of the methods of the present invention, downhole burners may be used to heat a targeted oil shale zone. Downhole burners of various design have been discussed in the patent literature for use in oil shale and other largely solid hydrocarbon deposits. Examples include U.S. Pat. No. 2,887,160; U.S. Pat. No. 2,847,071; U.S. Pat. No. 2,895,555; U.S. Pat. No. 3,109,482; U.S. Pat. No. 3,225,829; U.S. Pat. No. 3,241,615; U.S. Pat. No. 3,254,721; U.S. Pat. No. 3,127,936; U.S. Pat. No. 3,095,031; U.S. Pat. No. 5,255,742; and U.S. Pat. No. 5,899,269. Downhole burners operate through the transport of a combustible fuel (typically natural gas) and an oxidizer (typically air) to a subsurface position in a wellbore. The fuel and oxidizer react downhole to generate heat. The combustion gases are removed (typically by transport to the surface, but possibly via injection into the formation). Oftentimes, downhole burners utilize pipe-in-pipe arrangements to transport fuel and oxidizer downhole, and then to remove the flue gas back up to the surface. Some downhole burners generate a flame, while others may not.

The use of downhole burners is an alternative to another form of downhole heat generation called steam generation. In downhole steam generation, a combustor in the well is used to boil water placed in the wellbore for injection into the formation. Applications of the downhole heat technology have been described in F. M. Smith, “A Down-hole burner—Versatile tool for well heating,” 25^{th} Technical Conference on Petroleum Production, Pennsylvania State University, pp 275-285 (Oct. 19-21, 1966); H. Brandt, W. G. Poynter, and J. D. Hummell, “Stimulating Heavy Oil Reservoirs with Downhole Air-Gas Burners,” World Oil, pp. 91-95 (September 1965); and C. I. DePriester and A. J. Pantaleo, “Well Stimulation by Downhole Gas-Air Burner,” Journal of Petroleum Technology, pp. 1297-1302 (December 1963).

Downhole burners have advantages over electrical heating methods due to the reduced infrastructure cost. In this respect, there is no need for an expensive electrical power plant and distribution system. Moreover, there is increased thermal efficiency because the energy losses inherently experienced during electrical power generation are avoided.

Few applications of downhole burners exist. Downhole burner design issues include temperature control and metallurgy limitations. In this respect, the flame temperature can overheat the tubular and burner hardware and cause them to fail via melting, thermal stresses, severe loss of tensile strength, or creep. Certain stainless steels, typically with high chromium content, can tolerate temperatures up to ˜700° C. for extended periods. (See for example H. E. Boyer and T. L. Gall (eds.), Metals Handbook, “Chapter 16: Heat-Resistant Materials”, American Society for Metals, (1985.) The existence of flames can cause hot spots within the burner and in the formation surrounding the burner. This is due to radiant heat transfer from the luminous portion of the flame. However, a typical gas flame can produce temperatures up to about 1,650° C. Materials of construction for the burners must be sufficient to withstand the temperatures of these hot spots. The heaters are therefore more expensive than a comparable heater without flames.

For downhole burner applications, heat transfer can occur in one of several ways. These include conduction, convection, and radiative methods. Radiative heat transfer can be particularly strong for an open flame. Additionally, the flue gases can be corrosive due to the CO₂ and water content. Use of refractory metals or ceramics can help solve these problems, but typically at a higher cost. Ceramic materials with acceptable strength at temperatures in excess of 900° C. are generally high alumina content ceramics. Other ceramics that may be useful include chrome oxide, zirconia oxide, and magnesium oxide based ceramics. Additionally, depending on the nature of the downhole combustion NOₓ generation may be significant.

Heat transfer in a pipe-in-pipe arrangement for a downhole burner can also lead to difficulties. The down going fuel and air will heat exchange with the up going hot flue gases. In a well there is minimal room for a high degree of insulation and hence significant heat transfer is typically expected. This cross heat exchange can lead to higher flame temperatures as the fuel and air become preheated. Additionally, the cross heat exchange can limit the transport of heat downstream of the burner since the hot flue gases may rapidly lose heat energy to the rising cooler flue gases.

In the production of oil and gas resources, it may be desirable to use the produced hydrocarbons as a source of power for ongoing operations. This may be applied to the development of oil and gas resources from oil shale. In this respect, when electrically resistive heaters are used in connection with in situ shale oil recovery, large amounts of power are required.

Electrical power may be obtained from turbines that turn generators. It may be economically advantageous to power the gas turbines by utilizing produced gas from the field. However, such produced gas must be carefully controlled so not to damage the turbine, cause the turbine to misfire, or generate excessive pollutants (e.g., NOₓ).

One source of problems for gas turbines is the presence of contaminants within the fuel. Contaminants include solids, water, heavy components present as liquids, and hydrogen sulfide. Additionally, the combustion behavior of the fuel is important. Combustion parameters to consider include heating value, specific gravity, adiabatic flame temperature, flammability limits, autoignition temperature, autoignition delay time, and flame velocity. Wobbe Index (WI) is often used as a key measure of fuel quality. WI is equal to the ratio of the lower heating value to the square root of the gas specific gravity. Control of the fuel's Wobbe Index to a target value and range of, for example, ±10% or ±20% can allow simplified turbine design and increased optimization of performance.

Fuel quality control may be useful for shale oil developments where the produced gas composition may change over the life of the field and where the gas typically has significant amounts of CO₂, CO, and H₂ in addition to light hydrocarbons. Commercial scale oil shale retorting is expected to produce a gas composition that changes with time.

Inert gases in the turbine fuel can increase power generation by increasing mass flow while maintaining a flame temperature in a desirable range. Moreover inert gases can lower flame temperature and thus reduce NOₓ pollutant generation. Gas generated from oil shale maturation may have significant CO₂ content. Therefore, in certain embodiments of the production processes, the CO₂ content of the fuel gas is adjusted via separation or addition in the surface facilities to optimize turbine performance.

Achieving a certain hydrogen content for low-BTU fuels may also be desirable to achieve appropriate burn properties. In certain embodiments of the processes herein, the H₂ content of the fuel gas is adjusted via separation or addition in the surface facilities to optimize turbine performance. Adjustment of H₂ content in non-shale oil surface facilities utilizing low BTU fuels has been discussed in the patent literature (e.g., U.S. Pat. No. 6,684,644 and U.S. Pat. No. 6,858,049, the entire disclosures of which are hereby incorporated by reference).

The process of heating formation hydrocarbons within an organic-rich rock formation, for example, by pyrolysis, may generate fluids. The heat-generated fluids may include water which is vaporized within the formation. In addition, the action of heating kerogen produces pyrolysis fluids which tend to expand upon heating. The produced pyrolysis fluids may include not only water, but also, for example, hydrocarbons, oxides of carbon, ammonia, molecular nitrogen, and molecular hydrogen. Therefore, as temperatures within a heated portion of the formation increase, a pressure within the heated portion may also increase as a result of increased fluid generation, molecular expansion, and vaporization of water. Thus, some corollary exists between subsurface pressure in an oil shale formation and the fluid pressure generated during pyrolysis. This, in turn, indicates that formation pressure may be monitored to detect the progress of a kerogen conversion process.

The pressure within a heated portion of an organic-rich rock formation depends on other reservoir characteristics. These may include, for example, formation depth, distance from a heater well, a richness of the formation hydrocarbons within the organic-rich rock formation, the degree of heating, and/or a distance from a producer well.

It may be desirable for the developer of an oil shale field to monitor formation pressure during development. Pressure within a formation may be determined at a number of different locations. Such locations may include, but may not be limited to, at a wellhead and at varying depths within a wellbore. In some embodiments, pressure may be measured at a producer well. In an alternate embodiment, pressure may be measured at a heater well. In still another embodiment, pressure may be measured downhole of a dedicated monitoring well.

The process of heating an organic-rich rock formation to a pyrolysis temperature range not only will increase formation pressure, but will also increase formation permeability. The pyrolysis temperature range should be reached before substantial permeability has been generated within the organic-rich rock formation. An initial lack of permeability may prevent the transport of generated fluids from a pyrolysis zone within the formation. In this manner, as heat is initially transferred from a heater well to an organic-rich rock formation, a fluid pressure within the organic-rich rock formation may increase proximate to that heater well. Such an increase in fluid pressure may be caused by, for example, the generation of fluids during pyrolysis of at least some formation hydrocarbons in the formation.

Alternatively, pressure generated by expansion of pyrolysis fluids or other fluids generated in the formation may be allowed to increase. This assumes that an open path to a production well or other pressure sink does not yet exist in the formation. In one aspect, a fluid pressure may be allowed to increase to or above a lithostatic stress. In this instance, fractures in the hydrocarbon containing formation may form when the fluid pressure equals or exceeds the lithostatic stress. For example, fractures may form from a heater well to a production well. The generation of fractures within the heated portion may reduce pressure within the portion due to the production of produced fluids through a production well.

Once pyrolysis has begun within an organic-rich rock formation, fluid pressure may vary depending upon various factors. These include, for example, thermal expansion of hydrocarbons, generation of pyrolysis fluids, rate of conversion, and withdrawal of generated fluids from the formation. For example, as fluids are generated within the formation, fluid pressure within the pores may increase. Removal of generated fluids from the formation may then decrease the fluid pressure within the near wellbore region of the formation.

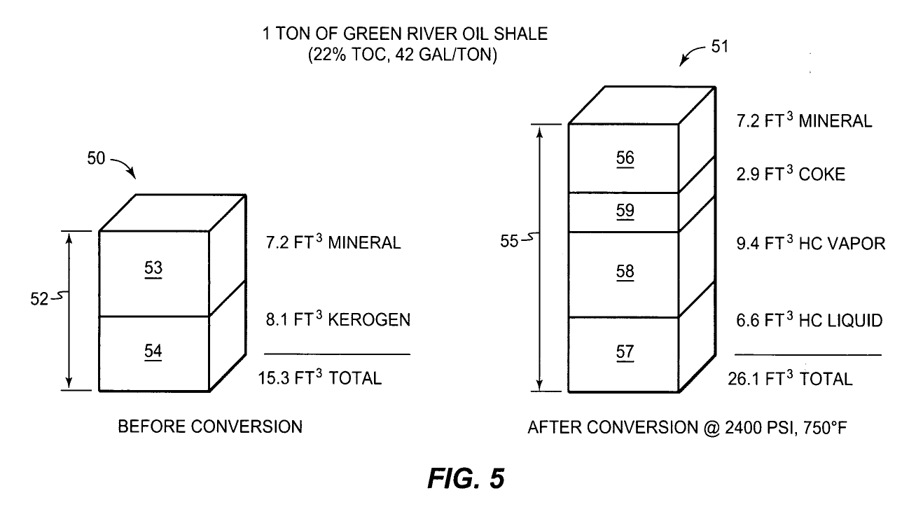

In certain embodiments, a mass of at least a portion of an organic-rich rock formation may be reduced due, for example, to pyrolysis of formation hydrocarbons and the production of hydrocarbon fluids from the formation. As such, the permeability and porosity of at least a portion of the formation may increase. Any in situ method that effectively produces oil and gas from oil shale will create permeability in what was originally a very low permeability rock. The extent to which this will occur is illustrated by the large amount of expansion that must be accommodated if fluids generated from kerogen are unable to flow. The concept is illustrated in FIG. 5.

FIG. 5 provides a bar chart comparing one ton of Green River oil shale before 50 and after 51 a simulated in situ, retorting process. The simulated process was carried out at 2,400 psi and 750° F. on oil shale having a total organic carbon content of 22 wt. % and a Fisher assay of 42 gallons/ton. Before the conversion, a total of 15.3 ft³ of rock matrix 52 existed. This matrix comprised 7.2 ft³ of mineral 53, i.e., dolomite, limestone, etc., and 8.1 ft³ of kerogen 54 imbedded within the shale. As a result of the conversion the material expanded to 26.1 ft³ 55. This represented 7.2 ft³ of mineral 56 (the same number as before the conversion), 6.6 ft³ of hydrocarbon liquid 57, 9.4 ft³ of hydrocarbon vapor 58, and 2.9 ft³ of coke 59. It can be seen that substantial volume expansion occurred during the conversion process. This, in turn, increases permeability of the rock structure.

In an embodiment, heating a portion of an organic-rich rock formation in situ to a pyrolysis temperature may increase permeability of the heated portion. For example, permeability may increase due to formation of thermal fractures within the heated portion caused by application of heat. As the temperature of the heated portion increases, water may be removed due to vaporization. The vaporized water may escape and/or be removed from the formation. In addition, permeability of the heated portion may also increase as a result of production of hydrocarbon fluids from pyrolysis of at least some of the formation hydrocarbons within the heated portion on a macroscopic scale.

Certain systems and methods described herein may be used to treat formation hydrocarbons in at least a portion of a relatively low permeability formation (e.g., in “tight” formations that contain formation hydrocarbons). Such formation hydrocarbons may be heated to pyrolyze at least some of the formation hydrocarbons in a selected zone of the formation. Heating may also increase the permeability of at least a portion of the selected zone. Hydrocarbon fluids generated from pyrolysis may be produced from the formation, thereby further increasing the formation permeability.

Permeability of a selected zone within the heated portion of the organic-rich rock formation may also rapidly increase while the selected zone is heated by conduction. For example, permeability of an impermeable organic-rich rock formation may be less than about 0.1 millidarcy before heating. In some embodiments, pyrolyzing at least a portion of organic-rich rock formation may increase permeability within a selected zone of the portion to greater than about 10 millidarcies, 100 millidarcies, 1 Darcy, 10 Darcies, 20 Darcies, or 50 Darcies. Therefore, a permeability of a selected zone of the portion may increase by a factor of more than about 10, 100, 1,000, 10,000, or 100,000. In one embodiment, the organic-rich rock formation has an initial total permeability less than 1 millidarcy, alternatively less than 0.1 or 0.01 millidarcies, before heating the organic-rich rock formation. In one embodiment, the organic-rich rock formation has a post heating total permeability of greater than 1 millidarcy, alternatively, greater than 10, 50 or 100 millidarcies, after heating the organic-rich rock formation.

In connection with heating the organic-rich rock formation, the organic-rich rock formation may optionally be fractured to aid heat transfer or hydrocarbon fluid production. In one instance, fracturing may be accomplished naturally by creating thermal fractures within the formation through application of heat. Thermal fracture formation is caused by thermal expansion of the rock and fluids and by chemical expansion of kerogen transforming into oil and gas. Thermal fracturing can occur both in the immediate region undergoing heating, and in cooler neighboring regions. The thermal fracturing in the neighboring regions is due to propagation of fractures and tension stresses developed due to the expansion in the hotter zones. Thus, by both heating the organic-rich rock and transforming the kerogen to oil and gas, the permeability is increased not only from fluid formation and vaporization, but also via thermal fracture formation. The increased permeability aids fluid flow within the formation and production of the hydrocarbon fluids generated from the kerogen.

In addition, a process known as hydraulic fracturing may be used. Hydraulic fracturing is a process known in the art of oil and gas recovery where a fracture fluid is pressurized within the wellbore above the fracture pressure of the formation, thus developing fracture planes within the formation to relieve the pressure generated within the wellbore. Hydraulic fractures may be used to create additional permeability and/or be used to provide an extended geometry for a heater well. The WO 2005/010320 patent publication incorporated above describes one such method.

In connection with the production of hydrocarbons from a rock matrix, particularly those of shallow depth, a concern may exist with respect to earth subsidence. This is particularly true in the in situ heating of organic-rich rock where a portion of the matrix itself is thermally converted and removed. Initially, the formation may contain formation hydrocarbons in solid form, such as, for example, kerogen. The formation may also initially contain water-soluble minerals. Initially, the formation may also be substantially impermeable to fluid flow.

The in situ heating of the matrix pyrolyzes at least a portion of the formation hydrocarbons to create hydrocarbon fluids. This, in turn, creates permeability within a matured (pyrolyzed) organic-rich rock zone in the organic-rich rock formation. The combination of pyrolyzation and increased permeability permits hydrocarbon fluids to be produced from the formation. At the same time, the loss of supporting matrix material also creates the potential for subsidence relative to the earth surface.

In some instances, subsidence is sought to be minimized in order to avoid environmental or hydrogeological impact. In this respect, changing the contour and relief of the earth surface, even by a few inches, can change runoff patterns, affect vegetation patterns, and impact watersheds. In addition, subsidence has the potential of damaging production or heater wells formed in a production area. Such subsidence can create damaging hoop and compressional stresses on wellbore casings, cement jobs, and equipment downhole.

In order to avoid or minimize subsidence, it is proposed to leave selected portions of the formation hydrocarbons substantially unpyrolyzed. This serves to preserve one or more unmatured, organic-rich rock zones. In some embodiments, the unmatured organic-rich rock zones may be shaped as substantially vertical pillars extending through a substantial portion of the thickness of the organic-rich rock formation.

The heating rate and distribution of heat within the formation may be designed and implemented to leave sufficient unmatured pillars to prevent subsidence. In one aspect, heat injection wellbores are formed in a pattern such that untreated pillars of oil shale are left therebetween to support the overburden and prevent subsidence.

It is preferred that thermal recovery of oil and gas be conducted before any solution mining of nahcolite or other water-soluble minerals present in the formation. Solution mining can generate large voids in a rock formation and collapse breccias in an oil shale development area. These voids and brecciated zones may pose problems for in situ and mining recovery of oil shale, further increasing the utility of supporting pillars.

In some embodiments, compositions and properties of the hydrocarbon fluids produced by an in situ conversion process may vary depending on, for example, conditions within an organic-rich rock formation. Controlling heat and/or heating rates of a selected section in an organic-rich rock formation may increase or decrease production of selected produced fluids.

In one embodiment, operating conditions may be determined by measuring at least one property of the organic-rich rock formation. The measured properties may be input into a computer executable program. At least one property of the produced fluids selected to be produced from the formation may also be input into the computer executable program. The program may be operable to determine a set of operating conditions from at least the one or more measured properties. The program may also be configured to determine the set of operating conditions from at least one property of the selected produced fluids. In this manner, the determined set of operating conditions may be configured to increase production of selected produced fluids from the formation.

Certain heater well embodiments may include an operating system that is coupled to any of the heater wells such as by insulated conductors or other types of wiring. The operating system may be configured to interface with the heater well. The operating system may receive a signal (e.g., an electromagnetic signal) from a heater that is representative of a temperature distribution of the heater well. Additionally, the operating system may be further configured to control the heater well, either locally or remotely. For example, the operating system may alter a temperature of the heater well by altering a parameter of equipment coupled to the heater well. Therefore, the operating system may monitor, alter, and/or control the heating of at least a portion of the formation.

In some embodiments, a heater well may be turned down and/or off after an average temperature in a formation may have reached a selected temperature. Turning down and/or off the heater well may reduce input energy costs, substantially inhibit overheating of the formation, and allow heat to substantially transfer into colder regions of the formation.

Temperature (and average temperatures) within a heated organic-rich rock formation may vary, depending on, for example, proximity to a heater well, thermal conductivity and thermal diffusivity of the formation, type of reaction occurring, type of formation hydrocarbon, and the presence of water within the organic-rich rock formation. At points in the field where monitoring wells are established, temperature measurements may be taken directly in the wellbore. Further, at heater wells the temperature of the immediately surrounding formation is fairly well understood. However, it is desirable to interpolate temperatures to points in the formation intermediate temperature sensors and heater wells.

In accordance with one aspect of the production processes of the present inventions, a temperature distribution within the organic-rich rock formation may be computed using a numerical simulation model. The numerical simulation model may calculate a subsurface temperature distribution through interpolation of known data points and assumptions of formation conductivity. In addition, the numerical simulation model may be used to determine other properties of the formation under the assessed temperature distribution. For example, the various properties of the formation may include, but are not limited to, permeability of the formation.

The numerical simulation model may also include assessing various properties of a fluid formed within an organic-rich rock formation under the assessed temperature distribution. For example, the various properties of a formed fluid may include, but are not limited to, a cumulative volume of a fluid formed in the formation, fluid viscosity, fluid density, and a composition of the fluid formed in the formation. Such a simulation may be used to assess the performance of a commercial-scale operation or small-scale field experiment. For example, a performance of a commercial-scale development may be assessed based on, but not limited to, a total volume of product that may be produced from a research-scale operation.

Some embodiments include producing at least a portion of the hydrocarbon fluids from the organic-rich rock formation. The hydrocarbon fluids may be produced through production wells. Production wells may be cased or uncased wells and drilled and completed through methods known in the art.

Some embodiments further include producing a production fluid from the organic-rich rock formation where the production fluid contains the hydrocarbon fluids and an aqueous fluid. The aqueous fluid may contain water-soluble minerals and/or migratory contaminant species. In such case, the production fluid may be separated into a hydrocarbon stream and an aqueous stream at a surface facility. Thereafter the water-soluble minerals and/or migratory contaminant species may be recovered from the aqueous stream. This embodiment may be combined with any of the other aspects of the invention discussed herein.

The produced hydrocarbon fluids may include a pyrolysis oil component (or condensable component) and a pyrolysis gas component (or non-condensable component). Condensable hydrocarbons produced from the formation will typically include paraffins, cycloalkanes, mono-aromatics, and di-aromatics as components. Such condensable hydrocarbons may also include other components such as tri-aromatics and other hydrocarbon species.

In certain embodiments, a majority of the hydrocarbons in the produced fluid may have a carbon number of less than approximately 25. Alternatively, less than about 15 weight % of the hydrocarbons in the fluid may have a carbon number greater than approximately 25. The non-condensable hydrocarbons may include, but are not limited to, hydrocarbons having carbon numbers less than 5.

In certain embodiments, the API gravity of the condensable hydrocarbons in the produced fluid may be approximately 20 or above (e.g., 25, 30, 40, 50, etc.). In certain embodiments, the hydrogen to carbon atomic ratio in produced fluid may be at least approximately 1.7 (e.g., 1.8, 1.9, etc.).

One embodiment of the invention includes an in situ method of producing hydrocarbon fluids with improved properties from an organic-rich rock formation. Applicants have surprisingly discovered that the quality of the hydrocarbon fluids produced from in situ heating and pyrolysis of an organic-rich rock formation may be improved by selecting sections of the organic-rich rock formation with higher lithostatic stress for in situ heating and pyrolysis.

The method may include in situ heating of a section of the organic-rich rock formation that has a high lithostatic stress to form hydrocarbon fluids with improved properties. The method may include creating the hydrocarbon fluid by pyrolysis of a solid hydrocarbon and/or a heavy hydrocarbon present in the organic-rich rock formation. Embodiments may include the hydrocarbon fluid being partially, predominantly or substantially completely created by pyrolysis of the solid hydrocarbon and/or heavy hydrocarbon present in the organic-rich rock formation. The method may include heating the section of the organic-rich rock formation by any method, including any of the methods described herein. For example, the method may include heating the section of the organic-rich rock formation by electrical resistance heating. Further, the method may include heating the section of the organic-rich rock formation through use of a heated heat transfer fluid. The method may include heating the section of the organic-rich rock formation to above 270° C. Alternatively, the method may include heating the section of the organic-rich rock formation between 270° C. and 500° C.

The method may include heating in situ a section of the organic-rich rock formation having a lithostatic stress greater than 200 psi and producing a hydrocarbon fluid from the heated section of the organic-rich rock formation. In alternative embodiments, the heated section of the organic-rich rock formation may have a lithostatic stress greater than 400 psi. In alternative embodiments, the heated section of the organic-rich rock formation may have a lithostatic stress greater than 800 psi, greater than 1,000 psi, greater than 1,200 psi, greater than 1,500 psi or greater than 2,000 psi. Applicants have found that in situ heating and pyrolysis of organic-rich rock formations with increasing amounts of stress lead to the production of hydrocarbon fluids with improved properties.

The lithostatic stress of a section of an organic-rich formation can normally be estimated by recognizing that it will generally be equal to the weight of the rocks overlying the formation. The density of the overlying rocks can be expressed in units of psi/ft. Generally, this value will fall between 0.8 and 1.1 psi/ft and can often be approximated as 0.9 psi/ft. As a result the lithostatic stress of a section of an organic-rich formation can be estimated by multiplying the depth of the organic-rich rock formation interval by 0.9 psi/ft. Thus the lithostatic stress of a section of an organic-rich formation occurring at about 1,000 ft can be estimated to be about (0.9 psi/ft) multiplied by (1,000 ft) or about 900 psi. If a more precise estimate of lithostatic stress is desired the density of overlying rocks can be measured using wireline logging techniques or by making laboratory measurements on samples recovered from coreholes. The method may include heating a section of the organic-rich rock formation that is located at a depth greater than 200 ft below the earth's surface. Alternatively, the method may include heating a section of the organic-rich rock formation that is located at a depth greater than 500 ft below the earth's surface, greater than 1,000 ft below the earth's surface, greater than 1,200 ft below the earth's surface, greater than 1,500 ft below the earth's surface, or greater than 2,000 ft below the earth's surface.

The organic-rich rock formation may be, for example, a heavy hydrocarbon formation or a solid hydrocarbon formation. Particular examples of such formations may include an oil shale formation, a tar sands formation or a coal formation. Particular formation hydrocarbons present in such formations may include oil shale, kerogen, coal, and/or bitumen.

The hydrocarbon fluid produced from the organic-rich rock formation may include both a condensable hydrocarbon portion (e.g. liquid) and a non-condensable hydrocarbon portion (e.g. gas). The hydrocarbon fluid may additionally be produced together with non-hydrocarbon fluids. Exemplary non-hydrocarbon fluids include, for example, water, carbon dioxide, hydrogen sulfide, hydrogen, ammonia, and/or carbon monoxide.

The condensable hydrocarbon portion of the hydrocarbon fluid may be a fluid present within different locations associated with an organic-rich rock development project. For example, the condensable hydrocarbon portion of the hydrocarbon fluid may be a fluid present within a production well that is in fluid communication with the organic-rich rock formation. The production well may serve as a device for withdrawing the produced hydrocarbon fluids from the organic-rich rock formation. Alternatively, the condensable hydrocarbon portion may be a fluid present within processing equipment adapted to process hydrocarbon fluids produced from the organic-rich rock formation. Exemplary processing equipment is described herein. Alternatively, the condensable hydrocarbon portion may be a fluid present within a fluid storage vessel. Fluid storage vessels may include, for example, fluid storage tanks with fixed or floating roofs, knock-out vessels, and other intermediate, temporary or product storage vessels. Alternatively, the condensable hydrocarbon portion may be a fluid present within a fluid transportation pipeline. A fluid transportation pipeline may include, for example, piping from production wells to processing equipment or fluid storage vessels, piping from processing equipment to fluid storage vessels, or pipelines associated with collection or transportation of fluids to or from intermediate or centralized storage locations.

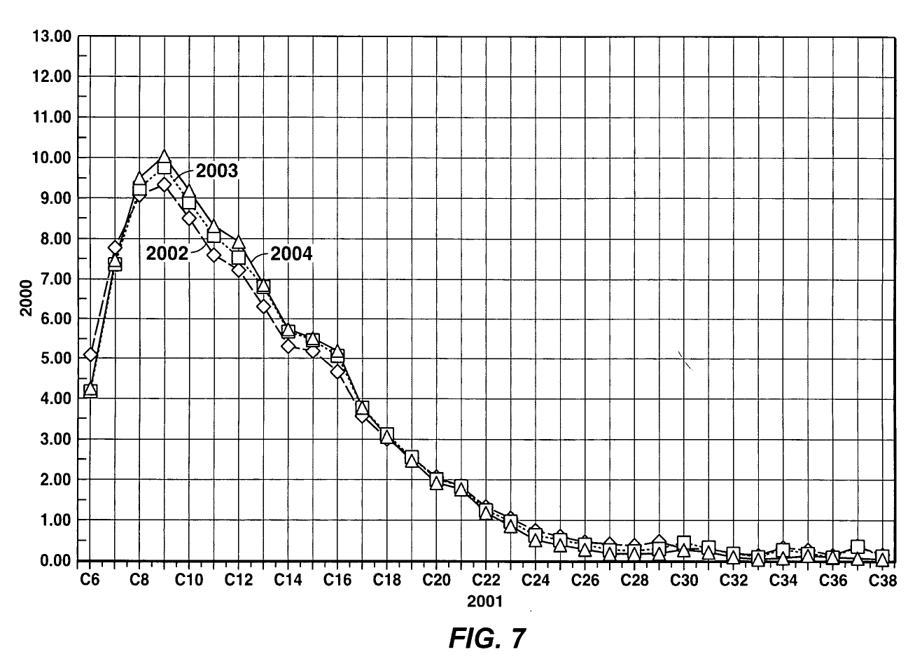

The following discussion of FIG. 7-16 concerns data obtained in Examples 1-5 which are discussed in the section labeled “Experiments”. The data was obtained through the experimental procedures, gas and liquid sample collection procedures, hydrocarbon gas sample gas chromatography (GC) analysis methodology, gas sample GC peak integration methodology, gas sample GC peak identification methodology, whole oil gas chromatography (WOGC) analysis methodology, whole oil gas chromatography (WOGC) peak integration methodology, whole oil gas chromatography (WOGC) peak identification methodology, and pseudo component analysis methodology discussed in the Experiments section. For clarity, when referring to gas chromatography chromatograms of hydrocarbon gas samples, graphical data is provided for one unstressed experiment through Example 1, two 400 psi stressed experiments through Examples 2 and 3, and two 1,000 psi stressed experiments through Examples 4 and 5. When referring to whole oil gas chromatography (WOGC) chromatograms of liquid hydrocarbon samples, graphical data is provided for one unstressed experiment through Example 1, one 400 psi stressed experiments through Example 3, and one 1,000 psi stressed experiment through Example 4.

FIG. 7 is a graph of the weight percent of each carbon number pseudo component occurring from C6 to C38 for each of the three stress levels tested and analyzed in the laboratory experiments discussed herein. The pseudo component weight percentages were obtained through the experimental procedures, liquid sample collection procedures, whole oil gas chromatography (WOGC) analysis methodology, whole oil gas chromatography (WOGC) peak identification and integration methodology, and pseudo component analysis methodology discussed in the Experiments section. For clarity, the pseudo component weight percentages are taken as a percentage of the entire C3 to pseudo C38 whole oil gas chromatography areas and calculated weights. Thus the graphed C6 to C38 weight percentages do not include the weight contribution of the associated gas phase product from any of the experiments which was separately treated. Further, the graphed weight percentages do not include the weight contribution of any liquid hydrocarbon compounds heavier than (i.e. having a longer retention time than) the C38 pseudo component. The y-axis 2000 represents the concentration in terms of weight percent of each C6 to C38 pseudo component in the liquid phase. The x-axis 2001 contains the identity of each hydrocarbon pseudo component from C6 to C38. The data points occurring on line 2002 represent the weight percent of each C6 to C38 pseudo component for the unstressed experiment of Example 1. The data points occurring on line 2003 represent the weight percent of each C6 to C38 pseudo component for the 400 psi stressed experiment of Example 3. While the data points occurring on line 2004 represent the weight percent of each C6 to C38 pseudo component for the 1,000 psi stressed experiment of Example 4. From FIG. 7 it can be seen that the hydrocarbon liquid produced in the unstressed experiment, represented by data points on line 2002, contains a lower weight percentage of lighter hydrocarbon components in the C8 to C17 pseudo component range and a greater weight percentage of heavier hydrocarbon components in the C20 to C29 pseudo component range, both as compared to the 400 psi stress experiment hydrocarbon liquid and the 1,000 psi stress experiment hydrocarbon liquid. Looking now at the data points occurring on line 2003, it is apparent that the intermediate level 400 psi stress experiment produced a hydrocarbon liquid having C8 to C17 pseudo component concentrations between the unstressed experiment represented by line 2002 and the 1,000 psi stressed experiment represented by line 2004. It is noted that the C17 pseudo component data for both the 400 psi and 1,000 psi stressed experiments are about equal. Further, it is apparent that the weight percentage of heavier hydrocarbon components in the C20 to C29 pseudo component range for the intermediate stress level experiment represented by line 2003 falls between the unstressed experiment (Line 2002) hydrocarbon liquid and the 1,000 psi stress experiment (Line 2004) hydrocarbon liquid. Lastly, it is apparent that the high level 1,000 psi stress experiment produced a hydrocarbon liquid having C8 to C17 pseudo component concentrations greater than both the unstressed experiment represented by line 2002 and the 400 psi stressed experiment represented by line 2003. Further, it is apparent that the weight percentage of heavier hydrocarbon components in the C20 to C29 pseudo component range for the high level stress experiment represented by line 2004 are less than both the unstressed experiment (Line 2002) hydrocarbon liquid and the 400 psi stress experiment (Line 2003) hydrocarbon liquid. Thus pyrolyzing oil shale under increasing levels of lithostatic stress appears to produce hydrocarbon liquids having increasingly lighter carbon number distributions.

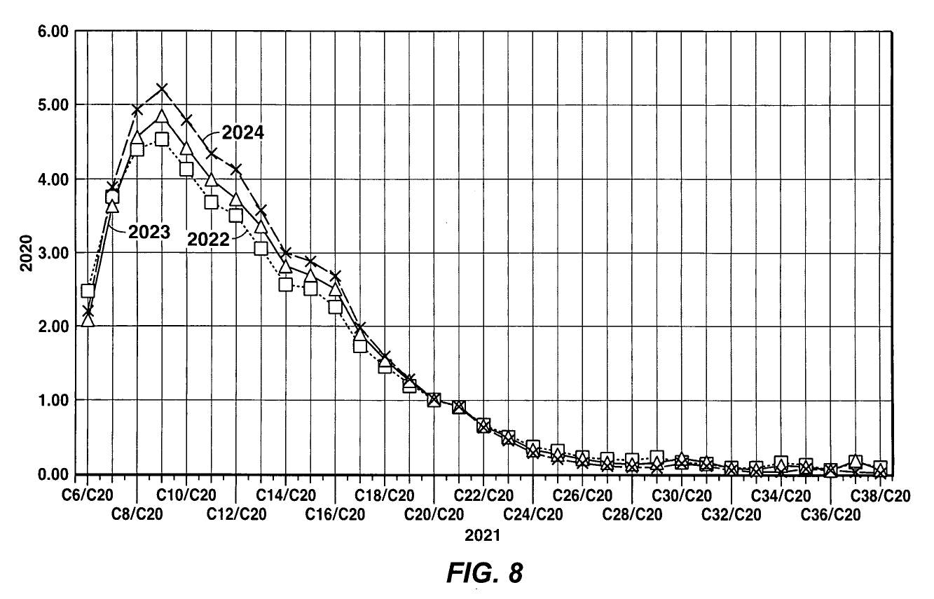

FIG. 8 is a graph of the weight percent ratios of each carbon number pseudo component occurring from C6 to C38 as compared to the C20 pseudo component for each of the three stress levels tested and analyzed in the laboratory experiments discussed herein. The pseudo component weight percentages were obtained as described for FIG. 7. The y-axis 2020 represents the weight ratio of each C6 to C38 pseudo component compared to the C20 pseudo component in the liquid phase. The x-axis 2021 contains the identity of each hydrocarbon pseudo component ratio from C6/C20 to C38/C20. The data points occurring on line 2022 represent the weight ratio of each C6 to C38 pseudo component to C20 pseudo component for the unstressed experiment of Example 1. The data points occurring on line 2023 represent the weight ratio of each C6 to C38 pseudo component to C20 pseudo component for the 400 psi stressed experiment of Example 3. While the data points occurring on line 2024 represent the weight ratio of each C6 to C38 pseudo component to C20 pseudo component for the 1,000 psi stressed experiment of Example 4. From FIG. 8 it can be seen that the hydrocarbon liquid produced in the unstressed experiment, represented by data points on line 2022, contains a lower weight percentage of lighter hydrocarbon components in the C8 to C18 pseudo component range as compared to the C20 pseudo component and a greater weight percentage of heavier hydrocarbon components in the C22 to C29 pseudo component range as compared to the C20 pseudo component, both as compared to the 400 psi stress experiment hydrocarbon liquid and the 1,000 psi stress experiment hydrocarbon liquid. Looking now at the data points occurring on line 2023, it is apparent that the intermediate level 400 psi stress experiment produced a hydrocarbon liquid having C8 to C18 pseudo component concentrations as compared to the C20 pseudo component between the unstressed experiment represented by line 2022 and the 1,000 psi stressed experiment represented by line 2024. Further, it is apparent that the weight percentage of heavier hydrocarbon components in the C22 to C29 pseudo component range as compared to the C20 pseudo component for the intermediate stress level experiment represented by line 2023 falls between the unstressed experiment (Line 2022) hydrocarbon liquid and the 1,000 psi stress experiment (Line 2024) hydrocarbon liquid. Lastly, it is apparent that the high level 1,000 psi stress experiment produced a hydrocarbon liquid having C8 to C18 pseudo component concentrations as compared to the C20 pseudo component greater than both the unstressed experiment represented by line 2022 and the 400 psi stressed experiment represented by line 2023. Further, it is apparent that the weight percentage of heavier hydrocarbon components in the C22 to C29 pseudo component range as compared to the C20 pseudo component for the high level stress experiment represented by line 2024 are less than both the unstressed experiment (Line 2022) hydrocarbon liquid and the 400 psi stress experiment (Line 2023) hydrocarbon liquid. This analysis further supports the relationship that pyrolizing oil shale under increasing levels of lithostatic stress produces hydrocarbon liquids having increasingly lighter carbon number distributions.

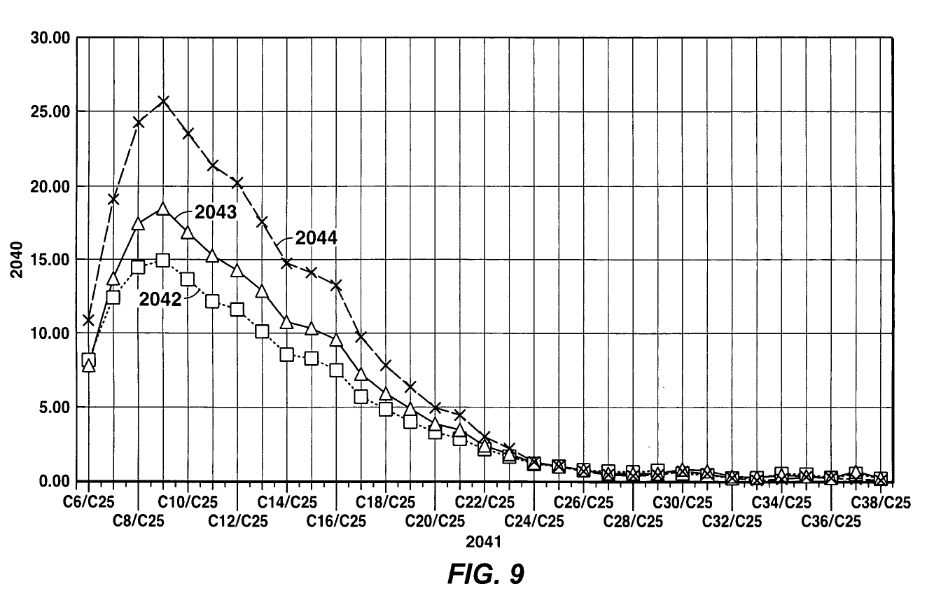

FIG. 9 is a graph of the weight percent ratios of each carbon number pseudo component occurring from C6 to C38 as compared to the C25 pseudo component for each of the three stress levels tested and analyzed in the laboratory experiments discussed herein. The pseudo component weight percentages were obtained as described for FIG. 7. The y-axis 2040 represents the weight ratio of each C6 to C38 pseudo component compared to the C25 pseudo component in the liquid phase. The x-axis 2041 contains the identity of each hydrocarbon pseudo component ratio from C6/C25 to C38/C25. The data points occurring on line 2042 represent the weight ratio of each C6 to C38 pseudo component to C25 pseudo component for the unstressed experiment of Example 1. The data points occurring on line 2043 represent the weight ratio of each C6 to C38 pseudo component to C25 pseudo component for the 400 psi stressed experiment of Example 3. While the data points occurring on line 2044 represent the weight ratio of each C6 to C38 pseudo component to C25 pseudo component for the 1,000 psi stressed experiment of Example 4. From FIG. 9 it can be seen that the hydrocarbon liquid produced in the unstressed experiment, represented by data points on line 2042, contains a lower weight percentage of lighter hydrocarbon components in the C7 to C24 pseudo component range as compared to the C25 pseudo component and a greater weight percentage of heavier hydrocarbon components in the C26 to C29 pseudo component range as compared to the C25 pseudo component, both as compared to the 400 psi stress experiment hydrocarbon liquid and the 1,000 psi stress experiment hydrocarbon liquid. Looking now at the data points occurring on line 2043, it is apparent that the intermediate level 400 psi stress experiment produced a hydrocarbon liquid having C7 to C24 pseudo component concentrations as compared to the C25 pseudo component between the unstressed experiment represented by line 2042 and the 1,000 psi stressed experiment represented by line 2044. Further, it is apparent that the weight percentage of heavier hydrocarbon components in the C26 to C29 pseudo component range as compared to the C25 pseudo component for the intermediate stress level experiment represented by line 2043 falls between the unstressed experiment (Line 2042) hydrocarbon liquid and the 1,000 psi stress experiment (Line 2044) hydrocarbon liquid. Lastly, it is apparent that the high level 1,000 psi stress experiment produced a hydrocarbon liquid having C7 to C24 pseudo component concentrations as compared to the C25 pseudo component greater than both the unstressed experiment represented by line 2042 and the 400 psi stressed experiment represented by line 2043. Further, it is apparent that the weight percentage of heavier hydrocarbon components in the C26 to C29 pseudo component range as compared to the C25 pseudo component for the high level stress experiment represented by line 2044 are less than both the unstressed experiment (Line 2042) hydrocarbon liquid and the 400 psi stress experiment (Line 2043) hydrocarbon liquid. This analysis further supports the relationship that pyrolizing oil shale under increasing levels of lithostatic stress produces hydrocarbon liquids having increasingly lighter carbon number distributions.

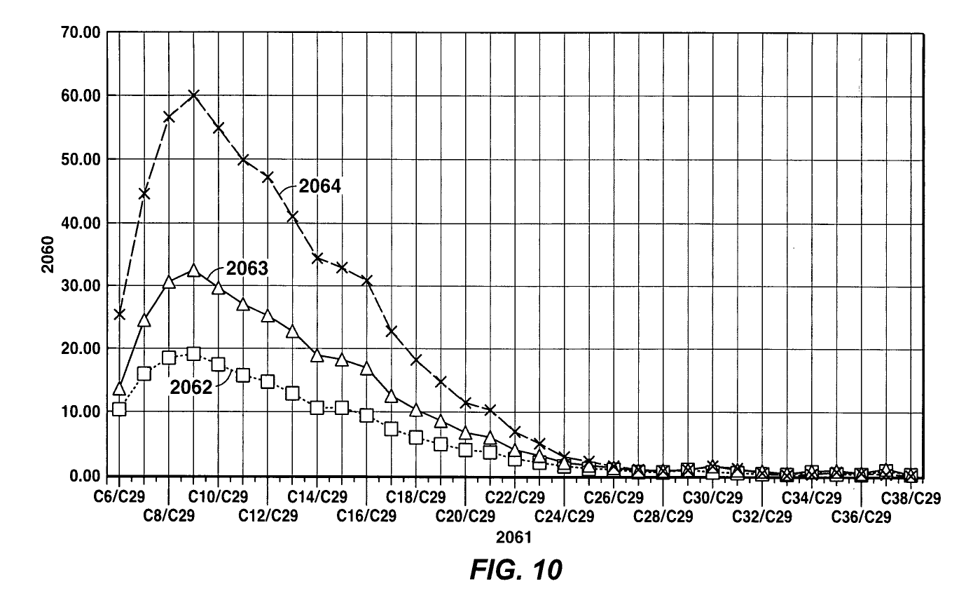

FIG. 10 is a graph of the weight percent ratios of each carbon number pseudo component occurring from C6 to C38 as compared to the C29 pseudo component for each of the three stress levels tested and analyzed in the laboratory experiments discussed herein. The pseudo component weight percentages were obtained as described for FIG. 7. The y-axis 2060 represents the weight ratio of each C6 to C38 pseudo component compared to the C29 pseudo component in the liquid phase. The x-axis 2061 contains the identity of each hydrocarbon pseudo component ratio from C6/C29 to C38/C29. The data points occurring on line 2062 represent the weight ratio of each C6 to C38 pseudo component to C29 pseudo component for the unstressed experiment of Example 1. The data points occurring on line 2063 represent the weight ratio of each C6 to C38 pseudo component to C29 pseudo component for the 400 psi stressed experiment of Example 3. While the data points occurring on line 2064 represent the weight ratio of each C6 to C38 pseudo component to C29 pseudo component for the 1,000 psi stressed experiment of Example 4. From FIG. 10 it can be seen that the hydrocarbon liquid produced in the unstressed experiment, represented by data points on line 2062, contains a lower weight percentage of lighter hydrocarbon components in the C6 to C28 pseudo component range as compared to the C29 pseudo component, both as compared to the 400 psi stress experiment hydrocarbon liquid and the 1,000 psi stress experiment hydrocarbon liquid. Looking now at the data points occurring on line 2063, it is apparent that the intermediate level 400 psi stress experiment produced a hydrocarbon liquid having C6 to C28 pseudo component concentrations as compared to the C29 pseudo component between the unstressed experiment represented by line 2062 and the 1,000 psi stressed experiment represented by line 2064. Lastly, it is apparent that the high level 1,000 psi stress experiment produced a hydrocarbon liquid having C6 to C28 pseudo component concentrations as compared to the C29 pseudo component greater than both the unstressed experiment represented by line 2062 and the 400 psi stressed experiment represented by line 2063. This analysis further supports the relationship that pyrolizing oil shale under increasing levels of lithostatic stress produces hydrocarbon liquids having increasingly lighter carbon number distributions.

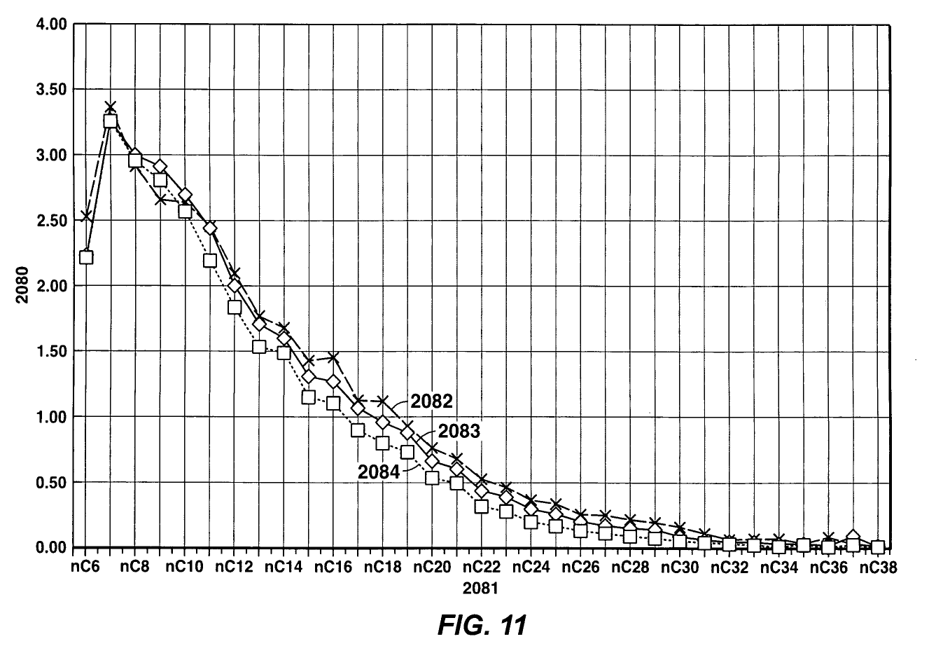

FIG. 11 is a graph of the weight percent of normal alkane hydrocarbon compounds occurring from the normal-C6 alkane to the normal-C38 alkane for each of the three stress levels tested and analyzed in the laboratory experiments discussed herein. The normal alkane compound weight percentages were obtained as described for FIG. 7, except that each individual normal alkane compound peak area integration was used to determine each respective normal alkane compound weight percentage. For clarity, the normal alkane hydrocarbon weight percentages are taken as a percentage of the entire C3 to pseudo C38 whole oil gas chromatography areas and calculated weights as used in the pseudo compound data presented in FIG. 7. The y-axis 2080 represents the concentration in terms of weight percent of each normal-C6 to normal-C38 compound found in the liquid phase. The x-axis 2081 contains the identity of each normal alkane hydrocarbon compound from normal-C6 to normal-C38. The data points occurring on line 2082 represent the weight percent of each normal-C6 to normal-C38 hydrocarbon compound for the unstressed experiment of Example 1. The data points occurring on line 2083 represent the weight percent of each normal-C6 to normal-C38 hydrocarbon compound for the 400 psi stressed experiment of Example 3. While the data points occurring on line 2084 represent the weight percent of each normal-C6 to normal-C38 hydrocarbon compound for the 1,000 psi stressed experiment of Example 4. From FIG. 11 it can be seen that the hydrocarbon liquid produced in the unstressed experiment, represented by data points on line 2082, contains a greater weight percentage of hydrocarbon compounds in the normal-C12 to normal-C30 compound range, both as compared to the 400 psi stress experiment hydrocarbon liquid and the 1,000 psi stress experiment hydrocarbon liquid. Looking now at the data points occurring on line 2083, it is apparent that the intermediate level 400 psi stress experiment produced a hydrocarbon liquid having normal-C12 to normal-C30 compound concentrations between the unstressed experiment represented by line 2082 and the 1,000 psi stressed experiment represented by line 2084. Lastly, it is apparent that the high level 1,000 psi stress experiment produced a hydrocarbon liquid having normal-C12 to normal-C30 compound concentrations less than both the unstressed experiment represented by line 2082 and the 400 psi stressed experiment represented by line 2083. Thus pyrolyzing oil shale under increasing levels of lithostatic stress appears to produce hydrocarbon liquids having lower concentrations of normal alkane hydrocarbons.

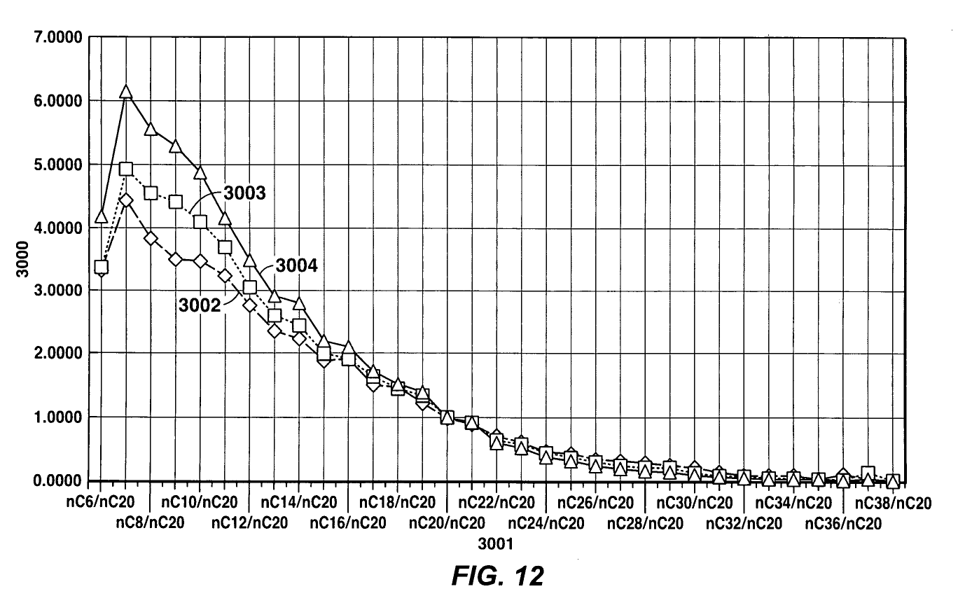

FIG. 12 is a graph of the weight percent of normal alkane hydrocarbon compounds occurring from normal-C6 to normal-C38 as compared to the normal-C20 hydrocarbon compound for each of the three stress levels tested and analyzed in the laboratory experiments discussed herein. The normal compound weight percentages were obtained as described for FIG. 11. The y-axis 3000 represents the concentration in terms of weight ratio of each normal-C6 to normal-C38 compound as compared to the normal-C20 compound found in the liquid phase. The x-axis 3001 contains the identity of each normal alkane hydrocarbon compound ratio from normal-C6/normal-C20 to normal-C38/normal-C20. The data points occurring on line 3002 represent the weight ratio of each normal-C6 to normal-C38 hydrocarbon compound as compared to the normal-C20 compound for the unstressed experiment of Example 1. The data points occurring on line 3003 represent the weight ratio of each normal-C6 to normal-C38 hydrocarbon compound as compared to the normal-C20 compound for the 400 psi stressed experiment of Example 3. While the data points occurring on line 3004 represent the weight ratio of each normal-C6 to normal-C38 hydrocarbon compound as compared to the normal-C20 compound for the 1,000 psi stressed experiment of Example 4. From FIG. 12 it can be seen that the hydrocarbon liquid produced in the unstressed experiment, represented by data points on line 3002, contains a lower weight percentage of lighter normal alkane hydrocarbon components in the normal-C6 to normal-C17 compound range as compared to the normal-C20 compound and a greater weight percentage of heavier hydrocarbon components in the normal-C22 to normal-C34 compound range as compared to the normal-C20 compound, both as compared to the 400 psi stress experiment hydrocarbon liquid and the 1,000 psi stress experiment hydrocarbon liquid. Looking now at the data points occurring on line 3003, it is apparent that the intermediate level 400 psi stress experiment produced a hydrocarbon liquid having normal-C6 to normal-C17 compound concentrations as compared to the normal-C20 compound between the unstressed experiment represented by line 3002 and the 1,000 psi stressed experiment represented by line 3004. Further, it is apparent that the weight percentage of heavier hydrocarbon components in the normal-C22 to normal-C34 compound range as compared to the normal-C20 compound for the intermediate stress level experiment represented by line 3003 falls between the unstressed experiment (Line 3002) hydrocarbon liquid and the 1,000 psi stress experiment (Line 3004) hydrocarbon liquid. Lastly, it is apparent that the high level 1,000 psi stress experiment produced a hydrocarbon liquid having normal-C6 to normal-C17 compound concentrations as compared to the normal-C20 compound greater than both the unstressed experiment represented by line 3002 and the 400 psi stressed experiment represented by line 3003. Further, it is apparent that the weight percentage of heavier hydrocarbon components in the normal-C22 to normal-C34 compound range as compared to the normal-C20 compound for the high level stress experiment represented by line 3004 are less than both the unstressed experiment (Line 3002) hydrocarbon liquid and the 400 psi stress experiment (Line 3003) hydrocarbon liquid. This analysis further supports the relationship that pyrolizing oil shale under increasing levels of lithostatic stress produces hydrocarbon liquids having lower concentrations of normal alkane hydrocarbons.

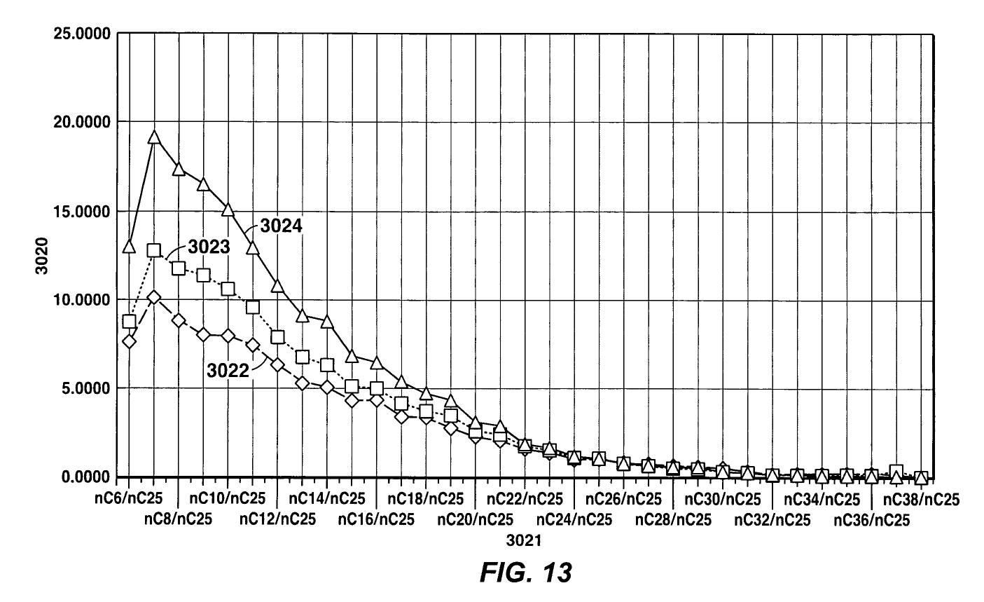

FIG. 13 is a graph of the weight percent of normal alkane hydrocarbon compounds occurring from normal-C6 to normal-C38 as compared to the normal-C25 hydrocarbon compound for each of the three stress levels tested and analyzed in the laboratory experiments discussed herein. The normal compound weight percentages were obtained as described for FIG. 11. The y-axis 3020 represents the concentration in terms of weight ratio of each normal-C6 to normal-C38 compound as compared to the normal-C25 compound found in the liquid phase. The x-axis 3021 contains the identity of each normal alkane hydrocarbon compound ratio from normal-C6/normal-C25 to normal-C38/normal-C25. The data points occurring on line 3022 represent the weight ratio of each normal-C6 to normal-C38 hydrocarbon compound as compared to the normal-C25 compound for the unstressed experiment of Example 1. The data points occurring on line 3023 represent the weight ratio of each normal-C6 to normal-C38 hydrocarbon compound as compared to the normal-C25 compound for the 400 psi stressed experiment of Example 3. While the data points occurring on line 3024 represent the weight ratio of each normal-C6 to normal-C38 hydrocarbon compound as compared to the normal-C25 compound for the 1,000 psi stressed experiment of Example 4. From FIG. 13 it can be seen that the hydrocarbon liquid produced in the unstressed experiment, represented by data points on line 3022, contains a lower weight percentage of lighter normal alkane hydrocarbon components in the normal-C6 to normal-C24 compound range as compared to the normal-C25 compound and a greater weight percentage of heavier hydrocarbon components in the normal-C26 to normal-C30 compound range as compared to the normal-C25 compound, both as compared to the 400 psi stress experiment hydrocarbon liquid and the 1,000 psi stress experiment hydrocarbon liquid. Looking now at the data points occurring on line 3023, it is apparent that the intermediate level 400 psi stress experiment produced a hydrocarbon liquid having normal-C6 to normal-C24 compound concentrations as compared to the normal-C25 compound between the unstressed experiment represented by line 3022 and the 1,000 psi stressed experiment represented by line 3024. Further, it is apparent that the weight percentage of heavier hydrocarbon components in the normal-C26 to normal-C30 compound range as compared to the normal-C25 compound for the intermediate stress level experiment represented by line 3023 falls between the unstressed experiment (Line 3022) hydrocarbon liquid and the 1,000 psi stress experiment (Line 3024) hydrocarbon liquid. Lastly, it is apparent that the high level 1,000 psi stress experiment produced a hydrocarbon liquid having normal-C6 to normal-C24 compound concentrations as compared to the normal-C25 compound greater than both the unstressed experiment represented by line 3022 and the 400 psi stressed experiment represented by line 3023. Further, it is apparent that the weight percentage of heavier hydrocarbon components in the normal-C26 to normal-C30 compound range as compared to the normal-C25 compound for the high level stress experiment represented by line 3024 are less than both the unstressed experiment (Line 3022) hydrocarbon liquid and the 400 psi stress experiment (Line 3023) hydrocarbon liquid. This analysis further supports the relationship that pyrolizing oil shale under increasing levels of lithostatic stress produces hydrocarbon liquids having lower concentrations of normal alkane hydrocarbons.

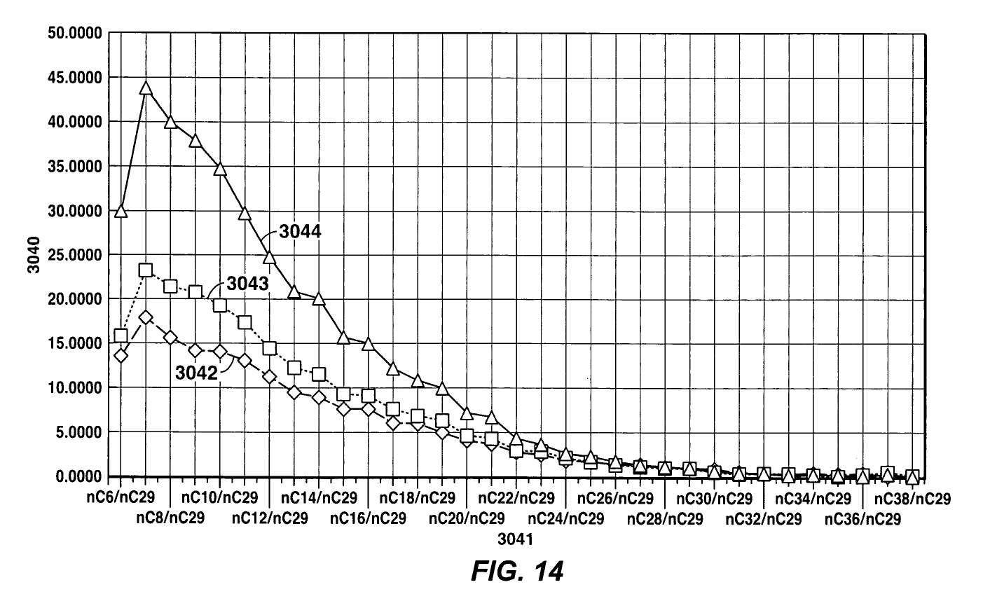

FIG. 14 is a graph of the weight percent of normal alkane hydrocarbon compounds occurring from normal-C6 to normal-C38 as compared to the normal-C29 hydrocarbon compound for each of the three stress levels tested and analyzed in the laboratory experiments discussed herein. The normal compound weight percentages were obtained as described for FIG. 11. The y-axis 3040 represents the concentration in terms of weight ratio of each normal-C6 to normal-C38 compound as compared to the normal-C29 compound found in the liquid phase. The x-axis 3041 contains the identity of each normal alkane hydrocarbon compound ratio from normal-C6/normal-C29 to normal-C38/normal-C29. The data points occurring on line 3042 represent the weight ratio of each normal-C6 to normal-C38 hydrocarbon compound as compared to the normal-C29 compound for the unstressed experiment of Example 1. The data points occurring on line 3043 represent the weight ratio of each normal-C6 to normal-C38 hydrocarbon compound as compared to the normal-C29 compound for the 400 psi stressed experiment of Example 3. While the data points occurring on line 3044 represent the weight ratio of each normal-C6 to normal-C38 hydrocarbon compound as compared to the normal-C29 compound for the 1,000 psi stressed experiment of Example 4. From FIG. 14 it can be seen that the hydrocarbon liquid produced in the unstressed experiment, represented by data points on line 3042, contains a lower weight percentage of lighter normal alkane hydrocarbon components in the normal-C6 to normal-C26 compound range as compared to the normal-C29 compound, both as compared to the 400 psi stress experiment hydrocarbon liquid and the 1,000 psi stress experiment hydrocarbon liquid. Looking now at the data points occurring on line 3043, it is apparent that the intermediate level 400 psi stress experiment produced a hydrocarbon liquid having normal-C6 to normal-C26 compound concentrations as compared to the normal-C29 compound between the unstressed experiment represented by line 3042 and the 1,000 psi stressed experiment represented by line 3044. Lastly, it is apparent that the high level 1,000 psi stress experiment produced a hydrocarbon liquid having normal-C6 to normal-C26 compound concentrations as compared to the normal-C29 compound greater than both the unstressed experiment represented by line 3042 and the 400 psi stressed experiment represented by line 3043. This analysis further supports the relationship that pyrolizing oil shale under increasing levels of lithostatic stress produces hydrocarbon liquids having lower concentrations of normal alkane hydrocarbons.

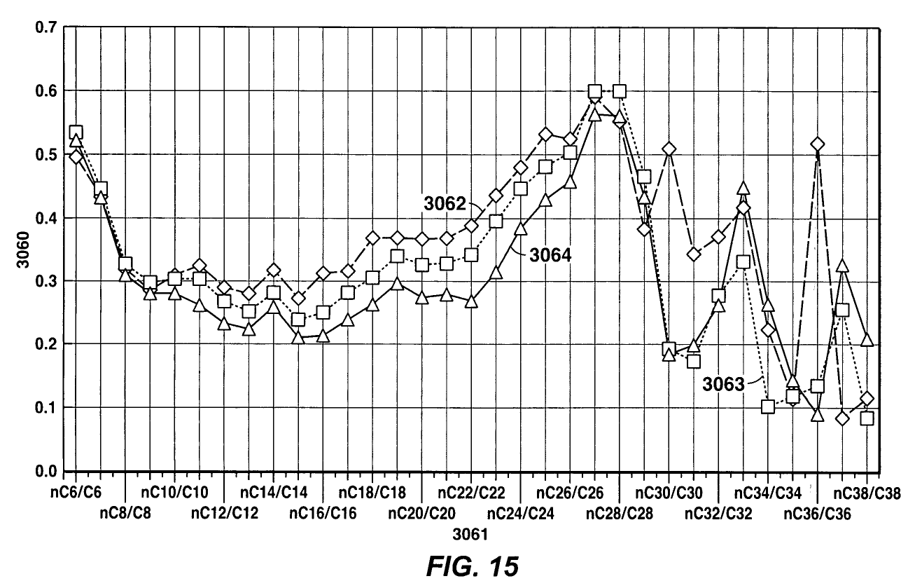

FIG. 15 is a graph of the weight ratio of normal alkane hydrocarbon compounds to pseudo components for each carbon number from C6 to C38 for each of the three stress levels tested and analyzed in the laboratory experiments discussed herein. The normal compound and pseudo component weight percentages were obtained as described for FIGS. 7 & 11. For clarity, the normal alkane hydrocarbon and pseudo component weight percentages are taken as a percentage of the entire C3 to pseudo C38 whole oil gas chromatography areas and calculated weights as used in the pseudo compound data presented in FIG. 7. The y-axis 3060 represents the concentration in terms of weight ratio of each normal-C6/pseudo C6 to normal-C38/pseudo C38 compound found in the liquid phase. The x-axis 3061 contains the identity of each normal alkane hydrocarbon compound to pseudo component ratio from normal-C6/pseudo C6 to normal-C38/pseudo C38. The data points occurring on line 3062 represent the weight ratio of each normal-C6/pseudo C6 to normal-C38/pseudo C38 ratio for the unstressed experiment of Example 1. The data points occurring on line 3063 represent the weight ratio of each normal-C6/pseudo C6 to normal-C38/pseudo C38 ratio for the 400 psi stressed experiment of Example 3. While the data points occurring on line 3064 represent the weight ratio of each normal-C6/pseudo C6 to normal-C38/pseudo C38 ratio for the 1,000 psi stressed experiment of Example 4. From FIG. 15 it can be seen that the hydrocarbon liquid produced in the unstressed experiment, represented by data points on line 3062, contains a greater weight percentage of normal alkane hydrocarbon compounds to pseudo components in the C10 to C26 range, both as compared to the 400 psi stress experiment hydrocarbon liquid and the 1,000 psi stress experiment hydrocarbon liquid. Looking now at the data points occurring on line 3063, it is apparent that the intermediate level 400 psi stress experiment produced a hydrocarbon liquid having normal alkane hydrocarbon compound to pseudo component ratios in the C10 to C26 range between the unstressed experiment represented by line 3062 and the 1,000 psi stressed experiment represented by line 3064. Lastly, it is apparent that the high level 1,000 psi stress experiment produced a hydrocarbon liquid having normal alkane hydrocarbon compound to pseudo component ratios in the C10 to C26 range less than both the unstressed experiment represented by line 3062 and the 400 psi stressed experiment represented by line 3063. Thus pyrolizing oil shale under increasing levels of lithostatic stress appears to produce hydrocarbon liquids having lower concentrations of normal alkane hydrocarbons as compared to the total hydrocarbons for a given carbon number occurring between C10 and C26.

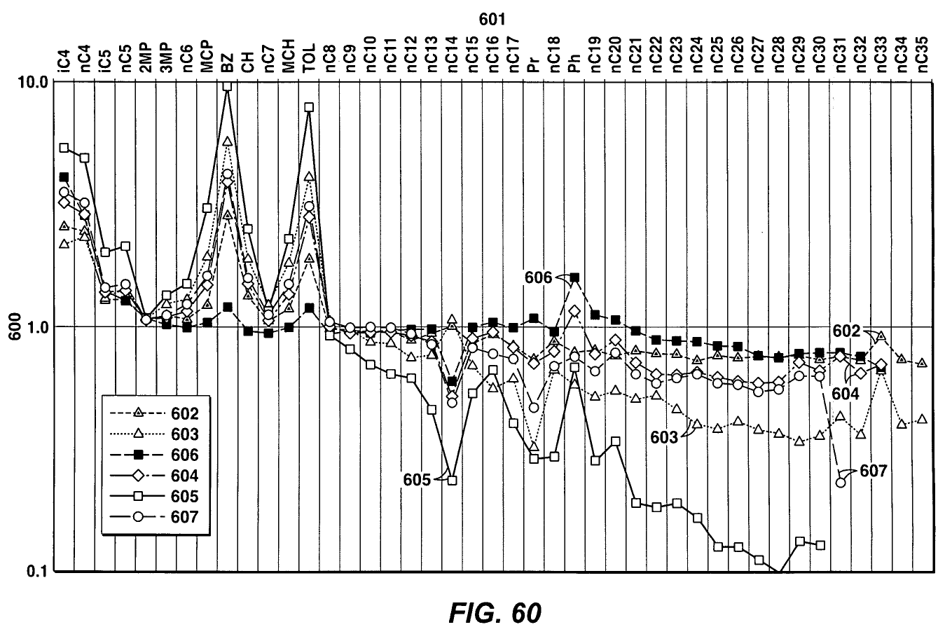

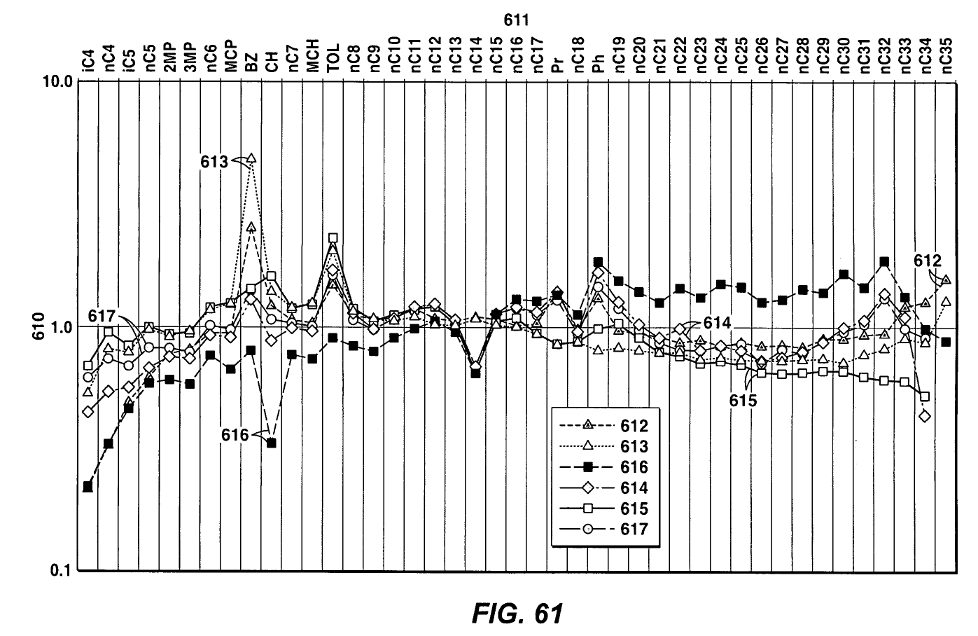

The following discussion of FIG. 60-61 concerns data obtained in Examples 6-19 which are discussed in the section labeled “Experiments”. The data was obtained through the experimental procedures, gas and liquid sample collection procedures, whole oil gas chromatography (WOGC) analysis methodology, whole oil gas chromatography (WOGC) peak integration methodology, whole oil gas chromatography (WOGC) peak identification methodology, and analysis methodology discussed in the Experiments section.

FIG. 60 is a graph of the weight ratio of each WOGC identified compound occurring from i-C4 to n-C35 for each of the six 393° C. experiments tested and analyzed by WOGC in the laboratory experiments (Examples 13-19) discussed herein compared to the weight ratio of each identified compound occurring from i-C4 to n-C35 for Example 13 conducted at 393° C., 500 psig initial argon pressure and 0 psi stress. The compound weight ratios were obtained through the experimental procedures, liquid sample collection procedures, whole oil gas chromatography (WOGC) analysis methodology, whole oil gas chromatography (WOGC) peak integration methodology, and whole oil gas chromatography (WOGC) peak identification methodology discussed in the Experiments section. For clarity, the compound weight ratios were derived as a ratio of a particular compound's percentage of the total peak area in one experiment to the same compound's percentage of the total peak area for the 393/500/0 experiment (Experiment 13). When referring to experimental conditions herein, the notational format “Temperature (° C.)/Initial Argon Pressure (psig)/Stress load (psi)” will be used as a shorthand to refer to the temperature, initial argon pressure and stress loading of a particular experiment. For example, the notation “393/500/0” refers to an experiment conducted at 393° C., 500 psig initial argon pressure and 0 psi stress load as present in Example 13. Thus the graphed i-C4 to n-C35 weight ratios do not include the weight contribution of the associated gas phase product from any of the experiments. Further, the graphed weight ratios do not include the weight contribution of any liquid hydrocarbon compounds heavier than (i.e. having a longer retention time than) n-C35 or any unidentified (i.e., not listed in FIG. 60) compounds from the WOGC data. The y-axis 600 represents the weight ratio of a particular compound for a given experiment to the same compound for the 393/500/0 experiment (Experiment 13). The x-axis 601 contains the identity of each identified compound from i-C4 to n-C35. The data points occurring on line 602 represent the weight ratio of each identified i-C4 to n-C35 compound for the 393/500/400 experiment of Example 15 to the 393/500/0 experiment of Experiment 13. The data points occurring on line 603 represent the weight ratio of each identified i-C4 to n-C35 compound for the 393/500/1000 experiment of Example 18 to the 393/500/0 experiment of Experiment 13. The data points occurring on line 604 represent the weight ratio of each identified i-C4 to n-C35 compound for the 393/200/400 experiment of Example 16 to the 393/500/0 experiment of Experiment 13. The data points occurring on line 605 represent the weight ratio of each identified i-C4 to n-C35 compound for the 393/200/1000 experiment of Example 19 to the 393/500/0 experiment of Experiment 13. The data points occurring on line 606 represent the weight ratio of each identified i-C4 to n-C35 compound for the 393/200/0 experiment of Example 14 to the 393/500/0 experiment of Experiment 13. The data points occurring on line 607 represent the weight ratio of each identified i-C4 to n-C35 compound for the 393/50/400 experiment of Example 17 to the 393/500/0 experiment of Experiment 13.

From FIG. 60 it can also be seen that the hydrocarbon liquids produced in the two 1,000 psi stressed experiments, represented by data points on line 603 & 605, generally contain a decreased weight ratio of normal alkane hydrocarbon compounds for n-C8 and heavier normal alkane hydrocarbon compounds, including for example n-C9 through n-C35. It also can be seen that for the two 1,000 psi stress experiments, the lower initial argon pressure (200 psig argon) experiment represented by line 605 is generally more depleted of normal hydrocarbon compounds relative to the higher initial argon pressure (500 psig argon) experiment represented by line 603. From FIG. 60 it can also be seen that the hydrocarbon liquid produced in the three 400 psi stressed experiments, represented by data points on line 602, 604 & 607, generally contain a decreased amount of normal hydrocarbon compounds for n-C8 and heavier relative to the unstressed experiments (i.e., line 606 & the “1” line on the y-axis representing Experiments 13 & 14) but a less depleted weight ratio of normal hydrocarbon compounds relative to the 1,000 psi stressed experiments (Lines 603 & 605). It also can be seen that for the three 400 psi stress experiments, the lowest initial argon pressure (50 psig argon) experiment represented by line 607 is generally more depleted of normal compounds for n-C8 and heavier relative to the middle initial argon pressures (200 psig argon) experiment represented by line 604 and the highest initial argon pressures (500 psig argon) experiment represented by line 602, with the middle initial argon pressures (200 psig argon) experiment represented by line 604 generally falling between the highest and lowest initial argon pressure experiments. It is also apparent that for normal hydrocarbon compounds lighted than n-C8 (e.g., n-C5, n-C6 & n-C7), the above described trends go in the opposite direction with increasing stress and decreasing pressure. Thus pyrolyzing oil shale under increasing levels of stress appears to deplete the produced hydrocarbon liquid in normal hydrocarbon compounds for n-C8 and heavier while decreasing pressure also appears to decrease normal hydrocarbon compound for n-C8 and heavier production. Further, pyrolyzing oil shale under increasing levels of stress appears to enrich the produced hydrocarbon liquid in normal hydrocarbon compounds for n-C7 and lighter while decreasing pressure also appears to increase normal hydrocarbon compound for n-C7 and lighter production. Trends apparent for aromatic hydrocarbon compounds (e.g., benzene & toluene) and cyclic hydrocarbon compounds (e.g., methyl cyclohexane & methyl cyclopentane) will be discussed further with regard to the C4-C19 GC data described herein.

FIG. 61 is a graph of the weight ratio of each WOGC identified compound occurring from i-C4 to n-C35 for each of the six 375° C. experiments tested and analyzed by WOGC in the laboratory experiments (Examples 7-12) discussed herein compared to the weight ratio of each identified compound occurring from i-C4 to n-C35 for Example 6 conducted at 375° C., 500 psig initial argon pressure and 0 psi stress. The data was obtained in a similar manner as discussed above for FIG. 60. The y-axis 610 represents the weight ratio of a particular compound for a given experiment to the same compound for the 375/500/0 experiment (Experiment 6). The x-axis 611 contains the identity of each identified compound from i-C4 to n-C35. The data points occurring on line 612 represent the weight ratio of each identified i-C4 to n-C35 compound for the 375/500/400 experiment of Example 8 to the 375/500/0 experiment of Experiment 6. The data points occurring on line 613 represent the weight ratio of each identified i-C4 to n-C35 compound for the 375/500/1000 experiment of Example 11 to the 375/500/0 experiment of Experiment 6. The data points occurring on line 614 represent the weight ratio of each identified i-C4 to n-C35 compound for the 375/200/400 experiment of Example 9 to the 375/500/0 experiment of Experiment 6. The data points occurring on line 615 represent the weight ratio of each identified i-C4 to n-C35 compound for the 375/200/1000 experiment of Example 12 to the 375/500/0 experiment of Experiment 6. The data points occurring on line 616 represent the weight ratio of each identified i-C4 to n-C35 compound for the 375/200/0 experiment of Example 7 to the 375/500/0 experiment of Experiment 6. The data points occurring on line 617 represent the weight ratio of each identified i-C4 to n-C35 compound for the 375/50/400 experiment of Example 10 to the 375/500/0 experiment of Experiment 6. While the trends for the 375° C. data are not as consistent as the trends discussed above for the 393° C. data, the same general relationships as discussed above for the 393° C. data are apparent for the 375° C. data. Further, it is apparent that the magnitude of the deviations from the zero line are not as great as for the 393° C. data. Thus it is apparent that temperature also has a significant effect on the above discussed compositional changes.

From the above-described data, it can be seen that heating and pyrolysis of oil shale under increasing levels of stress results in a condensable hydrocarbon fluid product that is lighter (i.e., greater proportion of lower carbon number compounds or components relative to higher carbon number compounds or components) and contains a lower concentration of normal alkane hydrocarbon compounds. Such a product may be suitable for refining into gasoline and distillate products. Further, such a product, either before or after further fractionation, may have utility as a feed stock for certain chemical processes.

In some embodiments, the produced hydrocarbon fluid includes a condensable hydrocarbon portion. In some embodiments the condensable hydrocarbon portion may have one or more of a total C7 to total C20 weight ratio greater than 0.8, a total C8 to total C20 weight ratio greater than 1.7, a total C9 to total C20 weight ratio greater than 2.5, a total C10 to total C20 weight ratio greater than 2.8, a total C11 to total C20 weight ratio greater than 2.3, a total C12 to total C20 weight ratio greater than 2.3, a total C13 to total C20 weight ratio greater than 2.9, a total C14 to total C20 weight ratio greater than 2.2, a total C15 to total C20 weight ratio greater than 2.2, and a total C16 to total C20 weight ratio greater than 1.6. In alternative embodiments the condensable hydrocarbon portion has one or more of a total C7 to total C20 weight ratio greater than 2.5, a total C8 to total C20 weight ratio greater than 3.0, a total C9 to total C20 weight ratio greater than 3.5, a total C10 to total C20 weight ratio greater than 3.5, a total C11 to total C20 weight ratio greater than 3.0, and a total C12 to total C20 weight ratio greater than 3.0. In alternative embodiments the condensable hydrocarbon portion has one or more of a total C7 to total C20 weight ratio greater than 3.5, a total C8 to total C20 weight ratio greater than 4.3, a total C9 to total C20 weight ratio greater than 4.5, a total C10 to total C20 weight ratio greater than 4.2, a total C11 to total C20 weight ratio greater than 3.7, and a total C12 to total C20 weight ratio greater than 3.5. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C7 to total C20 weight ratio greater than 0.8. Alternatively, the condensable hydrocarbon portion may have a total C7 to total C20 weight ratio greater than 1.0, greater than 1.5, greater than 2.0, greater than 2.5, greater than 3.5 or greater than 3.7. In alternative embodiments, the condensable hydrocarbon portion may have a total C7 to total C20 weight ratio less than 10.0, less than 7.0, less than 5.0 or less than 4.0. In some embodiments the condensable hydrocarbon portion has a total C8 to total C20 weight ratio greater than 1.7. Alternatively, the condensable hydrocarbon portion may have a total C8 to total C20 weight ratio greater than 2.0, greater than 2.5, greater than 3.0, greater than 4.0, greater than 4.4, or greater than 4.6. In alternative embodiments, the condensable hydrocarbon portion may have a total C8 to total C20 weight ratio less than 7.0 or less than 6.0. In some embodiments the condensable hydrocarbon portion has a total C9 to total C20 weight ratio greater than 2.5. Alternatively, the condensable hydrocarbon portion may have a total C9 to total C20 weight ratio greater than 3.0, greater than 4.0, greater than 4.5, or greater than 4.7. In alternative embodiments, the condensable hydrocarbon portion may have a total C9 to total C20 weight ratio less than 7.0 or less than 6.0. In some embodiments the condensable hydrocarbon portion has a total C10 to total C20 weight ratio greater than 2.8. Alternatively, the condensable hydrocarbon portion may have a total C10 to total C20 weight ratio greater than 3.0, greater than 3.5, greater than 4.0, or greater than 4.3. In alternative embodiments, the condensable hydrocarbon portion may have a total C10 to total C20 weight ratio less than 7.0 or less than 6.0. In some embodiments the condensable hydrocarbon portion has a total C11 to total C20 weight ratio greater than 2.3. Alternatively, the condensable hydrocarbon portion may have a total C11 to total C20 weight ratio greater than 2.5, greater than 3.5, greater than 3.7, greater than 4.0. In alternative embodiments, the condensable hydrocarbon portion may have a total C11 to total C20 weight ratio less than 7.0 or less than 6.0. In some embodiments the condensable hydrocarbon portion has a total C12 to total C20 weight ratio greater than 2.3. Alternatively, the condensable hydrocarbon portion may have a total C12 to total C20 weight ratio greater than 2.5, greater than 3.0, greater than 3.5, or greater than 3.7. In alternative embodiments, the condensable hydrocarbon portion may have a total C12 to total C20 weight ratio less than 7.0 or less than 6.0. In some embodiments the condensable hydrocarbon portion has a total C13 to total C20 weight ratio greater than 2.9. Alternatively, the condensable hydrocarbon portion may have a total C13 to total C20 weight ratio greater than 3.0, greater than 3.1, or greater than 3.2. In alternative embodiments, the condensable hydrocarbon portion may have a total C13 to total C20 weight ratio less than 6.0 or less than 5.0. In some embodiments the condensable hydrocarbon portion has a total C14 to total C20 weight ratio greater than 2.2. Alternatively, the condensable hydrocarbon portion may have a total C14 to total C20 weight ratio greater than 2.5, greater than 2.6, or greater than 2.7. In alternative embodiments, the condensable hydrocarbon portion may have a total C14 to total C20 weight ratio less than 6.0 or less than 4.0. In some embodiments the condensable hydrocarbon portion has a total C15 to total C20 weight ratio greater than 2.2. Alternatively, the condensable hydrocarbon portion may have a total C15 to total C20 weight ratio greater than 2.3, greater than 2.4, or greater than 2.6. In alternative embodiments, the condensable hydrocarbon portion may have a total C15 to total C20 weight ratio less than 6.0 or less than 4.0. In some embodiments the condensable hydrocarbon portion has a total C16 to total C20 weight ratio greater than 1.6. Alternatively, the condensable hydrocarbon portion may have a total C16 to total C20 weight ratio greater than 1.8, greater than 2.3, or greater than 2.5. In alternative embodiments, the condensable hydrocarbon portion may have a total C16 to total C20 weight ratio less than 5.0 or less than 4.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have the one or more of a total C7 to total C25 weight ratio greater than 2.0, a total C8 to total C25 weight ratio greater than 4.5, a total C9 to total C25 weight ratio greater than 6.5, a total C10 to total C25 weight ratio greater than 7.5, a total C11 to total C25 weight ratio greater than 6.5, a total C12 to total C25 weight ratio greater than 6.5, a total C13 to total C25 weight ratio greater than 8.0, a total C14 to total C25 weight ratio greater than 6.0, a total C15 to total C25 weight ratio greater than 6.0, a total C16 to total C25 weight ratio greater than 4.5, a total C17 to total C25 weight ratio greater than 4.8, and a total C18 to total C25 weight ratio greater than 4.5. In alternative embodiments the condensable hydrocarbon portion has one or more of a total C7 to total C25 weight ratio greater than 7.0, a total C8 to total C25 weight ratio greater than 10.0, a total C9 to total C25 weight ratio greater than 10.0, a total C10 to total C25 weight ratio greater than 10.0, a total C11 to total C25 weight ratio greater than 8.0, and a total C12 to total C25 weight ratio greater than 8.0. In alternative embodiments the condensable hydrocarbon portion has one or more of a total C7 to total C25 weight ratio greater than 13.0, a total C8 to total C25 weight ratio greater than 17.0, a total C9 to total C25 weight ratio greater than 17.0, a total C10 to total C25 weight ratio greater than 15.0, a total C11 to total C25 weight ratio greater than 14.0, and a total C12 to total C25 weight ratio greater than 13.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C7 to total C25 weight ratio greater than 2.0. Alternatively, the condensable hydrocarbon portion may have a total C7 to total C25 weight ratio greater than 3.0, greater than 5.0, greater than 10.0, greater than 13.0, or greater than 15.0. In alternative embodiments, the condensable hydrocarbon portion may have a total C7 to total C25 weight ratio less than 30.0 or less than 25.0. In some embodiments the condensable hydrocarbon portion has a total C8 to total C25 weight ratio greater than 4.5. Alternatively, the condensable hydrocarbon portion may have a total C8 to total C25 weight ratio greater than 5.0, greater than 7.0, greater than 10.0, greater than 15.0, or greater than 17.0. In alternative embodiments, the condensable hydrocarbon portion may have a total C8 to total C25 weight ratio less than 35.0, or less than 30.0. In some embodiments the condensable hydrocarbon portion has a total C9 to total C25 weight ratio greater than 6.5. Alternatively, the condensable hydrocarbon portion may have a total C9 to total C25 weight ratio greater than 8.0, greater than 10.0, greater than 15.0, greater than 17.0, or greater than 19.0. In alternative embodiments, the condensable hydrocarbon portion may have a total C9 to total C25 weight ratio less than 40.0 or less than 35.0. In some embodiments the condensable hydrocarbon portion has a total C10 to total C25 weight ratio greater than 7.5. Alternatively, the condensable hydrocarbon portion may have a total C10 to total C25 weight ratio greater than 10.0, greater than 14.0, or greater than 17.0. In alternative embodiments, the condensable hydrocarbon portion may have a total C10 to total C25 weight ratio less than 35.0 or less than 30.0. In some embodiments the condensable hydrocarbon portion has a total C11 to total C25 weight ratio greater than 6.5. Alternatively, the condensable hydrocarbon portion may have a total C11 to total C25 weight ratio greater than 8.5, greater than 10.0, greater than 12.0, or greater than 14.0. In alternative embodiments, the condensable hydrocarbon portion may have a total C11 to total C25 weight ratio less than 35.0 or less than 30.0. In some embodiments the condensable hydrocarbon portion has a total C12 to total C25 weight ratio greater than 6.5. Alternatively, the condensable hydrocarbon portion may have a total C12 to total C25 weight ratio greater than 8.5, a total C12 to total C25 weight ratio greater than 10.0, greater than 12.0, or greater than 14.0. In alternative embodiments, the condensable hydrocarbon portion may have a total C12 to total C25 weight ratio less than 30.0 or less than 25.0. In some embodiments the condensable hydrocarbon portion has a total C13 to total C25 weight ratio greater than 8.0. Alternatively, the condensable hydrocarbon portion may have a total C13 to total C25 weight ratio greater than 10.0, greater than 12.0, or greater than 14.0. In alternative embodiments, the condensable hydrocarbon portion may have a total C13 to total C25 weight ratio less than 25.0 or less than 20.0. In some embodiments the condensable hydrocarbon portion has a total C14 to total C25 weight ratio greater than 6.0. Alternatively, the condensable hydrocarbon portion may have a total C14 to total C25 weight ratio greater than 8.0, greater than 10.0, or greater than 12.0. In alternative embodiments, the condensable hydrocarbon portion may have a total C14 to total C25 weight ratio less than 25.0 or less than 20.0. In some embodiments the condensable hydrocarbon portion has a total C15 to total C25 weight ratio greater than 6.0. Alternatively, the condensable hydrocarbon portion may have a total C15 to total C25 weight ratio greater than 8.0, or greater than 10.0. In alternative embodiments, the condensable hydrocarbon portion may have a total C15 to total C25 weight ratio less than 25.0 or less than 20.0. In some embodiments the condensable hydrocarbon portion has a total C16 to total C25 weight ratio greater than 4.5. Alternatively, the condensable hydrocarbon portion may have a total C16 to total C25 weight ratio greater than 6.0, greater than 8.0, or greater than 10.0. In alternative embodiments, the condensable hydrocarbon portion may have a total C16 to total C25 weight ratio less than 20.0 or less than 15.0. In some embodiments the condensable hydrocarbon portion has a total C17 to total C25 weight ratio greater than 4.8. Alternatively, the condensable hydrocarbon portion may have a total C17 to total C25 weight ratio greater than 5.5 or greater than 7.0. In alternative embodiments, the condensable hydrocarbon portion may have a total C17 to total C25 weight ratio less than 20.0. In some embodiments the condensable hydrocarbon portion has a total C18 to total C25 weight ratio greater than 4.5. Alternatively, the condensable hydrocarbon portion may have a total C18 to total C25 weight ratio greater than 5.0 or greater than 5.5. In alternative embodiments, the condensable hydrocarbon portion may have a total C18 to total C25 weight ratio less than 15.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have the one or more of a total C7 to total C29 weight ratio greater than 3.5, a total C8 to total C29 weight ratio greater than 9.0, a total C9 to total C29 weight ratio greater than 12.0, a total C10 to total C29 weight ratio greater than 15.0, a total C11 to total C29 weight ratio greater than 13.0, a total C12 to total C29 weight ratio greater than 12.5, and a total C13 to total C29 weight ratio greater than 16.0, a total C14 to total C29 weight ratio greater than 12.0, a total C15 to total C29 weight ratio greater than 12.0, a total C16 to total C29 weight ratio greater than 9.0, a total C17 to total C29 weight ratio greater than 10.0, a total C18 to total C29 weight ratio greater than 8.8, a total C19 to total C29 weight ratio greater than 7.0, a total C20 to total C29 weight ratio greater than 6.0, a total C21 to total C29 weight ratio greater than 5.5, and a total C22 to total C29 weight ratio greater than 4.2. In alternative embodiments the condensable hydrocarbon portion has one or more of a total C7 to total C29 weight ratio greater than 16.0, a total C8 to total C29 weight ratio greater than 19.0, a total C9 to total C29 weight ratio greater than 20.0, a total C10 to total C29 weight ratio greater than 18.0, a total C11 to total C29 weight ratio greater than 16.0, a total C12 to total C29 weight ratio greater than 15.0, and a total C13 to total C29 weight ratio greater than 17.0, a total C14 to total C29 weight ratio greater than 13.0, a total C15 to total C29 weight ratio greater than 13.0, a total C16 to total C29 weight ratio greater than 10.0, a total C17 to total C29 weight ratio greater than 11.0, a total C18 to total C29, weight ratio greater than 9.0, a total C19 to total C29 weight ratio greater than 8.0, a total C20 to total C29 weight ratio greater than 6.5, and a total C21 to total C29 weight ratio greater than 6.0. In alternative embodiments the condensable hydrocarbon portion has one or more of a total C7 to total C29 weight ratio greater than 24.0, a total C8 to total C29 weight ratio greater than 30.0, a total C9 to total C29 weight ratio greater than 32.0, a total C10 to total C29 weight ratio greater than 30.0, a total C11 to total C29 weight ratio greater than 27.0, a total C12 to total C29 weight ratio greater than 25.0, and a total C13 to total C29 weight ratio greater than 22.0, a total C14 to total C29 weight ratio greater than 18.0, a total C15 to total C29 weight ratio greater than 18.0, a total C16 to total C29 weight ratio greater than 16.0, a total C17 to total C29 weight ratio greater than 13.0, a total C18 to total C29 weight ratio greater than 10.0, a total C19 to total C29 weight ratio greater than 9.0, and a total C20 to total C29 weight ratio greater than 7.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C7 to total C29 weight ratio greater than 3.5. Alternatively, the condensable hydrocarbon portion may have a total C7 to total C29 weight ratio greater than 5.0, greater than 10.0, greater than 18.0, greater than 20.0, or greater than 24.0. In alternative embodiments, the condensable hydrocarbon portion may have a total C7 to total C29 weight ratio less than 60.0 or less than 50.0. In some embodiments the condensable hydrocarbon portion has a total C8 to total C29 weight ratio greater than 9.0. Alternatively, the condensable hydrocarbon portion may have a total C8 to total C29 weight ratio greater than 10.0, greater than 18.0, greater than 20.0, greater than 25.0, or greater than 30.0. In alternative embodiments, the condensable hydrocarbon portion may have a total C8 to total C29 weight ratio less than 85.0 or less than 75.0. In some embodiments the condensable hydrocarbon portion has a total C9 to total C29 weight ratio greater than 12.0. Alternatively, the condensable hydrocarbon portion may have a total C9 to total C29 weight ratio greater than 15.0, greater than 20.0, greater than 23.0, greater than 27.0, or greater than 32.0. In alternative embodiments, the condensable hydrocarbon portion may have a total C9 to total C29 weight ratio less than 85.0 or less than 75.0. In some embodiments the condensable hydrocarbon portion has a total C10 to total C29 weight ratio greater than 15.0. Alternatively, the condensable hydrocarbon portion may have a total C10 to total C29 weight ratio greater than 18.0, greater than 22.0, or greater than 28.0. In alternative embodiments, the condensable hydrocarbon portion may have a total C10 to total C29 weight ratio less than 80.0 or less than 70.0. In some embodiments the condensable hydrocarbon portion has a total C11 to total C29 weight ratio greater than 13.0. Alternatively, the condensable hydrocarbon portion may have a total C11 to total C29 weight ratio greater than 16.0, greater than 18.0, greater than 24.0, or greater than 27.0. In alternative embodiments, the condensable hydrocarbon portion may have a total C11 to total C29 weight ratio less than 75.0 or less than 65.0. In some embodiments the condensable hydrocarbon portion has a total C12 to total C29 weight ratio greater than 12.5. Alternatively, the condensable hydrocarbon portion may have a total C12 to total C29 weight ratio greater than 14.5, greater than 18.0, greater than 22.0, or greater than 25.0. In alternative embodiments, the condensable hydrocarbon portion may have a total C12 to total C29 weight ratio less than 75.0 or less than 65.0. In some embodiments the condensable hydrocarbon portion has a total C13 to total C29 weight ratio greater than 16.0. Alternatively, the condensable hydrocarbon portion may have a total C13 to total C29 weight ratio greater than 18.0, greater than 20.0, or greater than 22.0. In alternative embodiments, the condensable hydrocarbon portion may have a total C13 to total C29 weight ratio less than 70.0 or less than 60.0. In some embodiments the condensable hydrocarbon portion has a total C14 to total C29 weight ratio greater than 12.0. Alternatively, the condensable hydrocarbon portion may have a total C14 to total C29 weight ratio greater than 14.0, greater than 16.0, or greater than 18.0. In alternative embodiments, the condensable hydrocarbon portion may have a total C14 to total C29 weight ratio less than 60.0 or less than 50.0. In some embodiments the condensable hydrocarbon portion has a total C15 to total C29 weight ratio greater than 12.0. Alternatively, the condensable hydrocarbon portion may have a total C15 to total C29 weight ratio greater than 15.0 or greater than 18.0. In alternative embodiments, the condensable hydrocarbon portion may have a total C15 to total C29 weight ratio less than 60.0 or less than 50.0. In some embodiments the condensable hydrocarbon portion has a total C16 to total C29 weight ratio greater than 9.0. Alternatively, the condensable hydrocarbon portion may have a total C16 to total C29 weight ratio greater than 10.0, greater than 13.0, or greater than 16.0. In alternative embodiments, the condensable hydrocarbon portion may have a total C16 to total C29 weight ratio less than 55.0 or less than 45.0. In some embodiments the condensable hydrocarbon portion has a total C17 to total C29 weight ratio greater than 10.0. Alternatively, the condensable hydrocarbon portion may have a total C17 to total C29 weight ratio greater than 11.0 or greater than 12.0. In alternative embodiments, the condensable hydrocarbon portion may have a total C17 to total C29 weight ratio less than 45.0. In some embodiments the condensable hydrocarbon portion has a total C18 to total C29 weight ratio greater than 8.8. Alternatively, the condensable hydrocarbon portion may have a total C18 to total C29 weight ratio greater than 9.0 or greater than 10.0. In alternative embodiments, the condensable hydrocarbon portion may have a total C18 to total C29 weight ratio less than 35.0. In some embodiments the condensable hydrocarbon portion has a total C19 to total C29 weight ratio greater than 7.0. Alternatively, the condensable hydrocarbon portion may have a total C19 to total C29 weight ratio greater than 8.0 or greater than 9.0. In alternative embodiments, the condensable hydrocarbon portion may have a total C19 to total C29 weight ratio less than 30.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have the one or more of a total C9 to total C20 weight ratio between 2.5 and 6.0, a total C10 to total C20 weight ratio between 2.8 and 7.3, a total C11 to total C20 weight ratio between 2.6 and 6.5, a total C12 to total C20 weight ratio between 2.6 and 6.4 and a total C13 to total C20 weight ratio between 3.2 and 8.0. In alternative embodiments the condensable hydrocarbon portion has one or more of a total C9 to total C20 weight ratio between 3.0 and 5.5, a total C10 to total C20 weight ratio between 3.2 and 7.0, a total C11 to total C20 weight ratio between 3.0 and 6.0, a total C12 to total C20 weight ratio between 3.0 and 6.0, and a total C13 to total C20 weight ratio between 3.3 and 7.0. In alternative embodiments the condensable hydrocarbon portion has one or more of a total C9 to total C20 weight ratio between 4.6 and 5.5, a total C10 to total C20 weight ratio between 4.2 and 7.0, a total C11 to total C20 weight ratio between 3.7 and 6.0, a total C12 to total C20 weight ratio between 3.6 and 6.0, and a total C13 to total C20 weight ratio between 3.4 and 7.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C9 to total C20 weight ratio between 2.5 and 6.0. Alternatively, the condensable hydrocarbon portion may have a total C9 to total C20 weight ratio between 3.0 and 5.8, between 3.5 and 5.8, between 4.0 and 5.8, between 4.5 and 5.8, between 4.6 and 5.8, or between 4.7 and 5.8. In some embodiments the condensable hydrocarbon portion has a total C10 to total C20 weight ratio between 2.8 and 7.3. Alternatively, the condensable hydrocarbon portion may have a total C10 to total C20 weight ratio between 3.0 and 7.2, between 3.5 and 7.0, between 4.0 and 7.0, between 4.2 and 7.0, between 4.3 and 7.0, or between 4.4 and 7.0. In some embodiments the condensable hydrocarbon portion has a total C11 to total C20 weight ratio between 2.6 and 6.5. Alternatively, the condensable hydrocarbon portion may have a total C11 to total C20 weight ratio between 2.8 and 6.3, between 3.5 and 6.3, between 3.7 and 6.3, between 3.8 and 6.3, between 3.9 and 6.2, or between 4.0 and 6.2. In some embodiments the condensable hydrocarbon portion has a total C12 to total C20 weight ratio between 2.6 and 6.4. Alternatively, the condensable hydrocarbon portion may have a total C12 to total C20 weight ratio between 2.8 and 6.2, between 3.2 and 6.2, between 3.5 and 6.2, between 3.6 and 6.2, between 3.7 and 6.0, or between 3.8 and 6.0. In some embodiments the condensable hydrocarbon portion has a total C13 to total C20 weight ratio between 3.2 and 8.0. Alternatively, the condensable hydrocarbon portion may have a total C13 to total C20 weight ratio between 3.3 and 7.8, between 3.3 and 7.0, between 3.4 and 7.0, between 3.5 and 6.5, or between 3.6 and 6.0. The embodiments described in this paragraph may be combined with any of the other aspects of the invention discussed herein.

In some embodiments the condensable hydrocarbon portion may have one or more of a total C10 to total C25 weight ratio between 7.1 and 24.5, a total C11 to total C25 weight ratio between 6.5 and 22.0, a total C12 to total C25 weight ratio between 6.5 and 22.0, and a total C13 to total C25 weight ratio between 8.0 and 27.0. In alternative embodiments the condensable hydrocarbon portion has one or more of a total C10 to total C25 weight ratio between 10.0 and 24.0, a total C11 to total C25 weight ratio between 10.0 and 21.5, a total C12 to total C25 weight ratio between 10.0 and 21.5, and a total C13 to total C25 weight ratio between 9.0 and 25.0. In alternative embodiments the condensable hydrocarbon portion has one or more of a total C10 to total C25 weight ratio between 14.0 and 24.0, a total C11 to total C25 weight ratio between 12.5 and 21.5, a total C12 to total C25 weight ratio between 12.0 and 21.5, and a total C13 to total C25 weight ratio between 10.5 and 25.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C10 to total C25 weight ratio between 7.1 and 24.5. Alternatively, the condensable hydrocarbon portion may have a total C10 to total C25 weight ratio between 7.5 and 24.5, between 12.0 and 24.5, between 13.8 and 24.5, between 14.0 and 24.5, or between 15.0 and 24.5. In some embodiments the condensable hydrocarbon portion has a total C11 to total C25 weight ratio between 6.5 and 22.0. Alternatively, the condensable hydrocarbon portion may have a total C11 to total C25 weight ratio between 7.0 and 21.5, between 10.0 and 21.5, between 12.5 and 21.5, between 13.0 and 21.5, between 13.7 and 21.5, or between 14.5 and 21.5. In some embodiments the condensable hydrocarbon portion has a total C12 to total C25 weight ratio between 10.0 and 21.5. Alternatively, the condensable hydrocarbon portion may have a total C12 to total C25 weight ratio between 10.5 and 21.0, between 11.0 and 21.0, between 12.0 and 21.0, between 12.5 and 21.0, between 13.0 and 21.0, or between 13.5 and 21.0. In some embodiments the condensable hydrocarbon portion has a total C13 to total C25 weight ratio between 8.0 and 27.0. Alternatively, the condensable hydrocarbon portion may have a total C13 to total C25 weight ratio between 9.0 and 26.0, between 10.0 and 25.0, between 10.5 and 25.0, between 11.0 and 25.0, or between 11.5 and 25.0. The embodiments described in this paragraph may be combined with any of the other aspects of the invention discussed herein.

In some embodiments the condensable hydrocarbon portion may have one or more of a total C10 to total C29 weight ratio between 15.0 and 60.0, a total C11 to total C29 weight ratio between 13.0 and 54.0, a total C12 to total C29 weight ratio between 12.5 and 53.0, and a total C13 to total C29 weight ratio between 16.0 and 65.0. In alternative embodiments the condensable hydrocarbon portion has one or more of a total C10 to total C29 weight ratio between 17.0 and 58.0, a total C11 to total C29 weight ratio between 15.0 and 52.0, a total C12 to total C29 weight ratio between 14.0 and 50.0, and a total C13 to total C29 weight ratio between 17.0 and 60.0. In alternative embodiments the condensable hydrocarbon portion has one or more of a total C10 to total C29 weight ratio between 20.0 and 58.0, a total C11 to total C29 weight ratio between 18.0 and 52.0, a total C12 to total C29 weight ratio between 18.0 and 50.0, and a total C13 to total C29 weight ratio between 18.0 and 50.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a total C10 to total C29 weight ratio between 15.0 and 60.0. Alternatively, the condensable hydrocarbon portion may have a total C10 to total C29 weight ratio between 18.0 and 58.0, between 20.0 and 58.0, between 24.0 and 58.0, between 27.0 and 58.0, or between 30.0 and 58.0. In some embodiments the condensable hydrocarbon portion has a total C11 to total C29 weight ratio between 13.0 and 54.0. Alternatively, the condensable hydrocarbon portion may have a total C11 to total C29 weight ratio between 15.0 and 53.0, between 18.0 and 53.0, between 20.0 and 53.0, between 22.0 and 53.0, between 25.0 and 53.0, or between 27.0 and 53.0. In some embodiments the condensable hydrocarbon portion has a total C12 to total C29 weight ratio between 12.5 and 53.0. Alternatively, the condensable hydrocarbon portion may have a total C12 to total C29 weight ratio between 14.5 and 51.0, between 16.0 and 51.0, between 18.0 and 51.0, between 20.0 and 51.0, between 23.0 and 51.0, or between 25.0 and 51.0. In some embodiments the condensable hydrocarbon portion has a total C13 to total C29 weight ratio between 16.0 and 65.0. Alternatively, the condensable hydrocarbon portion may have a total C13 to total C29 weight ratio between 17.0 and 60.0, between 18.0 and 60.0, between 20.0 and 60.0, between 22.0 and 60.0, or between 25.0 and 60.0. The embodiments described in this paragraph may be combined with any of the other aspects of the invention discussed herein.

In some embodiments the condensable hydrocarbon portion may have one or more of a normal-C7 to normal-C20 weight ratio greater than 0.9, a normal-C8 to normal-C20 weight ratio greater than 2.0, a normal-C9 to normal-C20 weight ratio greater than 1.9, a normal-C10 to normal-C20 weight ratio greater than 2.2, a normal-C11 to normal-C20 weight ratio greater than 1.9, a normal-C12 to normal-C20 weight ratio greater than 1.9, a normal-C13 to normal-C20 weight ratio greater than 2.3, a normal-C14 to normal-C20 weight ratio greater than 1.8, a normal-C15 to normal-C20 weight ratio greater than 1.8, and normal-C16 to normal-C20 weight ratio greater than 1.3. In alternative embodiments the condensable hydrocarbon portion has one or more of a normal-C7 to normal-C20 weight ratio greater than 4.4, a normal-C8 to normal-C20 weight ratio greater than 3.7, a normal-C9 to normal-C20 weight ratio greater than 3.5, a normal-C10 to normal-C20 weight ratio greater than 3.4, a normal-C11 to normal-C20 weight ratio greater than 3.0, and a normal-C12 to normal-C20 weight ratio greater than 2.7. In alternative embodiments the condensable hydrocarbon portion has one or more of a normal-C7 to normal-C20 weight ratio greater than 4.9, a normal-C8 to normal-C20 weight ratio greater than 4.5, a normal-C9 to normal-C20 weight ratio greater than 4.4, a normal-C10 to normal-C20 weight ratio greater than 4.1, a normal-C11 to normal-C20 weight ratio greater than 3.7, and a normal-C12 to normal-C20 weight ratio greater than 3.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a normal-C7 to normal-C20 weight ratio greater than 0.9. Alternatively, the condensable hydrocarbon portion may have a normal-C7 to normal-C20 weight ratio greater than 1.0, than 2.0, greater than 3.0, greater than 4.0, greater than 4.5, or greater than 5.0. In alternative embodiments, the condensable hydrocarbon portion may have a normal-C7 to normal-C20 weight ratio less than 8.0 or less than 7.0. In some embodiments the condensable hydrocarbon portion has a normal-C8 to normal-C20 weight ratio greater than 1.7. Alternatively, the condensable hydrocarbon portion may have a normal-C8 to normal-C20 weight ratio greater than 2.0, greater than 2.5, greater than 3.0, greater than 3.5, greater than 4.0, or greater than 4.4. In alternative embodiments, the condensable hydrocarbon portion may have a normal-C8 to normal-C20 weight ratio less than 8.0 or less than 7.0. In some embodiments the condensable hydrocarbon portion has a normal-C9 to normal-C20 weight ratio greater than 1.9. Alternatively, the condensable hydrocarbon portion may have a normal-C9 to normal-C20 weight ratio greater than 2.0, greater than 3.0, greater than 4.0, or greater than 4.5. In alternative embodiments, the condensable hydrocarbon portion may have a normal-C9 to normal-C20 weight ratio less than 7.0 or less than 6.0. In some embodiments the condensable hydrocarbon portion has a normal-C10 to normal-C20 weight ratio greater than 2.2. Alternatively, the condensable hydrocarbon portion may have a normal-C10 to normal-C20 weight ratio greater than 2.8, greater than 3.3, greater than 3.5, or greater than 4.0. In alternative embodiments, the condensable hydrocarbon portion may have a normal-C10 to normal-C20 weight ratio less than 7.0 or less than 6.0. In some embodiments the condensable hydrocarbon portion has a normal-C11 to normal-C20 weight ratio greater than 1.9. Alternatively, the condensable hydrocarbon portion may have a normal-C11 to normal-C20 weight ratio greater than 2.5, greater than 3.0, greater than 3.5, or greater than 3.7. In alternative embodiments, the condensable hydrocarbon portion may have a normal-C11 to normal-C20 weight ratio less than 7.0 or less than 6.0. In some embodiments the condensable hydrocarbon portion has a normal-C12 to normal-C20 weight ratio greater than 1.9. Alternatively, the condensable hydrocarbon portion may have a normal-C12 to normal-C20 weight ratio greater than 2.0, greater than 2.2, greater than 2.6, or greater than 3.0. In alternative embodiments, the condensable hydrocarbon portion may have a normal-C12 to normal-C20 weight ratio less than 7.0 or less than 6.0. In some embodiments the condensable hydrocarbon portion has a normal-C13 to normal-C20 weight ratio greater than 2.3. Alternatively, the condensable hydrocarbon portion may have a normal-C13 to normal-C20 weight ratio greater than 2.5, greater than 2.7, or greater than 3.0. In alternative embodiments, the condensable hydrocarbon portion may have a normal-C13 to normal-C20 weight ratio less than 6.0 or less than 5.0. In some embodiments the condensable hydrocarbon portion has a normal-C14 to normal-C20 weight ratio greater than 1.8. Alternatively, the condensable hydrocarbon portion may have a normal-C14 to normal-C20 weight ratio greater than 2.0, greater than 2.2, or greater than 2.5. In alternative embodiments, the condensable hydrocarbon portion may have a normal-C14 to normal-C20 weight ratio less than 6.0 or less than 4.0. In some embodiments the condensable hydrocarbon portion has a normal-C15 to normal-C20 weight ratio greater than 1.8. Alternatively, the condensable hydrocarbon portion may have a normal-C15 to normal-C20 weight ratio greater than 2.0, greater than 2.2, or greater than 2.4. In alternative embodiments, the condensable hydrocarbon portion may have a normal-C15 to normal-C20 weight ratio less than 6.0 or less than 4.0. In some embodiments the condensable hydrocarbon portion has a normal-C16 to normal-C20 weight ratio greater than 1.3. Alternatively, the condensable hydrocarbon portion may have a normal-C16 to normal-C20 weight ratio greater than 1.5, greater than 1.7, or greater than 2.0. In alternative embodiments, the condensable hydrocarbon portion may have a normal-C16 to normal-C20 weight ratio less than 5.0 or less than 4.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have one or more of a normal-C7 to normal-C25 weight ratio greater than 1.9, a normal-C8 to normal-C25 weight ratio greater than 3.9, a normal-C9 to normal-C25 weight ratio greater than 3.7, a normal-C10 to normal-C25 weight ratio greater than 4.4, a normal-C11 to normal-C25 weight ratio greater than 3.8, a normal-C12 to normal-C25 weight ratio greater than 3.7, a normal-C13 to normal-C25 weight ratio greater than 4.7, a normal-C14 to normal-C25 weight ratio greater than 3.7, a normal-C15 to normal-C25 weight ratio greater than 3.7, a normal-C16 to normal-C25 weight ratio greater than 2.5, a normal-C17 to normal-C25 weight ratio greater than 3.0, and a normal-C18 to normal-C25 weight ratio greater than 3.4. In alternative embodiments the condensable hydrocarbon portion has one or more of a normal-C7 to normal-C25 weight ratio greater than 10, a normal-C8 to normal-C25 weight ratio greater than 8.0, a normal-C9 to normal-C25 weight ratio greater than 7.0, a normal-C10 to normal-C25 weight ratio greater than 7.0, a normal-C11 to normal-C25 weight ratio greater than 7.0, and a normal-C12 to normal-C25 weight ratio greater than 6.0. In alternative embodiments the condensable hydrocarbon portion has one or more of a normal-C7 to normal-C25 weight ratio greater than 10.0, a normal-C8 to normal-C25 weight ratio greater than 12.0, a normal-C9 to normal-C25 weight ratio greater than 11.0, a normal-C10 to normal-C25 weight ratio greater than 11.0, a normal-C11 to normal-C25 weight ratio greater than 9.0, and a normal-C12 to normal-C25 weight ratio greater than 8.0. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a normal-C7 to normal-C25 weight ratio greater than 1.9. Alternatively, the condensable hydrocarbon portion may have a normal-C7 to normal-C25 weight ratio greater than 3.0, greater than 5.0, greater than 8.0, greater than 10.0, or greater than 13.0. In alternative embodiments, the condensable hydrocarbon portion may have a normal-C7 to normal-C25 weight ratio less than 35.0 or less than 25.0. In some embodiments the condensable hydrocarbon portion has a normal-C8 to normal-C25 weight ratio greater than 3.9. Alternatively, the condensable hydrocarbon portion may have a normal-C8 to normal-C25 weight ratio greater than 4.5, greater than 6.0, greater than 8.0, greater than 10.0, or greater than 13.0. In alternative embodiments, the condensable hydrocarbon portion may have a normal-C8 to normal-C25 weight ratio less than 35.0 or less than 25.0. In some embodiments the condensable hydrocarbon portion has a normal-C9 to normal-C25 weight ratio greater than 3.7. Alternatively, the condensable hydrocarbon portion may have a normal-C9 to normal-C25 weight ratio greater than 4.5, greater than 7.0, greater than 10.0, greater than 12.0, or greater than 13.0. In alternative embodiments, the condensable hydrocarbon portion may have a normal-C9 to normal-C25 weight ratio less than 35.0 or less than 25.0. In some embodiments the condensable hydrocarbon portion has a normal-C10 to normal-C25 weight ratio greater than 4.4. Alternatively, the condensable hydrocarbon portion may have a normal-C10 to normal-C25 weight ratio greater than 6.0, greater than 8.0, or greater than 11.0. In alternative embodiments, the condensable hydrocarbon portion may have a normal-C10 to normal-C25 weight ratio less than 35.0 or less than 25.0. In some embodiments the condensable hydrocarbon portion has a normal-C11 to normal-C25 weight ratio greater than 3.8. Alternatively, the condensable hydrocarbon portion may have a normal-C11 to normal-C25 weight ratio greater than 4.5, greater than 7.0, greater than 8.0, or greater than 10.0. In alternative embodiments, the condensable hydrocarbon portion may have a normal-C11 to normal-C25 weight ratio less than 35.0 or less than 25.0. In some embodiments the condensable hydrocarbon portion has a normal-C12 to normal-C25 weight ratio greater than 3.7. Alternatively, the condensable hydrocarbon portion may have a normal-C12 to normal-C25 weight ratio greater than 4.5, greater than 6.0, greater than 7.0, or greater than 8.0. In alternative embodiments, the condensable hydrocarbon portion may have a normal-C12 to normal-C25 weight ratio less than 30.0 or less than 20.0. In some embodiments the condensable hydrocarbon portion has a normal-C13 to normal-C25 weight ratio greater than 4.7. Alternatively, the condensable hydrocarbon portion may have a normal-C13 to normal-C25 weight ratio greater than 5.0, greater than 6.0, or greater than 7.5. In alternative embodiments, the condensable hydrocarbon portion may have a normal-C13 to normal-C25 weight ratio less than 25.0 or less than 20.0. In some embodiments the condensable hydrocarbon portion has a normal-C14 to normal-C25 weight ratio greater than 3.7. Alternatively, the condensable hydrocarbon portion may have a normal-C14 to normal-C25 weight ratio greater than 4.5, greater than 5.5, or greater than 7.0. In alternative embodiments, the condensable hydrocarbon portion may have a normal-C14 to normal-C25 weight ratio less than 25.0 or less than 20.0. In some embodiments the condensable hydrocarbon portion has a normal-C15 to normal-C25 weight ratio greater than 3.7. Alternatively, the condensable hydrocarbon portion may have a normal-C15 to normal-C25 weight ratio greater than 4.2 or greater than 5.0. In alternative embodiments, the condensable hydrocarbon portion may have a normal-C15 to normal-C25 weight ratio less than 25.0 or less than 20.0. In some embodiments the condensable hydrocarbon portion has a normal-C16 to normal-C25 weight ratio greater than 2.5. Alternatively, the condensable hydrocarbon portion may have a normal-C16 to normal-C25 weight ratio greater than 3.0, greater than 4.0, or greater than 5.0. In alternative embodiments, the condensable hydrocarbon portion may have a normal-C16 to normal-C25 weight ratio less than 20.0 or less than 15.0. In some embodiments the condensable hydrocarbon portion has a normal-C17 to normal-C25 weight ratio greater than 3.0. Alternatively, the condensable hydrocarbon portion may have a normal-C17 to normal-C25 weight ratio greater than 3.5 or greater than 4.0. In alternative embodiments, the condensable hydrocarbon portion may have a normal-C17 to normal-C25 weight ratio less than 20.0. In some embodiments the condensable hydrocarbon portion has a normal-C18 to normal-C25 weight ratio greater than 3.4. Alternatively, the condensable hydrocarbon portion may have a normal-C18 to normal-C25 weight ratio greater than 3.6 or greater than 4.0. In alternative embodiments, the condensable hydrocarbon portion may have a normal-C18 to normal-C25 weight ratio less than 15.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have one or more of a normal-C7 to normal-C29 weight ratio greater than 18.0, a normal-C8 to normal-C29 weight ratio greater than 16.0, a normal-C9 to normal-C29 weight ratio greater than 14.0, a normal-C10 to normal-C29 weight ratio greater than 14.0, a normal-C11 to normal-C29 weight ratio greater than 13.0, a normal-C12 to normal-C29 weight ratio greater than 11.0, a normal-C13 to normal-C29 weight ratio greater than 10.0, a normal-C14 to normal-C29 weight ratio greater than 9.0, a normal-C15 to normal-C29 weight ratio greater than 8.0, a normal-C16 to normal-C29 weight ratio greater than 8.0, a normal-C17 to normal-C29 weight ratio greater than 6.0, a normal-C18 to normal-C29 weight ratio greater than 6.0, a normal-C19 to normal-C29 weight ratio greater than 5.0, a normal-C20 to normal-C29 weight ratio greater than 4.0, a normal-C21 to normal-C29 weight ratio greater than 3.6, and a normal-C22 to normal-C29 weight ratio greater than 2.8. In alternative embodiments the condensable hydrocarbon portion has one or more of a normal-C7 to normal-C29 weight ratio greater than 20.0, a normal-C8 to normal-C29 weight ratio greater than 18.0, a normal-C9 to normal-C29 weight ratio greater than 17.0, a normal-C10 to normal-C29 weight ratio greater than 16.0, a normal-C11 to normal-C29 weight ratio greater than 15.0, a normal-C12 to normal-C29 weight ratio greater than 12.5, a normal-C13 to normal-C29 weight ratio greater than 11.0, a normal-C14 to normal-C29 weight ratio greater than 10.0, a normal-C15 to normal-C29 weight ratio greater than 8.0, a normal-C16 to normal-C29 weight ratio greater than 8.0, a normal-C17 to normal-C29 weight ratio greater than 7.0, a normal-C18 to normal-C29 weight ratio greater than 6.5, a normal-C19 to normal-C29 weight ratio greater than 5.5, a normal-C20 to normal-C29 weight ratio greater than 4.5, and a normal-C21 to normal-C29 weight ratio greater than 4.0. In alternative embodiments the condensable hydrocarbon portion has one or more of a normal-C7 to normal-C29 weight ratio greater than 23.0, a normal-C8 to normal-C29 weight ratio greater than 21.0, a normal-C9 to normal-C29 weight ratio greater than 20.0, a normal-C10 to normal-C29 weight ratio greater than 19.0, a normal-C11 to normal-C29 weight ratio greater than 17.0, a normal-C12 to normal-C29 weight ratio greater than 14.0, a normal-C13 to normal-C29 weight ratio greater than 12.0, a normal-C14 to normal-C29 weight ratio greater than 11.0, a normal-C15 to normal-C29 weight ratio greater than 9.0, a normal-C16 to normal-C29 weight ratio greater than 9.0, a normal-C17 to normal-C29 weight ratio greater than 7.5, a normal-C18 to normal-C29 weight ratio greater than 7.0, a normal-C19 to normal-C29 weight ratio greater than 6.5, a normal-C20 to normal-C29 weight ratio greater than 4.8, and a normal-C21 to normal-C29 weight ratio greater than 4.5. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a normal-C7 to normal-C29 weight ratio greater than 18.0. Alternatively, the condensable hydrocarbon portion may have a normal-C7 to normal-C29 weight ratio greater than 20.0, greater than 22.0, greater than 25.0, greater than 30.0, or greater than 35.0. In alternative embodiments, the condensable hydrocarbon portion may have a normal-C7 to normal-C29 weight ratio less than 70.0 or less than 60.0. In some embodiments the condensable hydrocarbon portion has a normal-C8 to normal-C29 weight ratio greater than 16.0. Alternatively, the condensable hydrocarbon portion may have a normal-C8 to normal-C29 weight ratio greater than 18.0, greater than 22.0, greater than 25.0, greater than 27.0, or greater than 30.0. In alternative embodiments, the condensable hydrocarbon portion may have a normal-C8 to normal-C29 weight ratio less than 85.0 or less than 75.0. In some embodiments the condensable hydrocarbon portion has a normal-C9 to normal-C29 weight ratio greater than 14.0. Alternatively, the condensable hydrocarbon portion may have a normal-C9 to normal-C29 weight ratio greater than 18.0, greater than 20.0, greater than 23.0, greater than 27.0, or greater than 30.0. In alternative embodiments, the condensable hydrocarbon portion may have a normal-C9 to normal-C29 weight ratio less than 85.0 or less than 75.0. In some embodiments the condensable hydrocarbon portion has a normal-C10 to normal-C29 weight ratio greater than 14.0. Alternatively, the condensable hydrocarbon portion may have a normal-C10 to normal-C29 weight ratio greater than 20.0, greater than 25.0, or greater than 30.0. In alternative embodiments, the condensable hydrocarbon portion may have a normal-C10 to normal-C29 weight ratio less than 80.0 or less than 70.0. In some embodiments the condensable hydrocarbon portion has a normal-C11 to normal-C29 weight ratio greater than 13.0. Alternatively, the condensable hydrocarbon portion may have a normal-C11 to normal-C29 weight ratio greater than 16.0, greater than 18.0, greater than 24.0, or greater than 27.0. In alternative embodiments, the condensable hydrocarbon portion may have a normal-C11 to normal-C29 weight ratio less than 75.0 or less than 65.0. In some embodiments the condensable hydrocarbon portion has a normal-C12 to normal-C29 weight ratio greater than 11.0. Alternatively, the condensable hydrocarbon portion may have a normal-C12 to normal-C29 weight ratio greater than 14.5, greater than 18.0, greater than 22.0, or greater than 25.0. In alternative embodiments, the condensable hydrocarbon portion may have a normal-C12 to normal-C29 weight ratio less than 75.0 or less than 65.0. In some embodiments the condensable hydrocarbon portion has a normal-C13 to normal-C29 weight ratio greater than 10.0. Alternatively, the condensable hydrocarbon portion may have a normal-C13 to normal-C29 weight ratio greater than 18.0, greater than 20.0, or greater than 22.0. In alternative embodiments, the condensable hydrocarbon portion may have a normal-C13 to normal-C29 weight ratio less than 70.0 or less than 60.0. In some embodiments the condensable hydrocarbon portion has a normal-C14 to normal-C29 weight ratio greater than 9.0. Alternatively, the condensable hydrocarbon portion may have a normal-C14 to normal-C29 weight ratio greater than 14.0, greater than 16.0, or greater than 18.0. In alternative embodiments, the condensable hydrocarbon portion may have a normal-C14 to normal-C29 weight ratio less than 60.0 or less than 50.0. In some embodiments the condensable hydrocarbon portion has a normal-C15 to normal-C29 weight ratio greater than 8.0. Alternatively, the condensable hydrocarbon portion may have a normal-C15 to normal-C29 weight ratio greater than 12.0 or greater than 16.0. In alternative embodiments, the condensable hydrocarbon portion may have a normal-C15 to normal-C29 weight ratio less than 60.0 or less than 50.0. In some embodiments the condensable hydrocarbon portion has a normal-C16 to normal-C29 weight ratio greater than 8.0. Alternatively, the condensable hydrocarbon portion may have a normal-C16 to normal-C29 weight ratio greater than 10.0, greater than 13.0, or greater than 15.0. In alternative embodiments, the condensable hydrocarbon portion may have a normal-C16 to normal-C29 weight ratio less than 55.0 or less than 45.0. In some embodiments the condensable hydrocarbon portion has a normal-C17 to normal-C29 weight ratio greater than 6.0. Alternatively, the condensable hydrocarbon portion may have a normal-C17 to normal-C29 weight ratio greater than 8.0 or greater than 12.0. In alternative embodiments, the condensable hydrocarbon portion may have a normal-C17 to normal-C29 weight ratio less than 45.0. In some embodiments the condensable hydrocarbon portion has a normal-C18 to normal-C29 weight ratio greater than 6.0. Alternatively, the condensable hydrocarbon portion may have a normal-C18 to normal-C29 weight ratio greater than 8.0 or greater than 10.0. In alternative embodiments, the condensable hydrocarbon portion may have a normal-C18 to normal-C29 weight ratio less than 35.0. In some embodiments the condensable hydrocarbon portion has a normal-C19 to normal-C29 weight ratio greater than 5.0. Alternatively, the condensable hydrocarbon portion may have a normal-C19 to normal-C29 weight ratio greater than 7.0 or greater than 9.0. In alternative embodiments, the condensable hydrocarbon portion may have a normal-C19 to normal-C29 weight ratio less than 30.0. In some embodiments the condensable hydrocarbon portion has a normal-C20 to normal-C29 weight ratio greater than 4.0. Alternatively, the condensable hydrocarbon portion may have a normal-C20 to normal-C29 weight ratio greater than 6.0 or greater than 8.0. In alternative embodiments, the condensable hydrocarbon portion may have a normal-C20 to normal-C29 weight ratio less than 30.0. In some embodiments the condensable hydrocarbon portion has a normal-C21 to normal-C29 weight ratio greater than 3.6. Alternatively, the condensable hydrocarbon portion may have a normal-C21 to normal-C29 weight ratio greater than 4.0 or greater than 6.0. In alternative embodiments, the condensable hydrocarbon portion may have a normal-C21 to normal-C29 weight ratio less than 30.0. In some embodiments the condensable hydrocarbon portion has a normal-C22 to normal-C29 weight ratio greater than 2.8. Alternatively, the condensable hydrocarbon portion may have a normal-C22 to normal-C29 weight ratio greater than 3.0. In alternative embodiments, the condensable hydrocarbon portion may have a normal-C22 to normal-C29 weight ratio less than 30.0. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

In some embodiments the condensable hydrocarbon portion may have one or more of a normal-C10 to total C10 weight ratio less than 0.31, a normal-C11 to total C11 weight ratio less than 0.32, a normal-C12 to total C12 weight ratio less than 0.29, a normal-C13 to total C13 weight ratio less than 0.28, a normal-C14 to total C14 weight ratio less than 0.31, a normal-C15 to total C15 weight ratio less than 0.27, a normal-C16 to total C16 weight ratio less than 0.31, a normal-C17 to total C17 weight ratio less than 0.31, a normal-C18 to total C18 weight ratio less than 0.37, normal-C19 to total C19 weight ratio less than 0.37, a normal-C20 to total C20 weight ratio less than 0.37, a normal-C21 to total C21 weight ratio less than 0.37, a normal-C22 to total C22 weight ratio less than 0.38, normal-C23 to total C23 weight ratio less than 0.43, a normal-C24 to total C24 weight ratio less than 0.48, and a normal-C25 to total C25 weight ratio less than 0.53. In alternative embodiments the condensable hydrocarbon portion has one or more of a normal-C11 to total C11 weight ratio less than 0.30, a normal-C12 to total C12 weight ratio less than 0.27, a normal-C13 to total C13 weight ratio less than 0.26, a normal-C14 to total C14 weight ratio less than 0.29, a normal-C15 to total C15 weight ratio less than 0.24, a normal-C16 to total C16 weight ratio less than 0.25, a normal-C17 to total C17 weight ratio less than 0.29, a normal-C18 to total C18 weight ratio less than 0.31, normal-C19 to total C19 weight ratio less than 0.35, a normal-C20 to total C20 weight ratio less than 0.33, a normal-C21 to total C21 weight ratio less than 0.33, a normal-C22 to total C22 weight ratio less than 0.35, normal-C23 to total C23 weight ratio less than 0.40, a normal-C24 to total C24 weight ratio less than 0.45, and a normal-C25 to total C25 weight ratio less than 0.49. In alternative embodiments the condensable hydrocarbon portion has one or more of a normal-C11 to total C11 weight ratio less than 0.28, a normal-C12 to total C12 weight ratio less than 0.25, a normal-C13 to total C13 weight ratio less than 0.24, a normal-C14 to total C14 weight ratio less than 0.27, a normal-C15 to total C15 weight ratio less than 0.22, a normal-C16 to total C16 weight ratio less than 0.23, a normal-C17 to total C17 weight ratio less than 0.25, a normal-C18 to total C18 weight ratio less than 0.28, normal-C19 to total C19 weight ratio less than 0.31, a normal-C20 to total C20 weight ratio less than 0.29, a normal-C21 to total C21 weight ratio less than 0.30, a normal-C22 to total C22 weight ratio less than 0.28, normal-C23 to total C23 weight ratio less than 0.33, a normal-C24 to total C24 weight ratio less than 0.40, and a normal-C25 to total C25 weight ratio less than 0.45. As used in this paragraph and in the claims, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in this paragraph may be combined with any of the other aspects of the invention discussed herein.

In some embodiments the condensable hydrocarbon portion has a normal-C10 to total C10 weight ratio less than 0.31. Alternatively, the condensable hydrocarbon portion may have a normal-C10 to total C10 weight ratio less than 0.30 or less than 0.29. In alternative embodiments, the condensable hydrocarbon portion may have a normal-C10 to total C10 weight ratio greater than 0.15 or greater than 0.20. In some embodiments the condensable hydrocarbon portion has a normal-C11 to total C11 weight ratio less than 0.32. Alternatively, the condensable hydrocarbon portion may have a normal-C11 to total C11 weight ratio less than 0.31, less than 0.30, or less than 0.29. In alternative embodiments, the condensable hydrocarbon portion may have a normal-C11 to total C11 weight ratio greater than 0.15 or greater than 0.20. In some embodiments the condensable hydrocarbon portion has a normal-C12 to total C12 weight ratio less than 0.29. Alternatively, the condensable hydrocarbon portion may have a normal-C12 to total C12 weight ratio less than 0.26, or less than 0.24. In alternative embodiments, the condensable hydrocarbon portion may have a normal-C12 to total C12 weight ratio greater than 0.10 or greater than 0.15. In some embodiments the condensable hydrocarbon portion has a normal-C13 to total C13 weight ratio less than 0.28. Alternatively, the condensable hydrocarbon portion may have a normal-C13 to total C13 weight ratio less than 0.27, less than 0.25, or less than 0.23. In alternative embodiments, the condensable hydrocarbon portion may have a normal-C13 to total C13 weight ratio greater than 0.10 or greater than 0.15. In some embodiments the condensable hydrocarbon portion has a normal-C14 to total C14 weight ratio less than 0.31. Alternatively, the condensable hydrocarbon portion may have a normal-C14 to total C14 weight ratio less than 0.30, less than 0.28, or less than 0.26. In alternative embodiments, the condensable hydrocarbon portion may have a normal-C14 to total C14 weight ratio greater than 0.10 or greater than 0.15. In some embodiments the condensable hydrocarbon portion has a normal-C15 to total C15 weight ratio less than 0.27. Alternatively, the condensable hydrocarbon portion may have a normal-C15 to total C15 weight ratio less than 0.26, less than 0.24, or less than 0.22. In alternative embodiments, the condensable hydrocarbon portion may have a normal-C15 to total C15 weight ratio greater than 0.10 or greater than 0.15. In some embodiments the condensable hydrocarbon portion has a normal-C16 to total C16 weight ratio less than 0.31. Alternatively, the condensable hydrocarbon portion may have a normal-C16 to total C16 weight ratio less than 0.29, less than 0.26, or less than 0.24. In alternative embodiments, the condensable hydrocarbon portion may have a normal-C16 to total C16 weight ratio greater than 0.10 or greater than 0.15. In some embodiments the condensable hydrocarbon portion has a normal-C17 to total C17 weight ratio less than 0.31. Alternatively, the condensable hydrocarbon portion may have a normal-C17 to total C17 weight ratio less than 0.29, less than 0.27, or less than 0.25. In alternative embodiments, the condensable hydrocarbon portion may have a normal-C17 to total C17 weight ratio greater than 0.10 or greater than 0.15. In some embodiments the condensable hydrocarbon portion has a normal-C18 to total C18 weight ratio less than 0.37. Alternatively, the condensable hydrocarbon portion may have a normal-C18 to total C18 weight ratio less than 0.35, less than 0.31, or less than 0.28. In alternative embodiments, the condensable hydrocarbon portion may have a normal-C18 to total C18 weight ratio greater than 0.10 or greater than 0.15. In some embodiments the condensable hydrocarbon portion has a normal-C19 to total C19 weight ratio less than 0.37. Alternatively, the condensable hydrocarbon portion may have a normal-C19 to total C19 weight ratio less than 0.36, less than 0.34, or less than 0.31. In alternative embodiments, the condensable hydrocarbon portion may have a normal-C19 to total C19 weight ratio greater than 0.10 or greater than 0.15. In some embodiments the condensable hydrocarbon portion has a normal-C20 to total C20 weight ratio less than 0.37. Alternatively, the condensable hydrocarbon portion may have a normal-C20 to total C20 weight ratio less than 0.35, less than 0.32, or less than 0.29. In alternative embodiments, the condensable hydrocarbon portion may have a normal-C20 to total C20 weight ratio greater than 0.10 or greater than 0.15. In some embodiments the condensable hydrocarbon portion has a normal-C21 to total C21 weight ratio less than 0.37. Alternatively, the condensable hydrocarbon portion may have a normal-C21 to total C21 weight ratio less than 0.35, less than 0.32, or less than 0.30. In alternative embodiments, the condensable hydrocarbon portion may have a normal-C21 to total C21 weight ratio greater than 0.10 or greater than 0.15. In some embodiments the condensable hydrocarbon portion has a normal-C22 to total C22 weight ratio less than 0.38. Alternatively, the condensable hydrocarbon portion may have a normal-C22 to total C22 weight ratio less than 0.36, less than 0.34, or less than 0.30. In alternative embodiments, the condensable hydrocarbon portion may have a normal-C22 to total C22 weight ratio greater than 0.10 or greater than 0.15. In some embodiments the condensable hydrocarbon portion has a normal-C23 to total C23 weight ratio less than 0.43. Alternatively, the condensable hydrocarbon portion may have a normal-C23 to total C23 weight ratio less than 0.40, less than 0.35, or less than 0.29. In alternative embodiments, the condensable hydrocarbon portion may have a normal-C23 to total C23 weight ratio greater than 0.15 or greater than 0.20. In some embodiments the condensable hydrocarbon portion has a normal-C24 to total C24 weight ratio less than 0.48. Alternatively, the condensable hydrocarbon portion may have a normal-C24 to total C24 weight ratio less than 0.46, less than 0.42, or less than 0.40. In alternative embodiments, the condensable hydrocarbon portion may have a normal-C24 to total C24 weight ratio greater than 0.15 or greater than 0.20. In some embodiments the condensable hydrocarbon portion has a normal-C25 to total C25 weight ratio less than 0.48. Alternatively, the condensable hydrocarbon portion may have a normal-C25 to total C25 weight ratio less than 0.46, less than 0.42, or less than 0.40. In alternative embodiments, the condensable hydrocarbon portion may have a normal-C25 to total C25 weight ratio greater than 0.20 or greater than 0.25. Certain features of the present invention are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in this paragraph may be combined with any of the other aspects of the invention discussed herein.

The use of “total C_” (e.g., total C10) herein and in the claims is meant to refer to the amount of a particular pseudo component found in a condensable hydrocarbon fluid determined as described herein, particularly as described in the section labeled “Experiments” herein. That is “total C_” is determined using the whole oil gas chromatography (WOGC) analysis methodology according to the procedure described in the Experiments section of this application. Further, “total C_” is determined from the whole oil gas chromatography (WOGC) peak integration methodology and peak identification methodology used for identifying and quantifying each pseudo-component as described in the Experiments section herein. Further, “total C_” weight percent and mole percent values for the pseudo components were obtained using the pseudo component analysis methodology involving correlations developed by Katz and Firoozabadi (Katz, D. L., and A. Firoozabadi, 1978. Predicting phase behavior of condensate/crude-oil systems using methane interaction coefficients, J. Petroleum Technology (November 1978), 1649-1655) as described in the Experiments section, including the exemplary molar and weight percentage determinations.

The use of “normal-C_” (e.g., normal-C10) herein and in the claims is meant to refer to the amount of a particular normal alkane hydrocarbon compound found in a condensable hydrocarbon fluid determined as described herein, particularly in the section labeled “Experiments” herein. That is “normal-C_” is determined from the GC peak areas determined using the whole oil gas chromatography (WOGC) analysis methodology according to the procedure described in the Experiments section of this application. Further, “total C_” is determined from the whole oil gas chromatography (WOGC) peak identification and integration methodology used for identifying and quantifying individual compound peaks as described in the Experiments section herein. Further, “normal-C_” weight percent and mole percent values for the normal alkane compounds were obtained using methodology analogous to the pseudo component exemplary molar and weight percentage determinations explained in the Experiments section, except that the densities and molecular weights for the particular normal alkane compound of interest were used and then compared to the totals obtained in the pseudo component methodology to obtain weight and molar percentages.

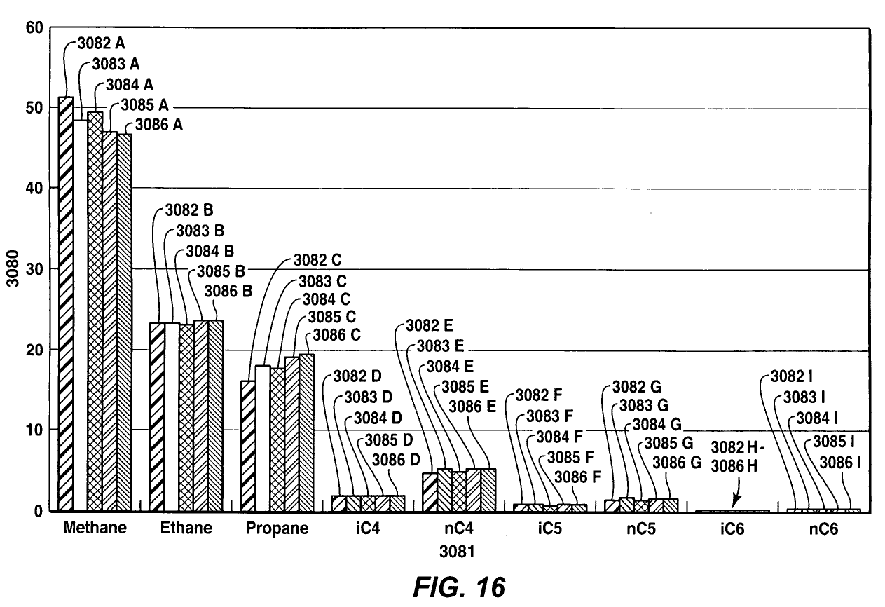

The following discussion of FIG. 16 concerns data obtained in Examples 1-5 which are discussed in the section labeled “Experiments”. The data was obtained through the experimental procedures, gas sample collection procedures, hydrocarbon gas sample gas chromatography (GC) analysis methodology, and gas sample GC peak identification and integration methodology discussed in the Experiments section. For clarity, when referring to gas chromatograms of gaseous hydrocarbon samples, graphical data is provided for one unstressed experiment through Example 1, two 400 psi stressed experiments through Examples 2 and 3, and two 1,000 psi stressed experiments through Examples 4 and 5.

FIG. 16 is a bar graph showing the concentration, in molar percentage, of the hydrocarbon species present in the gas samples taken from each of the three stress levels tested and analyzed in the laboratory experiments discussed herein. The gas compound molar percentages were obtained through the experimental procedures, gas sample collection procedures, hydrocarbon gas sample gas chromatography (GC) analysis methodology, gas sample GC peak integration methodology and molar concentration determination procedures described herein. For clarity, the hydrocarbon molar percentages are taken as a percentage of the total of all identified hydrocarbon gas GC areas (i.e., methane, ethane, propane, iso-butane, n-butane, iso-pentane, n-pentane, 2-methyl pentane, and n-hexane) and calculated molar concentrations. Thus the graphed methane to normal C6 molar percentages for all of the experiments do not include the molar contribution of any associated non-hydrocarbon gas phase product (e.g., hydrogen, CO₂ or H₂S), any of the unidentified hydrocarbon gas species listed in Tables 2, 4, 5, 7, or 9 (e.g., peak numbers 2, 6, 8-11, 13, 15-22, 24-26, and 28-78 in Table 2) or any of the gas species dissolved in the liquid phase which were separately treated in the liquid GC's. The y-axis 3080 represents the concentration in terms of molar percent of each gaseous compound in the gas phase. The x-axis 3081 contains the identity of each hydrocarbon compound from methane to normal hexane. The bars 3082A-I represent the molar percentage of each gaseous compound for the unstressed experiment of Example 1. That is 3082A represents methane, 3082B represents ethane, 3082C represents propane, 3082D represents iso-butane, 3082E represents normal butane, 3082F represents iso-pentane, 3082G represents normal pentane, 3082H represents 2-methyl pentane, and 3082I represents normal hexane. The bars 3083A-I and 3084A-I represent the molar percent of each gaseous compound for samples from the duplicate 400 psi stressed experiments of Examples 2 and 3, with the letters assigned in the manner described for the unstressed experiment. While the bars 3085A-I and 3086A-I represent the molar percent of each gaseous compound for the duplicate 1,000 psi stressed experiments of Examples 4 and 5, with the letters assigned in the manner described for the unstressed experiment. From FIG. 16 it can be seen that the hydrocarbon gas produced in all the experiments is primarily methane, ethane and propane on a molar basis. It is further apparent that the unstressed experiment, represented by bars 3082A-I, contains the most methane 3082A and least propane 3082C, both as compared to the 400 psi stress experiments hydrocarbon gases and the 1,000 psi stress experiments hydrocarbon gases. Looking now at bars 3083A-I and 3084A-I, it is apparent that the intermediate level 400 psi stress experiments produced a hydrocarbon gas having methane 3083A & 3084A and propane 3083C & 3084C concentrations between the unstressed experiment represented by bars 3082A & 3082C and the 1,000 psi stressed experiment represented by bars 3085A & 3085C and 3086A & 3086C. Lastly, it is apparent that the high level 1,000 psi stress experiments produced hydrocarbon gases having the lowest methane 3085A & 3086A concentration and the highest propane concentrations 3085C & 3086C, as compared to both the unstressed experiments represented by bars 3082A & 3082C and the 400 psi stressed experiment represented by bars 3083A & 3084A and 3083C & 3084C. Thus pyrolizing oil shale under increasing levels of lithostatic stress appears to produce hydrocarbon gases having decreasing concentrations of methane and increasing concentrations of propane.

The hydrocarbon fluid produced from the organic-rich rock formation may include both a condensable hydrocarbon portion (e.g. liquid) and a non-condensable hydrocarbon portion (e.g. gas). In some embodiments the non-condensable hydrocarbon portion includes methane and propane. In some embodiments the molar ratio of propane to methane in the non-condensable hydrocarbon portion is greater than 0.32. In alternative embodiments, the molar ratio of propane to methane in the non-condensable hydrocarbon portion is greater than 0.34, 0.36 or 0.38. As used herein “molar ratio of propane to methane” is the molar ratio that may be determined as described herein, particularly as described in the section labeled “Experiments” herein. That is “molar ratio of propane to methane” is determined using the hydrocarbon gas sample gas chromatography (GC) analysis methodology, gas sample GC peak identification and integration methodology and molar concentration determination procedures described in the Experiments section of this application.

In some embodiments the condensable hydrocarbon portion of the hydrocarbon fluid includes benzene. In some embodiments the condensable hydrocarbon portion has a benzene content between 0.1 and 0.8 weight percent. Alternatively, the condensable hydrocarbon portion may have a benzene content between 0.15 and 0.6 weight percent, a benzene content between 0.15 and 0.5, or a benzene content between 0.15 and 0.5.

In some embodiments the condensable hydrocarbon portion of the hydrocarbon fluid includes cyclohexane. In some embodiments the condensable hydrocarbon portion has a cyclohexane content less than 0.8 weight percent. Alternatively, the condensable hydrocarbon portion may have a cyclohexane content less than 0.6 weight percent or less than 0.43 weight percent. Alternatively, the condensable hydrocarbon portion may have a cyclohexane content greater than 0.1 weight percent or greater than 0.2 weight percent.

In some embodiments the condensable hydrocarbon portion of the hydrocarbon fluid includes methyl-cyclohexane. In some embodiments the condensable hydrocarbon portion has a methyl-cyclohexane content greater than 0.5 weight percent. Alternatively, the condensable hydrocarbon portion may have a methyl-cyclohexane content greater than 0.7 weight percent or greater than 0.75 weight percent. Alternatively, the condensable hydrocarbon portion may have a methyl-cyclohexane content less than 1.2 or 1.0 weight percent.

The use of weight percentage contents of benzene, cyclohexane, and methyl-cyclohexane herein and in the claims is meant to refer to the amount of benzene, cyclohexane, and methyl-cyclohexane found in a condensable hydrocarbon fluid determined as described herein, particularly as described in the section labeled “Experiments” herein. That is, respective compound weight percentages are determined from the whole oil gas chromatography (WOGC) analysis methodology whole oil gas chromatography (WOGC) peak identification and integration methodology discussed in the Experiments section herein. Further, the respective compound weight percentages were obtained as described for FIG. 11, except that each individual respective compound peak area integration was used to determine each respective compound weight percentage. For clarity, the compound weight percentages are taken as a percentage of the entire C3 to pseudo C38 whole oil gas chromatography areas and calculated weights as used in the pseudo compound data presented in FIG. 7.

In some embodiments the condensable hydrocarbon portion of the hydrocarbon fluid has an API gravity greater than 30. Alternatively, the condensable hydrocarbon portion may have an API gravity greater than 30, 32, 34, 36, 40, 42 or 44. As used herein and in the claims, API gravity may be determined by any generally accepted method for determining API gravity.

In some embodiments the condensable hydrocarbon portion of the hydrocarbon fluid has a basic nitrogen to total nitrogen ratio between 0.1 and 0.50. Alternatively, the condensable hydrocarbon portion may have a basic nitrogen to total nitrogen ratio between 0.15 and 0.40. As used herein and in the claims, basic nitrogen and total nitrogen may be determined by any generally accepted method for determining basic nitrogen and total nitrogen. Where results conflict, the generally accepted more accurate methodology shall control.

One embodiment of the invention includes an in situ method of producing hydrocarbon fluids with improved properties from an organic-rich rock formation. Applicants have surprisingly discovered that the quality of the hydrocarbon fluids produced from in situ heating and pyrolysis of an organic-rich rock formation may be improved by selecting sections of the organic-rich rock formation with a certain lithostatic stress for in situ heating and pyrolysis. Further, applicants have discovered that the temperature at which the in situ pyrolysis is accomplished has an effect on the composition of the produced fluid, that the effect of increasing temperature generally affects the composition of the produced fluid in the same direction as increasing lithostatic stress, and that the effect of decreasing temperature generally affects the composition of the produced fluid in the same direction as decreasing lithostatic stress. Further, applicants have discovered that the pressure at which the in situ pyrolysis is conducted affects the composition of the produced fluid, that the compositional effect of increasing pressure is generally in a direction opposite to the effects of lithostatic stress and temperature and that the compositional effect of pressure is generally of a much lower magnitude than the effects of temperature and lithostatic stress.

The method may include creating the hydrocarbon fluid by pyrolysis of a solid hydrocarbon and/or a heavy hydrocarbon present in the organic-rich rock formation. Embodiments may include the hydrocarbon fluid being partially, predominantly or substantially completely created by pyrolysis of the solid hydrocarbon and/or heavy hydrocarbon present in the organic-rich rock formation.

Applicants have found that in situ heating and pyrolysis of organic-rich rock formations with differing amounts of stress lead to the production of hydrocarbon fluids with changed properties. The method may include in situ heating of a section of the organic-rich rock formation that has a selected lithostatic stress to form hydrocarbon fluids with desired properties. Selecting or maintaining a higher lithostatic stress will increase the production of aromatic and cyclic hydrocarbon compounds, while decreasing the production of normal and isoprenoid (or branched) hydrocarbon compounds. Alternatively, maintaining a lower lithostatic stress will decrease the production of aromatic and cyclic hydrocarbon compounds, while increasing the production of normal and isoprenoid (or branched) hydrocarbon compounds. The method may include heating in situ a section of the organic-rich rock formation having a lithostatic stress greater than 200 psi and producing a hydrocarbon fluid from the heated section of the organic-rich rock formation. In alternative embodiments, the heated section of the organic-rich rock formation may have a lithostatic stress greater than 400 psi. In alternative embodiments, the heated section of the organic-rich rock formation may have a lithostatic stress greater than 800 psi, greater than 1,000 psi, greater than 1,200 psi, greater than 1,500 psi or greater than 2,000 psi depending on the composition desired. In alternative embodiments, the heated section of the organic-rich rock formation may have a lithostatic stress less than 800 psi, less than 1,000 psi, less than 1,500 psi, less than 2,500 psi or less than 3,000 psi depending on the composition desired. In alternative embodiments, the heated section of the organic-rich rock formation may have a lithostatic stress between 200 psi and 1,000 psi, between 200 psi and 900 psi, between 200 psi and 800 psi, between 200 psi and 700 psi or between 200 psi and 600 psi depending on the composition desired. In alternative embodiments, the heated section of the organic-rich rock formation may have a lithostatic stress between 800 psi and 3,000 psi, between 900 psi and 3,000 psi, between 1,000 psi and 3,000 psi, between 1,200 psi and 3,000 psi or between 1,500 psi and 3,000 psi depending on the composition desired.

Further, the method may include controlling the temperature or range of temperatures the section of the organic-rich rock formation experiences in order to effect the composition of the produced hydrocarbon fluids. For example, the heating rate of sources of in situ heat may be set or adjusted to affect the temperature profile of the section of the organic-rich rock formation. Further, the density or configuration of the sources of in situ heat may be implemented or adjusted to effect the composition of the produced hydrocarbon fluid. Higher temperatures will favor the production of aromatics and cyclic hydrocarbon compounds, while lower temperatures will favor the production of normal and isoprenoid (or branched) hydrocarbon compounds. Alternatively, lower temperatures will tend to decrease aromatic and cyclic hydrocarbon compound production while higher temperatures will tend to decrease production of normal and isoprenoid (or branched) hydrocarbon compounds. Thus, the method may include heating a section of the organic-rich rock formation to a maximum temperature above 270° C. Alternatively, the method may include heating the section of the organic-rich rock formation to a maximum temperature between 270° C. and 600° C., between 270° C. to 550° C., between 270° C. to 500° C., between 270° C. to 450° C., between 270° C. to 400° C. or between 270° C. to 350° C. depending on the composition desired. Alternatively, the method may include heating the section of the organic-rich rock formation to a maximum temperature between 350° C. and 500° C., between 350° C. to 550° C., between 350° C. to 600° C., between 350° C. to 650° C., between 350° C. to 700° C. or between 350° C. to 750° C. depending on the composition desired. The method may include heating the section of the organic-rich rock formation by any method, including any of the methods described herein. For example, the method may include heating the section of the organic-rich rock formation by electrical resistance heating. Further, the method may include heating the section of the organic-rich rock formation through use of a heated heat transfer fluid.

Further, the method may include maintaining a range of pressures in the section of the organic-rich rock formation in order to effect the composition of the produced hydrocarbon fluid. One method of maintaining a range of pressures in the section of the organic-rich rock formation includes selecting the section by estimating the section's lithostatic stress in order to limit the maximum pressure that such a section is likely to experience by relying on the creation of fractures to relieve the pressure force due to in situ heating. The effect of pressure when combined with lithostatic stress will tend to alter the effect of lithostatic stress on the composition of the produced fluid. Lower pressures when combined with lithostatic stress will tend to enhance production of aromatic and cyclic hydrocarbon compounds and decrease production of normal and isoprenoid (or branched) hydrocarbon compounds. Alternatively, higher pressures when combined with lithostatic stress will tend to incrementally reduce production of aromatic and cyclic hydrocarbon compounds and increase production of normal and isoprenoid (or branched) hydrocarbon compounds. Thus, the method may include maintaining the pressure of a heated section of an organic-rich rock formation above 200 psig and producing a hydrocarbon fluid from the heated section of the organic-rich rock formation. In alternative embodiments, the method may include maintaining the pressure of a heated section of the organic-rich rock formation below 3,000 psig. In alternative embodiments, the method may include maintaining the pressure of a heated section of the organic-rich rock formation below 2,500 psig, below 2,000 psig, below 2,500 psig, below 2,000 psig or below 1,500 psig depending on the composition desired. In alternative embodiments, the method may include allowing the pressure of a heated section of the organic-rich rock formation to reach a maximum pressure above 400 psig, above 500 psig, above 800 psig, above 1,000 psig, above 1,500 psig or above 2,000 psig depending on the composition desired. In alternative embodiments, the method may include allowing the pressure of a heated section of the organic-rich rock formation to reach a maximum pressure between 200 psig and 1,000 psig, between 200 psig and 900 psig, between 200 psig and 800 psig, between 200 psig and 700 psig or between 200 psig and 600 psig depending on the composition desired. In alternative embodiments, the method may include allowing the pressure of a heated section of the organic-rich rock formation to reach a maximum pressure between 800 psig and 3,000 psig, between 900 psig and 3,000 psig, between 1,000 psig and 3,000 psig, between 1,200 psig and 3,000 psig or between 1,500 psig and 3,000 psig depending on the composition desired.

The organic-rich rock formation may be, for example, a heavy hydrocarbon formation or a solid hydrocarbon formation. Particular examples of such formations may include an oil shale formation, a tar sands formation or a coal formation. Particular formation hydrocarbons present in such formations may include oil shale, kerogen, coal, and/or bitumen.

The hydrocarbon fluid produced from the organic-rich rock formation may include both a condensable hydrocarbon portion (e.g. liquid) and a non-condensable hydrocarbon portion (e.g. gas). The hydrocarbon fluid may additionally be produced together with non-hydrocarbon fluids. Exemplary non-hydrocarbon fluids include, for example, water, carbon dioxide, hydrogen sulfide, hydrogen, ammonia, and/or carbon monoxide.

The condensable hydrocarbon portion of the hydrocarbon fluid may be a fluid present within different locations associated with an organic-rich rock development project. For example, the condensable hydrocarbon portion of the hydrocarbon fluid may be a fluid present within a production well that is in fluid communication with the organic-rich rock formation. The production well may serve as a device for withdrawing the produced hydrocarbon fluids from the organic-rich rock formation. Alternatively, the condensable hydrocarbon portion may be a fluid present within processing equipment adapted to process hydrocarbon fluids produced from the organic-rich rock formation. Exemplary processing equipment is described herein. Alternatively, the condensable hydrocarbon portion may be a fluid present within a fluid storage vessel. Fluid storage vessels may include, for example, fluid storage tanks with fixed or floating roofs, knock-out vessels, and other intermediate, temporary or product storage vessels. Alternatively, the condensable hydrocarbon portion may be a fluid present within a fluid transportation pipeline. A fluid transportation pipeline may include, for example, piping from production wells to processing equipment or fluid storage vessels, piping from processing equipment to fluid storage vessels, or pipelines associated with collection or transportation of fluids to or from intermediate or centralized storage locations.

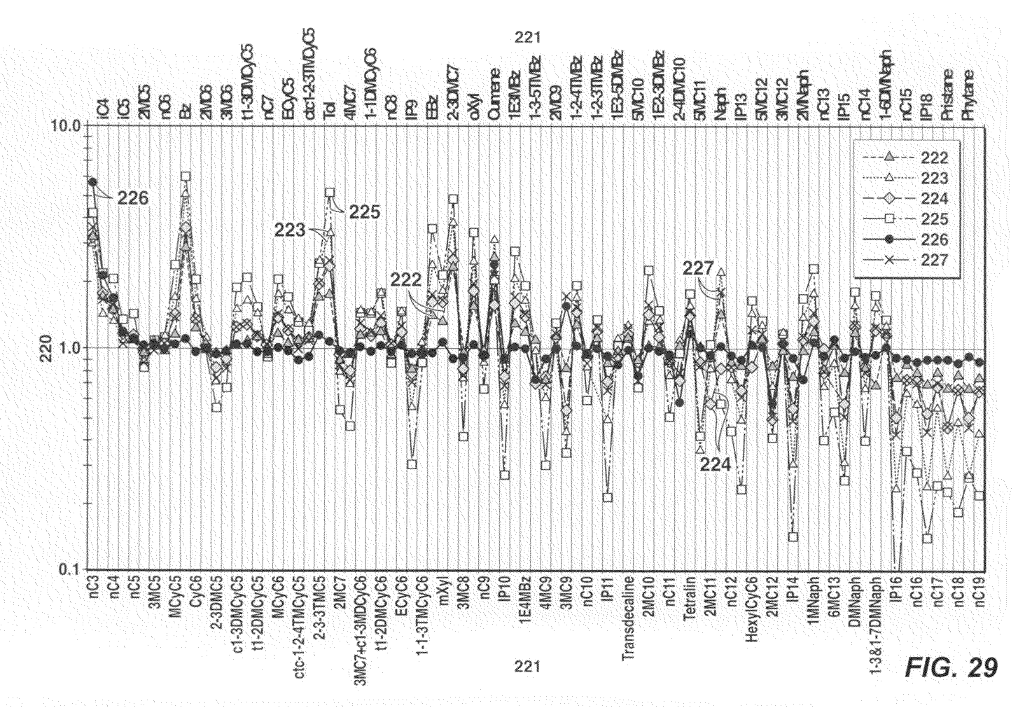

The following discussion of FIG. 29-38 concerns data obtained in Examples 6-19 which are discussed in the section labeled “Experiments”. The data was obtained through the experimental procedures, gas and liquid sample collection procedures, C4-C19 liquid sample gas chromatography (C4-C19 GC) analysis methodology, and C4-C19 liquid sample gas chromatography (C4-C19 GC) peak integration methodology discussed in the Experiments section. For clarity, when referring to C4-C19 liquid sample gas chromatography (C4-C19 GC) chromatograms of liquid hydrocarbon samples, graphical data is provided for Examples 6-19 in FIGS. 29-52 while peak area information may be found in Table 16 in the Experiments section.

FIG. 29 is a graph of the weight ratio of each identified compound occurring from n-C4 to n-C19 for each of the six 393° C. experiments tested and analyzed in the laboratory experiments (Examples 13-19) discussed herein compared to the weight ratio of each identified compound occurring from n-C4 to n-C19 for Example 13 conducted at 393° C., 500 psig initial argon pressure and 0 psi stress. The compound weight ratios were obtained through the experimental procedures, liquid sample collection procedures, C4-C19 liquid sample gas chromatography (C4-C19 GC) analysis methodology, C4-C19 gas chromatography peak identification and integration methodology, and C4-C19 compound analysis methodology discussed in the Experiments section. For clarity, the compound weight ratios were derived as a ratio of a particular compound's percentage of the total peak area in one experiment to the same compound's percentage of the total peak area for the 393/500/0 experiment (Experiment 13). When referring to experimental conditions herein, the notational format “Temperature (° C.)/Initial Argon Pressure (psig)/Stress load (psi)” will be used as a shorthand to refer to the temperature, initial argon pressure and stress loading of a particular experiment. For example, the notation “393/500/0” refers to an experiment conducted at 393° C., 500 psig initial argon pressure and 0 psi stress load as present in Example 13. Thus the graphed n-C4 to n-C19 weight ratios do not include the weight contribution of the associated gas phase product from any of the experiments. Further, the graphed weight ratios do not include the weight contribution of any liquid hydrocarbon compounds heavier than (i.e. having a longer retention time than) n-C19 or any unidentified (i.e., not listed in FIG. 29 or Table 16) compounds from the C4-C19 GC data. The y-axis 220 represents the weight ratio of a particular compound for a given experiment to the same compound for the 393/500/0 experiment (Experiment 13). The x-axis 221 contains the identity of each identified compound from n-C4 to n-C19. The data points occurring on line 222 represent the weight ratio of each identified n-C4 to n-C19 compound for the 393/500/400 experiment of Example 15 to the 393/500/0 experiment of Experiment 13. The data points occurring on line 223 represent the weight ratio of each identified n-C4 to n-C19 compound for the 393/500/1000 experiment of Example 18 to the 393/500/0 experiment of Experiment 13. The data points occurring on line 224 represent the weight ratio of each identified n-C4 to n-C19 compound for the 393/200/400 experiment of Example 16 to the 393/500/0 experiment of Experiment 13. The data points occurring on line 225 represent the weight ratio of each identified n-C4 to n-C19 compound for the 393/200/1000 experiment of Example 19 to the 393/500/0 experiment of Experiment 13. The data points occurring on line 226 represent the weight ratio of each identified n-C4 to n-C19 compound for the 393/200/0 experiment of Example 14 to the 393/500/0 experiment of Experiment 13. The data points occurring on line 227 represent the weight ratio of each identified n-C4 to n-C19 compound for the 393/50/400 experiment of Example 17 to the 393/500/0 experiment of Experiment 13.

From FIG. 29 it can be seen that the hydrocarbon liquids produced in the two 1,000 psi stressed experiments, represented by data points on line 223 & 225, generally contain an increased weight ratio of aromatic hydrocarbon compounds, including for example benzene (Bz), toluene (Tol), ethylbenzene (EBz), ortho-xylene (oXyl), meta-xylene (mXyl), 1-ethyl-3-methylbenzene (1E3 MBz), 1-ethyl-4-methylbenzene (1E4 MBz), 1,2,4-trimethylbenzene (1-2-4TMBz), 1-ethyl-2,3-dimethylbenzene (1E2-3DMBz), tetralin, 2-methylnaphthalene (2MNaph), 1-methylnaphthalene (1MNaph). It also can be seen that for the two 1,000 psi stress experiments, the lower initial argon pressure (200 psig argon) experiment represented by line 225 is generally more enriched in aromatic compounds relative to the higher initial argon pressure (500 psig argon) experiment represented by line 223. From FIG. 29 it can also be seen that the hydrocarbon liquids produced in the three 400 psi stressed experiments, represented by data points on line 222, 224 & 227, generally contain an increased weight ratio of aromatic hydrocarbon compounds relative to the unstressed experiments (i.e., line 226 & the “1” line on the y-axis representing Experiments 13 & 14) but a lower weight ratio of aromatic hydrocarbon compounds relative to the 1,000 psi stressed experiments (Lines 223 & 225). It also can be seen that for the three 400 psi stress experiments, the lowest initial argon pressure (50 psig argon) experiment represented by line 227 is generally more enriched in aromatic compounds relative to the middle initial argon pressures (200 psig argon) experiment represented by line 224 and the highest initial argon pressures (500 psig argon) experiment represented by line 222, with the middle initial argon pressures (200 psig argon) experiment represented by line 224 generally falling between the highest and lowest initial argon pressure experiments. Thus pyrolyzing oil shale under increasing levels of stress appears to enrich the produced hydrocarbon liquid in aromatic compounds while decreasing pressure appears to enhance aromatic compound production.

From FIG. 29 it can also be seen that the hydrocarbon liquids produced in the two 1,000 psi stressed experiments, represented by data points on line 223 & 225, generally contain an increased weight ratio of cyclic hydrocarbon compounds, including for example cis 1,3-dimethyl cyclopentane (cl-3DMCyC5), trans 1,3-dimethyl cyclopentane (t1-3DMCyC5), trans 1,2-dimethyl cyclopentane (t1-2DMCyC5), methyl cyclohexane (MCyC6), ethyl cyclopentane (ECyC5), 1,1-dimethyl cyclohexane (1-1DMCyC6), trans 1,2-dimethyl cyclohexane (1-2DMCyC6), and ethyl cyclohexane (ECyC6). It also can be seen that for the two 1,000 psi stress experiments, the lower initial argon pressure (200 psig argon) experiment represented by line 225 is generally more enriched in cyclic compounds relative to the higher initial argon pressure (500 psig argon) experiment represented by line 223. From FIG. 29 it can also be seen that the hydrocarbon liquid produced in the three 400 psi stressed experiments, represented by data points on line 222, 224 & 227, generally contain an increased weight ratio of cyclic hydrocarbon compounds relative to the unstressed experiments (i.e., line 226 & the “1” line on the y-axis representing Experiments 13 & 14) but a lower weight ratio of cyclic hydrocarbon compounds relative to the 1,000 psi stressed experiments (Lines 223 & 225). It also can be seen that for the three 400 psi stress experiments, the lowest initial argon pressure (50 psig argon) experiment represented by line 227 is generally more enriched in cyclic compounds relative to the middle initial argon pressures (200 psig argon) experiment represented by line 224 and the highest initial argon pressures (500 psig argon) experiment represented by line 222, with the middle initial argon pressures (200 psig argon) experiment represented by line 224 generally falling between the highest and lowest initial argon pressure experiments. Thus pyrolyzing oil shale under increasing levels of stress appears to enrich the produced hydrocarbon liquid in cyclic hydrocarbon compounds while decreasing pressure appears to enhance cyclic hydrocarbon compound production.

From FIG. 29 it can also be seen that the hydrocarbon liquids produced in the two 1,000 psi stressed experiments, represented by data points on line 223 & 225, generally contain a decreased weight ratio of normal alkane hydrocarbon compounds for n-C8 and heavier normal alkane hydrocarbon compounds, including for example n-C9 through n-C19. It also can be seen that for the two 1,000 psi stress experiments, the lower initial argon pressure (200 psig argon) experiment represented by line 225 is generally more depleted of normal hydrocarbon compounds relative to the higher initial argon pressure (500 psig argon) experiment represented by line 223. From FIG. 29 it can also be seen that the hydrocarbon liquid produced in the three 400 psi stressed experiments, represented by data points on line 222, 224 & 227, generally contain a decreased weight amount of normal hydrocarbon compounds relative to the unstressed experiments (i.e., line 226 & the “1” line on the y-axis representing Experiments 13 & 14) but a less depleted weight ratio of normal hydrocarbon compounds relative to the 1,000 psi stressed experiments (Lines 223 & 225). It also can be seen that for the three 400 psi stress experiments, the lowest initial argon pressure (50 psig argon) experiment represented by line 227 is generally more depleted of normal compounds relative to the middle initial argon pressures (200 psig argon) experiment represented by line 224 and the highest initial argon pressures (500 psig argon) experiment represented by line 222, with the middle initial argon pressures (200 psig argon) experiment represented by line 224 generally falling between the highest and lowest initial argon pressure experiments. Thus pyrolyzing oil shale under increasing levels of stress appears to deplete the produced hydrocarbon liquid in normal hydrocarbon compounds while decreasing pressure also appears to decrease normal hydrocarbon compound production.

From FIG. 29 it can also be seen that the hydrocarbon liquids produced in the two 1,000 psi stressed experiments, represented by data points on line 223 & 225, generally contain a decreased weight ratio of isoprenoid hydrocarbon compounds, including for example IP-9, IP-10, IP-11, IP-13, IP-14, IP-16, IP-18, pristane and phytane. It also can be seen that for the two 1,000 psi stress experiments, the lower initial argon pressure (200 psig argon) experiment represented by line 225 is generally more depleted of isoprenoid hydrocarbon compounds relative to the higher initial argon pressure (500 psig argon) experiment represented by line 223. From FIG. 29 it can also be seen that the hydrocarbon liquid produced in the three 400 psi stressed experiments, represented by data points on line 222, 224 & 227, generally contain a decreased weight amount of isoprenoid hydrocarbon compounds relative to the unstressed experiments (i.e., line 226 & the “1” line on the y-axis representing Experiment 13 & 14) but a less depleted weight ratio of isoprenoid hydrocarbon compounds relative to the 1,000 psi stressed experiments (Lines 223 & 225). It also can be seen that for the three 400 psi stress experiments, the lowest initial argon pressure (50 psig argon) experiment represented by line 227 is generally more depleted of isoprenoid compounds relative to the middle initial argon pressures (200 psig argon) experiment represented by line 224 and the highest initial argon pressures (500 psig argon) experiment represented by line 222, with the middle initial argon pressures (200 psig argon) experiment represented by line 224 generally falling between the highest and lowest initial argon pressure experiments. Thus pyrolyzing oil shale under increasing levels of stress appears to deplete the produced hydrocarbon liquid in isoprenoid hydrocarbon compounds while decreasing pressure also appears to decrease isoprenoid hydrocarbon compound production.

Isoprenoid hydrocarbon compounds are hydrocarbon compounds based on the isoprene structure. They are constructed by linking 2 or more 5 carbon isoprene units together building molecules with up to 40 or more carbon atoms. Isoprene is a diolefin but the double bonds are typically saturated during diagenesis so compounds built up from isoprene units are referred to as isoprenoids. Although the 5 carbon isoprene unit implies that isoprenoids should contain carbons in multiples of 5 this is not the case as carbons can be cleaved off during diagenesis. The use of “IP-_” (e.g., IP-10) herein and in the claims is meant to refer to a hydrocarbon structure based on isoprene with the number following the hyphen denoting the carbon number of a particular isoprenoid. For example, IP-10 means a hydrocarbon structure based on isoprene having 10 carbon atoms.

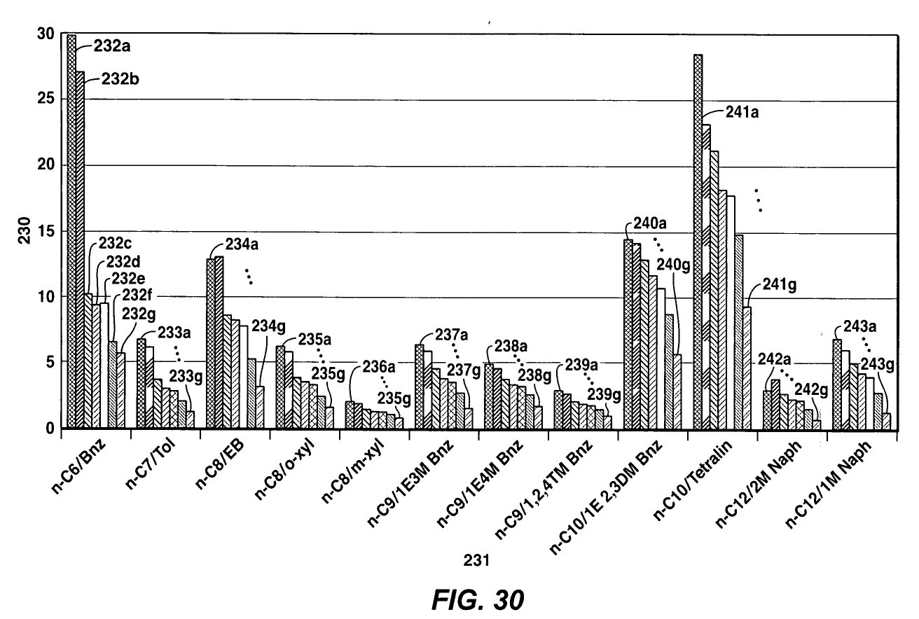

FIG. 30 is a bar graph of the weight ratio of several normal hydrocarbon compounds to like carbon number aromatic hydrocarbon compounds for each of the seven 393° C. experimental data discussed in Examples 13-19 in the Experimental section herein. The compound weight ratios were obtained through the experimental procedures, sample collection and analytical techniques discussed above for FIG. 29 and in the Experiments section. Except that, the normal hydrocarbon compound to aromatic hydrocarbon compound weight ratios are derived as a ratio of a particular normal hydrocarbon compound's peak area in one experiment to a particular aromatic hydrocarbon compound's peak area for the same particular experiment. Thus the graphed weight ratios represent a weight ratio of two different compounds produced in the same experiment. The y-axis 230 represents the weight ratio of two compounds for a given experiment. The x-axis 231 contains the identity of each depicted compound ratio. The bars 232a-g represent the weight ratio of n-C6/benzene (n-C6/Bnz). The bars 233a-g represent the weight ratio of n-C7/toluene (n-C7/Tol). The bars 234a-g represent the weight ratio of n-C8/ethylbenzene (n-C8/EB). The bars 235a-g represent the weight ratio of n-C8/ortho-xylene (n-C8%-xyl). The bars 236a-g represent the weight ratio of n-C8/meta-xylene (n-C8/m-xyl). The bars 237a-g represent the weight ratio of n-C9/1-ethyl-3-methylbenzene (1E3M Bnz). The bars 238a-g represent the weight ratio of n-C9/1-ethyl-4-methylbenzene (n-C9/1E4M Bnz). The bars 239a-g represent the weight ratio of n-C9/1,2,4-trimethylbenzene (n-C9/1,2,4™ Bnz). The bars 240a-g represent the weight ratio of n-C10/1-ethyl-2,3-dimethylbenzene (n-C10/1E 2,3DM Bnz). The bars 241a-g represent the weight ratio of n-C10/tetralin. The bars 242a-g represent the weight ratio of n-C12/2-methylnaphthalene (n-C12/2M Naph). The bars 243a-g represent the weight ratio of n-C12/1-methylnaphthalene (n-C12/1M Naph). For each of the compound ratio groups, the “a” designation denotes the 393/500/0 experiment, the “b” designation denotes the 393/200/0 experiment, the “c” designation denotes the 393/500/400 experiment, the “d” designation denotes the 393/200/400 experiment, the “e” designation denotes the 393/50/400 experiment, the “f” designation denotes the 393/500/1000 experiment, while the “g” designation denotes the 393/200/1000 experiment.

From FIG. 30 it can be seen that the hydrocarbon liquids produced in the two 1,000 psi stressed experiments, represented by the “f” and “g” bars of 232-243, generally contain the most respective aromatic hydrocarbon compounds, including for example benzene, toluene, ethylbenzene, ortho-xylene, meta-xylene, 1-ethyl-3-methylbenzene, 1-ethyl-4-methylbenzene, 1,2,4-trimethylbenzene, 1-ethyl-2,3-dimethylbenzene, tetralin, 2-methylnaphthalene, and 1-methylnaphthalene, relative to the respective corresponding same carbon number normal hydrocarbon compound. It also can be seen that for the two 1,000 psi stress experiments, the lower initial argon pressure (200 psig argon) experiment represented by the “g” bars is generally more enriched in aromatic compounds relative to the higher initial argon pressure (500 psig argon) experiment represented by the “f” bars. From FIG. 30 it can also be seen that the hydrocarbon liquids produced in the three 400 psi stressed experiments, represented by the “c”, “d” and “f” bars, generally contain an increased amount of aromatic hydrocarbon compounds relative to the unstressed experiments (i.e., bars “a” and “b”) but a lower amount of aromatic hydrocarbon compounds relative to the 1,000 psi stressed experiments (bars “f” and “g”). It also can be seen that for the three 400 psi stress experiments, the lowest initial argon pressure (50 psig argon) experiment represented by the “e” bars is generally more enriched in aromatic compounds relative to the middle initial argon pressures (200 psig argon) experiment represented by the “d” bars and the highest initial argon pressures (500 psigg argon) experiment represented by the “c” bars, with the middle initial argon pressures (200 psig argon) experiment represented the “d” bars generally falling between the highest and lowest initial argon pressure experiments. By comparing the effect of the 0, 400, and 1,000 psi stressed data at consistent initial argon pressures (i.e., the a, c, and f data all at 500 psig initial argon or the b, d, and g data all at 200 psig initial argon) to the three 400 psi stress at different initial argon pressures data (i.e., the c, d, and e data), it becomes apparent that the compositional changes due to step changes in stress (i.e., 0 psi to 400 psi and 400 psi to 1,000 psi) are much more pronounced than the compositional changes due to step changes in pressure (i.e., 50 psig to 200 psig and 200 psig to 500 psig). It is also noted that the effect of increased pressure appears to partially retard the effect of increased stress. Thus pyrolyzing oil shale under increasing levels of stress appears to enrich the produced hydrocarbon liquid in aromatic compounds while decreasing pressure appears to enhance aromatic compound production. Further, the magnitude of compositional changed due to changes in stress appears to be more significant than the magnitude of the compositional change due to changes in pressure.

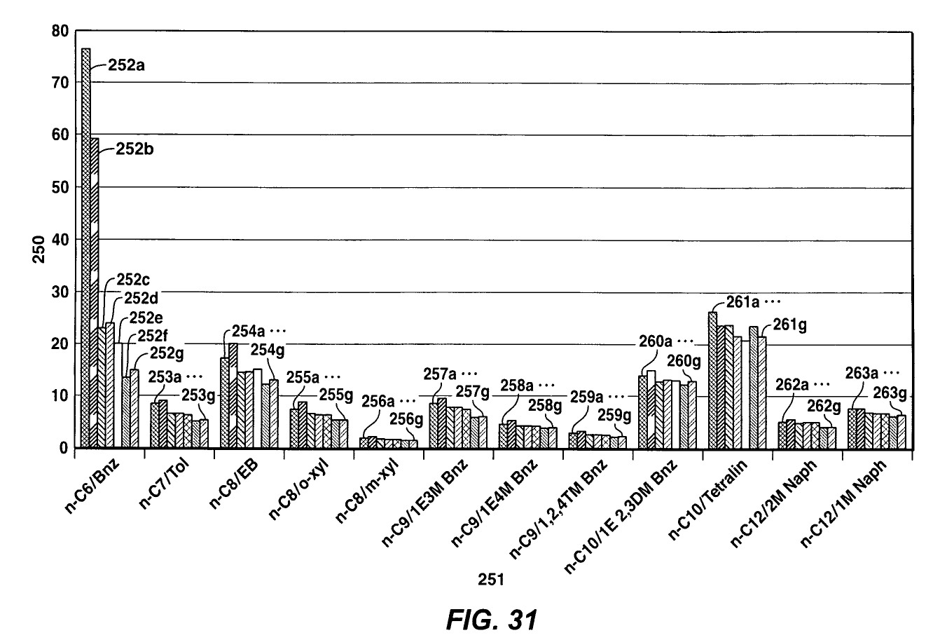

FIG. 31 is a bar graph of the weight ratio of the normal hydrocarbon compounds to like carbon number aromatic hydrocarbon compounds for each of the seven 375° C. experimental data discussed in Examples 6-12 in the Experimental section herein. The compound weight ratios were obtained through the experimental procedures, sample collection and analytical techniques discussed above for FIG. 30 and in the Experiments section. For clarity, the normal hydrocarbon compound to aromatic hydrocarbon compound weight ratios are derived as a ratio of a particular normal hydrocarbon compound's peak area in one experiment to a particular aromatic hydrocarbon compound's peak area for the same particular experiment. Thus the graphed weight ratios represent a weight ratio of two different compounds produced in the same experiment. The y-axis 250 represents the weight ratio of two compounds for a given experiment. The x-axis 251 contains the identity of each depicted compound ratio. The bars 252a-g represent the weight ratio of n-C6/benzene (n-C6/Bnz). The bars 253a-g represent the weight ratio of n-C7/toluene (n-C7/Tol). The bars 254a-g represent the weight ratio of n-C8/ethylbenzene (n-C8/EB). The bars 255a-g represent the weight ratio of n-C8/ortho-xylene (n-C8%-xyl). The bars 256a-g represent the weight ratio of n-C8/meta-xylene (n-C8/m-xyl). The bars 257a-g represent the weight ratio of n-C9/1-ethyl-3-methylbenzene (1E3M Bnz). The bars 258a-g represent the weight ratio of n-C9/1-ethyl-4-methylbenzene (n-C9/1E4M Bnz). The bars 259a-g represent the weight ratio of n-C9/1,2,4-trimethylbenzene (n-C9/1,2,4™ Bnz). The bars 260a-g represent the weight ratio of n-C10/1-ethyl-2,3-dimethylbenzene (n-C10/1E 2,3DM Bnz). The bars 261a-g represent the weight ratio of n-C10/tetralin. The bars 262a-g represent the weight ratio of n-C12/2-methylnaphthalene (n-C12/2M Naph). The bars 263a-g represent the weight ratio of n-C12/1-methylnaphthalene (n-C12/1M Naph). For each of the compound ratio groups, the “a” designation denotes the 375/500/0 experiment, the “b” designation denotes the 375/200/0 experiment, the “c” designation denotes the 375/500/400 experiment, the “d” designation denotes the 375/200/400 experiment, the “e” designation denotes the 375/50/400 experiment, the “f” designation denotes the 375/500/1000 experiment, while the “g” designation denotes the 375/200/1000 experiment.

From FIG. 31 it can be seen that the hydrocarbon liquids produced in the two 1,000 psi stressed experiments, represented by the “f” and “g” bars of 252-263, generally contain the most respective aromatic hydrocarbon compounds, including for example benzene, toluene, ethylbenzene, ortho-xylene, meta-xylene, 1-ethyl-3-methylbenzene, 1-ethyl-4-methylbenzene, 1,2,4-trimethylbenzene, 1-ethyl-2,3-dimethylbenzene, tetralin, 2-methylnaphthalene, and 1-methylnaphthalene, relative to the respective corresponding same carbon number normal hydrocarbon compound. It also can be seen that for the two 1,000 psi stress experiments, the lower initial argon pressure (200 psig argon) experiment represented by the “g” bars is generally more enriched in aromatic compounds relative to the higher initial argon pressure (500 psig argon) experiment represented by the “f” bars. From FIG. 31 it can also be seen that the hydrocarbon liquids produced in the three 400 psi stressed experiments, represented by the “c”, “d” and “f” bars, generally contain an increased amount of aromatic hydrocarbon compounds relative to the unstressed experiments (i.e., bars “a” and “b”) but a lower amount of aromatic hydrocarbon compounds relative to the 1,000 psi stressed experiments (bars “f” and “g”). It also can be seen that for the three 400 psi stress experiments, the lowest initial argon pressure (50 psig argon) experiment represented by the “e” bars is generally more enriched in aromatic compounds relative to the middle initial argon pressures (200 psig argon) experiment represented by the “d” bars and the highest initial argon pressures (500 psig argon) experiment represented by the “c” bars, with the middle initial argon pressures (200 psig argon) experiment represented the “d” bars generally falling between the highest and lowest initial argon pressure experiments. While the ordering of the 375° C. data is not as consistent as the 393° C. data and magnitude of the differentiation between the ratios for different experimental parameters is not as great for the 375° C. data, the same general trends are apparent for the 375° C. data as observed with respect to the 393° C. data. Thus pyrolyzing oil shale under increasing levels of stress at more moderate temperatures appears to enrich the produced hydrocarbon liquid in aromatic compounds while decreasing pressure appears to enhance aromatic compound production. However, the trends are less consistent and less pronounced at the reduced temperature.

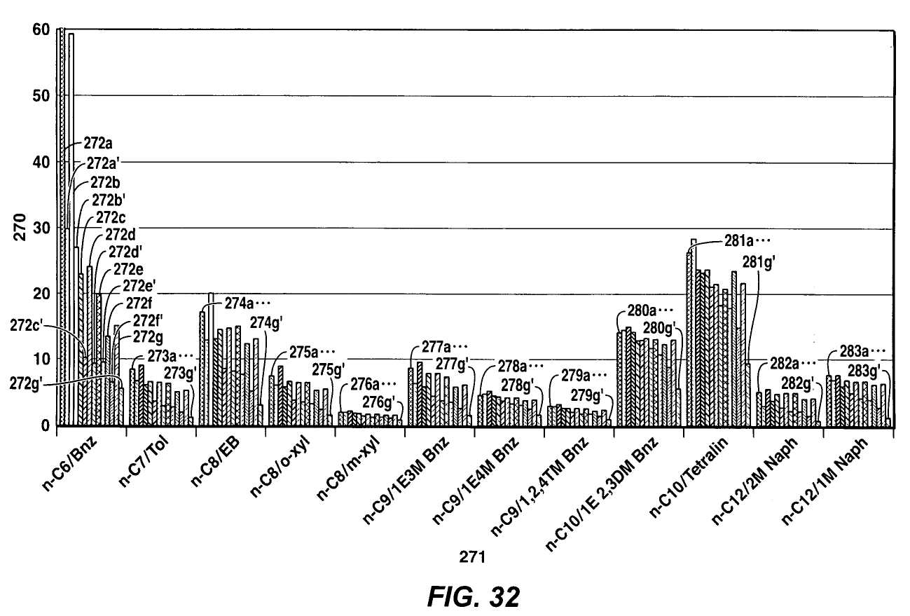

FIG. 32 is a bar graph of the weight ratio of the normal hydrocarbon compounds to like carbon number aromatic hydrocarbon compounds for each of the seven 375° C. and seven 393° C. experimental data discussed in Examples 6-19 in the Experimental section herein. The compound weight ratios were obtained through the experimental procedures, sample collection and analytical techniques discussed above for FIG. 30 and in the Experiments section. For clarity, the normal hydrocarbon compound to aromatic hydrocarbon compound weight ratios are derived as a ratio of a particular normal hydrocarbon compound's peak area in one experiment to a particular aromatic hydrocarbon compound's peak area for the same particular experiment. Thus the graphed weight ratios represent a weight ratio of two different compounds produced in the same experiment. The y-axis 270 represents the weight ratio of two compounds for a given experiment. The x-axis 271 contains the identity of each depicted compound ratio. The bars 272a-g represent the weight ratio of n-C6/benzene (n-C6/Bnz) at 375° C. while the bars 272a′-g′ represent the weight ratio of n-C6/benzene at 393° C. It can be seen that the 76.4 value of bar 272a exceeds the y-axis scale of 60.0. The bars 273a-g represent the weight ratio of n-C7/toluene (n-C7/Tol) at 375° C. while the bars 273a′-g′ represent the weight ratio of n-C7/toluene at 393° C. The bars 274a-g represent the weight ratio of n-C8/ethylbenzene (n-C8/EB) at 375° C. while the bars 274a′-g′ represent the weight ratio of n-C8/ethylbenzene at 393° C. The bars 275a-g represent the weight ratio of n-C8/ortho-xylene (n-C8%-xyl) at 375° C. while the bars 275a′-g′ represent the weight ratio of C8/ortho-xylene at 393° C. The bars 276a-g represent the weight ratio of n-C8/meta-xylene (n-C8/m-xyl) at 375° C. while the bars 276a′-g′ represent the weight ratio of n-C8/meta-xylene at 393° C. The bars 277a-g represent the weight ratio of n-C9/1-ethyl-3-methylbenzene (1E3M Bnz) at 375° C. while the bars 277a′-g′ represent the weight ratio of n-C9/1-ethyl-3-methylbenzene at 393° C. The bars 278a-g represent the weight ratio of n-C9/1-ethyl-4-methylbenzene (n-C9/1E4M Bnz) at 375° C. while the bars 278a′-g′ represent the weight ratio of n-C9/1-ethyl-4-methylbenzene at 393° C. The bars 279a-g represent the weight ratio of n-C9/1,2,4-trimethylbenzene (n-C9/1,2,4™ Bnz) at 375° C. while the bars 279a′-g′ represent the weight ratio of n-C9/1,2,4-trimethylbenzene at 393° C. The bars 280a-g represent the weight ratio of n-C10/1-ethyl-2,3-dimethylbenzene (n-C10/1E 2,3DM Bnz) at 375° C. while the bars 280a′-g′ represent the weight ratio of n-C10/1-ethyl-2,3-dimethylbenzene at 393° C. The bars 281a-g represent the weight ratio of n-C10/tetralin at 375° C. while the bars 281a′-g′represent the weight ratio of n-C10/tetralin at 393° C. The bars 282a-g represent the weight ratio of n-C12/2-methylnaphthalene (n-C12/2M Naph) at 375° C. while the bars 282a′-g′ represent the weight ratio of n-C12/2-methylnaphthalene at 393° C. The bars 283a-g represent the weight ratio of n-C12/1-methylnaphthalene (n-C12/1M Naph) at 375° C. while the bars 283a′-g′ represent the weight ratio of n-C12/1-methylnaphthalene at 393° C. For each of the compound ratio groups, the “a” designation denotes the 375/500/0 experiment, the “a′” designation denotes the 393/500/0 experiment, the “b” designation denotes the 375/200/0 experiment, the “b′” designation denotes the 393/200/0 experiment, the “c” designation denotes the 375/500/400 experiment, the “c′” designation denotes the 393/500/400 experiment, the “d” designation denotes the 375/200/400 experiment, the “d′” designation denotes the 393/200/400 experiment, the “e” designation denotes the 375/50/400 experiment, the “e′” designation denotes the 393/50/400 experiment, the “f” designation denotes the 375/500/1000 experiment, the “f” designation denotes the 393/500/1000 experiment, the “g” designation denotes the 375/200/1000 experiment, while the “g′” designation denotes the 393/200/1000 experiment.

From FIG. 32 it can be seen that the hydrocarbon liquids produced in the 393° C. experiments, represented by the “a′-g′” bars of 272-283, generally contain more of the respective aromatic hydrocarbon compounds as compared to the corresponding experiment completed at the same pressure and stress but at the lower 375° C. temperature, as represented by the “a-g” bars of 272-283. By comparing each respective neighboring pair of bars (e.g., a and a′, b and b′, etc.) for each corresponding pair of experiments differing only in temperature conditions it can also be seen that the effect of temperature is similar in magnitude, but more pronounced than the effect of stress and that the effect of temperature and stress are both much more pronounced than the effect of pressure on aromatic hydrocarbon compound production. It is also noted that the effect of increased pressure appears to partially retard the effect of stress.

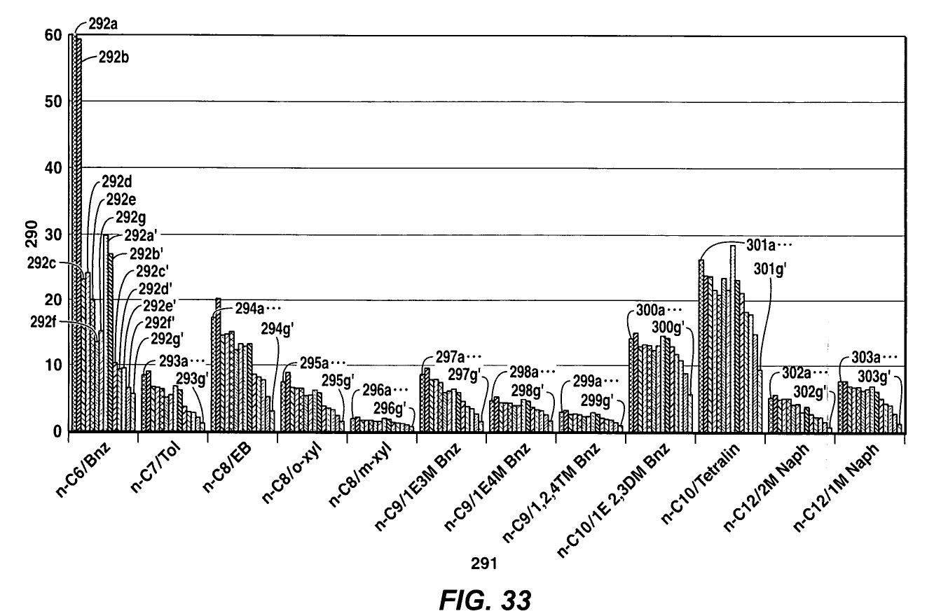

FIG. 33 is a bar graph of the weight ratio of the normal hydrocarbon compounds to like carbon number aromatic hydrocarbon compounds for each of the seven 375° C. and seven 393° C. experimental data discussed in Examples 6-19 in the Experimental section herein. The compound weight ratios were obtained through the experimental procedures, sample collection and analytical techniques discussed above for FIG. 30 and in the Experiments section. For clarity, the normal hydrocarbon compound to aromatic hydrocarbon compound weight ratios are derived as a ratio of a particular normal hydrocarbon compound's peak area in one experiment to a particular aromatic hydrocarbon compound's peak area for the same particular experiment. Thus the graphed weight ratios represent a weight ratio of two different compounds produced in the same experiment. The y-axis 290 represents the weight ratio of two compounds for a given experiment. The x-axis 291 contains the identity of each depicted compound ratio. The bars 292a-g represent the weight ratio of n-C6/benzene (n-C6/Bnz) at 375° C. while the bars 292a′-g′ represent the weight ratio of n-C6/benzene at 393° C. It can be seen that the 76.4 value of bar 292a exceeds the y-axis scale of 60.0. The bars 293a-g represent the weight ratio of n-C7/toluene (n-C7/Tol) at 375° C. while the bars 293a′-g′ represent the weight ratio of n-C7/toluene at 393° C. The bars 294a-g represent the weight ratio of n-C8/ethylbenzene (n-C8/EB) at 375° C. while the bars 294a′-g′ represent the weight ratio of n-C8/ethylbenzene at 393° C. The bars 295a-g represent the weight ratio of n-C8/ortho-xylene (n-C8%-xyl) at 375° C. while the bars 295a′-g′ represent the weight ratio of n-C8/ortho-xylene at 393° C. The bars 296a-g represent the weight ratio of n-C8/meta-xylene (n-C8/m-xyl) at 375° C. while the bars 296a′-g′ represent the weight ratio of n-C8/meta-xylene at 393° C. The bars 297a-g represent the weight ratio of n-C9/1-ethyl-3-methylbenzene (1E3M Bnz) at 375° C. while the bars 297a′-g′ represent the weight ratio of n-C9/1-ethyl-3-methylbenzene at 393° C. The bars 298a-g represent the weight ratio of n-C9/1-ethyl-4-methylbenzene (n-C9/1E4M Bnz) at 375° C. while the bars 298a′-g′ represent the weight ratio of n-C9/1-ethyl-4-methylbenzene at 393° C. The bars 279a-g represent the weight ratio of n-C9/1,2,4-trimethylbenzene (n-C9/1,2,4™ Bnz) at 375° C. while the bars 299a′-g′ represent the weight ratio of n-C9/1,2,4-trimethylbenzene at 393° C. The bars 300a-g represent the weight ratio of n-C10/1-ethyl-2,3-dimethylbenzene (n-C10/1E 2,3DM Bnz) at 375° C. while the bars 300a′-g′ represent the weight ratio of n-C10/1-ethyl-2,3-dimethylbenzene at 393° C. The bars 281a-g represent the weight ratio of n-C10/tetralin at 375° C. while the bars 301a′-g′represent the weight ratio of n-C10/tetralin at 393° C. The bars 302a-g represent the weight ratio of n-C12/2-methylnaphthalene (n-C12/2M Naph) at 375° C. while the bars 302a′-g′ represent the weight ratio of n-C12/2-methylnaphthalene at 393° C. The bars 303a-g represent the weight ratio of n-C12/1-methylnaphthalene (n-C12/1 M Naph) at 375° C. while the bars 303a′-g′ represent the weight ratio of n-C12/1-methylnaphthalene at 393° C. For each of the compound ratio groups, the “a” designation denotes the 375/500/0 experiment, the “a′” designation denotes the 393/500/0 experiment, the “b” designation denotes the 375/200/0 experiment, the “b′” designation denotes the 393/200/0 experiment, the “c” designation denotes the 375/500/400 experiment, the “c′” designation denotes the 393/500/400 experiment, the “d” designation denotes the 375/200/400 experiment, the “d′” designation denotes the 393/200/400 experiment, the “e” designation denotes the 375/50/400 experiment, the “e′” designation denotes the 393/50/400 experiment, the “f” designation denotes the 375/500/1000 experiment, the “f′” designation denotes the 393/500/1000 experiment, the “g” designation denotes the 375/200/1000 experiment, while the “g′” designation denotes the 393/200/1000 experiment.

From FIG. 33 it can be seen that the graphed bars have been generally ordered consecutively from highest ratio value to lowest ratio value. For each of the compound ratio groups, ratio bars are in the following order: the “a” designation denoting the 375/500/0 experiment, the “b” designation denoting the 375/200/0 experiment, the “c” designation denoting the 375/500/400 experiment, the “d” designation denoting the 375/200/400 experiment, the “e” designation denoting the 375/50/400 experiment, the “f” designation denoting the 375/500/1000 experiment, the “g” designation denoting the 375/200/1000 experiment, the “a′” designation denoting the 393/500/0 experiment, the “b′” designation denoting the 393/200/0 experiment, the “c′” designation denoting the 393/500/400 experiment, the “d′” designation denoting the 393/200/400 experiment, the “e′” designation denoting the 393/50/400 experiment, the “f” designation denoting the 393/500/1000 experiment, and the “g′” designation denotes the 393/200/1000 experiment. Thus the ordering includes the temperature difference having the greatest effect on the compositional change with the stress differences having the second largest effect on the compositional change. Further, the pressure difference has the smallest effect on composition and its effect is opposite to both temperature and stress.

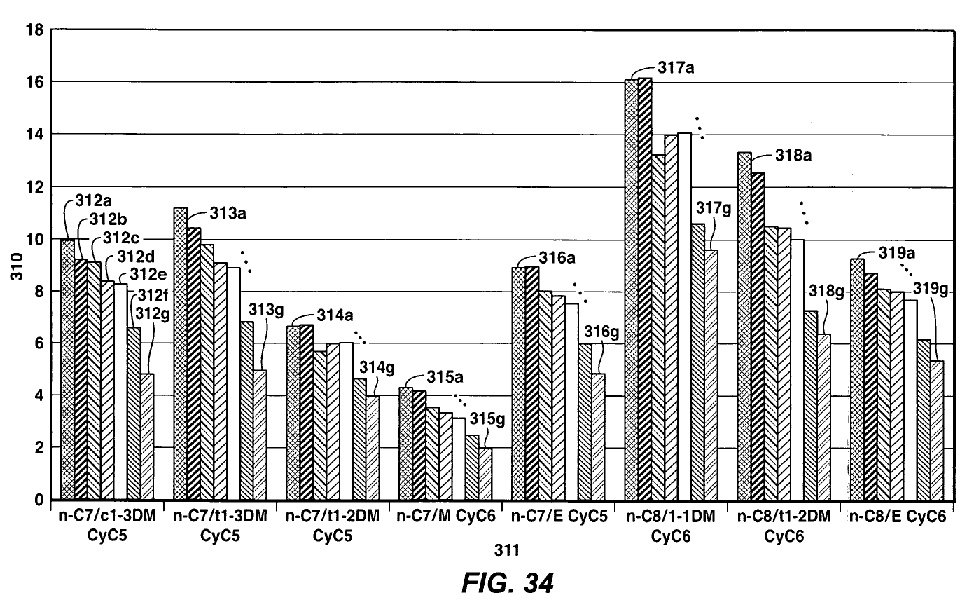

FIG. 34 is a bar graph of the weight ratio of several normal hydrocarbon compounds to like carbon number cyclic hydrocarbon compounds for each of the seven 393° C. experimental data discussed in Examples 13-19 in the Experimental section herein. The compound weight ratios were obtained through the experimental procedures, sample collection and analytical techniques discussed above for FIG. 30 and in the Experiments section. For clarity, the normal hydrocarbon compound to cyclic hydrocarbon compound weight ratios are derived as a ratio of a particular normal hydrocarbon compound's peak area in one experiment to a particular cyclic hydrocarbon compound's peak area for the same particular experiment. Thus the graphed weight ratios represent a weight ratio of two different compounds produced in the same experiment. The y-axis 310 represents the weight ratio of two compounds for a given experiment. The x-axis 311 contains the identity of each depicted compound ratio. The bars 312a-g represent the weight ratio of n-C7 to cis 1,3-dimethyl cyclopentane (n-C7/cl-3DM CyC5). The bars 313a-g represent the weight ratio of n-C7 to trans 1,3-dimethyl cyclopentane (n-C7/t1-3DM CyC5). The bars 314a-g represent the weight ratio of n-C7 to trans 1,2-dimethyl cyclopentane (n-C7/t1-2DM CyC5). The bars 315a-g represent the weight ratio of n-C7 to methyl cyclohexane (n-C7/M CyC6). The bars 316a-g represent the weight ratio of n-C7 to ethyl cyclopentane (n-C7/E CyC5). The bars 317a-g represent the weight ratio of n-C8 to 1,1-dimethyl cyclohexane (n-C8/1-1DM CyC6). The bars 318a-g represent the weight ratio of n-C8 to trans 1,2-dimethyl cyclohexane (n-C8/t1-2DM CyC6). The bars 319a-g represent the weight ratio of n-C8 to ethyl cyclohexane (n-C8/E CyC6). For each of the compound ratio groups, the “a” designation denotes the 393/500/0 experiment, the “b” designation denotes the 393/200/0 experiment, the “c” designation denotes the 393/500/400 experiment, the “d” designation denotes the 393/200/400 experiment, the “e” designation denotes the 393/50/400 experiment, the “f” designation denotes the 393/500/1000 experiment, while the “g” designation denotes the 393/200/1000 experiment.

From FIG. 34 it can be seen that the hydrocarbon liquids produced in the two 1,000 psi stressed experiments, represented by the “f” and “g” bars of 312-319, generally contain the most respective cyclic hydrocarbon compounds relative to the respective corresponding same carbon number normal hydrocarbon compound. It also can be seen that for the two 1,000 psi stress experiments, the lower initial argon pressure (200 psig argon) experiment represented by the “g” bars is generally more enriched in cyclic compounds relative to the higher initial argon pressure (500 psig argon) experiment represented by the “f” bars. From FIG. 34 it can also be seen that the hydrocarbon liquids produced in the three 400 psi stressed experiments, represented by the “c”, “d” and “f” bars, generally contain an increased amount of cyclic hydrocarbon compounds relative to the unstressed experiments (i.e., bars “a” and “b”) but a lower amount of cyclic hydrocarbon compounds relative to the 1,000 psi stressed experiments (bars “f” and “g”). It also can be seen that for the three 400 psi stress experiments, the lowest initial argon pressure (50 psig argon) experiment represented by the “e” bars is generally more enriched in cyclic compounds relative to the middle initial argon pressures (200 psig argon) experiment represented by the “d” bars and the highest initial argon pressures (500 psigg argon) experiment represented by the “c” bars, with the middle initial argon pressures (200 psig argon) experiment represented the “d” bars generally falling between the highest and lowest initial argon pressure experiments. By comparing the effect of the 0, 400, and 1,000 psi stressed data at consistent initial argon pressures (i.e., the a, c, and f data all at 500 psig initial argon or the b, d, and g data all at 200 psig initial argon) to the three 400 psi stress at different initial argon pressures data (i.e., the c, d, and e data), it becomes apparent that the compositional changes due to step changes in stress (i.e., 0 psi to 400 psi and 400 psi to 1,000 psi) are much more pronounced than the compositional changes due to step changes in pressure (i.e., 50 psig to 200 psig and 200 psig to 500 psig). It is also noted that the effect of increased pressure appears to partially retard the effect of increased stress. Thus pyrolyzing oil shale under increasing levels of stress appears to enrich the produced hydrocarbon liquid in cyclic compounds while decreasing pressure appears to enhance cyclic compound production. Further, the magnitude of compositional changed due to changes in stress appears to be more significant than the magnitude of the compositional change due to changes in pressure.

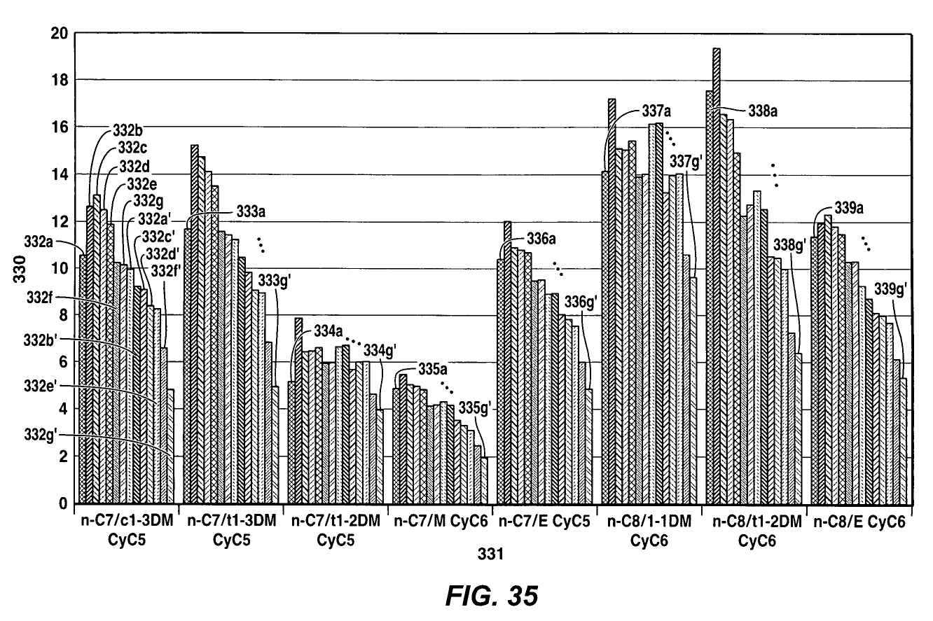

FIG. 35 is a bar graph of the weight ratio of the normal hydrocarbon compounds to like carbon number cyclic hydrocarbon compounds for each of the seven 375° C. and seven 393° C. experimental data discussed in Examples 6-19 in the Experimental section herein. The compound weight ratios were obtained through the experimental procedures, sample collection and analytical techniques discussed above for FIG. 30 and in the Experiments section. For clarity, the normal hydrocarbon compound to cyclic hydrocarbon compound weight ratios are derived as a ratio of a particular normal hydrocarbon compound's peak area in one experiment to a particular cyclic hydrocarbon compound's peak area for the same particular experiment. Thus the graphed weight ratios represent a weight ratio of two different compounds produced in the same experiment. The y-axis 330 represents the weight ratio of two compounds for a given experiment. The x-axis 331 contains the identity of each depicted compound ratio. The bars 332a-g represent the weight ratio of n-C7 to cis 1,3-dimethyl cyclopentane (n-C7/cl-3DM CyC5) at 375° C. while the bars 332a′-g′represent the weight ratio of n-C7 to cis 1,3-dimethyl cyclopentane at 393° C. The bars 333a-g represent the weight ratio of trans 1,3-dimethyl cyclopentane (n-C7/t1-3DM CyC5) at 375° C. while the bars 333a′-g′ represent the weight ratio of trans 1,3-dimethyl cyclopentane at 393° C. The bars 334a-g represent the weight ratio of n-C7 to trans 1,2-dimethyl cyclopentane (n-C7/t1-2DM CyC5) at 375° C. while the bars 334a′-g′ represent the weight ratio of n-C7 to trans 1,2-dimethyl cyclopentane at 393° C. The bars 335a-g represent the weight ratio of n-C7 to methyl cyclohexane (n-C7/M CyC6) at 375° C. while the bars 335a′-g′ represent the weight ratio of n-C7 to methyl cyclohexane at 393° C. The bars 336a-g represent the weight ratio of n-C7 to ethyl cyclopentane (n-C7/E CyC5) at 375° C. while the bars 336a′-g′ represent the weight ratio of n-C7 to ethyl cyclopentane at 393° C. The bars 337a-g represent the weight ratio of n-C8 to 1,1-dimethyl cyclohexane (n-C8/1-1DM CyC6) at 375° C. while the bars 337a′-g′ represent the weight ratio of n-C8 to 1,1-dimethyl cyclohexane at 393° C. The bars 338a-g represent the weight ratio of n-C8 to trans 1,2-dimethyl cyclohexane (n-C8/t1-2DM CyC6) at 375° C. while the bars 338a′-g′ represent the weight ratio of n-C8 to trans 1,2-dimethyl cyclohexane at 393° C. The bars 339a-g represent the weight ratio of n-C8 to ethyl cyclohexane (n-C8/E CyC6) at 375° C. while the bars 339a′-g′ represent the weight ratio of n-C8 to ethyl cyclohexane at 393° C. For each of the compound ratio groups, the “a” designation denotes the 375/500/0 experiment, the “a′” designation denotes the 393/500/0 experiment, the “b” designation denotes the 375/200/0 experiment, the “b′” designation denotes the 393/200/0 experiment, the “c” designation denotes the 375/500/400 experiment, the “c′” designation denotes the 393/500/400 experiment, the “d” designation denotes the 375/200/400 experiment, the “d′” designation denotes the 393/200/400 experiment, the “e” designation denotes the 375/50/400 experiment, the “e′” designation denotes the 393/50/400 experiment, the “f” designation denotes the 375/500/1000 experiment, the “f′” designation denotes the 393/500/1000 experiment, the “g” designation denotes the 375/200/1000 experiment, while the “g′” designation denotes the 393/200/1000 experiment.

From FIG. 35 it can be seen that the graphed bars have been generally ordered consecutively from highest ratio value to lowest ratio value. For each of the compound ratio groups, ratio bars are in the following order: the “a” designation denoting the 375/500/0 experiment, the “b” designation denoting the 375/200/0 experiment, the “c” designation denoting the 375/500/400 experiment, the “d” designation denoting the 375/200/400 experiment, the “e” designation denoting the 375/50/400 experiment, the “f” designation denoting the 375/500/1000 experiment, the “g” designation denoting the 375/200/1000 experiment, the “a′” designation denoting the 393/500/0 experiment, the “b′” designation denoting the 393/200/0 experiment, the “c′” designation denoting the 393/500/400 experiment, the “d′” designation denoting the 393/200/400 experiment, the “e′” designation denoting the 393/50/400 experiment, the “f′” designation denoting the 393/500/1000 experiment, and the “g′” designation denotes the 393/200/1000 experiment. From FIG. 34 it can be seen that the hydrocarbon liquids produced in the 393° C. experiments, represented by the “a′-g′” bars of 332-339, generally contain more of the respective cyclic hydrocarbon compounds as compared to the corresponding experiment completed at the same pressure and stress but at the lower 375° C. temperature, as represented by the “a-g” bars of 332-339. By comparing each respective pair of bars (e.g., a and a′, b and b′, etc.) for each corresponding pair of experiments differing only in temperature conditions it can also be seen that the effect of temperature is similar in magnitude, but more pronounced than the effect of stress and that the effect of temperature and stress are both much more pronounced than the effect of pressure on cyclic hydrocarbon compound production. It is also noted that the effect of increased pressure appears to partially retard the effect of stress. Thus the ordering in FIG. 35 includes the temperature difference having the greatest effect on the compositional change with the stress differences having the second largest effect on the compositional change. Further, the pressure difference has the smallest effect on composition and its effect is opposite to both temperature and stress.

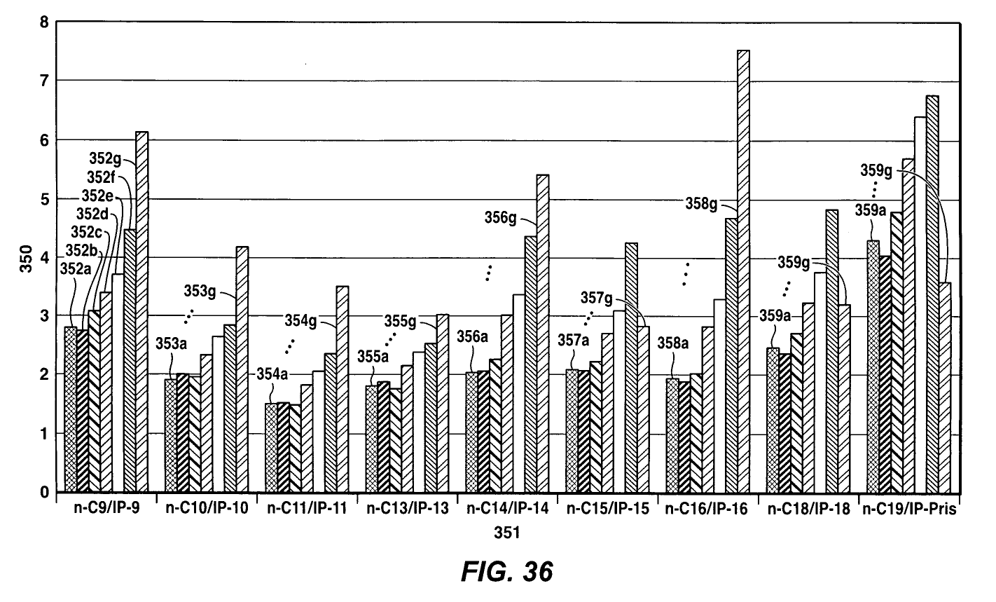

FIG. 36 is a bar graph of the weight ratio of several normal hydrocarbon compounds to like carbon number isoprenoid hydrocarbon compounds for each of the seven 393° C. experimental data discussed in Examples 13-19 in the Experimental section herein. The compound weight ratios were obtained through the experimental procedures, sample collection and analytical techniques discussed above for FIG. 30 and in the Experiments section. For clarity, the normal hydrocarbon compound to isoprenoid hydrocarbon compound weight ratios are derived as a ratio of a particular normal hydrocarbon compound's peak area in one experiment to a particular isoprenoid hydrocarbon compound's peak area for the same particular experiment. Thus the graphed weight ratios represent a weight ratio of two different compounds produced in the same experiment. The y-axis 350 represents the weight ratio of two compounds for a given experiment. The x-axis 351 contains the identity of each depicted compound ratio. The bars 352a-g represent the weight ratio of n-C9 to IP-9. The bars 353a-g represent the weight ratio of n-C10 to IP-10. The bars 354a-g represent the weight ratio of n-C11 to IP-11. The bars 355a-g represent the weight ratio of n-C13 to IP-13. The bars 356a-g represent the weight ratio of n-C14 to IP-14. The bars 357a-g represent the weight ratio of n-C15 to IP-15. The bars 358a-g represent the weight ratio of n-C16 to IP-16. The bars 359a-g represent the weight ratio of n-C18 to IP-18. The bars 360a-g represent the weight ratio of n-C19 to pristane. For each of the compound ratio groups, the “a” designation denotes the 393/500/0 experiment, the “b” designation denotes the 393/200/0 experiment, the “c” designation denotes the 393/500/400 experiment, the “d” designation denotes the 393/200/400 experiment, the “e” designation denotes the 393/50/400 experiment, the “f” designation denotes the 393/500/1000 experiment, while the “g” designation denotes the 393/200/1000 experiment.

From FIG. 36 it can be seen that the hydrocarbon liquids produced in the two 1,000 psi stressed experiments, represented by the “f” and “g” bars of 312-319, generally contain the least respective isoprenoid hydrocarbon compounds relative to the respective corresponding same carbon number normal hydrocarbon compound. It is however noted that for the IP-15, IP-18 and IP19 ratios, the 393/200/1000 data of the respective “g” experiment is lower than the 393/50/400 data of the respective “e” experiment. It also can be seen that for the two 1,000 psi stress experiments, the lower initial argon pressure (200 psig argon) experiment represented by the “g” bars is generally more depleted of isoprenoid compounds relative to the higher initial argon pressure (500 psig argon) experiment represented by the “f” bars. It is however again noted that for the IP-15, IP-18 and IP19 ratios, the 393/200/1000 data of the respective “g” experiment is lower than the 393/500/1000 data of the respective “f” experiment. From FIG. 36 it can also be seen that the hydrocarbon liquids produced in the three 400 psi stressed experiments, represented by the “c”, “d” and “f” bars, generally contain a decreased amount of isoprenoid hydrocarbon compounds relative to the unstressed experiments (i.e., bars “a” and “b”) but a greater amount of isoprenoid hydrocarbon compounds relative to the 1,000 psi stressed experiments (bars “f” and “g”). It also can be seen that for the three 400 psi stress experiments, the lowest initial argon pressure (50 psig argon) experiment represented by the “e” bars is generally more depleted of isoprenoid compounds relative to the middle initial argon pressures (200 psig argon) experiment represented by the “d” bars and the highest initial argon pressures (500 psig argon) experiment represented by the “c” bars, with the middle initial argon pressures (200 psig argon) experiment represented the “d” bars generally falling between the highest and lowest initial argon pressure experiments. By comparing the effect of the 0, 400, and 1,000 psi stressed data at consistent initial argon pressures (i.e., the a, c, and f data all at 500 psig initial argon or the b, d, and g data all at 200 psig initial argon) to the three 400 psi stress at different initial argon pressures data (i.e., the c, d, and e data), it becomes apparent that the compositional changes due to step changes in stress (i.e., 0 psi to 400 psi and 400 psi to 1,000 psi) are of a similar magnitude to the compositional changes due to step changes in pressure (i.e., 50 psig to 200 psig and 200 psig to 500 psig). It is also noted that the effect of increased pressure appears to partially retard the effect of increased stress. Thus pyrolyzing oil shale under increasing levels of stress appears to deplete the produced hydrocarbon liquid in isoprenoid compounds while decreasing pressure appears to deplete isoprenoid compound production. Further, the magnitude of compositional changed due to changes in stress appears to be of similar magnitude to the magnitude of the compositional change due to changes in pressure.

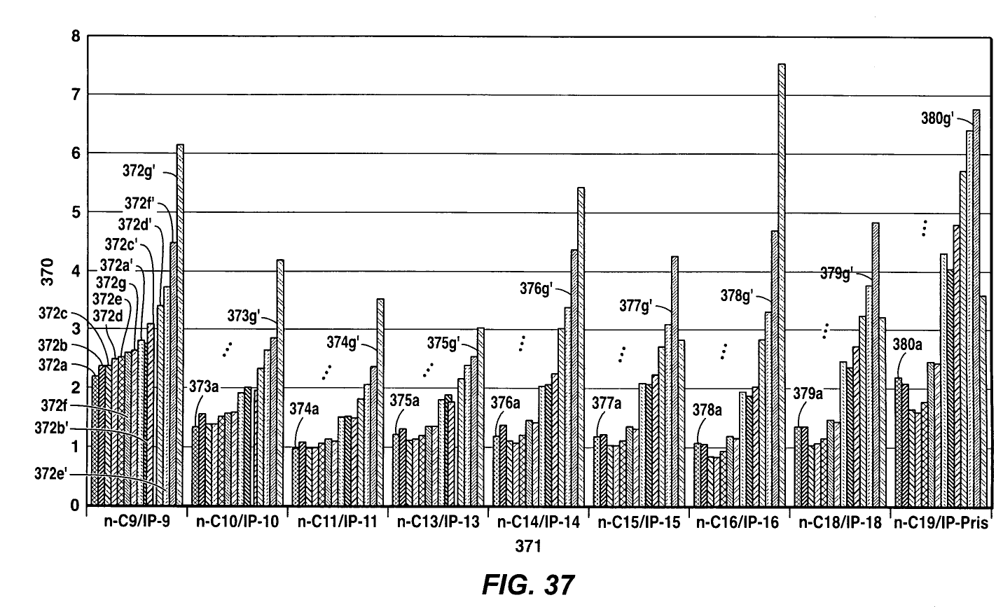

FIG. 37 is a bar graph of the weight ratio of the normal hydrocarbon compounds to like carbon number isoprenoid hydrocarbon compounds for each of the seven 375° C. and seven 393° C. experimental data discussed in Examples 6-19 in the Experimental section herein. The compound weight ratios were obtained through the experimental procedures, sample collection and analytical techniques discussed above for FIG. 30 and in the Experiments section. For clarity, the normal hydrocarbon compound to isoprenoid hydrocarbon compound weight ratios are derived as a ratio of a particular normal hydrocarbon compound's peak area in one experiment to a particular isoprenoid hydrocarbon compound's peak area for the same particular experiment. Thus the graphed weight ratios represent a weight ratio of two different compounds produced in the same experiment. The y-axis 370 represents the weight ratio of two compounds for a given experiment. The x-axis 371 contains the identity of each depicted compound ratio. The bars 372a-g′ represent the weight ratio of n-C9 to IP-9. The bars 373a-g′ represent the weight ratio of n-C10 to IP-10. The bars 374a-g′ represent the weight ratio of n-C11 to IP-11. The bars 375a-g′ represent the weight ratio of n-C13 to IP-13. The bars 376a-g′ represent the weight ratio of n-C14 to IP-14. The bars 377a-g′ represent the weight ratio of n-C15 to IP-15. The bars 378a-g′ represent the weight ratio of n-C16 to IP-16. The bars 379a-g′ represent the weight ratio of n-C18 to IP-18. The bars 380a-g′ represent the weight ratio of n-C19 to pristane. For each of the compound ratio groups, the “a” designation denotes the 375/500/0 experiment, the “a′” designation denotes the 393/500/0 experiment, the “b” designation denotes the 375/200/0 experiment, the “b′” designation denotes the 393/200/0 experiment, the “c” designation denotes the 375/500/400 experiment, the “c′” designation denotes the 393/500/400 experiment, the “d” designation denotes the 375/200/400 experiment, the “d′” designation denotes the 393/200/400 experiment, the “e” designation denotes the 375/50/400 experiment, the “e′” designation denotes the 393/50/400 experiment, the “f” designation denotes the 375/500/1000 experiment, the “f′” designation denotes the 393/500/1000 experiment, the “g” designation denotes the 375/200/1000 experiment, while the “g′” designation denotes the 393/200/1000 experiment.

From FIG. 37 it can be seen that the graphed bars have been generally ordered consecutively from lowest ratio value to highest ratio value. For each of the compound ratio groups, ratio bars are in the following order: the “a” designation denoting the 375/500/0 experiment, the “b” designation denoting the 375/200/0 experiment, the “c” designation denoting the 375/500/400 experiment, the “d” designation denoting the 375/200/400 experiment, the “e” designation denoting the 375/50/400 experiment, the “f” designation denoting the 375/500/1000 experiment, the “g” designation denoting the 375/200/1000 experiment, the “a′” designation denoting the 393/500/0 experiment, the “b′” designation denoting the 393/200/0 experiment, the “c′” designation denoting the 393/500/400 experiment, the “d′” designation denoting the 393/200/400 experiment, the “e′” designation denoting the 393/50/400 experiment, the “f′” designation denoting the 393/500/1000 experiment, and the “g′” designation denotes the 393/200/1000 experiment. From FIG. 37 it can be seen that the hydrocarbon liquids produced in the 393° C. experiments, represented by the “a′-g′” bars of 372-380, generally contain less of the respective isoprenoid hydrocarbon compounds as compared to the corresponding experiment completed at the same pressure and stress but at the lower 375° C. temperature, as represented by the “a-g” bars of 372-380. By comparing each respective pair of bars (e.g., a and a′, b and b′, etc.) for each corresponding pair of experiments differing only in temperature conditions it can also be seen that the effect of temperature is more pronounced than the effect of stress and more pronounced than the effect of pressure on isoprenoid hydrocarbon compound production. It is also noted that the effect of increased pressure appears to partially retard the effect of stress. Thus the ordering in FIG. 37 includes the temperature difference having the greatest effect on the compositional change with the stress differences having a reduced, but second largest effect on the compositional change. Further, the pressure difference has the smallest effect on composition and its effect is opposite to the effect of both temperature and stress.

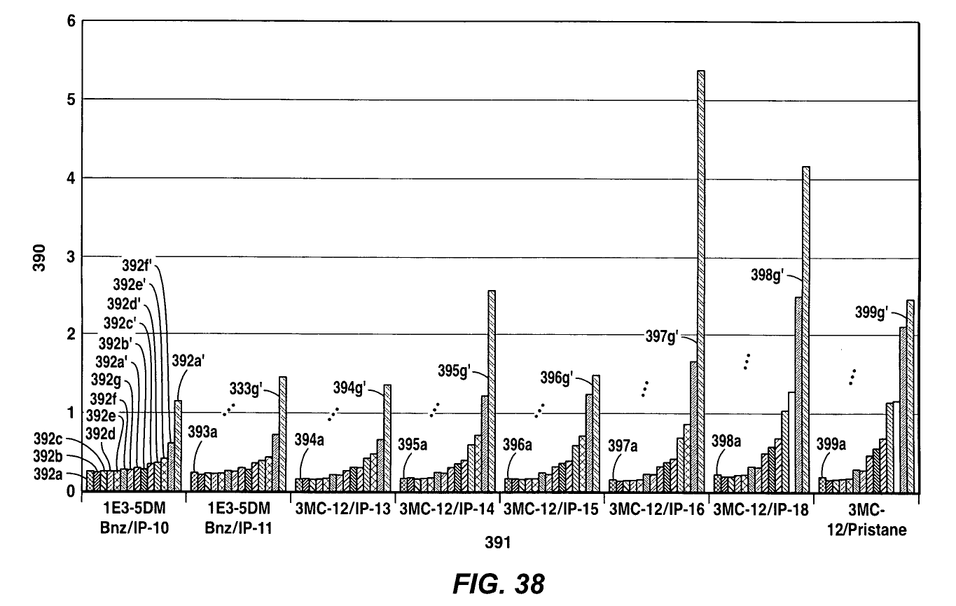

FIG. 38 is a bar graph of the weight ratio of the certain hydrocarbon compounds to similar carbon number isoprenoid hydrocarbon compounds for each of the seven 375° C. and seven 393° C. experimental data discussed in Examples 6-19 in the Experimental section herein. The compound weight ratios were obtained through the experimental procedures, sample collection and analytical techniques discussed above for FIG. 30 and in the Experiments section. For clarity, the hydrocarbon compound to isoprenoid hydrocarbon compound weight ratios are derived as a ratio of a particular hydrocarbon compound's peak area in one experiment to a particular isoprenoid hydrocarbon compound's peak area for the same particular experiment. Thus the graphed weight ratios represent a weight ratio of two different compounds produced in the same experiment. The y-axis 390 represents the weight ratio of two compounds for a given experiment. The x-axis 391 contains the identity of each depicted compound ratio. The bars 392a-g′ represent the weight ratio of 1-ethyl-3,5-dimethylbenzene (1E3-5DM Bnz) to IP-10. The bars 393a-g′ represent the weight ratio of 1-ethyl-3,5-dimethylbenzene to IP-11. The bars 394a-g′ represent the weight ratio of 3-methyldodecane (3MC-12) to IP-13. The bars 395a-g′ represent the weight ratio of 3-methyldodecane to IP-14. The bars 396a-g′ represent the weight ratio of 3-methyldodecane to IP-15. The bars 397a-g′ represent the weight ratio of 3-methyldodecane to IP-16. The bars 398a-g′ represent the weight ratio of 3-methyldodecane to IP-18. The bars 399a-g′ represent the weight ratio of 3-methyldodecane to pristane. For each of the compound ratio groups, the “a” designation denotes the 375/500/0 experiment, the “a′” designation denotes the 393/500/0 experiment, the “b” designation denotes the 375/200/0 experiment, the “b′” designation denotes the 393/200/0 experiment, the “c” designation denotes the 375/500/400 experiment, the “c′” designation denotes the 393/500/400 experiment, the “d” designation denotes the 375/200/400 experiment, the “d′” designation denotes the 393/200/400 experiment, the “e” designation denotes the 375/50/400 experiment, the “e′” designation denotes the 393/50/400 experiment, the “f” designation denotes the 375/500/1000 experiment, the “f′” designation denotes the 393/500/1000 experiment, the “g” designation denotes the 375/200/1000 experiment, while the “g′” designation denotes the 393/200/1000 experiment.

FIG. 38 was developed to provide an alternate comparison of the changes in isoprenoid production from changes in experimental conditions. FIGS. 36 & 37 compare isoprenoid production to like carbon number normal hydrocarbon compounds. However, from FIG. 29 it is apparent that normal hydrocarbon compound production is reduced by increasing stress, increasing temperature and decreasing pressure, just as isoprenoid hydrocarbon compound production is reduced by increasing stress, increasing temperature and decreasing pressure. Thus the ratio comparisons depicted in FIGS. 36 & 37 actually compare the reduction in normal hydrocarbon compound production relative to the reduction in isoprenoid hydrocarbon production, which both decrease with increasing stress, increasing temperature, and decreasing pressure. FIG. 38 provides an alternate comparison to better gauge the effect of stress, temperature and pressure changes on isoprenoid production. FIG. 38 provides a ratio of selective isoprenoid hydrocarbon compounds to either 1-ethyl-3,5-dimethylbenzene (1E3-5DM Bnz) or 3-methyldodecane (3MC-12). These two compounds were chosen for comparison because they are of a similar elution time to the respective isoprenoid compounds used in a particular ratio and have a fairly consistent concentration over the entire stress, temperature and pressure ranges tested in all the experiments, which can be seen by looking at the weight ratio concentration plots in FIG. 29 for each of 1-ethyl-3,5-dimethylbenzene (1E3-5DMBz) or 3-methyldodecane (3MC12).

From FIG. 38 it can be seen that the graphed bars have been generally ordered consecutively from lowest ratio value to highest ratio value. For each of the compound ratio groups, ratio bars are in the following order: the “a” designation denoting the 375/500/0 experiment, the “b” designation denoting the 375/200/0 experiment, the “c” designation denoting the 375/500/400 experiment, the “d” designation denoting the 375/200/400 experiment, the “e” designation denoting the 375/50/400 experiment, the “f” designation denoting the 375/500/1000 experiment, the “g” designation denoting the 375/200/1000 experiment, the “a′” designation denoting the 393/500/0 experiment, the “b′” designation denoting the 393/200/0 experiment, the “c′” designation denoting the 393/500/400 experiment, the “d′” designation denoting the 393/200/400 experiment, the “e′” designation denoting the 393/50/400 experiment, the “f′” designation denoting the 393/500/1000 experiment, and the “g′” designation denotes the 393/200/1000 experiment. From FIG. 38 it can be seen that the hydrocarbon liquids produced in the 393° C. experiments, represented by the “a′-g′” bars of 372-380, generally contain less of the respective isoprenoid hydrocarbon compounds as compared to the corresponding experiment completed at the same pressure and stress but at the lower 375° C. temperature, as represented by the “a-g” bars of 372-380. By comparing each respective pair of bars (e.g., a and a′, b and b′, etc.) for each corresponding pair of experiments differing only in temperature conditions it can also be seen that the effect of temperature is more pronounced than the effect of stress and more pronounced than the effect of pressure on isoprenoid hydrocarbon compound production. It is also noted that the effect of increased pressure appears to partially retard the effect of stress. Thus the ordering in FIG. 38 includes the temperature difference having the greatest effect on the compositional change with the stress differences having a reduced, but second largest effect on the compositional change. Further, the pressure difference has the smallest effect on composition and its effect is opposite to the effect of both temperature and stress.

From the above-described data discussed in FIGS. 29-38, it can be seen that temperature, pressure and lithostatic stress can affect the composition of produced fluids generated within an organic-rich rock via heating and pyrolysis. This implies that the composition of the produced hydrocarbon fluid from in situ heating and pyrolysis processes can also be influenced by selecting, maintaining or in some cases controlling one or more of in situ temperature, in situ pressure, and in situ lithostatic stress conditions of the organic-rich rock formation being heated in the in situ process. By selecting, maintaining, or in some cases controlling the heating and pyrolysis conditions of oil shale, a condensable hydrocarbon fluid product that has desired compositional properties may be obtained. Such a product may be suitable for refining into gasoline and distillate products. Further, such a product, either before or after further fractionation, may have utility as a feed stock for certain chemical processes.

In some embodiments, the produced hydrocarbon fluid includes a condensable hydrocarbon portion. In some embodiments the condensable hydrocarbon portion may have one or more of a n-C6 to benzene weight ratio less than 35.0, a n-C7 to toluene weight ratio less than 7.0, a n-C8 to ethylbenzene weight ratio less than 16.0, a n-C8 to ortho-xylene weight ratio less than 7.0, a n-C8 to meta-xylene weight ratio less than 1.9, a n-C9 to 1-ethyl-3-methylbenzene weight ratio less than 8.2, a n-C9 to 1-ethyl-4-methylbenzene weight ratio less than 4.4, a n-C9 to 1,2,4-trimethylbenzene weight ratio less than 2.7, a n-C10 to 1-ethyl-2,3-dimethylbenzene weight ratio less than 13.5, a n-C10 to tetralin weight ratio less than 25.0, a n-C12 to 2-methylnaphthalene weight ratio less than 4.9, and a n-C12 to 1-methylnaphthalene weight ratio less than 6.9. In some embodiments the condensable hydrocarbon portion may have two or more, three or more or four or more of the weight ratios described above in the paragraph.

In some embodiments the condensable hydrocarbon portion has a n-C6 to benzene weight ratio less than 35.0, less than 25, 15, 10 or 7. In some embodiments the condensable hydrocarbon portion has a n-C7 to toluene weight ratio less than 7.0, less than 6, 5, 4 or 3. In some embodiments the condensable hydrocarbon portion has a n-C8 to ethylbenzene weight ratio less than 16.0, less than 13, 10, 5 or 2. In some embodiments the condensable hydrocarbon portion has a n-C8 to ortho-xylene weight ratio less than 7.0, less than 6, 5, 4 or 2. In some embodiments the condensable hydrocarbon portion has a n-C8 to meta-xylene weight ratio less than 1.9, less than 1.8, 1.7, 1.6 or 1.5. In some embodiments the condensable hydrocarbon portion has a n-C9 to 1-ethyl-3-methylbenzene weight ratio less than 8.2, less than 7, 6, 4 or 2. In some embodiments the condensable hydrocarbon portion has a n-C9 to 1-ethyl-4-methylbenzene weight ratio less than 4.4, less than 4.0, 3.5, 3.0 or 2.0. In some embodiments the condensable hydrocarbon portion has a n-C9 to 1,2,4-trimethylbenzene weight ratio less than 2.7, less than 2.5, 2.0, 1.5 or 1.0. In some embodiments the condensable hydrocarbon portion has a n-C10 to 1-ethyl-2,3-dimethylbenzene weight ratio less than 13.5, less than 12, 10, 7 or 5. In some embodiments the condensable hydrocarbon portion has a n-C10 to tetralin weight ratio less than 25.0, less than 20, 15, or 10. In some embodiments the condensable hydrocarbon portion has a n-C12 to 2-methylnaphthalene weight ratio less than 4.9, less than 4.5, 4.0, 3.5 or 3. In some embodiments the condensable hydrocarbon portion has a n-C12 to 1-methylnaphthalene weight ratio less than 6.9, 6.0, 4.0, 3.0, or 2.5.

In some embodiments the condensable hydrocarbon portion may have one or more of a n-C6 to benzene weight ratio less than 24.0, a n-C7 to toluene weight ratio less than 6.6, a n-C8 to ethylbenzene weight ratio less than 15.0, a n-C8 to ortho-xylene weight ratio less than 6.6, a n-C8 to meta-xylene weight ratio less than 1.8, a n-C9 to 1-ethyl-3-methylbenzene weight ratio less than 7.9, a n-C9 to 1-ethyl-4-methylbenzene weight ratio less than 4.3, a n-C9 to 1,2,4-trimethylbenzene weight ratio less than 2.6, a n-C10 to 1-ethyl-2,3-dimethylbenzene weight ratio less than 13.1, a n-C10 to tetralin weight ratio less than 23.7, a n-C12 to 2-methylnaphthalene weight ratio less than 5.0, and a n-C12 to 1-methylnaphthalene weight ratio less than 6.8. In some embodiments the condensable hydrocarbon portion may have two or more, three or more or four or more of the weight ratios described above in the paragraph.

In some embodiments the condensable hydrocarbon portion may have one or more of a n-C6 to benzene weight ratio less than 13.4, a n-C7 to toluene weight ratio less than 5.1, a n-C8 to ethylbenzene weight ratio less than 12.3, a n-C8 to ortho-xylene weight ratio less than 5.3, a n-C8 to meta-xylene weight ratio less than 1.5, a n-C9 to 1-ethyl-3-methylbenzene weight ratio less than 5.9, a n-C9 to 1-ethyl-4-methylbenzene weight ratio less than 3.8, a n-C9 to 1,2,4-trimethylbenzene weight ratio less than 2.2, a n-C10 to 1-ethyl-2,3-dimethylbenzene weight ratio less than 12.2, a n-C10 to tetralin weight ratio less than 23.4, a n-C12 to 2-methylnaphthalene weight ratio less than 4.0, and a n-C12 to 1-methylnaphthalene weight ratio less than 6.1. In some embodiments the condensable hydrocarbon portion may have two or more, three or more or four or more of the weight ratios described above in the paragraph.

In some embodiments the condensable hydrocarbon portion may have one or more of a n-C7 to cis 1,3-dimethyl cyclopentane weight ratio less than 13.1, a n-C7 to trans 1,3-dimethyl cyclopentane weight ratio less than 14.9, a n-C7 to trans 1,2-dimethyl cyclopentane weight ratio less than 7.0, a n-C7 to methyl cyclohexane weight ratio less than 5.2, a n-C7 to ethyl cyclopentane weight ratio less than 11.3, a n-C8 to 1,1-dimethyl cyclohexane weight ratio less than 16.0, a n-C8 to trans 1,2-dimethyl cyclohexane weight ratio less than 17.5, and a n-C8 to ethyl cyclohexane weight ratio less than 12.3. In some embodiments the condensable hydrocarbon portion may have two or more, three or more or four or more of the weight ratios described above in the paragraph.

In some embodiments the condensable hydrocarbon portion may have one or more of a n-C7 to cis 1,3-dimethyl cyclopentane weight ratio less than 12.7, a n-C7 to trans 1,3-dimethyl cyclopentane weight ratio less than 14.7, a n-C7 to trans 1,2-dimethyl cyclopentane weight ratio less than 6.6, a n-C7 to methyl cyclohexane weight ratio less than 5.0, a n-C7 to ethyl cyclopentane weight ratio less than 10.9, a n-C8 to 1,1-dimethyl cyclohexane weight ratio less than 15.4, a n-C8 to trans 1,2-dimethyl cyclohexane weight ratio less than 16.5, and a n-C8 to ethyl cyclohexane weight ratio less than 12.0. In some embodiments the condensable hydrocarbon portion may have two or more, three or more or four or more of the weight ratios described above in the paragraph.

In some embodiments the condensable hydrocarbon portion may have one or more of a n-C7 to cis 1,3-dimethyl cyclopentane weight ratio less than 10.3, a n-C7 to trans 1,3-dimethyl cyclopentane weight ratio less than 11.6, a n-C7 to trans 1,2-dimethyl cyclopentane weight ratio less than 5.9, a n-C7 to methyl cyclohexane weight ratio less than 4.1, a n-C7 to ethyl cyclopentane weight ratio less than 9.5, a n-C8 to 1,1-dimethyl cyclohexane weight ratio less than 13.9, a n-C8 to trans 1,2-dimethyl cyclohexane weight ratio less than 12.3, and a n-C8 to ethyl cyclohexane weight ratio less than 10.3. In some embodiments the condensable hydrocarbon portion may have two or more, three or more or four or more of the weight ratios described above in the paragraph.

In some embodiments the condensable hydrocarbon portion has a n-C7 to c is 1,3-dimethyl cyclopentane weight ratio less than 13.1, 12, 10, 7, or 5. In some embodiments the condensable hydrocarbon portion has a n-C7 to trans 1,3-dimethyl cyclopentane weight ratio less than 14.9, 13, 10, 7 or 5. In some embodiments the condensable hydrocarbon portion has a n-C7 to trans 1,2-dimethyl cyclopentane weight ratio less than 7.0, 6.0, 5.0 or 4.0. In some embodiments the condensable hydrocarbon portion has a n-C7 to methyl cyclohexane weight ratio less than 5.2, 4.7, 4.2, 3.5 or 2.0. In some embodiments the condensable hydrocarbon portion has a n-C7 to ethyl cyclopentane weight ratio less than 11.3, 10.0, 8.0, 6.5 or 5.0. In some embodiments the condensable hydrocarbon portion has a n-C8 to 1,1-dimethyl cyclohexane weight ratio less than 15.4, 14.0, 12.0, 10.0 or 9.0. In some embodiments the condensable hydrocarbon portion has a n-C8 to trans 1,2-dimethyl cyclohexane weight ratio less than 16.5, 15.0, 12.0, 9.0 or 6.0. In some embodiments the condensable hydrocarbon portion has a n-C8 to ethyl cyclohexane weight ratio less than 12.0, 10.0, 8.0, 6.0 or 5.0.

In some embodiments the condensable hydrocarbon portion has a n-C7 to cis 1,3-dimethyl cyclopentane weight ratio greater than 0.5 or 1.0, a n-C7 to trans 1,3-dimethyl cyclopentane weight ratio greater than 0.5 or 1.0, a n-C7 to trans 1,2-dimethyl cyclopentane weight ratio greater than 0.5 or 1.0, a n-C7 to methyl cyclohexane weight ratio greater than 0.2 or 0.5, a n-C7 to ethyl cyclopentane weight ratio greater than 0.5 or 1.0, a n-C8 to 1,1-dimethyl cyclohexane weight ratio greater than 0.5 or 1.0, a n-C8 to trans 1,2-dimethyl cyclohexane weight ratio greater than 0.5 or 1.0, and/or a n-C8 to ethyl cyclohexane weight ratio greater than 0.5 or 1.0.

In some embodiments the condensable hydrocarbon portion may have one or more of a n-C9 to IP-9 weight ratio greater than 2.4, a n-C10 to IP-10 weight ratio greater than 1.4, a n-C11 to IP-11 weight ratio greater than 1.0, a n-C13 to IP-13 weight ratio greater than 1.1, a n-C14 to IP-14 weight ratio greater than 1.1, a n-C15 to IP-15 weight ratio greater than 1.0, a n-C16 to IP-16 weight ratio greater than 0.8, a n-C18 to IP-18 weight ratio greater than 1.0, and a n-C19 to pristane weight ratio greater than 1.6. In some embodiments the condensable hydrocarbon portion may have two or more, three or more or four or more of the weight ratios described above in the paragraph.

In some embodiments the condensable hydrocarbon portion may have one or more of a n-C9 to IP-9 weight ratio greater than 2.5, a n-C10 to IP-10 weight ratio greater than 1.5, a n-C11 to IP-11 weight ratio greater than 1.1, a n-C13 to IP-13 weight ratio greater than 1.2, a n-C14 to IP-14 weight ratio greater than 1.2, a n-C15 to IP-15 weight ratio greater than 1.1, a n-C16 to IP-16 weight ratio greater than 0.9, a n-C18 to IP-18 weight ratio greater than 1.1, and a n-C19 to pristane weight ratio greater than 1.8. In some embodiments the condensable hydrocarbon portion may have two or more, three or more or four or more of the weight ratios described above in the paragraph.

In some embodiments the condensable hydrocarbon portion may have one or more of a n-C9 to IP-9 weight ratio greater than 2.6, a n-C10 to IP-10 weight ratio greater than 1.6, a n-C11 to IP-1 weight ratio greater than 1.2, a n-C13 to IP-13 weight ratio greater than 1.3, a n-C14 to IP-14 weight ratio greater than 1.4, a n-C15 to IP-15 weight ratio greater than 1.4, a n-C16 to IP-16 weight ratio greater than 1.2, a n-C18 to IP-18 weight ratio greater than 1.5, and a n-C19 to pristane weight ratio greater than 2.4. In some embodiments the condensable hydrocarbon portion may have two or more, three or more or four or more of the weight ratios described above in the paragraph.

In some embodiments the condensable hydrocarbon portion has a n-C9 to IP-9 weight ratio greater than 2.4, 3.0, 4.0, 5.0 or 6.0. In some embodiments the condensable hydrocarbon portion has a n-C10 to IP-10 weight ratio greater than 1.4, 2.0, 2.5, 3.0 or 4.0. In some embodiments the condensable hydrocarbon portion has a n-C11 to IP-11 weight ratio greater than 1.0, 1.5, 2.0, 2.5 or 3.5. In some embodiments the condensable hydrocarbon portion has a n-C13 to IP-13 weight ratio greater than 1.1, 1.5, 2.0, 2.5 or 3.0. In some embodiments the condensable hydrocarbon portion has a n-C14 to IP-14 weight ratio greater than 1.1, 2.0, 3.0, 4.0 or 5.0. In some embodiments the condensable hydrocarbon portion has a n-C15 to IP-15 weight ratio greater than 1.0, 1.5, 2.0, 3.0 or 4.0. In some embodiments the condensable hydrocarbon portion has a n-C16 to IP-16 weight ratio greater than 0.8, 1.0, 1.5, 2.0, 3.0 or 7.0. In some embodiments the condensable hydrocarbon portion has a n-C18 to IP-18 weight ratio greater than 1.0, 1.5, 2.0, 2.5 or 5.0. In some embodiments the condensable hydrocarbon portion has a n-C19 to pristane weight ratio greater than 2.4, 3.0, 3.5, 4.0 or 6.0.

In some embodiments the condensable hydrocarbon portion has a n-C9 to IP-9 weight ratio less than 15.0 or 10.0, a n-C10 to IP-10 weight ratio less than 15.0 or 10.0, a n-C11 to IP-11 weight ratio less than 15.0 or 10.0, a n-C13 to IP-13 weight ratio less than 15.0 or 10.0, a n-C14 to IP-14 weight ratio less than 15.0 or 10.0, a n-C15 to IP-15 weight ratio less than 15.0 or 10.0, a n-C16 to IP-16 weight ratio less than 15.0 or 10.0, a n-C18 to IP-18 weight ratio less than 15.0 or 10.0, and/or a n-C19 to pristane weight ratio less than 15.0 or 10.0.

In some embodiments the condensable hydrocarbon portion may have one or more of a 1 ethyl-3,5-dimethylbenzene to IP-10 weight ratio greater than 0.3, a 1 ethyl-3,5-dimethylbenzene to IP-11 weight ratio greater than 0.2, a 3-methyldodecane to IP-13 weight ratio greater than 0.2, a 3-methyldodecane to IP-14 weight ratio greater than 0.2, a 3-methyldodecane to IP-15 weight ratio greater than 0.2, a 3-methyldodecane to IP-16 weight ratio greater than 0.2, a 3-methyldodecane to IP-18 weight ratio greater than 0.2, and a 3-methyldodecane to pristane weight ratio greater than 0.2. In some embodiments the condensable hydrocarbon portion may have two or more, three or more or four or more of the weight ratios described above in the paragraph.

As used in the preceding paragraphs and in the claims with respect to aromatic, cyclic, isoprenoid and normal hydrocarbon compounds, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in the preceding paragraphs may be combined with any of the other aspects of the invention discussed herein. Certain features of the present invention discussed in the preceding paragraphs are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in the preceding paragraphs may be combined with any of the other aspects of the invention discussed in such paragraphs or otherwise herein.

The use of “n-C_” (e.g., n-C10) herein and in the claims is meant to refer to the amount of a particular normal alkane hydrocarbon compound found in a condensable hydrocarbon fluid determined by C4-C19 liquid sample gas chromatography (C4-C19 GC) as described herein, particularly in the section labeled “Experiments” herein. That is “n-C_” is determined from the respective C4-C19 peak area determined using the C4-C19 analysis methodology according to the procedure described in the Experiments section of this application. Further, when a particular “n-C_” normal alkane hydrocarbon compound is compared to another hydrocarbon compound in a weight ratio herein and in the claims, such a weight ratio is obtained by the ratio of the particular “n-C_” normal alkane hydrocarbon compound's C4-C19 peak area to the other particular hydrocarbon compound's C4-C19 peak area. For example, an n-C8 to ethylbenzene weigh ratio is obtained by dividing the n-C8 C4-C19 GC peak area for a respective condensable hydrocarbon fluid by the C4-C19 GC peak area for ethylbenzene, where both of such respective C4-C19 GC peak areas are determined by the C4-C19 GC analysis procedures, C4-C19 GC peak identification methodologies, and C4-C19 GC peak integration methodologies discussed in the Experiments section herein.

As used herein and in the claims, weight ratios of aromatic hydrocarbon compounds (e.g., toluene, ortho-xylene, or 1,2,4-trimethylbenzene), cyclic hydrocarbon compounds (e.g., 1,3-dimethyl cyclopentane, ethyl cyclopentane, or ethyl cyclohexane) and isoprenoid compounds (e.g., IP-9, IP-11, or pristane) is meant to refer to the amount of a particular hydrocarbon compound found in a condensable hydrocarbon fluid as determined by C4-C19 liquid sample gas chromatography (C4-C19 GC) as described herein, particularly in the section labeled “Experiments” herein. That is the amount of a respective compound is determined from the respective C4-C19 peak area determined using the C4-C19 analysis methodology according to the procedure described in the Experiments section of this application. Further, when a first hydrocarbon compound is compared to a second hydrocarbon compound in a weight ratio herein and in the claims, such a weight ratio is obtained by the ratio of the first hydrocarbon compound's C4-C19 peak area to the second hydrocarbon compound's C4-C19 peak area. For example, an n-C9 to IP-9 weigh ratio is obtained by dividing the n-C9 C4-C19 GC peak area for a respective condensable hydrocarbon fluid by the C4-C19 GC peak area for IP-9, where both of such respective C4-C19 GC peak areas are determined by the C4-C19 GC analysis procedures, C4-C19 GC peak identification methodologies, and C4-C19 GC peak integration methodologies discussed in the Experiments section herein.

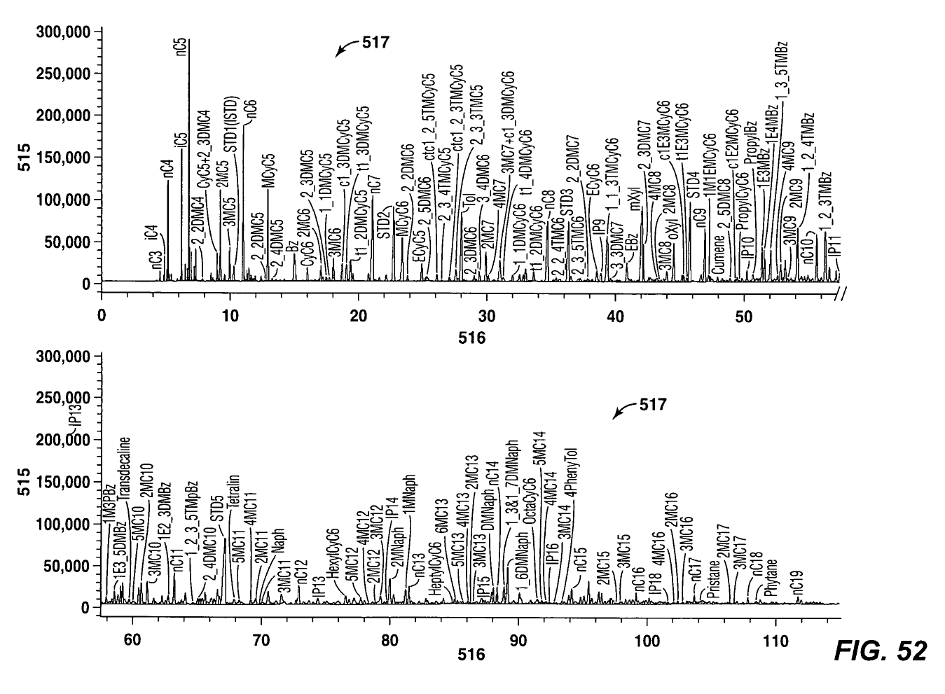

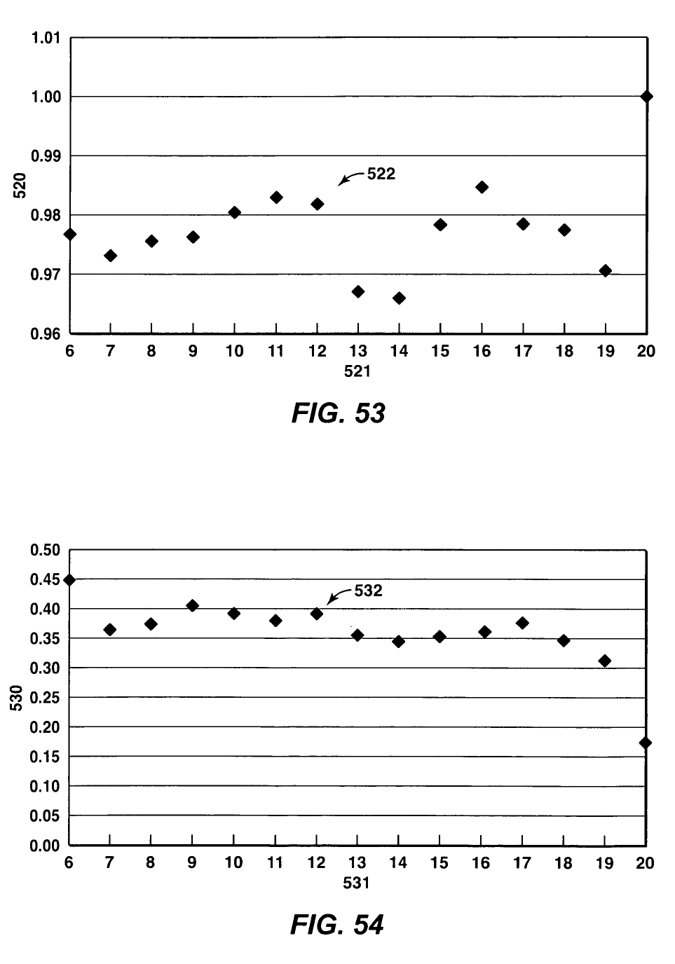

Certain hydrocarbon compounds, particularly certain stereoisomers of certain hydrocarbon compounds, can be used for a variety of purposes including, for example: determine the relative age of naturally occurring petroleum deposits, characterizing the source kerogen of naturally occurring oils, and estimating the level of thermal maturation of a naturally occurring oil or kerogen. Examples of such techniques can be found in Peters, K. E., Walters, C. C., and Moldowan, J. M., The Biomarker Guide, Vol. 1 & 2, Cambridge University Press (2005). Applicants have investigated certain biomarkers for the hydrocarbon fluids produced in Examples 6-19 and the liquid chromatographic extraction described in Example 20, and some of the biomarker data generated in such experiments is presented in FIGS. 53-59.

Hopanes, steranes and phenanthrenes are hydrocarbon molecules that show systematic isomerization reactions governed by thermal maturation in natural hydrocarbon systems. Hopanes, and steranes like many molecules of biologic origin, are generally found in certain stereoisomer forms in biological matter. The biological stereoisomers may be a less thermodynamically stable form of the compound, but are generated enzymatically for a specific biological function in a living organism. As the biological matter is altered by diagenesis, catagenesis, and metagenesis additional stereoisomeric compounds may be formed as the biological stereoisomeric form is transformed into more thermodynamically stable isomers. Moreover, the amount of a particular biological compound or derivative thereof present in a source rock or petroleum deposit may be reduced or eliminated by diagenesis, catagenesis, and metagenesis. Generally the proportion of a geologic, more thermodynamically stable stereoisomer relative to the proportion of the biological, less thermodynamically stable stereoisomer in a source rock or naturally occurring petroleum deposit can be used to gauge the amount of maturation of such source rock or petroleum deposit. Methylated phenanthrenes behave in a similar fashion during progressive maturation with certain attachment sites for the methyl group favored within certain maturation ranges.

FIG. 53 is a plot of the weight ratio of trisnorhopane maturable (Tm) to trisnorhopane maturable (Tm) plus trisnorhopane stable (Ts) or collectively (Tm to Tm+Ts) for examples 6-20. The y-axis 520 is the weight ratio of Tm to Tm+Ts which is a measure of the biological isomer Tm relative to the thermodynamically favored compound Ts. Thus a more geologically matured hydrocarbon substance would have a lower Tm to Tm+Ts ratio, while an immature biological hydrocarbon substance would be expected to have a Tm to Tm+Ts ratio of about 1. The x-axis 521 contains the experiment number from Examples 6-20. As can be seen the Example numbers have been consistently ordered on the x-axis from Example 6 to Example 19 in a manner consistent with the ordering of FIGS. 33, 35, 37 and 38 in order to relate the data in such figures to the data contained in FIGS. 53-59. In addition, following Example 19 is Example 20 which includes the unheated oil shale extraction described in Example 20 in the Experiments section. This same ordering of the x-axis will be used consistently in all of FIGS. 53-59. As can be seen from the graph points 522, all of the data for stressed Examples 8-12 at 375° C. and stressed Examples 15-19 at 393° C. generally fall in the same range of about 0.975 to 0.985. It is also evident that the 0 psi stressed experiments from Examples 6, 7, 13 and 14 are on the lower end of the scale, with Examples 13 and 14 being the only data points below 0.97. Further, it is apparent that the bitumen extracted from unheated oil shale in Example 20 is considerable higher at about 1.0 as would be expected for an immature petroleum hydrocarbon. From the literature, for example Peters, K. E., Walters, C. C., and Moldowan, J. M., The Biomarker Guide, Biomarkers and Isotopes in Petroleum Exploration and Earth History, Vol. 2, Cambridge University Press (2005), naturally occurring petroleum oil deposits generally have a Tm to Tm+Ts of 0.6 or less. Thus the hydrocarbon fluid produced in Examples 6-20 is between the immature extracted bitumen and the more matured naturally occurring petroleum hydrocarbon deposits. It is also apparent that the hydrocarbon fluids produced in Examples 6-20 are much more bitumen-like than like naturally occurring petroleum hydrocarbon deposits in terms of their Tm to Tm+Ts ratio. Further, the unstressed experiments produced a hydrocarbon fluid that is generally more matured than the stressed experiments.

FIG. 54 is a plot of the weight ratio of stereoisomers of the C-29 pentacyclic alkanes that are the most abundant triterpanes found in sediments and crude oils. Specifically the plot shows the weight ratio of C-29 17α(H), 21β(H) hopane to C-29 17α(H), 21β (H) hopane plus C-29 17β(H), 21β (H) hopane (29H αβ/29H αβ+29H ββ) for examples 6-20. The y-axis 530 is the weight ratio of 29H αβ to 29H αβ+29H ββ which is a measure of the thermodynamically stable isomer of C-29 hopane (29H αβ) relative to the biological form of C-29 hopane (29H ββ). Thus a more geologically matured hydrocarbon substance would have a higher 29H αβ to 29H αβ+29H ββ ratio of about 1 indicating little or none of the 29ββ isomer is present, while an immature biological hydrocarbon substance would be expected to have a lower 29H αβ to 29H αβ+29ββ ratio of less than 1. The x-axis 531 contains the experiment number from Example 6-20 in the order described for FIG. 53. As can be seen from the graph points 532, all of the data for the heating experiments of Examples 6-19 generally fall in the same range of about 0.3 to 0.45. Further, it is apparent that the bitumen extracted from unheated oil shale in Example 20 is considerable lower at about 0.17. From the literature, naturally occurring petroleum oil deposits generally have a 29H αβ to 29H αβ+29H ββ ratio of about 1, indicating that there is little or none of the 29H ββ isomer present. Thus the hydrocarbon fluids produced in Examples 6-19 are between the unmatured extracted bitumen and more matured naturally occurring petroleum hydrocarbon oil deposits with respect to the amount of the 29H ββ isomer present. Further, the hydrocarbon fluids produced in Examples 6-19 are more bitumen-like than like naturally occurring petroleum hydrocarbon oil deposits based on the amount of the 29H ββ isomer present.

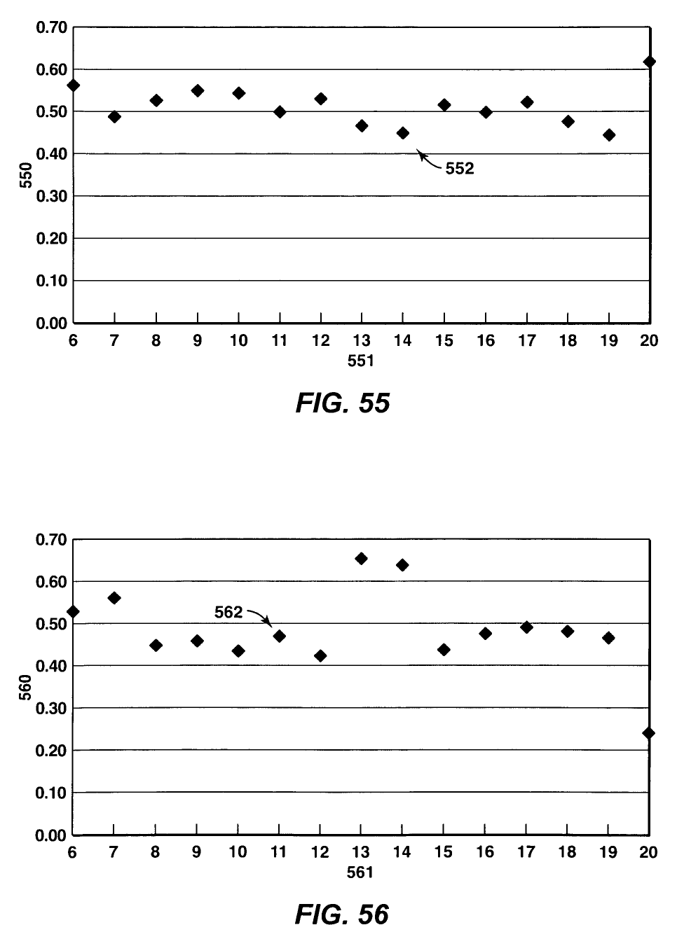

FIG. 55 is a plot of the stereoisomers of the C-30 pentacyclic alkanes that are the most abundant triterpanes found in sediments and crude oils. Specifically the plot shows the weight ratio of C-30 17α(H), 21β(H) hopane to C-30 17α(H), 21β(H) hopane plus C-30 17β(H), 21β(H) hopane (30H αβ/30H αβ+30H ββ) for examples 6-20. The y-axis 550 is the weight ratio of 30H αβ to 30H αβ+30H ββ which is a measure of the thermodynamically stable isomer of C-30 hopane (30H αβ) relative to the biological form of C-30 hopane (30H ββ). Thus a more matured hydrocarbon substance would have a higher 30H αβ to 30H αβ+30H ββ ratio of about 1 indicating little or none of the 30H ββ isomer is present, while an immature biological hydrocarbon substance would be expected to have a lower 30H αβ to 30H αβ+30H ββ ratio of less than 1. The x-axis 551 contains the experiment number from Example 6-20 in the order described for FIG. 53. As can be seen from the graph points 552, all of the data for the heating experiments of Examples 6-19 generally fall in the same range of about 0.44 to 0.57. Further, it is apparent that the bitumen extracted from unheated oil shale in Example 20 is slightly higher at about 0.62. From the literature, naturally occurring petroleum deposits generally have a 30H αβ to 30H αβ+30H ββ of about 1, indicating that there is little or none of the 30H ββ isomer present. Thus the hydrocarbon fluids produced in Examples 6-19 are similar to the less matured extracted bitumen in terms of the amount of the 30H ββ isomer present. Further, the hydrocarbon fluids produced in Examples 6-19 have more of the 30H ββ isomer present than a more matured naturally occurring petroleum hydrocarbon oil deposit. In addition the hydrocarbon fluids produced in Examples 6-19 are more bitumen-like than like naturally occurring petroleum hydrocarbon oil deposits.

FIG. 56 is a plot of the stereoisomers of the C-31 pentacyclic alkanes that are the most abundant triterpanes found in sediments and crude oils. Specifically the plot shows the weight ratio of C-31 17 α(H), 21β(H), 22S homohopane to C-31 17α(H), 21β(H), 22S homohopane plus C-31 17α(H), 21β(H), 22R homohopane (31H-S/31H-S+31H-R) for examples 6-20. The y-axis 560 is the weight ratio of 31H-S to 31H-S plus 31H-R which is a measure of the thermodynamically stable isomer of C-31 homohopane 31H-S relative to the biological isomer, 31H-R. Thus a more matured hydrocarbon substance would be expected to have a higher X31H-S to 31H-S plus 31H-R ratio, while a less matured biological hydrocarbon substance would be expected to have a lower 31H-S to 31H-S plus 31H-R ratio. The x-axis 561 contains the experiment number from Example 6-20 in the order described for FIG. 53. As can be seen from the graph points 562, all of the data for the stressed Examples 8-12 at 375° C. and stressed Examples 15-19 at 393° C. generally fall in the same range of about 0.42 to 0.5. It is also evident that the 0 psi stressed experiments from Examples 6, 7, 13 and 14 are on the upper end of the scale, with all such Examples falling above 0.52 all the way up to about 0.65 for Examples 13 & 14. Further, it is apparent that the bitumen extracted from unheated oil shale in Example 20 is considerable lower at about 0.25. From the literature, naturally occurring petroleum deposits generally have a (31H-S/31H-S+31H-R) ranging from about 0.58 to 0.63 (Seifert, W. K., and Moldowan, J. M., The Effect of Thermal Stress on Source-rock Quality as Measured by Hopane Stereochemistry, Physics and Chemistry of the Earth, 12, 229-237 (1980)). Thus with respect to the presence of 31H-S to 31H-S plus 31H-R ratio, the hydrocarbon fluids produced in the stressed Examples 8-12 and 15-19 are close to but less than what is expected for naturally occurring petroleum hydrocarbon oil deposits. Further, the unstressed experiments produced a hydrocarbon fluid that is most like naturally occurring oils. In addition, it is apparent that with respect to the presence of the 31H-R isomer of C-31 homohopane, the hydrocarbon fluids produced in Examples 6-19 are more like naturally occurring petroleum hydrocarbon oil deposits than like bitumen.

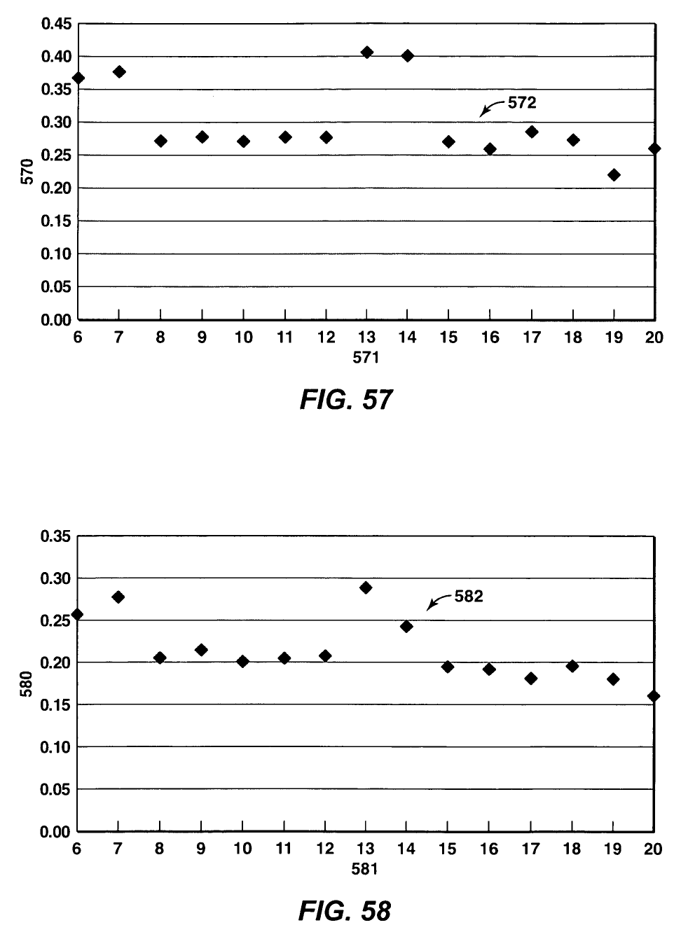

FIG. 57 is a plot of the weight ratio of the C-29 5 α, 14 α, 17 α (H) 20R steranes to the C-29 5 α, 14 α, 17α (H) 20R steranes plus the C-29 5 α, 14 α, 17 α (H) 20S steranes (C-29 ααα S/C-29 ααα S+C-29 ααα R) for examples 6-20. The y-axis 570 is the weight ratio of C-29 ααα S to C-29 ααα S plus C-29 ααα R which is a measure of the thermodynamically stable isomer of C-29 sterane (C-29 ααα S) relative to the biological isomer of C-29 sterane (C-29 ααα R). Thus a more matured hydrocarbon substance would be expected to have a higher C-29 ααα S to C-29 ααα S plus C-29 ααα R ratio, while an immature hydrocarbon substance would be expected to have a lower C-29 ααα S to C-29 ααα S plus C-29 ααα R ratio. The x-axis 571 contains the experiment number from Example 6-20 in the order described for FIG. 53. As can be seen from the graph points 572, all of the data for the for stressed Examples 8-12 at 375° C. and stressed Examples 15-19 at 393° C. generally fall in the same range of about 0.22 to 0.3. It is also evident that the 0 psi stressed experiments from Examples 6, 7, 13 and 14 are on the upper end of the scale, with all such Examples falling above 0.36 all the way up to about 0.41 for Examples 13 & 14. Further, it is apparent that the bitumen extracted from unheated oil shale in Example 20 is in the same range as the stressed experiments at about 0.26. From the literature, naturally occurring petroleum deposits generally have an equilibrium C-29 ααα S to C-29 ααα S plus C-29 ααα R ratio of about 0.5. Thus with respect to the presence of C-29 ααα S relative to C-29 ααα R, the hydrocarbon fluids produced in the stressed Examples 8-12 and 15-19 have ratios much lower than what is expected for naturally occurring petroleum hydrocarbon oil deposits and are essentially the same as the source rock bitumen. Further, the unstressed experiments produced hydrocarbon fluids have ratios that are more like naturally occurring oils.

FIG. 58 is a plot of the C-29 5 α, 14 β, 17β (H) 20S plus C-29 5 α, 14 β, 17 β (H) 20R steranes to the C-29 5 α, 14 β, 17 β (H) 20S plus C-29 5 α, 14 β, 17 β (H) 20R steranes plus C-29 5 α, 14 α, 17α (H) 20S plus C-29 5 α, 14 α, 17α (H) 20R steranes (C-29 αββ S&R/C-29 αββ S&R+C-29 ααα S&R) for examples 6-20. The y-axis 580 is the weight ratio of C-29 αββ S&R to C-29 αββ S&R plus C-29 ααα S&R which is a measure of the thermodynamically stable isomers of the C-29 steranes (C-29 αββ S&R) relative to the biological isomers of C-29 sterane (C-29 ααα S&R). Thus a more matured hydrocarbon substance would be expected to have a higher C-29 αββ S&R to C-29 αββ S&R plus C-29 ααα S&R ratio, while an immature biological hydrocarbon substance would be expected to have a lower C-29 αββ S&R to C-29 αββ S&R plus C-29 ααα S&R ratio. The x-axis 581 contains the experiment number from Example 6-20 in the order described for FIG. 53. As can be seen from the graph points 582, all of the data for the for stressed Examples 8-12 at 375° C. and stressed Examples 15-19 at 393° C. generally fall in the same range of about 0.17 to 0.22. It is also evident that the 0 psi stressed experiments from Examples 6, 7, 13 and 14 are on the upper end of the scale, with all such Examples falling above 0.24 all the way up to about 0.29 for Example 13. Further, it is apparent that the bitumen extracted from unheated oil shale in Example 20 is slightly lower than the stressed experiments at about 0.16. From the literature, naturally occurring oils reach an equilibrium C-29 αββ S&R to C-29 αββ S&R plus C-29 ααα S&R ratio of 0.75. Thus with respect to the C-29 αββ S&R to C-29αββ S&R plus C-29 ααα S&R ratio, the hydrocarbon fluid produced in the stressed Examples 8-12 and 15-19 have ratios much lower than what is expected for naturally occurring oils. Further, the unstressed experiments produced a hydrocarbon fluid that is more like naturally oils but still quite distinct.

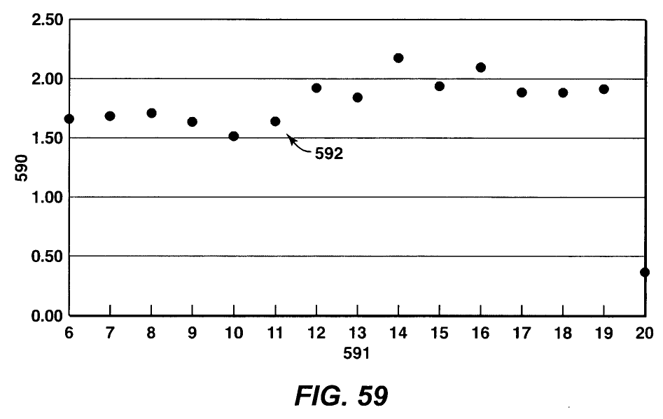

FIG. 59 is a plot of the weight ratio of 3-methyl phenanthrene (3-MP)+2-methyl phenanthrene (2-MP) to 1-methyl phenanthrene (1-MP)+9-methyl phenanthrene (9-MP) for examples 6-20. The y-axis 590 is the weight ratio of (3-MP+2-MP) to (1-MP+9-MP) which is a measure of the higher temperature stability forms of methyl phenanthrene (3-MP+2-MP) relative to the lower temperature stability forms of methyl phenanthrene (1-MP+9-MP). Thus a less matured hydrocarbon substance would be expected to have a lower (3-MP+2-MP) to (1-MP+9-MP) ratio, while a more matured hydrocarbon substance would be expected to have a higher (3-MP+2-MP) to (1-MP+9-MP) ratio—for maturities within the oil generation window. The x-axis 591 contains the experiment number from Example 6-20 in the order described for FIG. 53. As can be seen from the graph points 592, all of the data for the heating experiments of Examples 6-19 generally fall in the range of about 1.5 to 2.25. Further, it is apparent that the bitumen extracted from unheated oil shale in Example 20 is considerable lower at about 0.4. From the literature (e.g. Radke, M., Organic Geochemistry of Aromatic Hydrocarbons, Adv. in Petroleum Geochemistry vol. 2, Ed: Jim Brooks and Dietrich Welte, Academic Press, London (1987) p. 141-208.), naturally occurring petroleum deposits generally have a (3-MP+2-MP) to (1-MP+9-MP) of about 0.4-0.5 for immature materials like bitumen and about 0.6-1.5 for naturally occurring petroleum hydrocarbon oil deposits. Thus, indicating that there is more 3-MP+2-MP present as the naturally occurring petroleum hydrocarbon becomes more matured. Thus, the hydrocarbon fluids produced in Examples 6-19 have methyl-phenanthrene ratios unlike both bitumen and naturally occurring oils.

From the above-described data discussed in FIGS. 53-59, it can be seen that hydrocarbon fluids produced by pyrolysis under stress loading have certain maturity characteristics as judged by the stereochemical biomarker ratio and methyl phenanthrene relationships discussed above. In many cases the presence or absence of stress loading also correlates to step changes in particular biomarker relationships. Some of the hydrocarbon fluid produced from pyrolysis of oil shale shows a more bitumen-like characteristic for some of the above-described biomarker relationships, while for other relationships the produced hydrocarbon fluid is, more like a naturally occurring petroleum hydrocarbon oil. Further, some of the hydrocarbon fluid produced from pyrolysis of oil shale is neither bitumen-like nor like naturally occurring petroleum hydrocarbon oil for some of the above-described relationships. This implies that the composition of the produced hydrocarbon fluid from in situ heating and pyrolysis processes will be unlike naturally occurring petroleum hydrocarbon deposits and also unlike shale oil produced in an ex-situ retorting process.

In some embodiments, the produced hydrocarbon fluid includes a condensable hydrocarbon portion. In some embodiments the condensable hydrocarbon portion may have a (trisnorhopane maturable (Tm)) to (trisnorhopane maturable (Tm)+trisnorhopane stable (Ts)) or alternatively (Tm/Tm+Ts) weight ratio greater than 0.7. In alternate embodiments the condensable hydrocarbon portion may have a (trisnorhopane maturable) to (trisnorhopane maturable+trisnorhopane stable) weight ratio greater than 0.8, 0.9 or 0.95. In alternate embodiments the condensable hydrocarbon portion may have a (trisnorhopane maturable) to (trisnorhopane maturable+trisnorhopane stable) weight ratio between 0.7 and 0.995, between 0.8 and 0.990, or between 0.7 and 0.995.

In some embodiments, the produced hydrocarbon fluid includes a condensable hydrocarbon portion. In some embodiments the condensable hydrocarbon portion may have a [C-29 17α(H), 21β(H) hopane] to [C-29 17α(H), 21β(H) hopane+C-29 17β(H), 21β(H) hopane] or alternatively (29H αβ/29H αβ+29H ββ) weight ratio less than 0.9. In alternate embodiments the condensable hydrocarbon portion may have a [C-29 17α(H), 21β(H) hopane] to [C-29 17α(H), 21β(H) hopane+C-29 17β(H), 21β(H) hopane] weight ratio less than 0.8, 0.7 or 0.6. In alternate embodiments the condensable hydrocarbon portion may have a [C-29 17α(H), 21β(H) hopane] to [C-29 17α(H), 21β(H) hopane+C-29 17β(H), 21β(H) hopane] weight ratio between 0.2 and 0.9, between 0.25 and 0.6, or between 0.3 and 0.5.

In some embodiments, the produced hydrocarbon fluid includes a condensable hydrocarbon portion. In some embodiments the condensable hydrocarbon portion may have a [C-30 17α(H), 21β(H) hopane] to [C-30 17α(H), 21β(H) hopane+C-30 17β(H), 21β(H) hopane] or alternatively (30H αβ/30H αβ+30H ββ) weight ratio less than 0.9. In alternate embodiments the condensable hydrocarbon portion may have a [C-30 17α(H), 21β(H) hopane] to [C-30 17α(H), 21β(H) hopane+C-30 17β(H), 21β(H) hopane] weight ratio less than 0.8, 0.7 or 0.6. In alternate embodiments the condensable hydrocarbon portion may have a [C-30 17α(H), 21β(H) hopane] to [C-30 17α(H), 21β(H) hopane+C-30 17β(H), 21β(H) hopane] weight ratio between 0.3 and 0.62, between 0.35 and 0.60, or between 0.4 and 0.58.

In some embodiments, the produced hydrocarbon fluid includes a condensable hydrocarbon portion. In some embodiments the condensable hydrocarbon portion may have a [C-31 17α(H), 21β(H), 22S homohopane] to [C-31 17α(H), 21β(H), 22S homohopane+C-31 17α(H), 21β(H), 22R homohopane] or alternatively (31H-S/31H-S+31H-R) weight ratio less than 0.6. In alternate embodiments the condensable hydrocarbon portion may have a [C-31 17αX(H), 21β(H), 22S homohopane] to [C-31 17α(H), 21β(H), 22S homohopane+C-31 17α(H), 21β(H), 22R homohopane] weight ratio less than 0.58, 0.55 or 0.50. In alternate embodiments the condensable hydrocarbon portion may have a [C-31 17α(H), 21β(H), 22S homohopane] to [C-31 17α(H), 21β(H), 22S homohopane+C-31 17α(H), 21β(H), 22R homohopane] weight ratio between 0.25 and 0.6, between 0.3 and 0.58, or between 0.4 and 0.55.

In some embodiments, the produced hydrocarbon fluid includes a condensable hydrocarbon portion. In some embodiments the condensable hydrocarbon portion may have a [C-29 5 α, 14 α, 17 α (H) 20R steranes] to [C-29 5 α, 14 α, 17 α (H) 20R steranes+C-29 5 α, 14 α, 17 α (H) 20S steranes] or alternatively (C-29 ααα S/C-29 ααα S+C-29 ααα R) weight ratio less than 0.7. In alternate embodiments the condensable hydrocarbon portion may have a [C-29 5 α, 14 α, 17 α (H) 20R steranes] to [C-29 5 α, 14 α, 17 α (H) 20R steranes+C-29 5 α, 14 α, 17 α (H) 20S steranes] weight ratio less than 0.6, 0.5 or 0.4. In alternate embodiments the condensable hydrocarbon portion may have a [C-29 5 α, 14 α, 17 α (H) 20R steranes] to [C-29 5 α, 14 α, 17 α (H) 20R steranes+C-29 5 α, 14 α, 17 α (H) 20S steranes] weight ratio between 0.2 and 0.7, between 0.25 and 0.5, or between 0.25 and 0.3.

In some embodiments, the produced hydrocarbon fluid includes a condensable hydrocarbon portion. In some embodiments the condensable hydrocarbon portion may have a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] or alternatively (C-29αββ S&R/C-29αββ S&R+C-29 ααα S&R) weight ratio less than 0.7. In alternate embodiments the condensable hydrocarbon portion may have a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] weight ratio less than 0.6, 0.4, 0.25 or 0.24. In alternate embodiments the condensable hydrocarbon portion may have a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] weight ratio between 0.15 and 0.7, between 0.17 and 0.5, or between 0.17 and 0.25.

In some embodiments, the produced hydrocarbon fluid includes a condensable hydrocarbon portion. In some embodiments the condensable hydrocarbon portion may have a [3-methyl phenanthrene (3-MP)+2-methyl phenanthrene (2-MP)]/[1-methyl phenanthrene (1-MP)+9-methyl phenanthrene (9-MP)] weight ratio greater than 0.5. In alternate embodiments the condensable hydrocarbon portion may have a [3-methyl phenanthrene (3-MP)+2-methyl phenanthrene (2-MP)]/[1-methyl phenanthrene (1-MP)+9-methyl phenanthrene (9-MP)] weight ratio greater than 0.75, 1.0 or 1.25. In alternate embodiments the condensable hydrocarbon portion may have a [3-methyl phenanthrene (3-MP)+2-methyl phenanthrene (2-MP)]/[1-methyl phenanthrene (1-MP)+9-methyl phenanthrene (9-MP)] weight ratio between 0.5 and 3.0, between 1.0 and 2.5, or between 1.25 and 2.5.

In some embodiments the condensable hydrocarbon portion may have one or more of a (trisnorhopane maturable (Tm)) to (trisnorhopane maturable (Tm)+trisnorhopane stable (Ts)) or alternatively (Tm/Tm+Ts) weight ratio greater than 0.7, a [C-29 17α(H), 21β(H) hopane] to [C-29 17α(H), 21β(H) hopane+C-29 17β(H), 21β(H) hopane] or alternatively (29H αβ/29H αβ+29H ββ) weight ratio less than 0.9, a [C-30 17α(H), 21β(H) hopane] to [C-30 17α(H), 21p(H) hopane+C-30 17β(H), 21β(H) hopane] or alternatively (30H αβ/30H αβ+30H ββ) weight ratio less than 0.9, a [C-31 17α(H), 21β(H), 22S homohopane] to [C-31 17α(H), 21β(H), 22S homohopane+C-31 17α(H), 21β(H), 22R homohopane] or alternatively (31H-S/31H-S+31H-R) weight ratio less than 0.6, a [C-29 5 α, 14 α, 17 α (H) 20R steranes] to [C-29 5 α, 14 α, 17 α (H) 20R steranes+C-29 5 α, 14 α, 17 α (H) 20S steranes] or alternatively (C-29 ααα S/C-29 ααα S+C-29 ααα R) weight ratio less than 0.7, a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] or alternatively (C-29 ααα S&R/C-29αββ S&R+C-29 ααα S&R) weight ratio less than 0.7, and a [3-methyl phenanthrene (3-MP)+2-methyl phenanthrene (2-MP)] to [1-methyl phenanthrene (1-MP)+9-methyl phenanthrene (9-MP)] weight ratio greater than 0.5. In some embodiments the condensable hydrocarbon portion may have two or more, three or more or four or more of the weight ratios described above in the paragraph.

In some embodiments the condensable hydrocarbon portion may have one or more of a (trisnorhopane maturable (Tm)) to (trisnorhopane maturable (Tm)+trisnorhopane stable (Ts)) or alternatively (Tm/Tm+Ts) weight ratio greater than 0.8, a [C-29 17α(H), 21β(H) hopane] to [C-29 17α(H), 21β(H) hopane+C-29 17β(H), 21β(H) hopane] or alternatively (29H αβ/29H αβ+29H ββ) weight ratio less than 0.8, a [C-30 17α(H), 21β(H) hopane] to [C-30 17α(H), 21β(H) hopane+C-30 17β(H), 21β(H) hopane] or alternatively (30H αβ/30H αβ+30H ββ) weight ratio less than 0.8, a [C-31 17α(H), 21β(H), 22S homohopane] to [C-31 17α(H), 21β(H), 22S homohopane+C-31 17α(H), 21β(H), 22R homohopane] or alternatively (31H-S/31H-S+31H-R) weight ratio less than 0.58, a [C-29 5 α, 14 α, 17 α (H) 20R steranes] to [C-29 5 α, 14 α, 17 α (H) 20R steranes+C-29 5 α, 14 α, 17 α (H) 20S steranes] or alternatively (C-29 ααα S/C-29 ααα S+C-29 ααα R) weight ratio less than 0.6, a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] or alternatively (C-29 αββ S&R/C-29 αββ S&R+C-29 ααα S&R) weight ratio less than 0.6, and a [3-methyl phenanthrene (3-MP)+2-methyl phenanthrene (2-MP)] to [1-methyl phenanthrene (1-MP)+9-methyl phenanthrene (9-MP)] weight ratio greater than 0.75. In some embodiments the condensable hydrocarbon portion may have two or more, three or more or four or more of the weight ratios described above in the paragraph.

In some embodiments the condensable hydrocarbon portion may have one or more of a (trisnorhopane maturable (Tm)) to (trisnorhopane maturable (Tm)+trisnorhopane stable (Ts)) or alternatively (Tm/Tm+Ts) weight ratio greater than 0.7, a [C-29 17α(H), 21β(H) hopane] to [C-29 17α(H), 21β(H) hopane+C-29 17β(H), 21β(H) hopane] or alternatively (29H αβ/29H αβ+29H ββ) weight ratio less than 0.7, a [C-30 17α(H), 21β(H) hopane] to [C-30 17α(H), 21β(H) hopane+C-30 17β(H), 21β(H) hopane] or alternatively (30H αβ/30H αβ+30H ββ) weight ratio less than 0.7, a [C-31 17α(H), 21β(H), 22S homohopane] to [C-31 17α(H), 210(H), 22S homohopane+C-31 17α(H), 21β(H), 22R homohopane] or alternatively (31H-S/31H-S+31H-R) weight ratio less than 0.55, a [C-29 5 α, 14 α, 17 α (H) 20R steranes] to [C-29 5 α, 14 α, 17 α (H) 20R steranes+C-29 5 α, 14 α, 17 α (H) 20S steranes] or alternatively (C-29 ααα S/C-29 ααα S+C-29 ααα R) weight ratio less than 0.5, a [C-29 5 α, 14 β, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] or alternatively (C-29 αββ S&R/C-29 αββ S&R+C-29 ααα S&R) weight ratio less than 0.55, and a [3-methyl phenanthrene (3-MP)+2-methyl phenanthrene (2-MP)] to [1-methyl phenanthrene (1-MP)+9-methyl phenanthrene (9-MP)] weight ratio greater than 0.1.0. In some embodiments the condensable hydrocarbon portion may have two or more, three or more or four or more of the weight ratios described above in the paragraph.

As used in the preceding paragraphs and in the claims with respect to hopanes, steranes and phenanthrenes, the phrase “one or more” followed by a listing of different compound or component ratios with the last ratio introduced by the conjunction “and” is meant to include a condensable hydrocarbon portion that has at least one of the listed ratios or that has two or more, or three or more, or four or more, etc., or all of the listed ratios. Further, a particular condensable hydrocarbon portion may also have additional ratios of different compounds or components that are not included in a particular sentence or claim and still fall within the scope of such a sentence or claim. The embodiments described in the preceding paragraphs may be combined with any of the other aspects of the invention discussed herein. Certain features of the present invention discussed in the preceding paragraphs are described in terms of a set of numerical upper limits (e.g. “less than”) and a set of numerical lower limits (e.g. “greater than”) in the preceding paragraph. It should be appreciated that ranges formed by any combination of these limits are within the scope of the invention unless otherwise indicated. The embodiments described in the preceding paragraphs may be combined with any of the other aspects of the invention discussed in such paragraphs or otherwise herein.

As used herein and in the claims, weight ratios of hopanes (e.g., C-29 17α(H), 21β(H) hopane), steranes (e.g., C-29 5 α, 14 α, 17 α (H) 20R steranes) and phenanthrenes (e.g., 3-methyl phenanthrene) is meant to refer to the amount of a particular hydrocarbon compound found in a condensable hydrocarbon fluid as determined by liquid chromatography (LC) followed by gas chromatography/mass spectrometry (GC/MS) as described herein, particularly in the section labeled “Experiments” herein. That is, the amount of a respective compound is determined from the respective GC/MS peak height determined using the LC and GC/MS analysis methodology according to the procedure described in the Experiments section of this application. Further, when a first compound is compared to a second compound in a weight ratio herein and in the claims, such a weight ratio is obtained by the ratio of the first compound's GC/MS peak height to the second compound's GC/MS peak height. For example, a [C-29 17α(H), 21β(H) hopane] to [C-29 17α(H), 21β(H) hopane+C-29 17β(H), 21β(H) hopane] weigh ratio is obtained by dividing the [C-29 17α(H), 21β(H) hopane] GC/MS peak height for a respective condensable hydrocarbon fluid by the total GC/MS peak height for [C-29 17α(H), 21β(H) hopane] and [C-29 17β(H), 21β(H) hopane], where both of such respective GC/MS peak heights are determined by the by liquid chromatography (LC) and gas chromatography/mass spectrometry (GC/MS) analysis procedures, GC/MS peak identification methodologies, and GC/MS peak measurement methodologies discussed in the Experiments section herein.

The discovery that lithostatic stress can affect the composition of produced fluids generated within an organic-rich rock via heating and pyrolysis implies that the composition of the produced hydrocarbon fluid can also be influenced by altering the lithostatic stress of the organic-rich rock formation. For example, the lithostatic stress of the organic-rich rock formation may be altered by choice of pillar geometries and/or locations and/or by choice of heating and pyrolysis formation region thickness and/or heating sequencing.

Pillars are regions within the organic-rich rock formation left unpyrolized at a given time to lessen or mitigate surface subsidence. Pillars may be regions within a formation surrounded by pyrolysis regions within the same formation. Alternatively, pillars may be part of or connected to the unheated regions outside the general development area. Certain regions that act as pillars early in the life of a producing field may be converted to producing regions later in the life of the field.

Typically in its natural state, the weight of a formation's overburden is fairly uniformly distributed over the formation. In this state the lithostatic stress existing at particular point within a formation is largely controlled by the thickness and density of the overburden. A desired lithostatic stress may be selected by analyzing overburden geology and choosing a position with an appropriate depth and position.

Although lithostatic stresses are commonly assumed to be set by nature and not changeable short of removing all or part of the overburden, lithostatic stress at a specific location within a formation can be adjusted by redistributing the overburden weight so it is not uniformly supported by the formation. For example, this redistribution of overburden weight may be accomplished by two exemplary methods. One or both of these methods may be used within a single formation. In certain cases, one method may be primarily used earlier in time whereas the other may be primarily used at a later time. Favorably altering the lithostatic stress experienced by a formation region may be performed prior to instigating significant pyrolysis within the formation region and also before generating significant hydrocarbon fluids. Alternately, favorably altering the lithostatic stress may be performed simultaneously with the pyrolysis.

A first method of altering lithostatic stress involves making a region of a subsurface formation less stiff than its neighboring regions. Neighboring regions thus increasingly act as pillars supporting the overburden as a particular region becomes less stiff. These pillar regions experience increased lithostatic stress whereas the less stiff region experiences reduced lithostatic stress. The amount of change in lithostatic stress depends upon a number of factors including, for example, the change in stiffness of the treated region, the size of the treated region, the pillar size, the pillar spacing, the rock compressibility, and the rock strength. In an organic-rich rock formation, a region within a formation may be made to experience mechanical weakening by pyrolyzing the region and creating void space within the region by removing produced fluids. In this way a region within a formation may be made less stiff than neighboring regions that have not experienced pyrolysis or have experienced a lesser degree of pyrolysis or production.

A second method of altering lithostatic stress involves causing a region of a subsurface formation to expand and push against the overburden with greater force than neighboring regions. This expansion may remove a portion of the overburden weight from the neighboring regions thus increasing the lithostatic stress experienced by the heated region and reducing the lithostatic stress experienced by neighboring regions. If the expansion is sufficient, horizontal fractures will form in the neighboring regions and the contribution of these regions to supporting the overburden will decrease. The amount of change in lithostatic stress depends upon a number of factors including, for example, the amount of expansion in the treated region, the size of the treated region, the pillar size, the pillar spacing, the rock compressibility, and the rock strength. A region within a formation may be made to expand by heating it so to cause thermal expansion of the rock. Fluid expansion or fluid generation can also contribute to expansion if the fluids are largely trapped within the region. The total expansion amount may be proportional to the thickness of the heated region. It is noted that if pyrolysis occurs in the heated region and sufficient fluids are removed, the heated region may mechanically weaken and thus may alter the lithostatic stresses experienced by the neighboring regions as described in the first exemplary method.

Embodiments of the method may include controlling the composition of produced hydrocarbon fluids generated by heating and pyrolysis from a first region within an organic-rich rock formation by increasing the lithostatic stresses within the first region by first heating and pyrolyzing formation hydrocarbons present in the organic-rich rock formation and producing fluids from a second neighboring region within the organic-rich rock formation such that the Young's modulus (i.e., stiffness) of the second region is reduced.

Embodiments of the method may include controlling the composition of produced hydrocarbon fluids generated by heating and pyrolysis from a first region within an organic-rich rock formation by increasing the lithostatic stresses within the first region by heating the first region prior to or to a greater degree than neighboring regions within the organic-rich rock formation such that the thermal expansion within the first region is greater than that within the neighboring regions of the organic-rich rock formation.

Embodiments of the method may include controlling the composition of produced hydrocarbon fluids generated by heating and pyrolysis from a first region within an organic-rich rock formation by decreasing the lithostatic stresses within the first region by heating one or more neighboring regions of the organic-rich rock formation prior to or to a greater degree than the first region such that the thermal expansion within the neighboring regions is greater than that within the first region.

Embodiments of the method may include locating, sizing, and/or timing the heating of heated regions within an organic-rich rock formation so as to alter the in situ lithostatic stresses of current or future heating and pyrolysis regions within the organic-rich rock formation so as to control the composition of produced hydrocarbon fluids.

Some production procedures include in situ heating of an organic-rich rock formation that contains both formation hydrocarbons and formation water-soluble minerals prior to substantial removal of the formation water-soluble minerals from the organic-rich rock formation. In some embodiments of the invention there is no need to partially, substantially or completely remove the water-soluble minerals prior to in situ heating. For example, in an oil shale formation that contains naturally occurring nahcolite, the oil shale may be heated prior to substantial removal of the nahcolite by solution mining. Substantial removal of a water-soluble mineral may represent the degree of removal of a water-soluble mineral that occurs from any commercial solution mining operation as known in the art. Substantial removal of a water-soluble mineral may be approximated as removal of greater than 5 weight percent of the total amount of a particular water-soluble mineral present in the zone targeted for hydrocarbon fluid production in the organic-rich rock formation. In alternative embodiments, in situ heating of the organic-rich rock formation to pyrolyze formation hydrocarbons may be commenced prior to removal of greater than 3 weight percent, alternatively 7 weight percent, 10 weight percent or 13 weight percent of the formation water-soluble minerals from the organic-rich rock formation.

The impact of heating oil shale to produce oil and gas prior to producing nahcolite is to convert the nahcolite to a more recoverable form (soda ash), and provide permeability facilitating its subsequent recovery. Water-soluble mineral recovery may take place as soon as the retorted oil is produced, or it may be left for a period of years for later recovery. If desired, the soda ash can be readily converted back to nahcolite on the surface. The ease with which this conversion can be accomplished makes the two minerals effectively interchangeable.

In some production processes, heating the organic-rich rock formation includes generating soda ash by decomposition of nahcolite. The method may include processing an aqueous solution containing water-soluble minerals in a surface facility to remove a portion of the water-soluble minerals. The processing step may include removing the water-soluble minerals by precipitation caused by altering the temperature of the aqueous solution.

The water-soluble minerals may include sodium. The water-soluble minerals may also include nahcolite (sodium bicarbonate), soda ash (sodium carbonate), dawsonite (NaAl(CO₃)(OH)₂), or combinations thereof. The surface processing may further include converting the soda ash back to sodium bicarbonate (nahcolite) in the surface facility by reaction with CO₂. After partial or complete removal of the water-soluble minerals, the aqueous solution may be reinjected into a subsurface formation where it may be sequestered. The subsurface formation may be the same as or different from the original organic-rich rock formation.

In some production processes, heating of the organic-rich rock formation both pyrolyzes at least a portion of the formation hydrocarbons to create hydrocarbon fluids and makes available migratory contaminant species previously bound in the organic-rich rock formation. The migratory contaminant species may be formed through pyrolysis of the formation hydrocarbons, may be liberated from the formation itself upon heating, or may be made accessible through the creation of increased permeability upon heating of the formation. The migratory contaminant species may be soluble in water or other aqueous fluids present in or injected into the organic-rich rock formation.

Producing hydrocarbons from pyrolyzed oil shale will generally leave behind some migratory contaminant species which are at least partially water-soluble. Depending on the hydrological connectivity of the pyrolyzed shale oil to shallower zones, these components may eventually migrate into ground water in concentrations which are environmentally unacceptable. The types of potential migratory contaminant species depend on the nature of the oil shale pyrolysis and the composition of the oil shale being converted. If the pyrolysis is performed in the absence of oxygen or air, the contaminant species may include aromatic hydrocarbons (e.g. benzene, toluene, ethylbenzene, xylenes), polyaromatic hydrocarbons (e.g. anthracene, pyrene, naphthalene, chrysene), metal contaminants (e.g. As, Co, Pb, Mo, Ni, and Zn), and other species such as sulfates, ammonia, Al, K, Mg, chlorides, flourides and phenols. If oxygen or air is employed, contaminant species may also include ketones, alcohols, and cyanides. Further, the specific migratory contaminant species present may include any subset or combination of the above-described species.

It may be desirable for a field developer to assess the connectivity of the organic-rich rock formation to aquifers. This may be done to determine if, or to what extent, in situ pyrolysis of formation hydrocarbons in the organic-rich rock formation may create migratory species with the propensity to migrate into an aquifer. If the organic-rich rock formation is hydrologically connected to an aquifer, precautions may be taken to reduce or prevent species generated or liberated during pyrolysis from entering the aquifer. Alternatively, the organic-rich rock formation may be flushed with water or an aqueous fluid after pyrolysis as described herein to remove water-soluble minerals and/or migratory contaminant species. In other embodiments, the organic-rich rock formation may be substantially hydrologically unconnected to any source of ground water. In such a case, flushing the organic-rich rock formation may not be desirable for removal of migratory contaminant species but may nevertheless be desirable for recovery of water-soluble minerals.

Following production of hydrocarbons from an organic-rich formation, some migratory contaminant species may remain in the rock formation. In such case, it may be desirable to inject an aqueous fluid into the organic-rich rock formation and have the injected aqueous fluid dissolve at least a portion of the water-soluble minerals and/or the migratory contaminant species to form an aqueous solution. The aqueous solution may then be produced from the organic-rich rock formation through, for example, solution production wells. The aqueous fluid may be adjusted to increase the solubility of the migratory contaminant species and/or the water-soluble minerals. The adjustment may include the addition of an acid or base to adjust the pH of the solution. The resulting aqueous solution may then be produced from the organic-rich rock formation to the surface for processing.

After initial aqueous fluid production, it may further be desirable to flush the matured organic-rich rock zone and the unmatured organic-rich rock zone with an aqueous fluid. The aqueous fluid may be used to further dissolve water-soluble minerals and migratory contaminant species. The flushing may optionally be completed after a substantial portion of the hydrocarbon fluids have been produced from the matured organic-rich rock zone. In some embodiments, the flushing step may be delayed after the hydrocarbon fluid production step. The flushing may be delayed to allow heat generated from the heating step to migrate deeper into surrounding unmatured organic-rich rock zones to convert nahcolite within the surrounding unmatured organic-rich rock zones to soda ash. Alternatively, the flushing may be delayed to allow heat generated from the heating step to generate permeability within the surrounding unmatured organic-rich rock zones. Further, the flushing may be delayed based on current and/or forecast market prices of sodium bicarbonate, soda ash, or both as further discussed herein. This method may be combined with any of the other aspects of the invention as discussed herein

Upon flushing of an aqueous solution, it may be desirable to process the aqueous solution in a surface facility to remove at least some of the migratory contaminant species. The migratory contaminant species may be removed through use of, for example, an adsorbent material, reverse osmosis, chemical oxidation, bio-oxidation, and/or ion exchange. Examples of these processes are individually known in the art. Exemplary adsorbent materials may include activated carbon, clay, or fuller's earth.

In certain areas with oil shale resources, additional oil shale resources or other hydrocarbon resources may exist at lower depths. Other hydrocarbon resources may include natural gas in low permeability formations (so-called “tight gas”) or natural gas trapped in and adsorbed on coal (so called “coal bed methane”). In some embodiments with multiple shale oil resources it may be advantageous to develop deeper zones first and then sequentially shallower zones. In this way, wells will need not cross hot zones or zones of weakened rock. In other embodiments in may be advantageous to develop deeper zones by drilling wells through regions being utilized as pillars for shale oil development at a shallower depth.

Simultaneous development of shale oil resources and natural gas resources in the same area can synergistically utilize certain facility and logistic operations. For example, gas treating may be performed at a single plant. Likewise personnel may be shared among the developments.

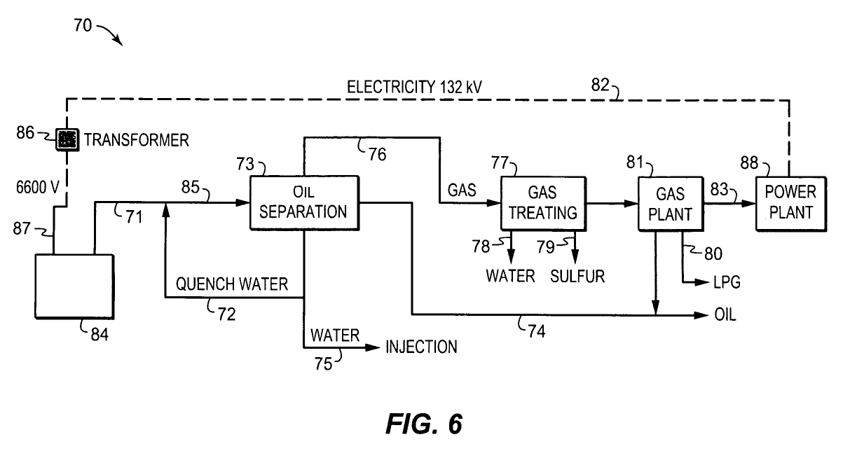

FIG. 6 illustrates a schematic diagram of an embodiment of surface facilities 70 that may be configured to treat a produced fluid. The produced fluid 85 may be produced from the subsurface formation 84 though a production well 71 as described herein. The produced fluid may include any of the produced fluids produced by any of the methods as described herein. The subsurface formation 84 may be any subsurface formation, including, for example, an organic-rich rock formation containing any of oil shale, coal, or tar sands for example. A production scheme may involve quenching 72 produced fluids to a temperature below 300° F., 200° F., or even 100° F., separating out condensable components (i.e., oil 74 and water 75) in an oil separator 73, treating the noncondensable components 76 (i.e. gas) in a gas treating unit 77 to remove water 78 and sulfur species 79, removing the heavier components from the gas (e.g., propane and butanes) in a gas plant 81 to form liquid petroleum gas (LPG) 80 for sale, and generating electrical power 82 in a power plant 88 from the remaining gas 83. The electrical power 82 may be used as an energy source for heating the subsurface formation 84 through any of the methods described herein. For example, the electrical power 82 may be feed at a high voltage, for example 132 kV, to a transformer 86 and let down to a lower voltage, for example 6600 V, before being fed to an electrical resistance heater element located in a heater well 87 located in the subsurface formation 84. In this way all or a portion of the power required to heat the subsurface formation 84 may be generated from the non-condensable portion of the produced fluids 85. Excess gas, if available, may be exported for sale.

Produced fluids from in situ oil shale production contain a number of components which may be separated in surface facilities. The produced fluids typically contain water, noncondensable hydrocarbon alkane species (e.g., methane, ethane, propane, n-butane, isobutane), noncondensable hydrocarbon alkene species (e.g., ethene, propene), condensable hydrocarbon species composed of (alkanes, olefins, aromatics, and polyaromatics among others), CO₂, CO, H₂, H₂S, and NH₃.

In a surface facility, condensable components may be separated from non-condensable components by reducing temperature and/or increasing pressure. Temperature reduction may be accomplished using heat exchangers cooled by ambient air or available water. Alternatively, the hot produced fluids may be cooled via heat exchange with produced hydrocarbon fluids previously cooled. The pressure may be increased via centrifugal or reciprocating compressors. Alternatively, or in conjunction, a diffuser-expander apparatus may be used to condense out liquids from gaseous flows. Separations may involve several stages of cooling and/or pressure changes.

Water in addition to condensable hydrocarbons may be dropped out of the gas when reducing temperature or increasing pressure. Liquid water may be separated from condensable hydrocarbons via gravity settling vessels or centrifugal separators. Demulsifiers may be used to aid in water separation.

Methods to remove CO₂, as well as other so-called acid gases (such as H₂S), from produced hydrocarbon gas include the use of chemical reaction processes and of physical solvent processes. Chemical reaction processes typically involve contacting the gas stream with an aqueous amine solution at high pressure and/or low temperature. This causes the acid gas species to chemically react with the amines and go into solution. By raising the temperature and/or lowering the pressure, the chemical reaction can be reversed and a concentrated stream of acid gases can be recovered. An alternative chemical reaction process involves hot carbonate solutions, typically potassium carbonate. The hot carbonate solution is regenerated and the concentrated stream of acid gases is recovered by contacting the solution with steam. Physical solvent processes typically involve contacting the gas stream with a glycol at high pressure and/or low temperature. Like the amine processes, reducing the pressure or raising the temperature allows regeneration of the solvent and recovery of the acid gases. Certain amines or glycols may be more or less selective in the types of acid gas species removed. Sizing of any of these processes requires determining the amount of chemical to circulate, the rate of circulation, the energy input for regeneration, and the size and type of gas-chemical contacting equipment. Contacting equipment may include packed or multi-tray countercurrent towers. Optimal sizing for each of these aspects is highly dependent on the rate at which gas is being produced from the formation and the concentration of the acid gases in the gas stream.

Acid gas removal may also be effectuated through the use of distillation towers. Such towers may include an intermediate freezing section wherein frozen CO₂ and H₂S particles are allowed to form. A mixture of frozen particles and liquids fall downward into a stripping section, where the lighter hydrocarbon gasses break out and rise within the tower. A rectification section may be provided at an upper end of the tower to further facilitate the cleaning of the overhead gas stream.

The hydrogen content of a gas stream may be adjusted by either removing all or a portion of the hydrogen or by removing all or a portion of the non-hydrogen species (e.g., CO₂, CH₄, etc.) Separations may be accomplished using cryogenic condensation, pressure-swing or temperature-swing adsorption, or selective diffusion membranes. If additional hydrogen is needed, hydrogen may be made by reforming methane via the classic water-shift reaction.

### EXPERIMENTS

Heating experiments were conducted on several different oil shale specimens and the liquids and gases released from the heated oil shale examined in detail. An oil shale sample from the Mahogany formation in the Piceance Basin in Colorado was collected. A solid, continuous block of the oil shale formation, approximately 1 cubic foot in size, was collected from the pilot mine at the Colony mine site on the eastern side of Parachute Creek. The oil shale block was designated CM-1B. The core specimens taken from this block, as described in the following examples, were all taken from the same stratigraphic interval. The heating tests were conducted using a Parr vessel, model number 243HC5, which is shown in FIG. 18 and is available from Parr Instrument Company.

### Example 1

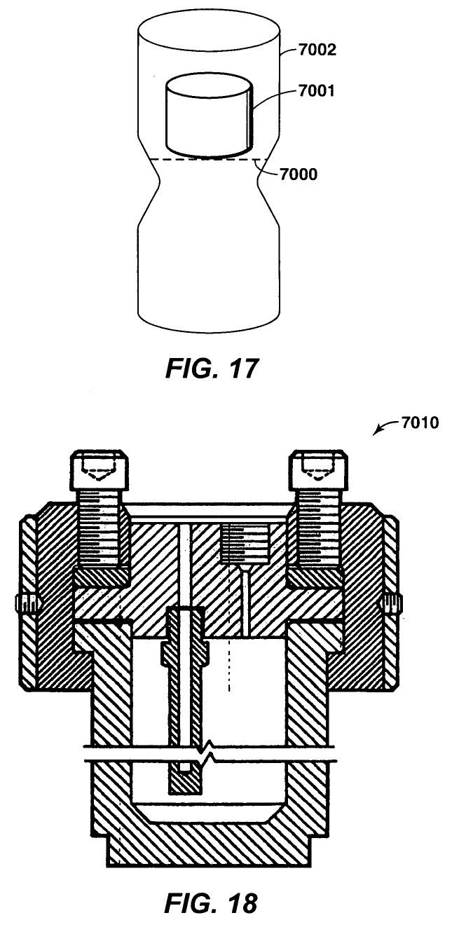

Oil shale block CM-1B was cored across the bedding planes to produce a cylinder 1.391 inches in diameter and approximately 2 inches long. A gold tube 7002 approximately 2 inches in diameter and 5 inches long was crimped and a screen 7000 inserted to serve as a support for the core specimen 7001 (FIG. 17). The oil shale core specimen 7001, 82.46 grams in weight, was placed on the screen 7000 in the gold tube 7002 and the entire assembly placed into a Parr heating vessel. The Parr vessel 7010, shown in FIG. 18, had an internal volume of 565 milliliters. Argon was used to flush the Parr vessel 7010 several times to remove air present in the chamber and the vessel pressurized to 500 psi with argon. The Parr vessel was then placed in a furnace which was designed to fit the Parr vessel. The furnace was initially at room temperature and was heated to 400° C. after the Parr vessel was placed in the furnace. The temperature of the Parr vessel achieved 400° C. after about 3 hours and remained in the 400° C. furnace for 24 hours. The Parr vessel was then removed from the furnace and allowed to cool to room temperature over a period of approximately 16 hours.

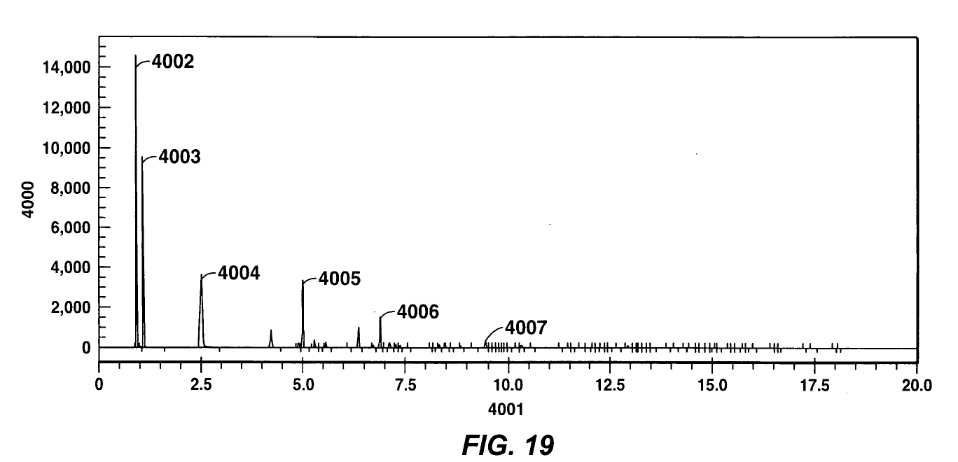

The room temperature Parr vessel was sampled to obtain a representative portion of the gas remaining in the vessel following the heating experiment. A small gas sampling cylinder 150 milliliters in volume was evacuated, attached to the Parr vessel and the pressure allowed to equilibrate. Gas chromatography (GC) analysis testing and non-hydrocarbon gas sample gas chromatography (GC) (GC not shown) of this gas sample yielded the results shown in FIG. 19, Table 2 and Table 1. In FIG. 19 the y-axis 4000 represents the detector response in pico-amperes (pA) while the x-axis 4001 represents the retention time in minutes. In FIG. 19 peak 4002 represents the response for methane, peak 4003 represents the response for ethane, peak 4004 represents the response for propane, peak 4005 represents the response for butane, peak 4006 represents the response for pentane and peak 4007 represents the response for hexane. From the GC results and the known volumes and pressures involved the total hydrocarbon content of the gas (2.09 grams), CO₂ content of the gas (3.35 grams), and H2S content of the gas (0.06 gram) were obtained.

TABLE 2
Peak and area details for FIG. 19 - Example 1 - 0 stress - gas GC

| Peak | RetTime | Area | |
|---|---|---|---|
| Number | [min] | [pA * s] | Name |
| 1 | 0.910 | 1.46868e4 | Methane |
| 2 | 0.999 | 148.12119 | ? |
| 3 | 1.077 | 1.26473e4 | Ethane |
| 4 | 2.528 | 1.29459e4 | Propane |
| 5 | 4.243 | 2162.93066 | iC4 |
| 6 | 4.922 | 563.11804 | ? |
| 7 | 5.022 | 5090.54150 | n-Butane |
| 8 | 5.301 | 437.92255 | ? |
| 9 | 5.446 | 4.67394 | ? |
| 10 | 5.582 | 283.92194 | ? |
| 11 | 6.135 | 15.47334 | ? |
| 12 | 6.375 | 1159.83130 | iC5 |
| 13 | 6.742 | 114.83960 | ? |
| 14 | 6.899 | 1922.98450 | n-Pentane |
| 15 | 7.023 | 2.44915 | ? |
| 16 | 7.136 | 264.34424 | ? |
| 17 | 7.296 | 127.60601 | ? |
| 18 | 7.383 | 118.79453 | ? |
| 19 | 7.603 | 3.99227 | ? |
| 20 | 8.138 | 13.15432 | ? |
| 21 | 8.223 | 13.01887 | ? |
| 22 | 8.345 | 103.15615 | ? |
| 23 | 8.495 | 291.26767 | 2-methyl pentane |
| 24 | 8.651 | 15.64066 | ? |
| 25 | 8.884 | 91.85989 | ? |
| 26 | 9.165 | 40.09448 | ? |
| 27 | 9.444 | 534.44507 | n-Hexane |
| 28 | 9.557 | 2.64731 | ? |
| 29 | 9.650 | 32.28295 | ? |
| 30 | 9.714 | 52.42796 | ? |
| 31 | 9.793 | 42.05001 | ? |
| 32 | 9.852 | 8.93775 | ? |
| 33 | 9.914 | 4.43648 | ? |
| 34 | 10.013 | 24.74299 | ? |
| 35 | 10.229 | 13.34387 | ? |
| 36 | 10.302 | 133.95892 | ? |
| 37 | 10.577 | 2.67224 | ? |
| 38 | 11.252 | 27.57400 | ? |
| 39 | 11.490 | 23.41665 | ? |
| 40 | 11.567 | 8.13992 | ? |
| 41 | 11.820 | 32.80781 | ? |
| 42 | 11.945 | 4.61821 | ? |
| 43 | 12.107 | 30.67044 | ? |
| 44 | 12.178 | 2.58269 | ? |
| 45 | 12.308 | 13.57769 | ? |
| 46 | 12.403 | 12.43018 | ? |
| 47 | 12.492 | 34.29918 | ? |
| 48 | 12.685 | 4.71311 | ? |
| 49 | 12.937 | 183.31729 | ? |
| 50 | 13.071 | 7.18510 | ? |
| 51 | 13.155 | 2.01699 | ? |
| 52 | 13.204 | 7.77467 | ? |
| 53 | 13.317 | 7.21400 | ? |
| 54 | 13.443 | 4.22721 | ? |
| 55 | 13.525 | 35.08374 | ? |
| 56 | 13.903 | 18.48654 | ? |
| 57 | 14.095 | 6.39745 | ? |
| 58 | 14.322 | 3.19935 | ? |
| 59 | 14.553 | 8.48772 | ? |
| 60 | 14.613 | 3.34738 | ? |
| 61 | 14.730 | 5.44062 | ? |
| 62 | 14.874 | 40.17010 | ? |
| 63 | 14.955 | 3.41596 | ? |
| 64 | 15.082 | 3.04766 | ? |
| 65 | 15.138 | 7.33028 | ? |
| 66 | 15.428 | 2.71734 | ? |
| 67 | 15.518 | 11.00256 | ? |
| 68 | 15.644 | 5.16752 | ? |
| 69 | 15.778 | 45.12025 | ? |
| 70 | 15.855 | 3.26920 | ? |
| 71 | 16.018 | 3.77424 | ? |
| 72 | 16.484 | 4.66657 | ? |
| 73 | 16.559 | 5.54783 | ? |
| 74 | 16.643 | 10.57255 | ? |
| 75 | 17.261 | 2.19534 | ? |
| 76 | 17.439 | 10.26123 | ? |
| 77 | 17.971 | 1.85618 | ? |
| 78 | 18.097 | 11.42077 | ? |

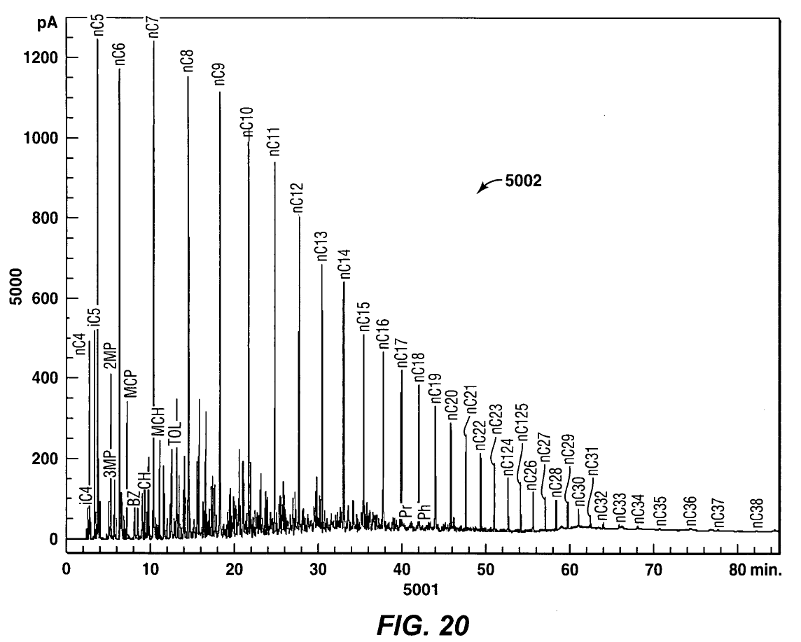

The Parr vessel was then vented to achieve atmospheric pressure, the vessel opened, and liquids collected from both inside the gold tube and in the bottom of the Parr vessel. Water was separated from the hydrocarbon layer and weighed. The amount collected is noted in Table 1. The collected hydrocarbon liquids were placed in a small vial, sealed and stored in the absence of light. No solids were observed on the walls of the gold tube or the walls of the Parr vessel. The solid core specimen was weighed and determined to have lost 19.21 grams as a result of heating. Whole oil gas chromatography (WOGC) testing of the liquid yielded the results shown in FIG. 20, Table 3, and Table 1. In FIG. 20 the y-axis 5000 represents the detector response in pico-amperes (pA) while the x-axis 5001 represents the retention time in minutes. The GC chromatogram is shown generally by label 5002 with individual identified peaks labeled with abbreviations.

TABLE 3
Peak and area details for FIG. 20 - Example 1 - 0 stress - liquid GC

| | Ret. Time | Peak Area | Compound |
|---|---|---|---|
| Peak # | [min] | [pA * s] | Name |
| 1 | 2.660 | 119.95327 | iC4 |
| 2 | 2.819 | 803.25989 | nC4 |
| 3 | 3.433 | 1091.80298 | iC5 |
| 4 | 3.788 | 2799.32520 | nC5 |
| 5 | 5.363 | 1332.67871 | 2-methyl pentane (2MP) |
| 6 | 5.798 | 466.35703 | 3-methyl pentane (3MP) |
| 7 | 6.413 | 3666.46240 | nC6 |
| 8 | 7.314 | 1161.70435 | Methyl cyclopentane (MCP) |
| 9 | 8.577 | 287.05969 | Benzene (BZ) |
| 10 | 9.072 | 530.19781 | Cyclohexane (CH) |
| 11 | 10.488 | 4700.48291 | nC7 |
| 12 | 11.174 | 937.38757 | Methyl cyclohexane (MCH) |
| 13 | 12.616 | 882.17358 | Toluene (TOL) |
| 14 | 14.621 | 3954.29687 | nC8 |
| 15 | 18.379 | 3544.52905 | nC9 |
| 16 | 21.793 | 3452.04199 | nC10 |
| 17 | 24.929 | 3179.11841 | nC11 |
| 18 | 27.843 | 2680.95459 | nC12 |
| 19 | 30.571 | 2238.89600 | nC13 |
| 20 | 33.138 | 2122.53540 | nC14 |
| 21 | 35.561 | 1773.59973 | nC15 |
| 22 | 37.852 | 1792.89526 | nC16 |
| 23 | 40.027 | 1394.61707 | nC17 |
| 24 | 40.252 | 116.81663 | Pristane (Pr) |
| 25 | 42.099 | 1368.02734 | nC18 |
| 26 | 42.322 | 146.96437 | Phytane (Ph) |
| 27 | 44.071 | 1130.63342 | nC19 |
| 28 | 45.956 | 920.52136 | nC20 |
| 29 | 47.759 | 819.92810 | nC21 |
| 30 | 49.483 | 635.42065 | nC22 |
| 31 | 51.141 | 563.24316 | nC23 |
| 32 | 52.731 | 432.74606 | nC24 |
| 33 | 54.261 | 397.36270 | nC25 |
| 34 | 55.738 | 307.56073 | nC26 |
| 35 | 57.161 | 298.70926 | nC27 |
| 36 | 58.536 | 252.60083 | nC28 |
| 37 | 59.867 | 221.84540 | nC29 |
| 38 | 61.154 | 190.29596 | nC30 |
| 39 | 62.539 | 123.65781 | nC31 |
| 40 | 64.133 | 72.47668 | nC32 |
| 41 | 66.003 | 76.84142 | nC33 |
| 42 | 68.208 | 84.35004 | nC34 |
| 43 | 70.847 | 36.68131 | nC35 |
| 44 | 74.567 | 87.62341 | nC36 |
| 45 | 77.798 | 33.30892 | nC37 |
| 46 | 82.361 | 21.99784 | nC38 |
| Totals: | | 5.32519e4 | |

### Example 2

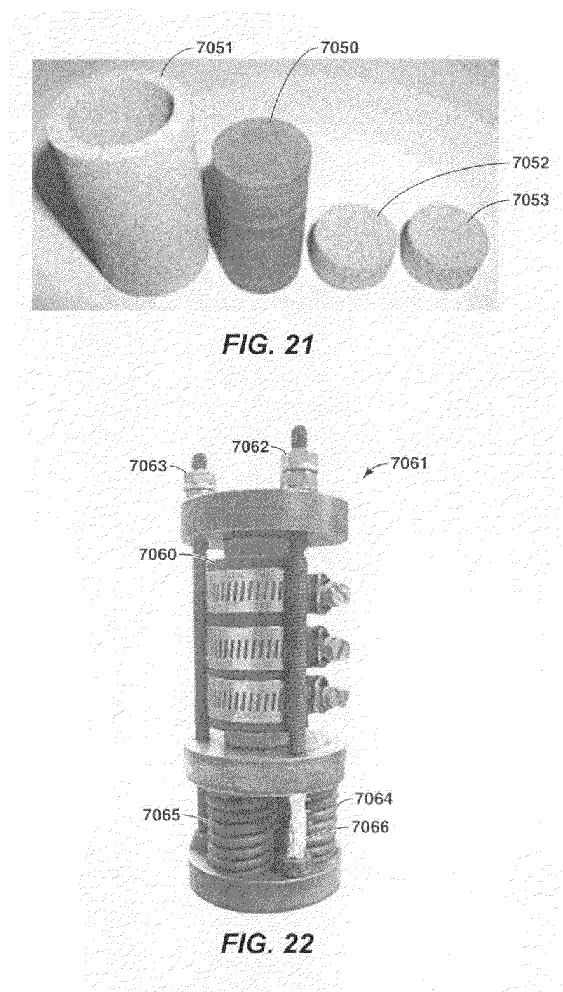

Oil shale block CM-1B was cored in a manner similar to that of Example 1 except that a 1 inch diameter core was created. With reference to FIG. 21, the core specimen 7050 was approximately 2 inches in length and weighed 42.47 grams. This core specimen 7050 was placed in a Berea sandstone cylinder 7051 with a 1-inch inner diameter and a 1.39 inch outer diameter. Berea plugs 7052 and 7053 were placed at each end of this assembly, so that the core specimen was completely surrounded by Berea. The Berea cylinders and plugs were fired at 500° C. for two hours prior to use with the mini load frame. The Berea cylinder 7051 along with the core specimen 7050 and the Berea end plugs 7052 and 7053 were placed in a slotted stainless steel sleeve and clamped into place. The sample assembly 7060 was placed in a spring-loaded mini-load-frame 7061 as shown in FIG. 22. Load was applied by tightening the nuts 7062 and 7063 at the top of the load frame 7061 to compress the springs 7064 and 7065. The springs 7064 and 7065 were high temperature, Inconel springs, which delivered 400 psi effective stress to the oil shale specimen 7060 when compressed. Sufficient travel of the springs 7064 and 7065 remained in order to accommodate any expansion of the core specimen 7060 during the course of heating. In order to ensure that this was the case, gold foil 7066 was placed on one of the legs of the apparatus to gauge the extent of travel. The entire spring loaded apparatus 7061 was placed in the Parr vessel (FIG. 18) and the heating experiment conducted as described in Example 1.

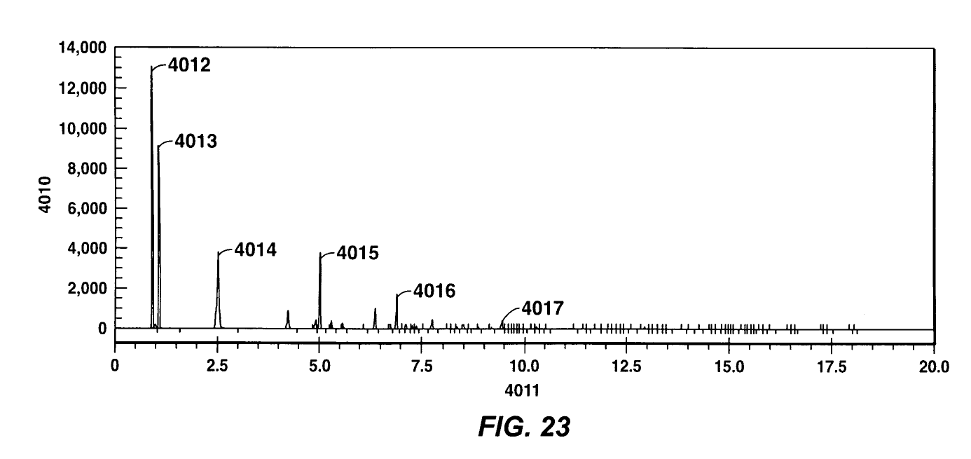

As described in Example 1, the room temperature Parr vessel was then sampled to obtain a representative portion of the gas remaining in the vessel following the heating experiment. Gas sampling, hydrocarbon gas sample gas chromatography (GC) testing, and non-hydrocarbon gas sample gas chromatography (GC) was conducted as in Example 1. Results are shown in FIG. 23, Table 4 and Table 1. In FIG. 23 the y-axis 4010 represents the detector response in pico-amperes (pA) while the x-axis 4011 represents the retention time in minutes. In FIG. 23 peak 4012 represents the response for methane, peak 4013 represents the response for ethane, peak 4014 represents the response for propane, peak 4015 represents the response for butane, peak 4016 represents the response for pentane and peak 4017 represents the response for hexane. From the gas chromatographic results and the known volumes and pressures involved the total hydrocarbon content of the gas was determined to be 1.33 grams and CO₂ content of the gas was 1.70 grams.

TABLE 4
Peak and area details for FIG. 23 - Example 2 - 400 psi stress - gas GC

| Peak | RetTime | Area | |
|---|---|---|---|
| Number | [min] | [pA * s] | Name |
| 1 | 0.910 | 1.36178e4 | Methane |
| 2 | 0.999 | 309.65613 | ? |
| 3 | 1.077 | 1.24143e4 | Ethane |
| 4 | 2.528 | 1.41685e4 | Propane |
| 5 | 4.240 | 2103.01929 | iC4 |
| 6 | 4.917 | 1035.25513 | ? |
| 7 | 5.022 | 5689.08887 | n-Butane |
| 8 | 5.298 | 450.26572 | ? |
| 9 | 5.578 | 302.56229 | ? |
| 10 | 6.125 | 33.82201 | ? |
| 11 | 6.372 | 1136.37097 | iC5 |
| 12 | 6.736 | 263.35754 | ? |
| 13 | 6.898 | 2254.86621 | n-Pentane |
| 14 | 7.066 | 7.12101 | ? |
| 15 | 7.133 | 258.31876 | ? |
| 16 | 7.293 | 126.54671 | ? |
| 17 | 7.378 | 155.60977 | ? |
| 18 | 7.598 | 6.73467 | ? |
| 19 | 7.758 | 679.95312 | ? |
| 20 | 8.133 | 27.13466 | ? |
| 21 | 8.216 | 24.77329 | ? |
| 22 | 8.339 | 124.70064 | ? |
| 23 | 8.489 | 289.12952 | 2-methyl pentane |
| 24 | 8.644 | 19.83309 | ? |
| 25 | 8.878 | 92.18938 | ? |
| 26 | 9.184 | 102.25701 | ? |
| 27 | 9.438 | 664.42584 | n-Hexane |
| 28 | 9.549 | 2.91525 | ? |
| 29 | 9.642 | 26.86672 | ? |
| 30 | 9.705 | 49.83235 | ? |
| 31 | 9.784 | 52.11239 | ? |
| 32 | 9.843 | 9.03158 | ? |
| 33 | 9.904 | 6.18217 | ? |
| 34 | 10.004 | 24.84150 | ? |
| 35 | 10.219 | 13.21182 | ? |
| 36 | 10.292 | 158.67511 | ? |
| 37 | 10.411 | 2.49094 | ? |
| 38 | 10.566 | 3.25252 | ? |
| 39 | 11.240 | 46.79988 | ? |
| 40 | 11.478 | 29.59438 | ? |
| 41 | 11.555 | 12.84377 | ? |
| 42 | 11.809 | 38.67433 | ? |
| 43 | 11.935 | 5.68525 | ? |
| 44 | 12.096 | 31.29068 | ? |
| 45 | 12.167 | 5.84513 | ? |
| 46 | 12.297 | 15.52042 | ? |
| 47 | 12.393 | 13.54158 | ? |
| 48 | 12.483 | 30.95983 | ? |
| 49 | 12.669 | 20.21915 | ? |
| 50 | 12.929 | 229.00655 | ? |
| 51 | 13.063 | 6.38678 | ? |
| 52 | 13.196 | 10.89876 | ? |
| 53 | 13.306 | 7.91553 | ? |
| 54 | 13.435 | 5.05444 | ? |
| 55 | 13.516 | 44.42806 | ? |
| 56 | 13.894 | 20.61910 | ? |
| 57 | 14.086 | 8.32365 | ? |
| 58 | 14.313 | 2.80677 | ? |
| 59 | 14.545 | 9.18198 | ? |
| 60 | 14.605 | 4.93703 | ? |
| 61 | 14.722 | 5.06628 | ? |
| 62 | 14.865 | 46.53282 | ? |
| 63 | 14.946 | 6.55945 | ? |
| 64 | 15.010 | 2.85594 | ? |
| 65 | 15.075 | 4.05371 | ? |
| 66 | 15.131 | 9.15954 | ? |
| 67 | 15.331 | 2.16523 | ? |
| 68 | 15.421 | 3.03294 | ? |
| 69 | 15.511 | 9.73797 | ? |
| 70 | 15.562 | 5.22962 | ? |
| 71 | 15.636 | 3.73105 | ? |
| 72 | 15.771 | 54.64651 | ? |
| 73 | 15.848 | 3.95764 | ? |
| 74 | 16.010 | 3.39639 | ? |
| 75 | 16.477 | 5.49586 | ? |
| 76 | 16.552 | 6.21470 | ? |
| 77 | 16.635 | 11.08140 | ? |
| 78 | 17.257 | 2.28673 | ? |
| 79 | 17.318 | 2.82284 | ? |
| 80 | 17.433 | 11.11376 | ? |
| 81 | 17.966 | 2.54065 | ? |
| 82 | 18.090 | 14.28333 | ? |

At this point, the Parr vessel was vented to achieve atmospheric pressure, the vessel opened, and liquids collected from inside the Parr vessel. Water was separated from the hydrocarbon layer and weighed. The amount collected is noted in Table 1. The collected hydrocarbon liquids were placed in a small vial, sealed and stored in the absence of light. Any additional liquid coating the surface of the apparatus or sides of the Parr vessel was collected with a paper towel and the weight of this collected liquid added to the total liquid collected. Any liquid remaining in the Berea sandstone was extracted with methylene chloride and the weight accounted for in the liquid total reported in Table 1. The Berea sandstone cylinder and end caps were clearly blackened with organic material as a result of the heating. The organic material in the Berea was not extractable with either toluene or methylene chloride, and was therefore determined to be coke formed from the cracking of hydrocarbon liquids. After the heating experiment, the Berea was crushed and its total organic carbon (TOC) was measured. This measurement was used to estimate the amount of coke in the Berea and subsequently how much liquid must have cracked in the Berea. A constant factor of 2.283 was used to convert the TOC measured to an estimate of the amount of liquid, which must have been present to produce the carbon found in the Berea. This liquid estimated is the “inferred oil” value shown in Table 1. The solid core specimen was weighed and determined to have lost 10.29 grams as a result of heating.

### Example 3

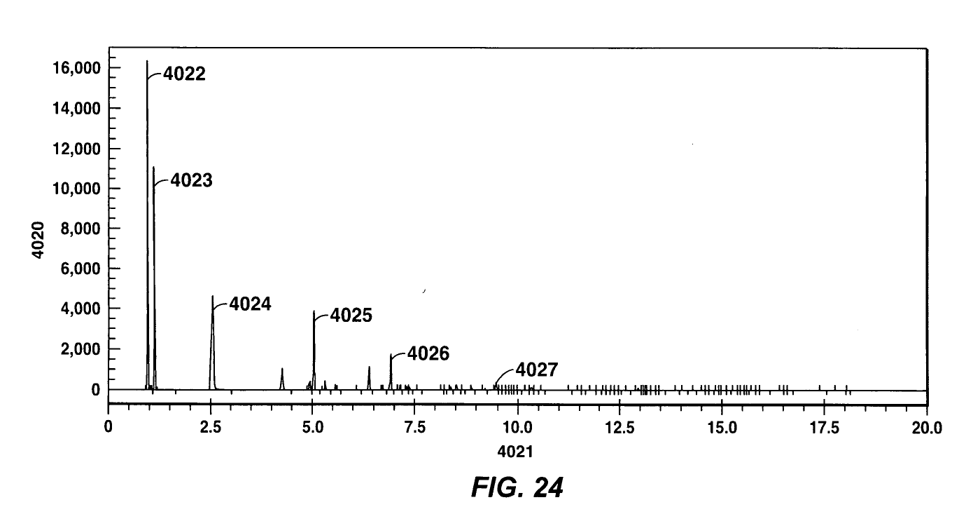

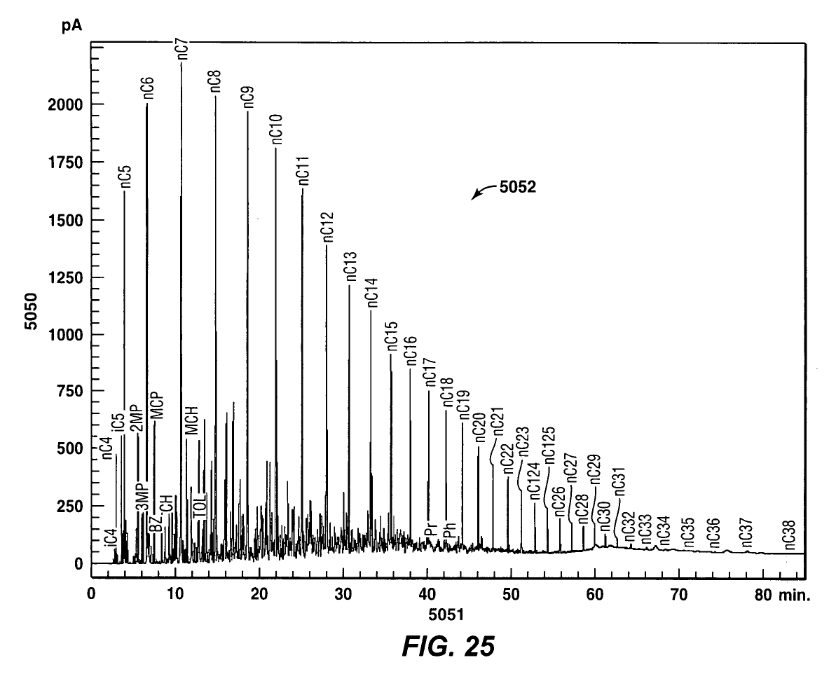

Conducted in a manner similar to that of Example 2 on a core specimen from oil shale block CM-1B, where the effective stress applied was 400 psi. Results for the gas sample collected and analyzed by hydrocarbon gas sample gas chromatography (GC) and non-hydrocarbon gas sample gas chromatography (GC) (GC not shown) are shown in FIG. 24, Table 5 and Table 1. In FIG. 24 the y-axis 4020 represents the detector response in pico-amperes (pA) while the x-axis 4021 represents the retention time in minutes. In FIG. 24 peak 4022 represents the response for methane, peak 4023 represents the response for ethane, peak 4024 represents the response for propane, peak 4025 represents the response for butane, peak 4026 represents the response for pentane and peak 4027 represents the response for hexane. Results for the liquid collected and analyzed by whole oil gas chromatography (WOGC) analysis are shown in FIG. 25, Table 6 and Table 1. In FIG. 25 the y-axis 5050 represents the detector response in pico-amperes (pA) while the x-axis 5051 represents the retention time in minutes. The GC chromatogram is shown generally by label 5052 with individual identified peaks labeled with abbreviations.

TABLE 5
Peak and area details for FIG. 24 - Example 3 - 400 psi stress - gas GC

| Peak | RetTime | Area | |
|---|---|---|---|
| Number | [min] | [pA * s] | Name |
| 1 | 0.910 | 1.71356e4 | Methane |
| 2 | 0.998 | 341.71646 | ? |
| 3 | 1.076 | 1.52621e4 | Ethane |
| 4 | 2.534 | 1.72319e4 | Propane |
| 5 | 4.242 | 2564.04077 | iC4 |
| 6 | 4.919 | 1066.90942 | ? |
| 7 | 5.026 | 6553.25244 | n-Butane |
| 8 | 5.299 | 467.88803 | ? |
| 9 | 5.579 | 311.65158 | ? |
| 10 | 6.126 | 33.61063 | ? |
| 11 | 6.374 | 1280.77869 | iC5 |
| 12 | 6.737 | 250.05510 | ? |
| 13 | 6.900 | 2412.40918 | n-Pentane |
| 14 | 7.134 | 249.80679 | ? |
| 15 | 7.294 | 122.60424 | ? |
| 16 | 7.379 | 154.40988 | ? |
| 17 | 7.599 | 6.87471 | ? |
| 18 | 8.132 | 25.50270 | ? |
| 19 | 8.216 | 22.33015 | ? |
| 20 | 8.339 | 129.17023 | ? |
| 21 | 8.490 | 304.97903 | 2-methyl pentane |
| 22 | 8.645 | 18.48411 | ? |
| 23 | 8.879 | 98.23043 | ? |
| 24 | 9.187 | 89.71329 | ? |
| 25 | 9.440 | 656.02161 | n-Hexane |
| 26 | 9.551 | 3.05892 | ? |
| 27 | 9.645 | 25.34058 | ? |
| 28 | 9.708 | 45.14915 | ? |
| 29 | 9.786 | 48.62077 | ? |
| 30 | 9.845 | 10.03335 | ? |
| 31 | 9.906 | 5.43165 | ? |
| 32 | 10.007 | 22.33582 | ? |
| 33 | 10.219 | 16.02756 | ? |
| 34 | 10.295 | 196.43715 | ? |
| 35 | 10.413 | 2.98115 | ? |
| 36 | 10.569 | 3.88067 | ? |
| 37 | 11.243 | 41.63386 | ? |
| 38 | 11.482 | 28.44063 | ? |
| 39 | 11.558 | 12.05196 | ? |
| 40 | 11.812 | 37.83630 | ? |
| 41 | 11.938 | 5.45990 | ? |
| 42 | 12.100 | 31.03111 | ? |
| 43 | 12.170 | 4.91053 | ? |
| 44 | 12.301 | 15.75041 | ? |
| 45 | 12.397 | 13.75454 | ? |
| 46 | 12.486 | 30.26099 | ? |
| 47 | 12.672 | 15.14775 | ? |
| 48 | 12.931 | 207.50433 | ? |
| 49 | 13.064 | 3.35393 | ? |
| 50 | 13.103 | 3.04880 | ? |
| 51 | 13.149 | 1.62203 | ? |
| 52 | 13.198 | 7.97665 | ? |
| 53 | 13.310 | 7.49605 | ? |
| 54 | 13.437 | 4.64921 | ? |
| 55 | 13.519 | 41.82572 | ? |
| 56 | 13.898 | 19.01739 | ? |
| 57 | 14.089 | 7.34498 | ? |
| 58 | 14.316 | 2.68912 | ? |
| 59 | 14.548 | 8.29593 | ? |
| 60 | 14.608 | 3.93147 | ? |
| 61 | 14.725 | 4.75483 | ? |
| 62 | 14.869 | 40.93447 | ? |
| 63 | 14.949 | 5.30140 | ? |
| 64 | 15.078 | 5.79979 | ? |
| 65 | 15.134 | 7.95179 | ? |
| 66 | 15.335 | 1.91589 | ? |
| 67 | 15.423 | 2.75893 | ? |
| 68 | 15.515 | 8.64343 | ? |
| 69 | 15.565 | 3.76481 | ? |
| 70 | 15.639 | 3.41854 | ? |
| 71 | 15.774 | 45.59035 | ? |
| 72 | 15.850 | 3.73501 | ? |
| 73 | 16.014 | 5.84199 | ? |
| 74 | 16.480 | 4.87036 | ? |
| 75 | 16.555 | 5.12607 | ? |
| 76 | 16.639 | 9.97469 | ? |
| 77 | 17.436 | 8.00434 | ? |
| 78 | 17.969 | 3.86749 | ? |
| 79 | 18.093 | 9.71661 | ? |

TABLE 6
Peak and area details from FIG. 25 - Example 3 - 400 psi stress -
liquid GC.

| | RetTime | Peak Area | Compound |
|---|---|---|---|
| Peak # | [min] | [pA * s] | Name |
| 1 | 2.744 | 102.90978 | iC4 |
| 2 | 2.907 | 817.57861 | nC4 |
| 3 | 3.538 | 1187.01831 | iC5 |
| 4 | 3.903 | 3752.84326 | nC5 |
| 5 | 5.512 | 1866.25342 | 2MP |
| 6 | 5.950 | 692.18964 | 3MP |
| 7 | 6.580 | 6646.48242 | nC6 |
| 8 | 7.475 | 2117.66919 | MCP |
| 9 | 8.739 | 603.21204 | BZ |
| 10 | 9.230 | 1049.96240 | CH |
| 11 | 10.668 | 9354.29590 | nC7 |
| 12 | 11.340 | 2059.10303 | MCH |
| 13 | 12.669 | 689.82861 | TOL |
| 14 | 14.788 | 8378.59375 | nC8 |
| 15 | 18.534 | 7974.54883 | nC9 |
| 16 | 21.938 | 7276.47705 | nC10 |
| 17 | 25.063 | 6486.47998 | nC11 |
| 18 | 27.970 | 5279.17187 | nC12 |
| 19 | 30.690 | 4451.49902 | nC13 |
| 20 | 33.254 | 4156.73389 | nC14 |
| 21 | 35.672 | 3345.80273 | nC15 |
| 22 | 37.959 | 3219.63745 | nC16 |
| 23 | 40.137 | 2708.28003 | nC17 |
| 24 | 40.227 | 219.38252 | Pr |
| 25 | 42.203 | 2413.01929 | nC18 |
| 26 | 42.455 | 317.17825 | Ph |
| 27 | 44.173 | 2206.65405 | nC19 |
| 28 | 46.056 | 1646.56616 | nC20 |
| 29 | 47.858 | 1504.49097 | nC21 |
| 30 | 49.579 | 1069.23608 | nC22 |
| 31 | 51.234 | 949.49316 | nC23 |
| 32 | 52.823 | 719.34735 | nC24 |
| 33 | 54.355 | 627.46436 | nC25 |
| 34 | 55.829 | 483.81885 | nC26 |
| 35 | 57.253 | 407.86371 | nC27 |
| 36 | 58.628 | 358.52216 | nC28 |
| 37 | 59.956 | 341.01791 | nC29 |
| 38 | 61.245 | 214.87863 | nC30 |
| 39 | 62.647 | 146.06461 | nC31 |
| 40 | 64.259 | 127.66831 | nC32 |
| 41 | 66.155 | 85.17574 | nC33 |
| 42 | 68.403 | 64.29253 | nC34 |
| 43 | 71.066 | 56.55088 | nC35 |
| 44 | 74.282 | 28.61854 | nC36 |
| 45 | 78.140 | 220.95929 | nC37 |
| 46 | 83.075 | 26.95426 | nC38 |
| Totals: | | 9.84518e4 | |

### Example 4

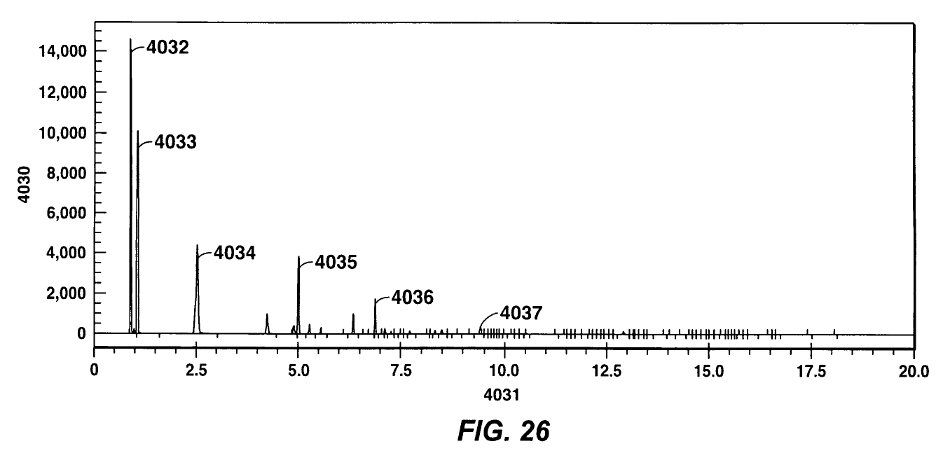

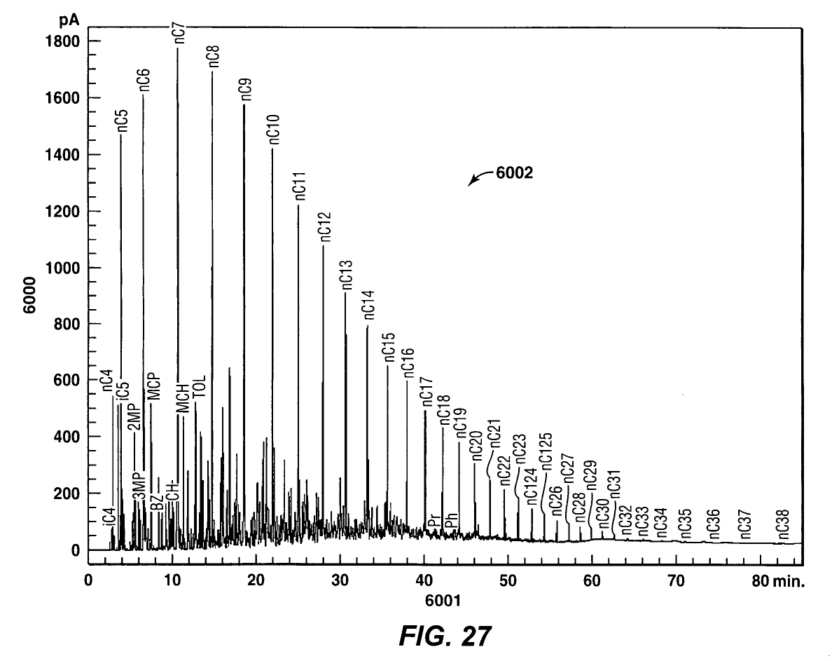

Conducted in a manner similar to that of Example 2 on a core specimen from oil shale block CM-1B; however, in this example the applied effective stress was 1,000 psi. Results for the gas collected and analyzed by hydrocarbon gas sample gas chromatography (GC) and non-hydrocarbon gas sample gas chromatography (GC) (GC not shown) are shown in FIG. 26, Table 7 and Table 1. In FIG. 26 the y-axis 4030 represents the detector response in pico-amperes (pA) while the x-axis 4031 represents the retention time in minutes. In FIG. 26 peak 4032 represents the response for methane, peak 4033 represents the response for ethane, peak 4034 represents the response for propane, peak 4035 represents the response for butane, peak 4036 represents the response for pentane and peak 4037 represents the response for hexane. Results for the liquid collected and analyzed by whole oil gas chromatography (WOGC) are shown in FIG. 27, Table 8 and Table 1. In FIG. 27 the y-axis 6000 represents the detector response in pico-amperes (pA) while the x-axis 6001 represents the retention time in minutes. The GC chromatogram is shown generally by label 6002 with individual identified peaks labeled with abbreviations.

TABLE 7
Peak and area details for FIG. 26 - Example 4 - 1000 psi stress -
gas GC

| Peak | RetTime | Area | |
|---|---|---|---|
| Number | [min] | [pA * s] | Name |
| 1 | 0.910 | 1.43817e4 | Methane |
| 2 | 1.000 | 301.69287 | ? |
| 3 | 1.078 | 1.37821e4 | Ethane |
| 4 | 2.541 | 1.64047e4 | Propane |
| 5 | 4.249 | 2286.08032 | iC4 |
| 6 | 4.924 | 992.04395 | ? |
| 7 | 5.030 | 6167.50000 | n-Butane |
| 8 | 5.303 | 534.37000 | ? |
| 9 | 5.583 | 358.96567 | ? |
| 10 | 6.131 | 27.44937 | ? |
| 11 | 6.376 | 1174.68872 | iC5 |
| 12 | 6.740 | 223.61662 | ? |
| 13 | 6.902 | 2340.79248 | n-Pentane |
| 14 | 7.071 | 5.29245 | ? |
| 15 | 7.136 | 309.94775 | ? |
| 16 | 7.295 | 154.59171 | ? |
| 17 | 7.381 | 169.53279 | ? |
| 18 | 7.555 | 2.80458 | ? |
| 19 | 7.601 | 5.22327 | ? |
| 20 | 7.751 | 117.69164 | ? |
| 21 | 8.134 | 29.41086 | ? |
| 22 | 8.219 | 19.39338 | ? |
| 23 | 8.342 | 133.52739 | ? |
| 24 | 8.492 | 281.61343 | 2-methyl pentane |
| 25 | 8.647 | 22.19704 | ? |
| 26 | 8.882 | 99.56919 | ? |
| 27 | 9.190 | 86.65676 | ? |
| 28 | 9.443 | 657.28754 | n-Hexane |
| 29 | 9.552 | 4.12572 | ? |
| 30 | 9.646 | 34.33701 | ? |
| 31 | 9.710 | 59.12064 | ? |
| 32 | 9.788 | 62.97972 | ? |
| 33 | 9.847 | 15.13559 | ? |
| 34 | 9.909 | 6.88310 | ? |
| 35 | 10.009 | 29.11555 | ? |
| 36 | 10.223 | 23.65434 | ? |
| 37 | 10.298 | 173.95422 | ? |
| 38 | 10.416 | 3.37255 | ? |
| 39 | 10.569 | 7.64592 | ? |
| 40 | 11.246 | 47.30062 | ? |
| 41 | 11.485 | 32.04262 | ? |
| 42 | 11.560 | 13.74583 | ? |
| 43 | 11.702 | 2.68917 | ? |
| 44 | 11.815 | 36.51670 | ? |
| 45 | 11.941 | 6.45255 | ? |
| 46 | 12.103 | 28.44484 | ? |
| 47 | 12.172 | 5.96475 | ? |
| 48 | 12.304 | 17.59856 | ? |
| 49 | 12.399 | 15.17446 | ? |
| 50 | 12.490 | 31.96492 | ? |
| 51 | 12.584 | 3.27834 | ? |
| 52 | 12.675 | 14.08259 | ? |
| 53 | 12.934 | 207.21574 | ? |
| 54 | 13.105 | 8.29743 | ? |
| 55 | 13.151 | 2.25476 | ? |
| 56 | 13.201 | 8.36965 | ? |
| 57 | 13.312 | 9.49917 | ? |
| 58 | 13.436 | 6.09893 | ? |
| 59 | 13.521 | 46.34579 | ? |
| 60 | 13.900 | 20.53506 | ? |
| 61 | 14.090 | 8.41120 | ? |
| 62 | 14.318 | 4.36870 | ? |
| 63 | 14.550 | 8.68951 | ? |
| 64 | 14.610 | 4.39150 | ? |
| 65 | 14.727 | 4.35713 | ? |
| 66 | 14.870 | 37.17881 | ? |
| 67 | 14.951 | 5.78219 | ? |
| 68 | 15.080 | 5.54470 | ? |
| 69 | 15.136 | 8.07308 | ? |
| 70 | 15.336 | 2.07075 | ? |
| 71 | 15.425 | 2.67118 | ? |
| 72 | 15.516 | 8.47004 | ? |
| 73 | 15.569 | 3.89987 | ? |
| 74 | 15.641 | 3.96979 | ? |
| 75 | 15.776 | 40.75155 | ? |
| 76 | 16.558 | 5.06379 | ? |
| 77 | 16.641 | 8.43767 | ? |
| 78 | 17.437 | 6.00180 | ? |
| 79 | 18.095 | 7.66881 | ? |
| 80 | 15.853 | 3.97375 | ? |
| 81 | 16.016 | 5.68997 | ? |
| 82 | 16.482 | 3.27234 | ? |

TABLE 8
Peak and area details from FIG. 27 - Example 4 - 1000 psi stress -
liquid GC.

| | RetTime | Peak Area | Compound |
|---|---|---|---|
| Peak # | [min] | [pA * s] | Name |
| 1 | 2.737 | 117.78948 | iC4 |
| 2 | 2.901 | 923.40125 | nC4 |
| 3 | 3.528 | 1079.83325 | iC5 |
| 4 | 3.891 | 3341.44604 | nC5 |
| 5 | 5.493 | 1364.53186 | 2MP |
| 6 | 5.930 | 533.68530 | 3MP |
| 7 | 6.552 | 5160.12207 | nC6 |
| 8 | 7.452 | 1770.29932 | MCP |
| 9 | 8.717 | 487.04718 | BZ |
| 10 | 9.206 | 712.61566 | CH |
| 11 | 10.634 | 7302.51123 | nC7 |
| 12 | 11. | 1755.92236 | MCH |
| 13 | 12.760 | 2145.57666 | TOL |
| 14 | 14.755 | 6434.40430 | nC8 |
| 15 | 18.503 | 6007.12891 | nC9 |
| 16 | 21.906 | 5417.67480 | nC10 |
| 17 | 25.030 | 4565.11084 | nC11 |
| 18 | 27.936 | 3773.91943 | nC12 |
| 19 | 30.656 | 3112.23950 | nC13 |
| 20 | 33.220 | 2998.37720 | nC14 |
| 21 | 35.639 | 2304.97632 | nC15 |
| 22 | 37.927 | 2197.88892 | nC16 |
| 23 | 40.102 | 1791.11877 | nC17 |
| 24 | 40.257 | 278.39423 | Pr |
| 25 | 42.171 | 1589.64233 | nC18 |
| 26 | 42.428 | 241.65131 | Ph |
| 27 | 44.141 | 1442.51843 | nC19 |
| 28 | 46.025 | 1031.68481 | nC20 |
| 29 | 47.825 | 957.65479 | nC21 |
| 30 | 49.551 | 609.59943 | nC22 |
| 31 | 51.208 | 526.53339 | nC23 |
| 32 | 52.798 | 383.01022 | nC24 |
| 33 | 54.329 | 325.93640 | nC25 |
| 34 | 55.806 | 248.12935 | nC26 |
| 35 | 57.230 | 203.21725 | nC27 |
| 36 | 58.603 | 168.78055 | nC28 |
| 37 | 59.934 | 140.40034 | nC29 |
| 38 | 61.222 | 95.47594 | nC30 |
| 39 | 62.622 | 77.49546 | nC31 |
| 40 | 64.234 | 49.08135 | nC32 |
| 41 | 66.114 | 33.61663 | nC33 |
| 42 | 68.350 | 27.46170 | nC34 |
| 43 | 71.030 | 35.89277 | nC35 |
| 44 | 74.162 | 16.87499 | nC36 |
| 45 | 78.055 | 29.21477 | nC37 |
| 46 | 82.653 | 9.88631 | nC38 |
| Totals: | | 7.38198e4 | |

### Example 5

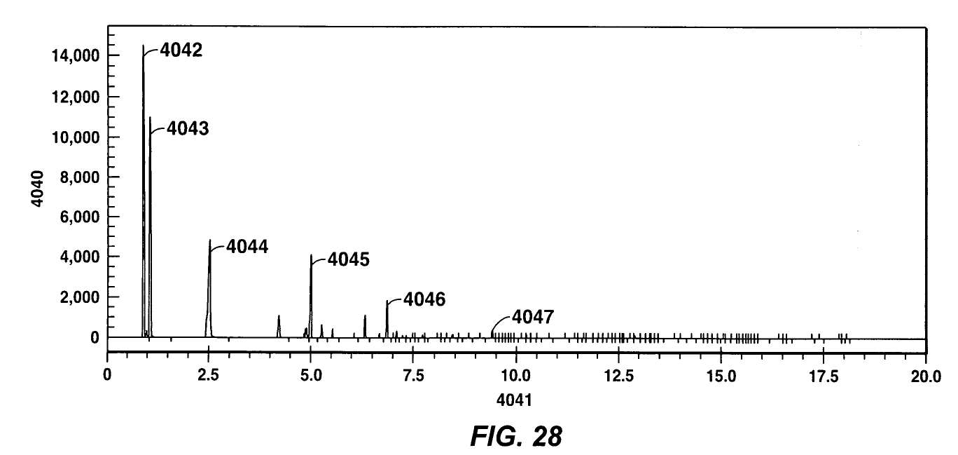

Conducted in a manner similar to that of Example 2 on a core specimen from oil shale block CM-1B; however, in this example the applied effective stress was 1,000 psi. Results for the gas collected and analyzed by hydrocarbon gas sample gas chromatography (GC) and non-hydrocarbon gas sample gas chromatography (GC) (GC not shown) are shown in FIG. 28, Table 9 and Table 1. In FIG. 28 the y-axis 4040 represents the detector response in pico-amperes (pA) while the x-axis 4041 represents the retention time in minutes. In FIG. 28 peak 4042 represents the response for methane, peak 4043 represents the response for ethane, peak 4044 represents the response for propane, peak 4045 represents the response for butane, peak 4046 represents the response for pentane and peak 4047 represents the response for hexane.

TABLE 9
Peak and area details for FIG. 28 - Example 5 - 1000 psi stress -
gas GC

| Peak | RetTime | Area | |
|---|---|---|---|
| Number | [min] | [pA * s] | Name |
| 1 | 0.910 | 1.59035e4 | Methane |
| 2 | 0.999 | 434.21375 | ? |
| 3 | 1.077 | 1.53391e4 | Ethane |
| 4 | 2.537 | 1.86530e4 | Propane |
| 5 | 4.235 | 2545.45850 | iC4 |
| 6 | 4.907 | 1192.68970 | ? |
| 7 | 5.015 | 6814.44678 | n-Butane |
| 8 | 5.285 | 687.83679 | ? |
| 9 | 5.564 | 463.25885 | ? |
| 10 | 6.106 | 30.02624 | ? |
| 11 | 6.351 | 1295.13477 | iC5 |
| 12 | 6.712 | 245.26985 | ? |
| 13 | 6.876 | 2561.11792 | n-Pentane |
| 14 | 7.039 | 4.50998 | ? |
| 15 | 7.109 | 408.32999 | ? |
| 16 | 7.268 | 204.45311 | ? |
| 17 | 7.354 | 207.92183 | ? |
| 18 | 7.527 | 4.02397 | ? |
| 19 | 7.574 | 5.65699 | ? |
| 20 | 7.755 | 2.35952 | ? |
| 21 | 7.818 | 2.00382 | ? |
| 22 | 8.107 | 38.23093 | ? |
| 23 | 8.193 | 20.54333 | ? |
| 24 | 8.317 | 148.54445 | ? |
| 25 | 8.468 | 300.31586 | 2-methyl pentane |
| 26 | 8.622 | 26.06131 | ? |
| 27 | 8.858 | 113.70123 | ? |
| 28 | 9.168 | 90.37163 | ? |
| 29 | 9.422 | 694.74438 | n-Hexane |
| 30 | 9.531 | 4.88323 | ? |
| 31 | 9.625 | 45.91505 | ? |
| 32 | 9.689 | 76.32931 | ? |
| 33 | 9.767 | 77.63214 | ? |
| 34 | 9.826 | 19.23768 | ? |
| 35 | 9.889 | 8.54605 | ? |
| 36 | 9.989 | 37.74959 | ? |
| 37 | 10.204 | 30.83943 | ? |
| 38 | 10.280 | 184.58420 | ? |
| 39 | 10.397 | 4.43609 | ? |
| 40 | 10.551 | 10.59880 | ? |
| 41 | 10.843 | 2.30370 | ? |
| 42 | 11.231 | 55.64666 | ? |
| 43 | 11.472 | 35.46931 | ? |
| 44 | 11.547 | 17.16440 | ? |
| 45 | 11.691 | 3.30460 | ? |
| 46 | 11.804 | 39.46368 | ? |
| 47 | 11.931 | 7.32969 | ? |
| 48 | 12.094 | 30.59748 | ? |
| 49 | 12.163 | 6.93754 | ? |
| 50 | 12.295 | 18.69523 | ? |
| 51 | 12.391 | 15.96837 | ? |
| 52 | 12.482 | 33.66422 | ? |
| 53 | 12.577 | 2.02121 | ? |
| 54 | 12.618 | 2.32440 | ? |
| 55 | 12.670 | 12.83803 | ? |
| 56 | 12.851 | 2.22731 | ? |
| 57 | 12.929 | 218.23195 | ? |
| 58 | 13.100 | 14.33166 | ? |
| 59 | 13.198 | 10.20244 | ? |
| 60 | 13.310 | 12.02551 | ? |
| 61 | 13.432 | 8.23884 | ? |
| 62 | 13.519 | 47.64641 | ? |
| 63 | 13.898 | 22.63760 | ? |
| 64 | 14.090 | 9.29738 | ? |
| 65 | 14.319 | 3.88012 | ? |
| 66 | 14.551 | 9.26884 | ? |
| 67 | 14.612 | 4.34914 | ? |
| 68 | 14.729 | 4.07543 | ? |
| 69 | 14.872 | 46.24465 | ? |
| 70 | 14.954 | 6.62461 | ? |
| 71 | 15.084 | 3.92423 | ? |
| 72 | 15.139 | 8.60328 | ? |
| 73 | 15.340 | 2.17899 | ? |
| 74 | 15.430 | 2.96646 | ? |
| 75 | 15.521 | 9.66407 | ? |
| 76 | 15.578 | 4.27190 | ? |
| 77 | 15.645 | 4.37904 | ? |
| 78 | 15.703 | 2.68909 | ? |
| 79 | 15.782 | 46.97895 | ? |
| 80 | 15.859 | 4.69475 | ? |
| 81 | 16.022 | 7.36509 | ? |
| 82 | 16.489 | 3.91073 | ? |
| 83 | 16.564 | 6.22445 | ? |
| 84 | 16.648 | 10.24660 | ? |
| 85 | 17.269 | 2.69753 | ? |
| 86 | 17.445 | 10.16989 | ? |
| 87 | 17.925 | 2.28341 | ? |
| 88 | 17.979 | 2.71101 | ? |
| 89 | 18.104 | 11.19730 | ? |

TABLE 1
Summary data for Examples 1-5.

| | Example 1 | Example 2 | Example 3 | Example 4 | Example 5 |
|---|---|---|---|---|---|
| Effective Stress (psi) | 0 | 400 | 400 | 1000 | 1000 |
| Sample weight (g) | 82.46 | 42.57 | 48.34 | 43.61 | 43.73 |
| Sample weight loss (g) | 19.21 | 10.29 | 11.41 | 10.20 | 9.17 |
| Fluids Recovered: | | | | | |
| Oil (g) | 10.91 | 3.63 | 3.77 | 3.02 | 2.10 |
| | 36.2 gal/ton | 23.4 gal/ton | 21.0 gal/ton | 19.3 gal/ton | 13/1 gal/ton |
| Water (g) | 0.90 | 0.30 | 0.34 | 0.39 | 0.28 |
| | 2.6 gal/ton | 1.7 gal/ton | 1.7 gal/ton | 2.1 gal/ton | 1.5 gal/ton |
| HC gas (g) | 2.09 | 1.33 | 1.58 | 1.53 | 1.66 |
| | 683 scf/ton | 811 scf/ton | 862 scf/ton | 905 scf/ton | 974 scf/ton |
| CO₂ (g) | 3.35 | 1.70 | 1.64 | 1.74 | 1.71 |
| | 700 scf/ton | 690 scf/ton | 586 scf/ton | 690 scf/ton | 673 scf/ton |
| H₂S (g) | 0.06 | 0.0 | 0.0 | 0.0 | 0.0 |
| Coke Recovered: | 0.0 | 0.73 | 0.79 | .47 | 0.53 |
| Inferred Oil (g) | 0.0 | 1.67 | 1.81 | 1.07 | 1.21 |
| | 0 gal/ton | 10.8 gal/ton | 10.0 gal/ton | 6.8 gal/ton | 7.6 gal/ton |
| Total Oil (g) | 10.91 | 5.31 | 5.58 | 4.09 | 3.30 |
| | 36.2 gal/ton | 34.1 gal/ton | 31.0 gal/ton | 26.1 gal/ton | 20.7 gal/ton |
| Balance (g) | 1.91 | 2.59 | 3.29 | 3.05 | 2.91 |

Heating experiments were conducted on several additional oil shale specimens and the liquids and gases released from the heated oil shale examined in detail. An oil shale sample from the Mahogany formation in the Uinta Basin in Utah was collected. A solid, continuous block of the oil shale formation, approximately 0.5 cubic foot in size, was collected from Hell's Hole Canyon in Utah. The oil shale block was designated HHC-2. The core specimens taken from this block, as described in the following examples, were all taken from the same stratigraphic interval. The heating tests were conducted using a Parr vessel, model number 243HC5, which is shown in FIG. 18 and is available from Parr Instrument Company.

### Example 6

Oil shale block HHC-2 was sampled across the bedding planes to produce samples with an approximately uniform distribution of laminae that were used in four zero effective stress experiments (Examples 6, 7, 13 & 14). This example describes the experimental methodology common to these experiments with individual details for each experiment summarized in Table 15.

For each unstressed experiment, a screen 7000 served as a support for each specimen 7001 (FIG. 17). The oil shale core specimen 7001, was placed on the screen and the entire assembly placed into a Parr heating vessel. The mass of each sample is indicated in Table 15. The Parr vessel 7010, shown in FIG. 18, has an internal volume of 565 milliliters. Argon was used to flush the Parr vessel 7010 several times to remove air present in the chamber and the vessel was then pressurized to 50, 200, or 500 psi with argon (see Table 15). After the vessel was pressurized its mass was determined and recorded. The Parr vessel was then placed in a furnace that was designed to fit the Parr vessel. The furnace was initially at room temperature and was heated to either 375 or 393° C. (see Table 15) after the Parr vessel was placed in it. The Parr vessel achieved the desired experimental temperature after about 3 hours and remained at that temperature for 24 hours. The Parr vessel was then removed from the furnace and allowed to cool to room temperature over a period of approximately 16 hours. Once the vessel reached room temperature its mass was determined and recorded. No measurable mass was lost or gained in any experiment described herein.

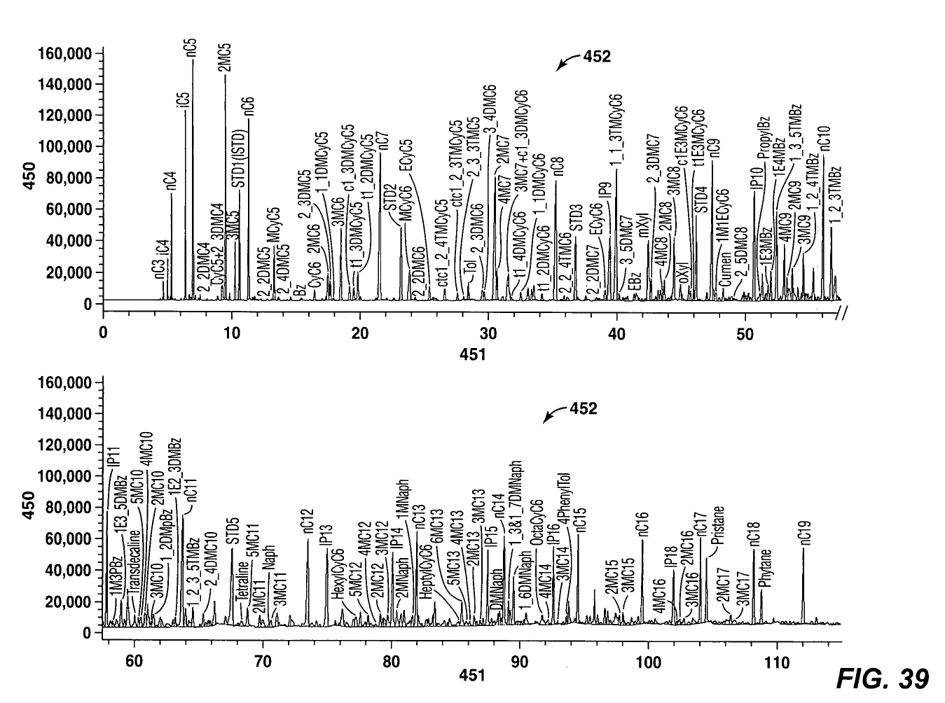

The room temperature Parr vessel was sampled to obtain a representative portion of the gas remaining in the vessel following the heating experiment. A gas sampling cylinder 150 milliliters in volume was evacuated, attached to the Parr vessel and the pressure allowed to equilibrate. The Parr vessel was then vented, to achieve atmospheric pressure, opened, and liquid collected from the bottom of the Parr vessel. Water was separated from the hydrocarbon layer and weighed. The amount collected is noted in Table 15. The collected hydrocarbon liquids were placed in a small vial, sealed and stored in the absence of light at a constant temperature of 7° C. The solid core specimen was weighed and its new mass recorded (see Table 15). All samples lost mass as a result of heating. C4-C19 liquid sample gas-chromatography (C4-C19 GC) testing of the liquid yielded the results shown in FIGS. 39-52, and Table 16 while liquid chromatography (LC) followed by gas chromatography/mass spectrometry (GC/MS) analysis for such samples is discussed with reference to FIGS. 53-59 and later herein. The C4-C19 GC chromatograms are shown and are generally label as discussed below with individual identified peaks labeled with abbreviations.

### Example 8

Oil shale block HHC-2 described in Example 6 was cored perpendicular to bedding to yield a 1 inch diameter cores for use in experiments subjected to effective stress conditions. With reference to FIG. 21, the core specimens 7050 were approximately 2 inches in length with the mass of each sample indicated in Table 15.

The following details the experimental procedures for the stressed experiments of Examples 8-12, & 15-19 insofar as they differed from the unstressed experiments described for Example 6.

The core specimens 7050 were then placed in Berea sandstone cylinders 7051 with a 1-inch inner diameter and a 1.39 inch outer diameter. Berea plugs 7052 and 7053 were placed at each end of this assembly, so that the core specimens were completely surrounded by Berea. The Berea cylinders and plugs were fired at 500° C. for two hours prior to use with the mini load frame. The Berea cylinder 7051 along with the core specimen 7050 and the Berea end plugs 7052 and 7053 were placed in a slotted stainless steel sleeve and clamped into place. The sample assembly 7060 was placed in a spring-loaded mini-load-frame 7061 as shown in FIG. 22. Load was applied by tightening the nuts 7062 and 7063 at the top of the load frame 7061 to compress the springs 7064 and 7065. The springs 7064 and 7065 were high temperature, Inconel springs, which delivered either 400 or 1000 psi effective stress to the oil shale specimen 7060 when compressed. Sufficient travel of the springs 7064 and 7065 remained in order to accommodate any expansion of the core specimen 7060 during the course of heating. In order to ensure that this was the case, gold foil 7066 was placed on one of the legs of the apparatus to gauge the extent of travel. The entire spring loaded apparatus 7061 was placed in the Parr vessel (FIG. 18) and the heating experiment conducted as described in Example 6.

As described in Example 6, the room temperature Parr vessel was then sampled to obtain a representative portion of the gas remaining in the vessel following the heating experiment. At this point, the Parr vessel was vented to achieve atmospheric pressure, the vessel opened, and liquids collected from inside the Parr vessel. Water was separated from the hydrocarbon layer and weighed. The amount collected is noted in Table 15. The collected hydrocarbon liquids were placed in a small vial, sealed and stored in the absence of light at a constant temperature of 7° C. C4-C19 liquid sample gas-chromatography (C4-C19 GC) testing of the liquid yielded the results shown in FIGS. 39-52, and Table 16 while liquid chromatography (LC) followed by gas chromatography/mass spectrometry (GC/MS) analysis for such samples is discussed with reference to FIGS. 53-59 and later herein. Any additional liquid coating the surface of the apparatus or sides of the Parr vessel was collected with a paper towel and the weight of this collected liquid added to the total liquid collected.

TABLE 15
Summary data for Examples 6-19.

| | | Initial | | Eff. | Wt. | | | wipe |
|---|---|---|---|---|---|---|---|---|
| Example | | Ar P | Wt. (g) | Stress | loss | Oil | Water | down |
| Number | TC | psi | (gms) | psi | (gms) | (gms) | (gms) | (gms) |
| 6 | 375 | 500 | 95.2761 | 0 | 11.43 | 5.17 | 0.63 | 0.78 |
| 7 | 375 | 200 | 66.8161 | 0 | 9.78 | 5.71 | 0.51 | 0.41 |
| 8 | 375 | 500 | 45.1197 | 400 | 6.15 | 2.27 | 0.32 | 0.78 |
| 9 | 375 | 200 | 47.7778 | 400 | 6.27 | 2.44 | 0.39 | 0.97 |
| 10 | 375 | 50 | 48.3162 | 400 | 6.31 | 2.88 | 0.45 | 0.48 |
| 11 | 375 | 500 | 44.6242 | 1000 | 6.58 | 2.08 | 0.35 | 0.93 |
| 12 | 375 | 200 | 48.201 | 1000 | 6.86 | 2.56 | 0.43 | 0.76 |
| 13 | 393 | 500 | 85.6623 | 0 | 15.06 | 8.25 | 0.69 | 0.17 |
| 14 | 393 | 200 | 67.4904 | 0 | 11.87 | 6.60 | 0.43 | 0.30 |
| 15 | 393 | 500 | 45.3920 | 400 | 8.73 | 3.67 | 0.38 | 0.35 |
| 16 | 393 | 200 | 48.164 | 400 | 8.35 | 3.15 | 0.32 | 0.81 |
| 17 | 393 | 50 | 48.9843 | 400 | 8.31 | 3.19 | 0.49 | 0.52 |
| 18 | 393 | 500 | 44.7570 | 1000 | 8.50 | 2.50 | 0.32 | 0.59 |
| 19 | 393 | 200 | 47.436 | 1000 | 8.26 | 2.11 | 0.13 | 0.98 |

### Example 20

This procedure describes the extraction method used to obtain soluble organic matter (bitumen) from the oil shale sample of Example 20 which was subsequently analyzed by the liquid chromatography (LC) followed by gas chromatography/mass spectrometry (GC/MS) procedures discussed later herein and discussed with reference to FIGS. 53-59.

Extraction Method: The Extraction procedure utilized the laboratory equipment and chemicals described in Table 17 as well as other typical laboratory supplies and equipment.

TABLE 17

| No. | Lab Requirements |
|---|---|
| 1 | Soxtec Tecator System (PERSTORP Analytical) |
| 2 | Methylene Chloride (Fisher Optima grade equivalent or better) |
| 3 | Methanol (Fisher Optima grade equivalent or better) |
| 4 | Cellulose thimbles (26 mm × 60 mm; Fisher Scientific) |
| 5 | Thimble adapters (Perstorp Analytical) |
| 6 | Glass extraction cups (small; Perstorp Analytical) |
| 7 | Nitrogen gas (UHP) (Air Liquide) |
| 8 | Retsch Mill (micronizer; Brinkman) |
| 9 | Retsch Crusher (Lemaire Instruments) |

Sample preparation: The Retsch mill and crusher were thoroughly cleaned and the sample was micronized to a particle size of 0.5 micron. About 30 grams of the micronized sample was weighed in the thimble to be used in the extraction.

Extraction procedure: Table 18 describes the extraction method.

TABLE 18

| Step | Action |
|---|---|
| 1 | Wash the Soxtec Tecator extraction system and clean the Teflon O-ring seals with |
| | methylene chloride. |
| 2 | Run a methylene chloride/methanol (9:1) blank through the system. |
| 3 | Place a clean Teflon O-ring seal into the groove at the bottom of condenser. |
| 4 | Turn on refrigerated bath/circulators and check flow. |
| 5 | Set the hot oil bath at 120° C. |
| 6 | Insert the thimble (with sample inside) into the condenser by fastening the thimble |
| | adapter to the condenser rod. |
| 7 | Place 2 glass beads and 50 mls (for small extraction cups) of Methylene Chloride: |
| | Methanol solution (9:1) in glass extraction cups. |
| 8 | Place the extraction cups on the Tecator system. |
| 9 | Bring solvent to a boil, lower thimble into solvent and extract as follows: |
| | 1. Extract 1 hour with thimble in extraction cup. |
| | 2. Rinse 1 hour with thimble above extraction cup. |
| | 3. Close stop cocks for faster evaporation. |
| | 4. Evaporate down to approximately 10 mls. |
| 10 | Remove extraction cups from Tecator System. |
| 11 | Transfer the extract to the pre-weighed 20 mls vial and bring to constant weight at |
| | 40° C. under nitrogen. |

Quality Control: To ensure reproducible results, a known sediment standard was maintained as the QC extraction standard and was tested in conjunction with the sample. The extraction yield for the standard was within the acceptable 2 standard deviation range.

### Core Plugs

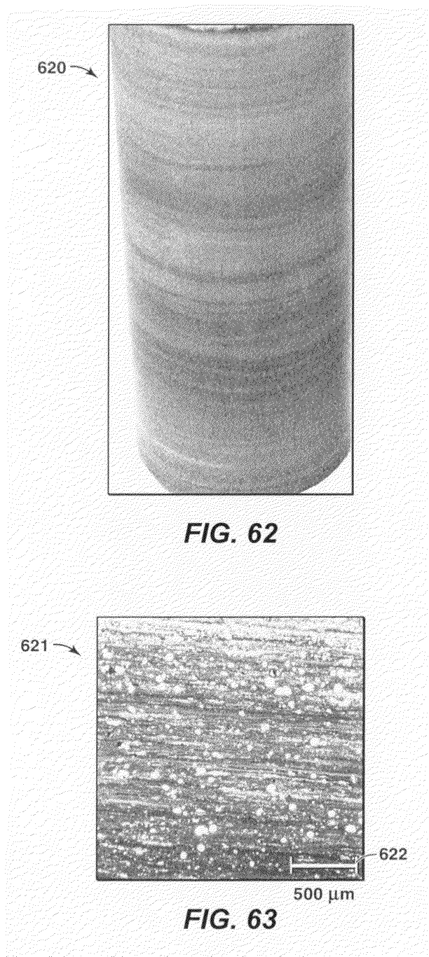

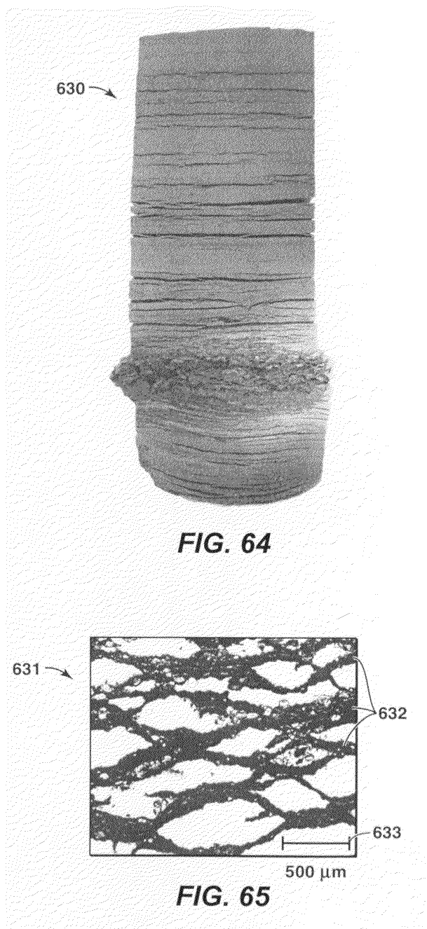

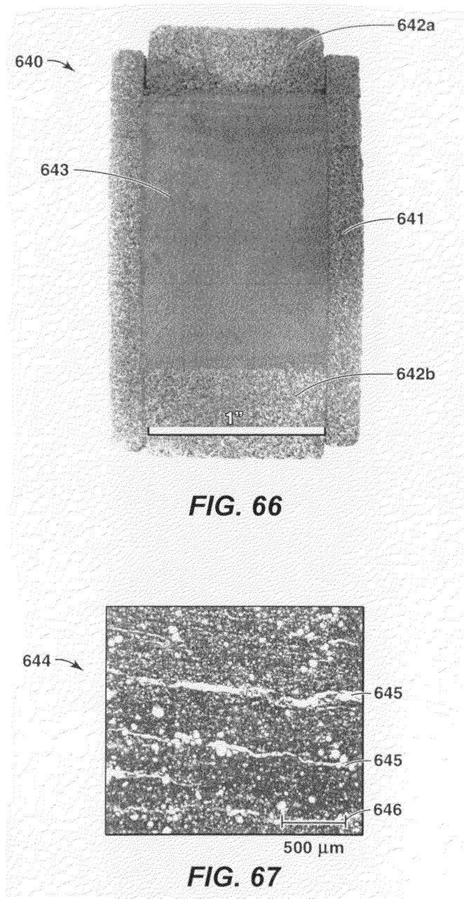

Heating experiments on oil shale at 375 deg. C. under stressed and unstressed conditions confirm that porosity is enhanced by the kerogen conversion process. Exemplary core plugs are provided in FIGS. 62, 64 and 66. An unheated oil shale core plug 620 is depicted in FIG. 62. An unheated thin section 621 from the core plug 620 is shown in FIG. 63 with a scale reference marker 622 denoting a size of 500 μm. Note the lack of porosity in the thin section 621. The thin section 621 is dominated by organic matter mixed with a matrix of clay and calcite dolomite. The larger white specs are quartz grains (rounded) or calcite rhombs. FIG. 64 depicts a core plug 630 that has been heated to 375° C. in a Parr vessel in an inert argon atmosphere with no simulated lithostatic stress applied. The plug 630 shows ample evidence of splitting along laminae presumably where organic material was most abundant. The thin section 631 depicted in FIG. 65 shows a network of large pores and cracks 632. FIG. 65 also includes a scale reference marker 633 denoting a size of 500 μm. Both the plug 630 and the thin section 631 exhibit the swelling associated with kerogen conversion and the resultant porous network developed. FIG. 66 depicts an oil shale plug 643 placed in a sleeve 641 and end caps 642a & 642b of sandstone. The jacketed core 640 was placed in a spring-loaded mini-load-frame assembly (not shown) to simulate overburden stresses of up to 1,000 psi, and then heated to 375° C. The plug photograph depicted in FIG. 66 is a slice through a heated oil shale plug 643 inside its sandstone sleeve 641 and end caps 642a & 642b. Note the lack of expansion cracks and the preservation of lamination. In the thin section 644 depicted in FIG. 67 small fractures 645 that occur in clusters within the oil shale can be observed. These fractures 645 are only 50-100 microns wide. FIG. 67 also includes a scale reference marker 646 denoting a size of 500 μm. Some fractures 645 are oriented parallel to lamination while others are oriented at various angles to lamination. In addition to the fractures, the groundmass of the oil shale contains numerous small pores that form a microporous network. These pores and microfractures are <50 microns in size. Thus kerogen conversion still takes place and porosity is created within the oil shale converted under stress but with a less substantial volume expansion. This set of experiments clearly indicates that even under overburden stress conditions, the kerogen conversion and expulsion process creates porosity and presumably, permeability that was not present in the original oil shale.

### Analysis

The gas and liquid samples obtained through the experimental procedures and gas and liquid sample collection procedures described for Examples 1-5, were analyzed by the following hydrocarbon gas sample gas chromatography (GC) analysis methodology, non-hydrocarbon gas sample gas chromatography (GC) analysis methodology, gas sample GC peak identification and integration methodology, whole oil gas chromatography (WOGC) analysis methodology, and whole oil gas chromatography (WOGC) peak identification and integration methodology.

Gas samples collected during the heating tests as described in Examples 1-5 were analyzed for both hydrocarbon and non-hydrocarbon gases, using an Agilent Model 6890 Gas Chromatograph coupled to an Agilent Model 5973 quadrapole mass selective detector. The 6890 GC was configured with two inlets (front and back) and two detectors (front and back) with two fixed volume sample loops for sample introduction. Peak identifications and integrations were performed using the Chemstation software (Revision A.03.01) supplied with the GC instrument. For hydrocarbon gases, the GC configuration consisted of the following:
a) split/splitless inlet (back position of the GC)
b) FID (Flame ionization detector) back position of the GC
c) HP Ultra-2 (5% Phenyl Methyl Siloxane) capillary columns (two) (25 meters×200 μm ID) one directed to the FID detector, the other to an Agilent 5973 Mass Selective Detector
d) 500 μl fixed volume sample loop
e) six-port gas sampling valve
f) cryogenic (liquid nitrogen) oven cooling capability
g) Oven program −80° C. for 2 mins., 20° C./min. to 0° C., then 4° C./min to 20° C., then 10° C./min. to 100° C., hold for 1 min.
h) Helium carrier gas flow rate of 2.2 ml/min
i) Inlet temperature 100° C.
j) Inlet pressure 19.35 psi
k) Split ratio 25:1
1) FID temperature 310° C.

For non-hydrocarbon gases (e.g., argon, carbon dioxide and hydrogen sulfide) the GC configuration consisted of the following:
a) PTV (programmable temperature vaporization) inlet (front position of the GC)
b) TCD (Thermal conductivity detector) front position of the GC
c) GS-GasPro capillary column (30 meters×0.32 mm ID)
d) 100 μl fixed volume sample loop
e) six port gas sampling valve
f) Oven program: 25° C. hold for 2 min., then 10° C./min to 200° C., hold 1 min.
g) Helium carrier gas flow rate of 4.1 ml/min.
h) Inlet temperature 200° C.
i) Inlet pressure 14.9 psi
j) Splitless mode
k) TCD temperature 250° C.

For Examples 1-5, a stainless steel sample cylinder containing gas collected from the Parr vessel (FIG. 18) was fitted with a two stage gas regulator (designed for lecture bottle use) to reduce gas pressure to approximately twenty pounds per square inch. A septum fitting was positioned at the outlet port of the regulator to allow withdrawal of gas by means of a Hamilton model 1005 gas-tight syringe. Both the septum fitting and the syringe were purged with gas from the stainless steel sample cylinder to ensure that a representative gas sample was collected. The gas sample was then transferred to a stainless steel cell (septum cell) equipped with a pressure transducer and a septum fitting. The septum cell was connected to the fixed volume sample loop mounted on the GC by stainless steel capillary tubing. The septum cell and sample loop were evacuated for approximately 5 minutes. The evacuated septum cell was then isolated from the evacuated sample loop by closure of a needle valve positioned at the outlet of the septum cell. The gas sample was introduced into the septum cell from the gas-tight syringe through the septum fitting and a pressure recorded. The evacuated sample loop was then opened to the pressurized septum cell and the gas sample allowed to equilibrate between the sample loop and the septum cell for one minute. The equilibrium pressure was then recorded, to allow calculation of the total moles of gas present in the sample loop before injection into the GC inlet. The sample loop contents were then swept into the inlet by Helium carrier gas and components separated by retention time in the capillary column, based upon the GC oven temperature program and carrier gas flow rates.

Calibration curves, correlating integrated peak areas with concentration, were generated for quantification of gas compositions using certified gas standards. For hydrocarbon gases, standards containing a mixture of methane, ethane, propane, butane, pentane and hexane in a helium matrix in varying concentrations (parts per million, mole basis) were injected into the GC through the fixed volume sample loop at atmospheric pressure. For non-hydrocarbon gases, standards containing individual components, i.e., carbon dioxide in helium and hydrogen sulfide in natural gas, were injected into the GC at varying pressures in the sample loop to generate calibration curves.

The hydrocarbon gas sample molar percentages reported in FIG. 16 were obtained using the following procedure. Gas standards for methane, ethane, propane, butane, pentane and hexane of at least three varying concentrations were run on the gas chromatograph to obtain peak area responses for such standard concentrations. The known concentrations were then correlated to the respective peak area responses within the Chemstation software to generate calibration curves for methane, ethane, propane, butane, pentane and hexane. The calibration curves were plotted in Chemstation to ensure good linearity (R2>0.98) between concentration and peak intensity. A linear fit was used for each calibrated compound, so that the response factor between peak area and molar concentration was a function of the slope of the line as determined by the Chemstation software. The Chemstation software program then determined a response factor relating GC peak area intensity to the amount of moles for each calibrated compound. The software then determined the number of moles of each calibrated compound from the response factor and the peak area. The peak areas used in Examples 1-5 are reported in Tables 2, 4, 5, 7, and 9. The number of moles of each identified compound for which a calibration curve was not determined (i.e., iso-butane, iso-pentane, and 2-methyl pentane) was then estimated using the response factor for the closest calibrated compound (i.e., butane for iso-butane; pentane for iso-pentane; and hexane for 2-methyl pentane) multiplied by the ratio of the peak area for the identified compound for which a calibration curve was not determined to the peak area of the calibrated compound. The values reported in FIG. 16 were then taken as a percentage of the total of all identified hydrocarbon gas GC areas (i.e., methane, ethane, propane, iso-butane, n-butane, iso-pentane, n-pentane, 2-methyl pentane, and n-hexane) and calculated molar concentrations. Thus the graphed methane to normal C6 molar percentages for all of the experiments do not include the molar contribution of the unidentified hydrocarbon gas species listed in Tables 2, 4, 5, 7, or 9 (e.g., peak numbers 2, 6, 8-11, 13, 15-22, 24-26, and 28-78 in Table 2).

Liquid samples collected during the heating tests as described in Examples 1, 3 and 4 were analyzed by whole oil gas chromatography (WOGC) according to the following procedure. Samples, QA/QC standards and blanks (carbon disulfide) were analyzed using an Ultra 1 Methyl Siloxane column (25 m length, 0.32 μm diameter, 0.52 μm film thickness) in an Agilent 6890 GC equipped with a split/splitless injector, autosampler and flame ionization detector (FID). Samples were injected onto the capillary column in split mode with a split ratio of 80:1. The GC oven temperature was kept constant at 20° C. for 5 min, programmed from 20° C. to 300° C. at a rate of 5° C.min⁻¹, and then maintained at 300° C. for 30 min (total run time=90 min.). The injector temperature was maintained at 300° C. and the FID temperature set at 310° C. Helium was used as carrier gas at a flow of 2.1 mL min⁻¹. Peak identifications and integrations were performed using Chemstation software Rev.A.10.02 [1757] (Agilent Tech. 1990-2003) supplied with the Agilent instrument.

Standard mixtures of hydrocarbons were analyzed in parallel by the WOGC method described above and by an Agilent 6890 GC equipped with a split/splitless injector, autosampler and mass selective detector (MS) under the same conditions. Identification of the hydrocarbon compounds was conducted by analysis of the mass spectrum of each peak from the GC-MS. Since conditions were identical for both instruments, peak identification conducted on the GC-MS could be transferred to the peaks obtained on the GC-FID. Using these data, a compound table relating retention time and peak identification was set up in the GC-FID Chemstation. This table was used for peak identification.

The gas chromatograms obtained on the liquid samples (FIGS. 4, 9 and 11) were integrated using a pseudo-component technique. The convention used for identifying each pseudo-component was to integrate all contributions from normal alkane to next occurring normal alkane with the pseudo-component being named by the late eluting n-alkane. For example, the C-10 pseudo-component would be obtained from integration beginning just past normal-C9 and continue just through normal-C10. The carbon number weight % and mole % values for the pseudo-components obtained in this manner were assigned using correlations developed by Katz and Firoozabadi (Katz, D. L., and A. Firoozabadi, 1978. Predicting phase behavior of condensate/crude-oil systems using methane interaction coefficients, J. Petroleum Technology (November 1978), 1649-1655). Results of the pseudo-component analyses for Examples 1, 3 and 4 are shown in Tables 10, 11 and 12.

An exemplary pseudo component weight percent calculation is presented below with reference to Table 10 for the C10 pseudo component for Example 1 in order to illustrate the technique. First, the C-10 pseudo-component total area is obtained from integration of the area beginning just past normal-C9 and continued just through normal-C10 as described above. The total integration area for the C10 pseudo component is 10551.700 pico-ampere-seconds (pAs). The total C10 pseudo component integration area (10551.700 pAs) is then multiplied by the C10 pseudo component density (0.7780 g/ml) to yield an “area X density” of 8209.22 pAs g/ml. Similarly, the peak integration areas for each pseudo component and all lighter listed compounds (i.e., nC3, iC4, nC4, iC5 & nC5) are determined and multiplied by their respective densities to yield “area X density” numbers for each respective pseudo component and listed compound. The respective determined “area X density” numbers for each pseudo component and listed compound is then summed to determine a “total area X density” number. The “total area X density” number for Example 1 is 96266.96 pAs g/ml. The C10 pseudo component weight percentage is then obtained by dividing the C10 pseudo component “area X density” number (8209.22 pAs g/ml) by the “total area X density” number (96266.96 pAs g/ml) to obtain the C10 pseudo component weight percentage of 8.53 weight percent.

An exemplary pseudo component molar percent calculation is presented below with reference to Table 10 for the C10 pseudo component for Example 1 in order to further illustrate the pseudo component technique. First, the C-10 pseudo-component total area is obtained from integration of the area beginning just past normal-C9 and continued just through normal-C10 as described above. The total integration area for the C10 pseudo component is 10551.700 pico-ampere-seconds (pAs). The total C10 pseudo component integration area (10551.700 pAs) is then multiplied by the C10 pseudo component density (0.7780 g/ml) to yield an “area X density” of 8209.22 pAs g/ml. Similarly, the integration areas for each pseudo component and all lighter listed compounds (i.e., nC3, iC4, nC4, iC5 & nC5) are determined and multiplied by their respective densities to yield “area X density” numbers for each respective pseudo component and listed compound. The C10 pseudo component “area X density” number (8209.22 pAs g/ml) is then divided by the C10 pseudo component molecular weight (134.00 g/mol) to yield a C10 pseudo component “area X density/molecular weight” number of 61.26 pAs mol/ml. Similarly, the “area X density” number for each pseudo component and listed compound is then divided by such components or compounds respective molecular weight to yield an “area X density/molecular weight” number for each respective pseudo component and listed compound. The respective determined “area X density/molecular weight” numbers for each pseudo component and listed compound is then summed to determine a “total area X density/molecular weight” number. The total “total area X density/molecular weight” number for Example 1 is 665.28 pAs mol/ml. The C10 pseudo component molar percentage is then obtained by dividing the C10 pseudo component “area X density/molecular weight” number (61.26 pAs mol/ml) by the “total area X density/molecular weight” number (665.28 pAs mol/ml) to obtain the C10 pseudo component molar percentage of 9.21 molar percent.

TABLE 10
Pseudo-components for Example 1 - GC of liquid - 0 stress

| | | | Avg. Boiling | Density | Molecular | | |
|---|---|---|---|---|---|---|---|
| Component | Area (cts.) | Area % | Pt. (° F.) | (g/ml) | Wt. (g/mol) | Wt. % | Mol % |
| nC₃ | 41.881 | 0.03 | −43.73 | 0.5069 | 44.10 | 0.02 | 0.07 |
| iC₄ | 120.873 | 0.10 | 10.94 | 0.5628 | 58.12 | 0.07 | 0.18 |
| nC₄ | 805.690 | 0.66 | 31.10 | 0.5840 | 58.12 | 0.49 | 1.22 |
| iC₅ | 1092.699 | 0.89 | 82.13 | 0.6244 | 72.15 | 0.71 | 1.42 |
| nC₅ | 2801.815 | 2.29 | 96.93 | 0.6311 | 72.15 | 1.84 | 3.68 |
| Pseudo C₆ | 7150.533 | 5.84 | 147.00 | 0.6850 | 84.00 | 5.09 | 8.76 |
| Pseudo C₇ | 10372.800 | 8.47 | 197.50 | 0.7220 | 96.00 | 7.78 | 11.73 |
| Pseudo C₈ | 11703.500 | 9.56 | 242.00 | 0.7450 | 107.00 | 9.06 | 12.25 |
| Pseudo C₉ | 11776.200 | 9.61 | 288.00 | 0.7640 | 121.00 | 9.35 | 11.18 |
| Pseudo C₁₀ | 10551.700 | 8.61 | 330.50 | 0.7780 | 134.00 | 8.53 | 9.21 |
| Pseudo C₁₁ | 9274.333 | 7.57 | 369.00 | 0.7890 | 147.00 | 7.60 | 7.48 |
| Pseudo C₁₂ | 8709.231 | 7.11 | 407.00 | 0.8000 | 161.00 | 7.24 | 6.50 |
| Pseudo C₁₃ | 7494.549 | 6.12 | 441.00 | 0.8110 | 175.00 | 6.31 | 5.22 |
| Pseudo C₁₄ | 6223.394 | 5.08 | 475.50 | 0.8220 | 190.00 | 5.31 | 4.05 |
| Pseudo C₁₅ | 6000.179 | 4.90 | 511.00 | 0.8320 | 206.00 | 5.19 | 3.64 |
| Pseudo C₁₆ | 5345.791 | 4.36 | 542.00 | 0.8390 | 222.00 | 4.66 | 3.04 |
| Pseudo C₁₇ | 4051.886 | 3.31 | 572.00 | 0.8470 | 237.00 | 3.57 | 2.18 |
| Pseudo C₁₈ | 3398.586 | 2.77 | 595.00 | 0.8520 | 251.00 | 3.01 | 1.73 |
| Pseudo C₁₉ | 2812.101 | 2.30 | 617.00 | 0.8570 | 263.00 | 2.50 | 1.38 |
| Pseudo C₂₀ | 2304.651 | 1.88 | 640.50 | 0.8620 | 275.00 | 2.06 | 1.09 |
| Pseudo C₂₁ | 2038.925 | 1.66 | 664.00 | 0.8670 | 291.00 | 1.84 | 0.91 |
| Pseudo C₂₂ | 1497.726 | 1.22 | 686.00 | 0.8720 | 305.00 | 1.36 | 0.64 |
| Pseudo C₂₃ | 1173.834 | 0.96 | 707.00 | 0.8770 | 318.00 | 1.07 | 0.49 |
| Pseudo C₂₄ | 822.762 | 0.67 | 727.00 | 0.8810 | 331.00 | 0.75 | 0.33 |
| Pseudo C₂₅ | 677.938 | 0.55 | 747.00 | 0.8850 | 345.00 | 0.62 | 0.26 |
| Pseudo C₂₆ | 532.788 | 0.43 | 766.00 | 0.8890 | 359.00 | 0.49 | 0.20 |
| Pseudo C₂₇ | 459.465 | 0.38 | 784.00 | 0.8930 | 374.00 | 0.43 | 0.16 |
| Pseudo C₂₈ | 413.397 | 0.34 | 802.00 | 0.8960 | 388.00 | 0.38 | 0.14 |
| Pseudo C₂₉ | 522.898 | 0.43 | 817.00 | 0.8990 | 402.00 | 0.49 | 0.18 |
| Pseudo C₃₀ | 336.968 | 0.28 | 834.00 | 0.9020 | 416.00 | 0.32 | 0.11 |
| Pseudo C₃₁ | 322.495 | 0.26 | 850.00 | 0.9060 | 430.00 | 0.30 | 0.10 |
| Pseudo C₃₂ | 175.615 | 0.14 | 866.00 | 0.9090 | 444.00 | 0.17 | 0.05 |
| Pseudo C₃₃ | 165.912 | 0.14 | 881.00 | 0.9120 | 458.00 | 0.16 | 0.05 |
| Pseudo C₃₄ | 341.051 | 0.28 | 895.00 | 0.9140 | 472.00 | 0.32 | 0.10 |
| Pseudo C₃₅ | 286.861 | 0.23 | 908.00 | 0.9170 | 486.00 | 0.27 | 0.08 |
| Pseudo C₃₆ | 152.814 | 0.12 | 922.00 | 0.9190 | 500.00 | 0.15 | 0.04 |
| Pseudo C₃₇ | 356.947 | 0.29 | 934.00 | 0.9220 | 514.00 | 0.34 | 0.10 |
| Pseudo C₃₈ | 173.428 | 0.14 | 947.00 | 0.9240 | 528.00 | 0.17 | 0.05 |
| Totals | 122484.217 | 100.00 | | | | 100.00 | 100.00 |

TABLE 11
Pseudo-components for Example 3 - GC of liquid - 400 psi stress

| | | | Avg. Boiling | Density | Molecular Wt. | | |
|---|---|---|---|---|---|---|---|
| Component | Area | Area % | Pt. (° F.) | (g/ml) | (g/mol) | Wt. % | Mol % |
| nC₃ | 35.845 | 0.014 | −43.730 | 0.5069 | 44.10 | 0.01 | 0.03 |
| iC₄ | 103.065 | 0.041 | 10.940 | 0.5628 | 58.12 | 0.03 | 0.07 |
| nC₄ | 821.863 | 0.328 | 31.100 | 0.5840 | 58.12 | 0.24 | 0.62 |
| iC₅ | 1187.912 | 0.474 | 82.130 | 0.6244 | 72.15 | 0.37 | 0.77 |
| nC₅ | 3752.655 | 1.498 | 96.930 | 0.6311 | 72.15 | 1.20 | 2.45 |
| Pseudo C₆ | 12040.900 | 4.805 | 147.000 | 0.6850 | 84.00 | 4.17 | 7.34 |
| Pseudo C₇ | 20038.600 | 7.997 | 197.500 | 0.7220 | 96.00 | 7.31 | 11.26 |
| Pseudo C₈ | 24531.500 | 9.790 | 242.000 | 0.7450 | 107.00 | 9.23 | 12.76 |
| Pseudo C₉ | 25315.000 | 10.103 | 288.000 | 0.7640 | 121.00 | 9.77 | 11.94 |
| Pseudo C₁₀ | 22640.400 | 9.035 | 330.500 | 0.7780 | 134.00 | 8.90 | 9.82 |
| Pseudo C₁₁ | 20268.100 | 8.089 | 369.000 | 0.7890 | 147.00 | 8.08 | 8.13 |
| Pseudo C₁₂ | 18675.600 | 7.453 | 407.000 | 0.8000 | 161.00 | 7.55 | 6.93 |
| Pseudo C₁₃ | 16591.100 | 6.621 | 441.000 | 0.8110 | 175.00 | 6.80 | 5.74 |
| Pseudo C₁₄ | 13654.000 | 5.449 | 475.500 | 0.8220 | 190.00 | 5.67 | 4.41 |
| Pseudo C₁₅ | 13006.300 | 5.191 | 511.000 | 0.8320 | 206.00 | 5.47 | 3.92 |
| Pseudo C₁₆ | 11962.200 | 4.774 | 542.000 | 0.8390 | 222.00 | 5.07 | 3.38 |
| Pseudo C₁₇ | 8851.622 | 3.533 | 572.000 | 0.8470 | 237.00 | 3.79 | 2.36 |
| Pseudo C₁₈ | 7251.438 | 2.894 | 595.000 | 0.8520 | 251.00 | 3.12 | 1.84 |
| Pseudo C₁₉ | 5946.166 | 2.373 | 617.000 | 0.8570 | 263.00 | 2.57 | 1.45 |
| Pseudo C₂₀ | 4645.178 | 1.854 | 640.500 | 0.8620 | 275.00 | 2.02 | 1.09 |
| Pseudo C₂₁ | 4188.168 | 1.671 | 664.000 | 0.8670 | 291.00 | 1.83 | 0.93 |
| Pseudo C₂₂ | 2868.636 | 1.145 | 686.000 | 0.8720 | 305.00 | 1.26 | 0.61 |
| Pseudo C₂₃ | 2188.895 | 0.874 | 707.000 | 0.8770 | 318.00 | 0.97 | 0.45 |
| Pseudo C₂₄ | 1466.162 | 0.585 | 727.000 | 0.8810 | 331.00 | 0.65 | 0.29 |
| Pseudo C₂₅ | 1181.133 | 0.471 | 747.000 | 0.8850 | 345.00 | 0.53 | 0.23 |
| Pseudo C₂₆ | 875.812 | 0.350 | 766.000 | 0.8890 | 359.00 | 0.39 | 0.16 |
| Pseudo C₂₇ | 617.103 | 0.246 | 784.000 | 0.8930 | 374.00 | 0.28 | 0.11 |
| Pseudo C₂₈ | 538.147 | 0.215 | 802.000 | 0.8960 | 388.00 | 0.24 | 0.09 |
| Pseudo C₂₉ | 659.027 | 0.263 | 817.000 | 0.8990 | 402.00 | 0.30 | 0.11 |
| Pseudo C₃₀ | 1013.942 | 0.405 | 834.000 | 0.9020 | 416.00 | 0.46 | 0.16 |
| Pseudo C₃₁ | 761.259 | 0.304 | 850.000 | 0.9060 | 430.00 | 0.35 | 0.12 |
| Pseudo C₃₂ | 416.031 | 0.166 | 866.000 | 0.9090 | 444.00 | 0.19 | 0.06 |
| Pseudo C₃₃ | 231.207 | 0.092 | 881.000 | 0.9120 | 458.00 | 0.11 | 0.03 |
| Pseudo C₃₄ | 566.926 | 0.226 | 895.000 | 0.9140 | 472.00 | 0.26 | 0.08 |
| Pseudo C₃₅ | 426.697 | 0.170 | 908.000 | 0.9170 | 486.00 | 0.20 | 0.06 |
| Pseudo C₃₆ | 191.626 | 0.076 | 922.000 | 0.9190 | 500.00 | 0.09 | 0.03 |
| Pseudo C₃₇ | 778.713 | 0.311 | 934.000 | 0.9220 | 514.00 | 0.36 | 0.10 |
| Pseudo C₃₈ | 285.217 | 0.114 | 947.000 | 0.9240 | 528.00 | 0.13 | 0.04 |
| Totals | 250574.144 | 100.000 | | | | 100.00 | 100.00 |

TABLE 12
Pseudo-components for Example 4 - GC of liquid - 1000 psi stress

| | | | Avg. Boiling | Density | Molecular Wt. | | |
|---|---|---|---|---|---|---|---|
| Component | Area | Area % | Pt. (° F.) | (g/ml) | (g/mol) | Wt. % | Mol % |
| nC₃ | 44.761 | 0.023 | −43.730 | 0.5069 | 44.10 | 0.01 | 0.05 |
| iC₄ | 117.876 | 0.060 | 10.940 | 0.5628 | 58.12 | 0.04 | 0.11 |
| nC₄ | 927.866 | 0.472 | 31.100 | 0.5840 | 58.12 | 0.35 | 0.87 |
| iC₅ | 1082.570 | 0.550 | 82.130 | 0.6244 | 72.15 | 0.44 | 0.88 |
| nC₅ | 3346.533 | 1.701 | 96.930 | 0.6311 | 72.15 | 1.37 | 2.74 |
| Pseudo C₆ | 9579.443 | 4.870 | 147.000 | 0.6850 | 84.00 | 4.24 | 7.31 |
| Pseudo C₇ | 16046.200 | 8.158 | 197.500 | 0.7220 | 96.00 | 7.49 | 11.29 |
| Pseudo C₈ | 19693.300 | 10.012 | 242.000 | 0.7450 | 107.00 | 9.48 | 12.83 |
| Pseudo C₉ | 20326.300 | 10.334 | 288.000 | 0.7640 | 121.00 | 10.04 | 12.01 |
| Pseudo C₁₀ | 18297.600 | 9.302 | 330.500 | 0.7780 | 134.00 | 9.20 | 9.94 |
| Pseudo C₁₁ | 16385.600 | 8.330 | 369.000 | 0.7890 | 147.00 | 8.36 | 8.23 |
| Pseudo C₁₂ | 15349.000 | 7.803 | 407.000 | 0.8000 | 161.00 | 7.94 | 7.14 |
| Pseudo C₁₃ | 13116.500 | 6.668 | 441.000 | 0.8110 | 175.00 | 6.88 | 5.69 |
| Pseudo C₁₄ | 10816.100 | 5.499 | 475.500 | 0.8220 | 190.00 | 5.75 | 4.38 |
| Pseudo C₁₅ | 10276.900 | 5.225 | 511.000 | 0.8320 | 206.00 | 5.53 | 3.88 |
| Pseudo C₁₆ | 9537.818 | 4.849 | 542.000 | 0.8390 | 222.00 | 5.17 | 3.37 |
| Pseudo C₁₇ | 6930.611 | 3.523 | 572.000 | 0.8470 | 237.00 | 3.79 | 2.32 |
| Pseudo C₁₈ | 5549.802 | 2.821 | 595.000 | 0.8520 | 251.00 | 3.06 | 1.76 |
| Pseudo C₁₉ | 4440.457 | 2.257 | 617.000 | 0.8570 | 263.00 | 2.46 | 1.35 |
| Pseudo C₂₀ | 3451.250 | 1.755 | 640.500 | 0.8620 | 275.00 | 1.92 | 1.01 |
| Pseudo C₂₁ | 3133.251 | 1.593 | 664.000 | 0.8670 | 291.00 | 1.76 | 0.87 |
| Pseudo C₂₂ | 2088.036 | 1.062 | 686.000 | 0.8720 | 305.00 | 1.18 | 0.56 |
| Pseudo C₂₃ | 1519.460 | 0.772 | 707.000 | 0.8770 | 318.00 | 0.86 | 0.39 |
| Pseudo C₂₄ | 907.473 | 0.461 | 727.000 | 0.8810 | 331.00 | 0.52 | 0.23 |
| Pseudo C₂₅ | 683.205 | 0.347 | 747.000 | 0.8850 | 345.00 | 0.39 | 0.16 |
| Pseudo C₂₆ | 493.413 | 0.251 | 766.000 | 0.8890 | 359.00 | 0.28 | 0.11 |
| Pseudo C₂₇ | 326.831 | 0.166 | 784.000 | 0.8930 | 374.00 | 0.19 | 0.07 |
| Pseudo C₂₈ | 272.527 | 0.139 | 802.000 | 0.8960 | 388.00 | 0.16 | 0.06 |
| Pseudo C₂₉ | 291.862 | 0.148 | 817.000 | 0.8990 | 402.00 | 0.17 | 0.06 |
| Pseudo C₃₀ | 462.840 | 0.235 | 834.000 | 0.9020 | 416.00 | 0.27 | 0.09 |
| Pseudo C₃₁ | 352.886 | 0.179 | 850.000 | 0.9060 | 430.00 | 0.21 | 0.07 |
| Pseudo C₃₂ | 168.635 | 0.086 | 866.000 | 0.9090 | 444.00 | 0.10 | 0.03 |
| Pseudo C₃₃ | 67.575 | 0.034 | 881.000 | 0.9120 | 458.00 | 0.04 | 0.01 |
| Pseudo C₃₄ | 95.207 | 0.048 | 895.000 | 0.9140 | 472.00 | 0.06 | 0.02 |
| Pseudo C₃₅ | 226.660 | 0.115 | 908.000 | 0.9170 | 486.00 | 0.13 | 0.04 |
| Pseudo C₃₆ | 169.729 | 0.086 | 922.000 | 0.9190 | 500.00 | 0.10 | 0.03 |
| Pseudo C₃₇ | 80.976 | 0.041 | 934.000 | 0.9220 | 514.00 | 0.05 | 0.01 |
| Pseudo C₃₈ | 42.940 | 0.022 | 947.000 | 0.9240 | 528.00 | 0.03 | 0.01 |
| Totals | 196699.994 | 100.000 | | | | 100.00 | 100.00 |

TOC and Rock-eval tests were performed on specimens from oil shale block CM-1B taken at the same stratigraphic interval as the specimens tested by the Parr heating method described in Examples 1-5. These tests resulted in a TOC of 21% and a Rock-eval Hydrogen Index of 872 mg/g-toc.

The TOC and rock-eval procedures described below were performed on the oil shale specimens remaining after the Parr heating tests described in Examples 1-5. Results are shown in Table 13.

The Rock-Eval pyrolysis analyses described above were performed using the following procedures. Rock-Eval pyrolysis analyses were performed on calibration rock standards (IFP standard #55000), blanks, and samples using a Delsi Rock-Eval II instrument. Rock samples were crushed, micronized, and air-dried before loading into Rock-Eval crucibles. Between 25 and 100 mg of powdered-rock samples were loaded into the crucibles depending on the total organic carbon (TOC) content of the sample. Two or three blanks were run at the beginning of each day to purge the system and stabilize the temperature. Two or three samples of IFP calibration standard #55000 with weight of 100+/−1 mg were run to calibrate the system. If the Rock-Eval Tₘₐₓ parameter was 419° C.+/−2° C. on these standards, analyses proceeded with samples. The standard was also run before and after every 10 samples to monitor the instrument's performance.

The Rock-Eval pyrolysis technique involves the rate-programmed heating of a powdered rock sample to a high temperature in an inert (helium) atmosphere and the characterization of products generated from the thermal breakdown of chemical bonds. After introduction of the sample the pyrolysis oven was held isothermally at 300° C. for three minutes. Hydrocarbons generated during this stage are detected by a flame-ionization detector (FID) yielding the S₁ peak. The pyrolysis-oven temperature was then increased at a gradient of 25° C./minute up to 550° C., where the oven was held isothermally for one minute. Hydrocarbons generated during this step were detected by the FID and yielded the S₂ peak.

Hydrogen Index (HI) is calculated by normalizing the S₂ peak (expressed as mg_{hydrocarbons}/g_{rock}) to weight % TOC (Total Organic Carbon determined independently) as follows:

HI=(S₂/TOC)*100

where HI is expressed as mg_{hydrocarbons}/g_{TOC}

Total Organic Carbon (TOC) was determined by well known methods suitable for geological samples—i.e., any carbonate rock present was removed by acid treatment followed by combustion of the remaining material to produce and measure organic based carbon in the form of CO₂.

TABLE 13
TOC and Rock-eval results on oil shale specimens after
the Parr heating tests.

| | Example 1 | Example 2 | Example 3 | Example 4 | Example 5 |
|---|---|---|---|---|---|
| TOC (%) | 12.07 | 10.83 | 10.62 | 11.22 | 11.63 |
| HI (mg/g- | 77 | 83 | 81 | 62 | 77 |
| toc) | | | | | |

The API gravity of Examples 1-5 was estimated by estimating the room temperature specific gravity (SG) of the liquids collected and the results are reported in Table 14. The API gravity was estimated from the determined specific gravity by applying the following formula:

API gravity=(141.5/SG)−131.5

The specific gravity of each liquid sample was estimated using the following procedure. An empty 50 μl Hamilton Model 1705 gastight syringe was weighed on a Mettler AE 163 digital balance to determine the empty syringe weight. The syringe was then loaded by filling the syringe with a volume of liquid. The volume of liquid in the syringe was noted. The loaded syringe was then weighed. The liquid sample weight was then estimated by subtracting the loaded syringe measured weight from the measured empty syringe weight. The specific gravity was then estimated by dividing the liquid sample weight by the syringe volume occupied by the liquid sample.

TABLE 14
Estimated API Gravity of liquid samples from Examples 1-5

| Example | Example 1 | Example 2 | Example 3 | Example 4 | Example 5 |
|---|---|---|---|---|---|
| API Gravity | 29.92 | 30.00 | 27.13 | 32.70 | 30.00 |

The liquid samples obtained through the experimental procedures and gas and liquid sample collection procedures described for Examples 6-19, were analyzed by the following hydrocarbon liquid sample C4-C19 gas chromatography (C4-C19 GC) analysis methodology, C4-C19 GC peak identification and integration methodology. While the liquid samples from Examples 6-19 were also analyzed using the whole oil gas chromatography (WOGC) analysis methodology, and whole oil gas chromatography (WOGC) peak identification and integration methodology discussed above with reference to Examples 1-5.

Liquid samples collected during the heating tests described in Examples 7-19 were analyzed by a gas chromatographic (GC) procedure designed to produce well separated GC peaks for materials having carbon numbers in the range C4-C19. The following procedure describes that analysis.

The gas chromatographic separation was accomplished in two stages—a first stage to separate the C4-C19 fraction from the sample, discarding those components present with higher carbon numbers. This C4-C19 fraction was then passed to the second stage of GC analysis, where a more complete, analytical separation was accomplished.

The gas chromatography equipment consisted of a Hewlett Packard 5890 (Series II) Gas Chromatograph equipped with an FID detector, an HP 6890 autosampler, a split injector and a computer supplied with Agilent ChemStation software. This GC was augmented with a pre-fractionator as described below.

The first stage of separation, the pre-fractionator, consisted of an injector port and oven, the oven containing one packed stainless steel column (20% OV101 on 80/100 mesh Chromosorb WHP, ⅛ inch I.D and 4 feet long), a second packed stainless steel column (5 A mole sieve, 60/80 mesh, ⅛ inch ID and 2 feet long), and a multi-port valve. Both the oven and the injector port were maintained at 300° C. UHP Helium carrier gas flowing at a rate of 25 ml/min was used. The OV101 column was used to isolate the C4-C19 fraction in the sample and the heavier compounds (C19+) were backflushed into the mole sieve column. The multi-port valve was used in a timed manner to isolate the C4-C19 fraction and provide that fraction as input to the second stage of separation.

The second stage of separation, the analytical separation, consisted of a split injector, an oven equipped with temperature control, an analytical column acquired from Agilent (PONA fused silica column 50 m in length, 0.2 mm ID and 0.5 μm film thickness) and flame ionization detector (FID). The injector and FID detector were maintained at 300° C.

Liquid samples to be analyzed were placed in sealed glass vials suitable for use in the autosampler and weighed. To each sample 1 μl of 1-hexene (GC grade from Sigma Aldrich) was added. The 1-hexene served as an internal standard with known amount and also as a retention time standard. 10 μl of a mixture of additional reference standards was added to each sample. These reference standards consisted of a mixture of equal volumes of the following four compounds: 2,3-dimethyl-2-pentene, cis-2-octene, 1-nonene, and 1,2,3,4-tetramethylbenzene. These reference standards were used as retention time standards.

The autosampler was set to inject 1 μl of sample (containing the standards). The second stage of separation was accomplished using a helium flow rate of 1 ml/min and a GC oven temperature program as follows:
Initial temp=35° C.
Initial hold time=15.00 min
Rate 1=1.50° C./min
Temp 1_{f}=70° C.
Hold Time 1_{f}=0.0 min
Rate 2=3.00° C./min
Temp 2_{f}=130° C.
Hold Time 2_{f}=12.0 min
Rate 3=3.00° C./min
Temp 3_{f}=300° C.
Hold Time 3_{f}=20.0 min
   Gas pressure settings on the GC were held at helium=64 psi, hydrogen=15 psi, and air=44 psi.

Agilent ChemStation Revision A.10.02 software was used to control the instrument and perform data integration. This software was used to make any necessary retention time adjustments based on the responses of the standard materials contained in each sample. Peak assignments were made based on retention times of known compounds and separate analyses using GC/mass spectrometry for compound identification.

A quality control sample of a previously collected C4-C19 fraction was maintained and run routinely to ensure instrument and data reproducibility.

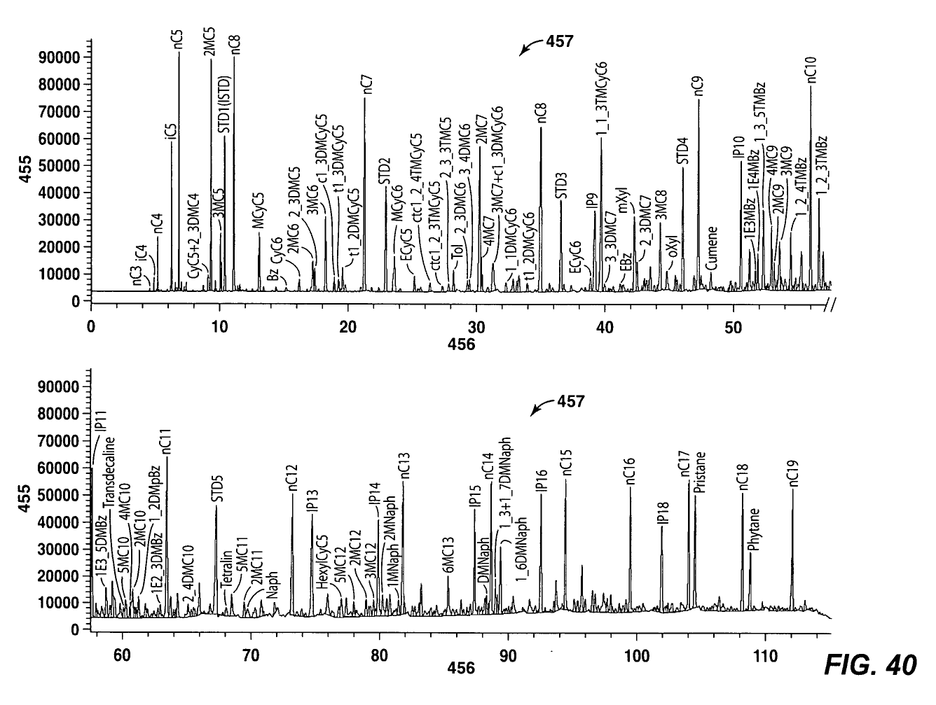

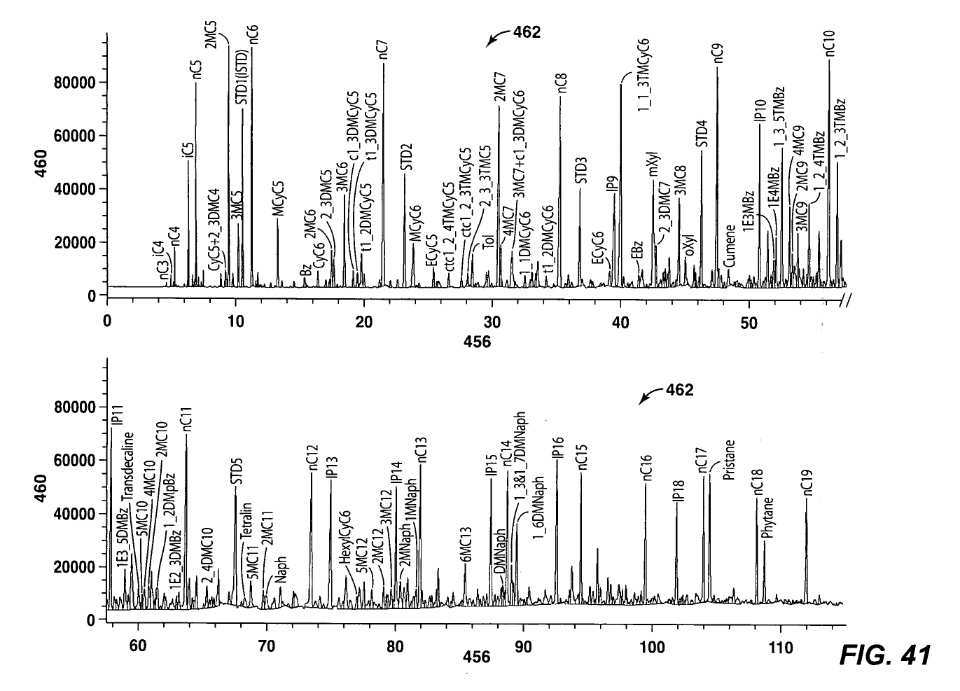

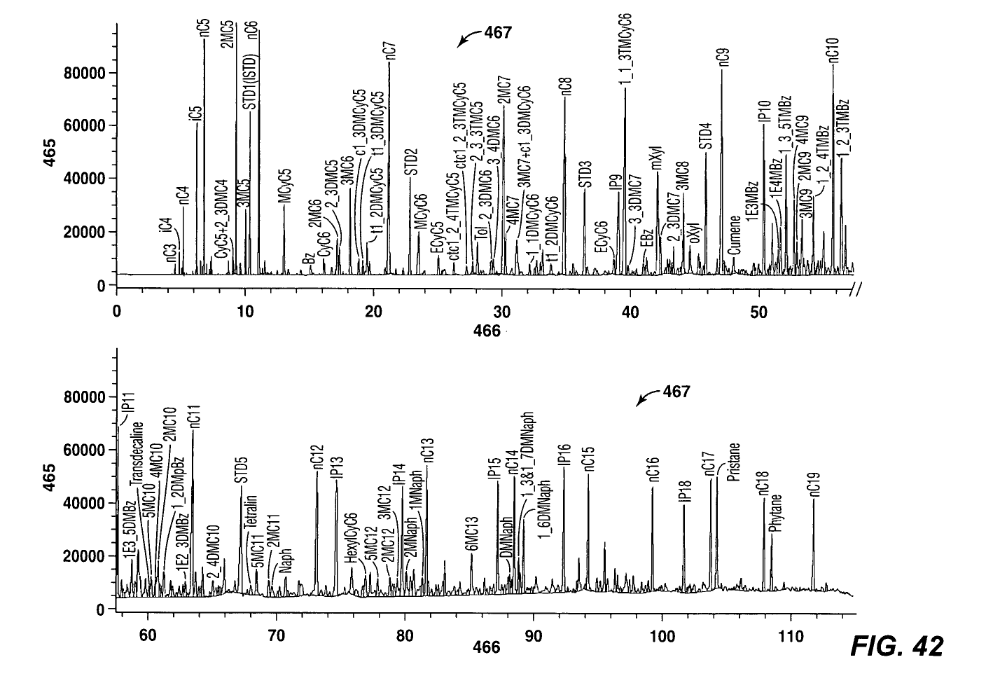

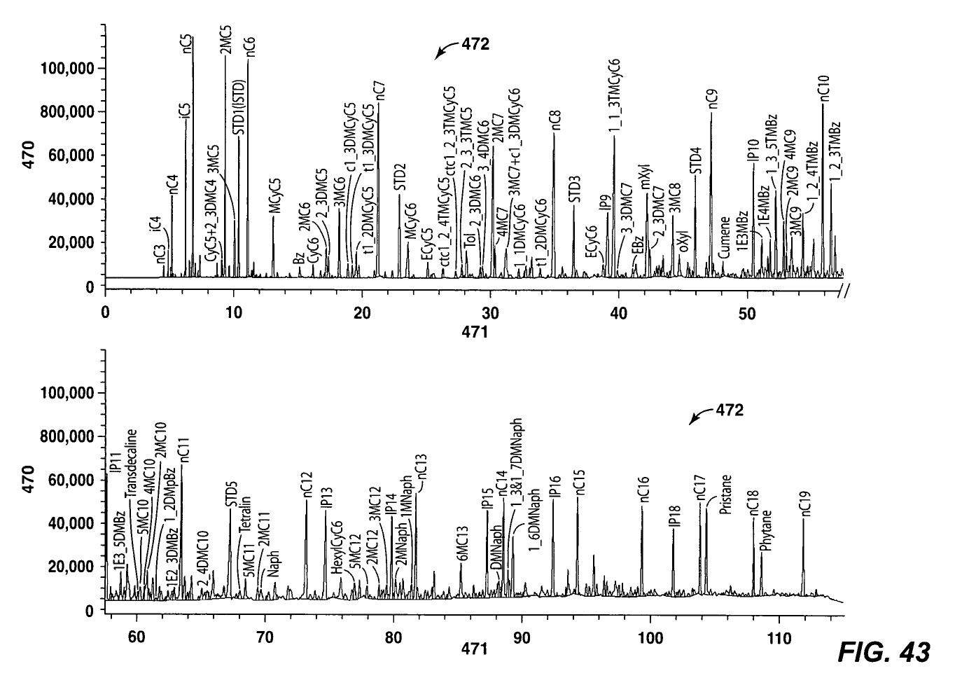

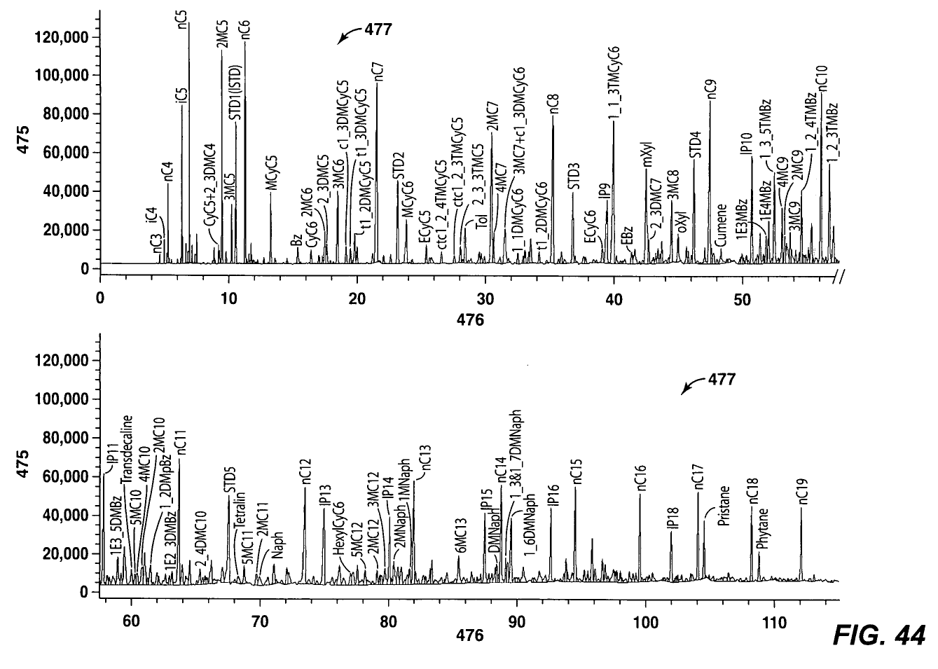

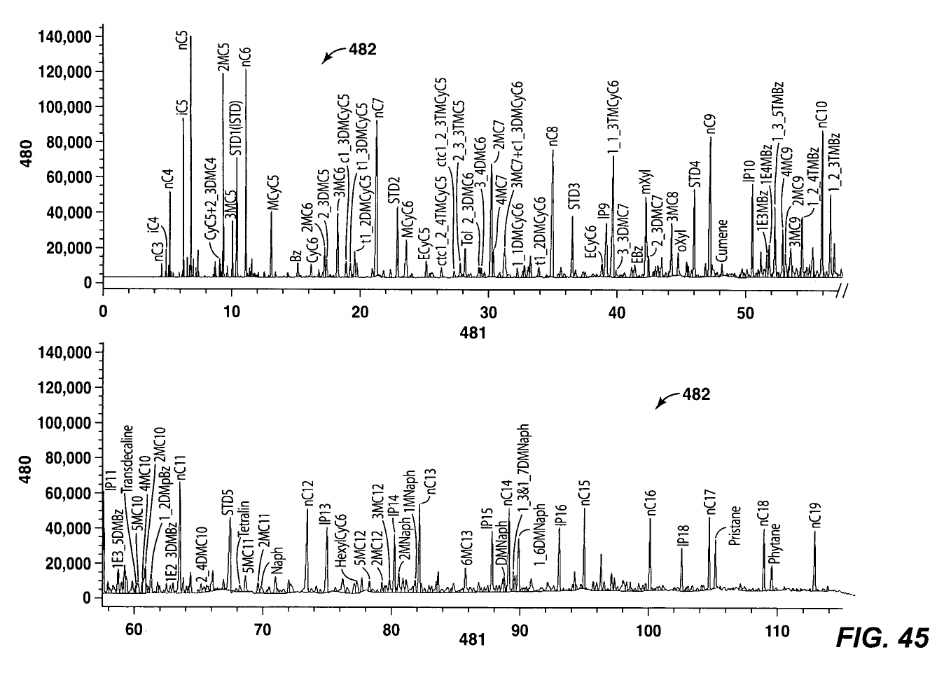

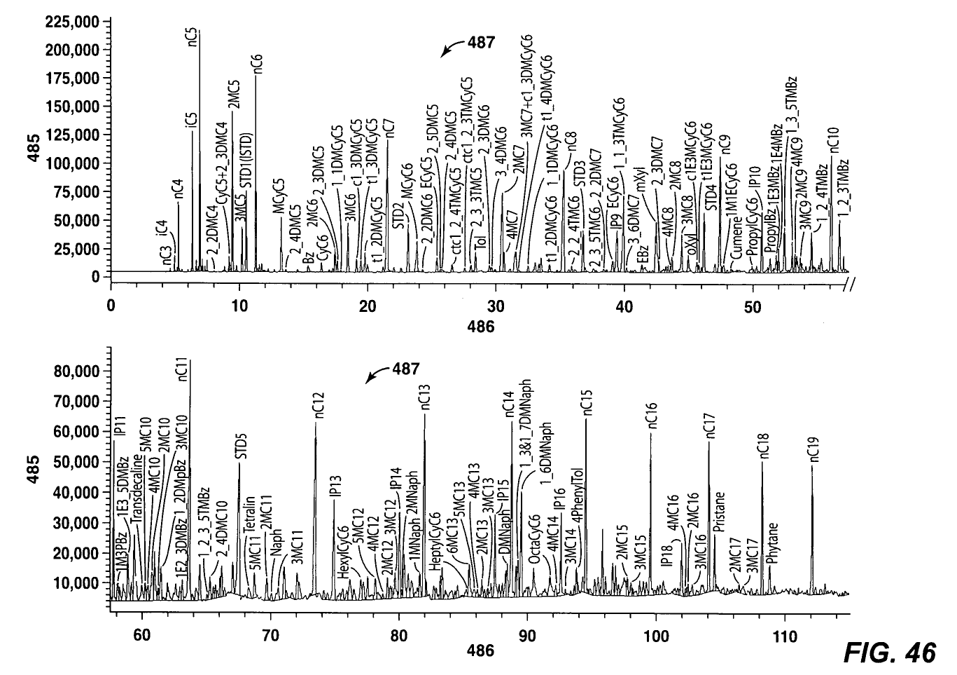

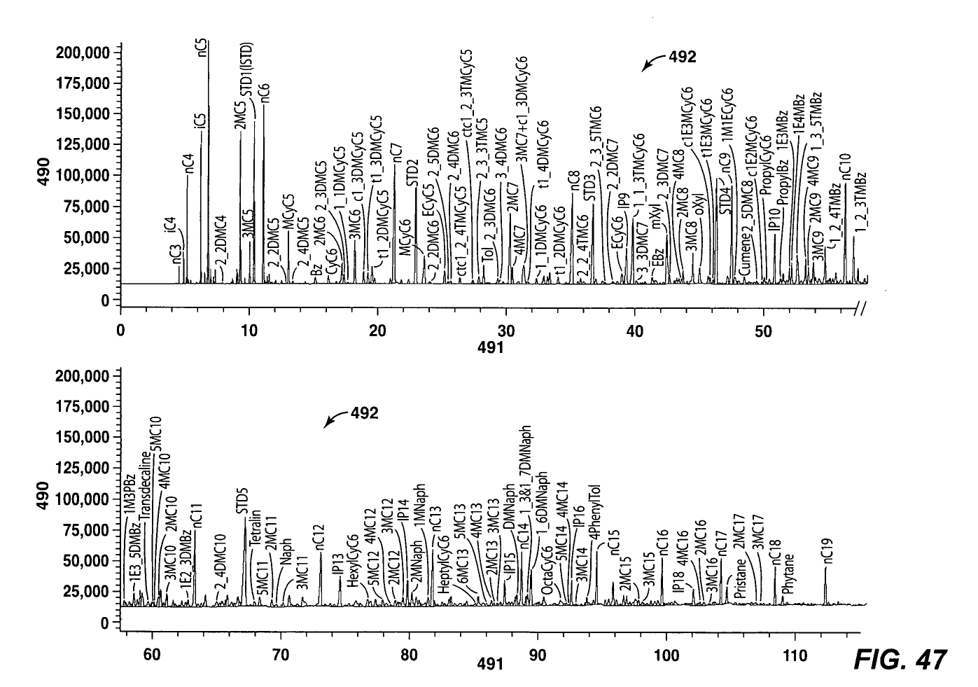

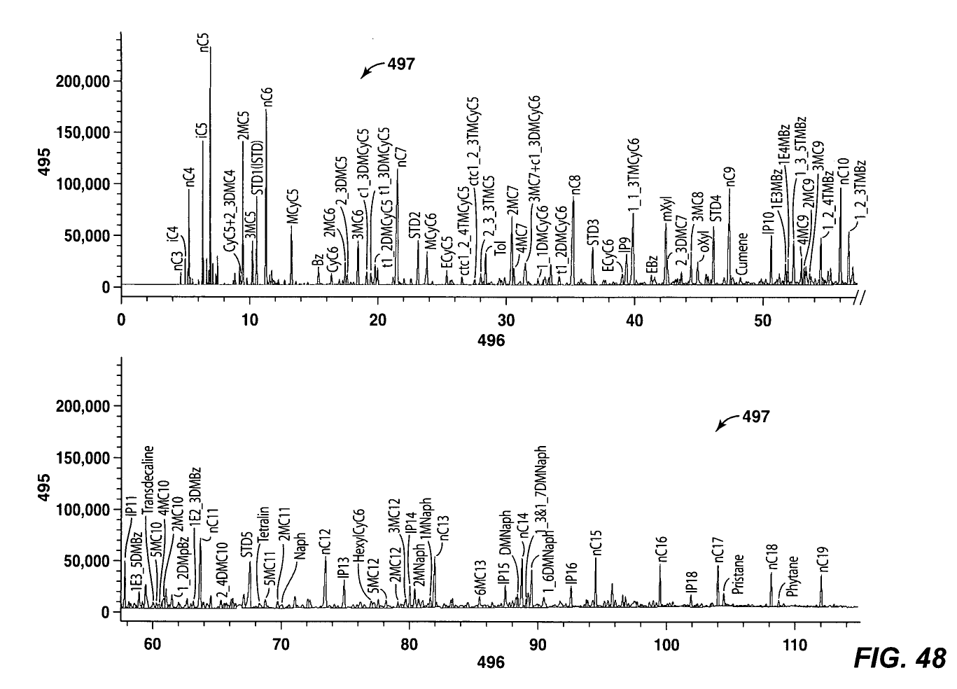

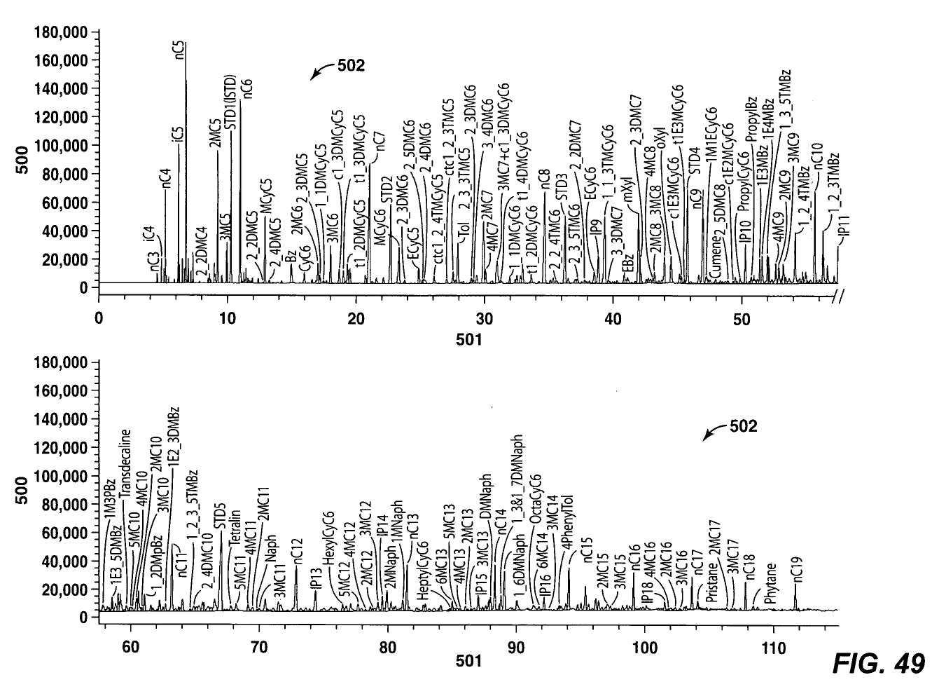

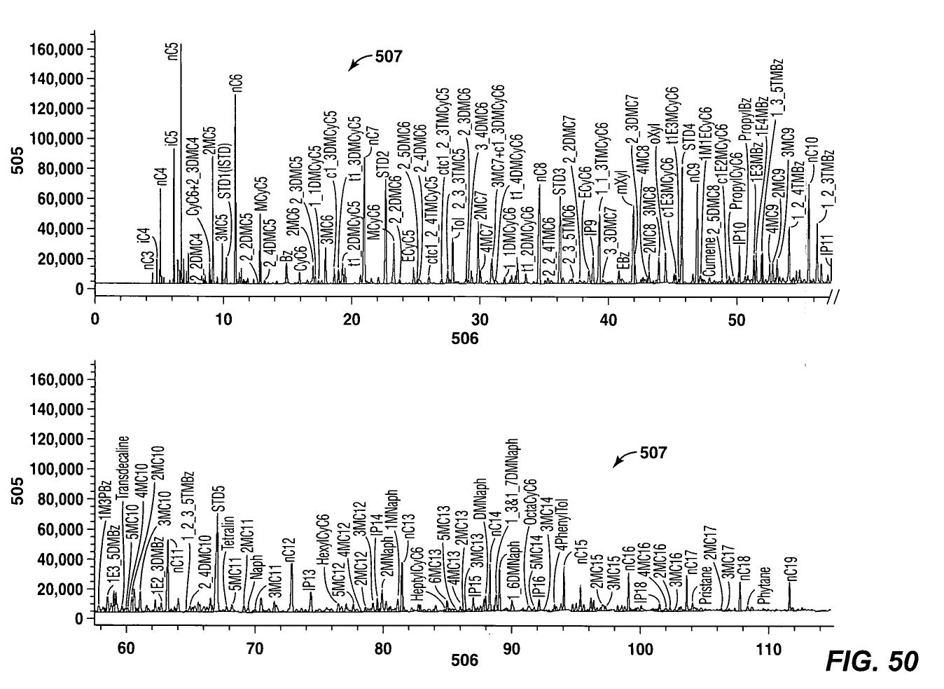

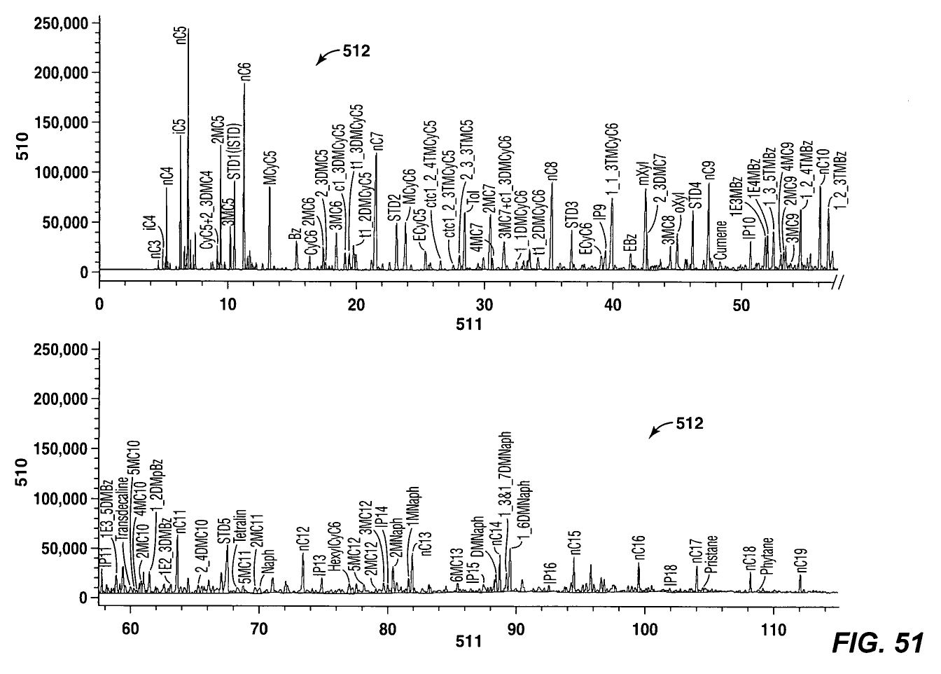

The C4-C19 GC chromatograms for Examples 6-19 are depicted in FIGS. 39-52, with FIG. 39 containing the C4-C19 GC chromatogram for Example 6, FIG. 40 containing the C4-C19 GC chromatogram for Example 7, FIG. 41 containing the C4-C19 GC chromatogram for Example 8, FIG. 42 containing the C4-C19 GC chromatogram for Example 9, FIG. 43 containing the C4-C19 GC chromatogram for Example 10, FIG. 44 containing the C4-C19 GC chromatogram for Example 11, FIG. 45 containing the C4-C19 GC chromatogram for Example 12, FIG. 46 containing the C4-C19 GC chromatogram for Example 13, FIG. 47 containing the C4-C19 GC chromatogram for Example 14, FIG. 48 containing the C4-C19 GC chromatogram for Example 15, FIG. 49 containing the C4-C19 GC chromatogram for Example 16, FIG. 50 containing the C4-C19 GC chromatogram for Example 17, FIG. 51 containing the C4-C19 GC chromatogram for Example 18, and FIG. 52 containing the C4-C19 GC chromatogram for Example 19. The y-axis (labeled 450, 455, 460, 465, 470, 475, 480, 485, 490, 495, 500, 505, 510 & 515 respectively) of each of the figures represents signal response in pico-amperes (pA) with the x-axis (labeled 451, 456, 461, 466, 471, 476, 481, 486, 491, 496, 501, 506, 511 & 516 respectively) representing retention time in minutes. Each respective chromatogram (labeled 452, 457, 462, 467, 472, 477, 482, 487, 492, 497, 502, 507, 512 & 517 respectively), including a series of peaks, is identified. Each identified peak for each respective chromatogram is labeled with abbreviations corresponding to compound names.

The C4-C19 GC chromatograms for Examples 6-19 were integrated to obtain individual peak areas for each identified compound as previously discussed. Some compounds, routinely identified by C4-C19 GC analysis, were not included in the analysis presented here. Compounds whose concentrations were sufficiently low that they were found to be below the detection limit, as determined by the automated peak integration techniques, for one or more of Examples 6-19 are not included in Table 16, nor were such compounds included in the calculations used to prepare FIG. 29. A summary of the calculated peak areas for the identified peaks for each of Examples 6-19 is included in Table 16 below.

In Table 16 below certain common abbreviations are used to denote particular compounds or compound elements. For example, “C_” refers to the carbon number of a referenced compound or portion of a compound as in C5 (pentane), “i” refers to “iso” as in iC5 (isopentane), “M” refers to a “methyl” substituient as in MCyC5 (methyl-cyclopentane), a number before a substituient refers to the position of attachment of the substituient as in 2MC6 (2-methyl-cyclohexane), “Cy” refers to “cyclo” as in MCyC6 (methyl-cyclohexane, “n” refers to “normal” as in nC5 (normal-pentane), “Bz” refers to benzene, “c” refers to “cis”, “t” refers to “trans”, “D” refers to “di” as in dimethyl (DM), “T” refers to “tri” as in “1_{—}3_{—}5 TMBz” (1,3,5-trimethylbenzene), “E” refers to “ethyl” as in ECyC5 (ethyl-cyclopentane), “Tol” refers to toluene, mXly and oXly refer to meta-xylene and ortho-xylene respectively, “IP_” refers to an isoprenoid compound with the number denoting the carbon number of the particular isoprenoid compound as in IP9, “naph” refers to naphthalene as in 2MNaph (2-methyl-naphthalene), and “Hexyl” refers to “hexyl” as in HexylCyC6 (hexyl-cyclohexane).

TABLE 16
Identified Compound Peak Areas for Examples 6-19

| | Ex. 13 | Ex. 15 | Ex. 18 | Ex. 8 | Ex. 11 | Ex. 6 | Ex. 9 |
|---|---|---|---|---|---|---|---|
| Compound | 393/500/0 | 393/500/400 | 393/500/1000 | 375/500/400 | 375/500/1000 | 375/500/0 | 375/200/400 |
| nC3 | 3070 | 12222 | 9036 | 1419 | 4945 | 11720 | 4097 |
| iC4 | 14577 | 25585 | 20701 | 4701 | 12466 | 26042 | 8750 |
| nC4 | 61525 | 91721 | 81541 | 14969 | 41191 | 67529 | 25806 |
| iC5 | 124389 | 139131 | 133808 | 47249 | 81797 | 120826 | 57404 |
| nC5 | 214498 | 231737 | 241934 | 76635 | 125249 | 153466 | 89649 |
| 2MC5 | 142240 | 138317 | 124548 | 90777 | 110559 | 143375 | 95052 |
| 3MC5 | 39883 | 41951 | 42934 | 23859 | 30630 | 36964 | 24672 |
| nC6 | 174315 | 169809 | 186830 | 89770 | 114925 | 114891 | 92728 |
| MCyC5 | 48600 | 56782 | 82829 | 26109 | 36572 | 34286 | 26678 |
| Bz | 5862 | 16631 | 28651 | 3893 | 8554 | 1504 | 3860 |
| CyC6 | 8392 | 10360 | 13915 | 6271 | 7303 | 6676 | 6233 |
| 2MC6 | 19543 | 20254 | 21566 | 14136 | 16135 | 17090 | 13723 |
| 2_3DMC5 | 10344 | 9911 | 7272 | 11126 | 10310 | 12506 | 10683 |
| 3MC6 | 43324 | 42270 | 37563 | 34674 | 37494 | 45301 | 32482 |
| c1_3DMCyC5 | 11785 | 12333 | 17798 | 6385 | 9029 | 8869 | 6423 |
| t1_3DMCyC5 | 10429 | 11425 | 17173 | 5684 | 7990 | 8003 | 5697 |
| t1_2DMCyC5 | 17611 | 19733 | 25300 | 13018 | 15606 | 18074 | 12433 |
| nC7 | 116961 | 112106 | 117194 | 83617 | 92582 | 93441 | 80277 |
| MCyC6 | 27206 | 31726 | 47753 | 16625 | 22451 | 19190 | 16212 |
| ECyC5 | 13120 | 13977 | 19543 | 7685 | 9785 | 8999 | 7439 |
| ctc1_2_4TMCyC5 | 6964 | 7626 | 9562 | 5324 | 6119 | 7467 | 4625 |
| 2_3_3TMC5 | 6183 | 10603 | 15281 | 5339 | 8706 | 2703 | 4944 |
| Tol | 17401 | 30634 | 57612 | 12597 | 18087 | 11070 | 12191 |
| 2MC7 | 70047 | 66469 | 54973 | 67860 | 67378 | 78358 | 63857 |
| 4MC7 | 17283 | 16136 | 11823 | 15943 | 15637 | 19999 | 15499 |
| 3MC7 + c1_3DMCyC6 | 18724 | 21594 | 28530 | 14002 | 17847 | 17053 | 13467 |
| 1_1DMCyC6 | 5616 | 6493 | 8267 | 4736 | 5500 | 5376 | 4467 |
| t1_2DMCyC6 | 6801 | 8170 | 12064 | 4314 | 6225 | 4334 | 4112 |
| nC8 | 90517 | 85874 | 87571 | 71371 | 76307 | 76042 | 67154 |
| ECyC6 | 9784 | 10612 | 14266 | 5798 | 7428 | 6680 | 5673 |
| IP9 | 36441 | 30255 | 19664 | 34977 | 32585 | 40633 | 31372 |
| 1_1_3TMCyC6 | 67451 | 69418 | 71685 | 76067 | 73961 | 83467 | 71038 |
| EBz | 7054 | 10031 | 16801 | 4925 | 6225 | 4423 | 4589 |
| mXyl | 44614 | 59863 | 81247 | 40493 | 49280 | 38201 | 38910 |
| 2_3DMC7 | 10223 | 24038 | 37529 | 15910 | 12554 | 14191 | 14575 |
| 3MC8 | 32855 | 32181 | 24051 | 32724 | 32243 | 39088 | 30359 |
| oXyl | 14653 | 22464 | 36267 | 10799 | 14267 | 10186 | 10375 |
| nC9 | 101858 | 93218 | 87912 | 82617 | 84254 | 88811 | 77742 |
| Cumene | 2901 | 7520 | 8888 | 6625 | 7935 | 8184 | 6224 |
| IP10 | 53783 | 47941 | 29598 | 61657 | 55678 | 69422 | 57218 |
| 1E3MBz | 15969 | 20713 | 33035 | 10584 | 14374 | 10348 | 9902 |
| 1E4MBz | 21094 | 25059 | 34620 | 19024 | 21991 | 19239 | 18078 |
| 1_3_5TMBz | 39623 | 42681 | 38619 | 52422 | 47097 | 56611 | 45270 |
| 4MC9 | 27600 | 25217 | 16439 | 30557 | 28718 | 35754 | 28362 |
| 2MC9 | 16654 | 16866 | 18834 | 12978 | 14080 | 15037 | 12495 |
| 3MC9 | 13553 | 11011 | 5639 | 20738 | 15934 | 20578 | 20959 |
| 1_2_4TMBz | 35405 | 45398 | 60770 | 31590 | 37630 | 30331 | 29563 |
| nC10 | 102614 | 93728 | 83963 | 85326 | 87420 | 92784 | 79365 |
| 1_2_3TMBz | 46530 | 50944 | 61138 | 47116 | 51099 | 46419 | 43969 |
| IP11 | 53131 | 46057 | 25081 | 68367 | 58432 | 74228 | 64169 |
| 1E3_5DMBz | 15979 | 16506 | 18142 | 15618 | 15219 | 17578 | 14581 |
| Transdecaline | 5321 | 6465 | 6992 | 8362 | 8321 | 8239 | 4659 |
| 5MC10 | 6122 | 5529 | 5244 | 5863 | 5778 | 6337 | 7966 |
| 2MC10 | 9595 | 9887 | 12762 | 9094 | 9395 | 9644 | 13853 |
| 1E2_3DMBz | 7095 | 7301 | 9624 | 6672 | 7159 | 6650 | 6045 |
| nC11 | 79804 | 68426 | 58919 | 66051 | 66120 | 71801 | 62784 |
| 2_4DMC10 | 7737 | 8199 | 8477 | 8504 | 8164 | 8696 | 5920 |
| Tetralin | 3615 | 4437 | 5676 | 3595 | 3733 | 3529 | 3686 |
| 5MC11 | 8535 | 8847 | 2974 | 10342 | 9739 | 11882 | 9685 |
| 2MC11 | 7526 | 6953 | 6077 | 7395 | 7263 | 7990 | 6461 |
| Naph | 2537 | 3680 | 5645 | 5156 | 4657 | 2561 | 4258 |
| nC12 | 57116 | 50173 | 40697 | 51047 | 50557 | 55519 | 46671 |
| IP13 | 33496 | 28236 | 15869 | 48783 | 40194 | 50238 | 43477 |
| HexylCyC6 | 6966 | 7165 | 9944 | 5000 | 6271 | 6893 | 8069 |
| 5MC12 | 7062 | 7740 | 8973 | 7560 | 7726 | 7775 | 6847 |
| 2MC12 | 7248 | 6077 | 4833 | 8617 | 7577 | 8916 | 7348 |
| 3MC12 | 8829 | 8603 | 10513 | 7585 | 8675 | 7855 | 6904 |
| IP14 | 28729 | 21527 | 8594 | 46307 | 35283 | 47416 | 41223 |
| 2MNaph | 19500 | 18806 | 26986 | 10559 | 12490 | 10992 | 9353 |
| 1MNaph | 8355 | 10124 | 14858 | 7499 | 8235 | 7287 | 7020 |
| nC13 | 60358 | 49540 | 40064 | 54266 | 54134 | 60844 | 49318 |
| IP15 | 27770 | 21873 | 8452 | 48540 | 36704 | 47360 | 42153 |
| DMNaph | 11505 | 13776 | 17725 | 8091 | 9799 | 8886 | 7468 |
| nC14 | 58327 | 48375 | 37495 | 51077 | 51104 | 56111 | 44159 |
| 1_3&1_7DMNaph | 12556 | 8488 | 18800 | 15493 | 13072 | 16007 | 13716 |
| 1_6DMNaph | 34937 | 36280 | 43517 | 31086 | 33687 | 30971 | 27896 |
| IP16 | 27618 | 20675 | 6337 | 54276 | 38608 | 49543 | 47297 |
| nC15 | 57947 | 48654 | 35978 | 49985 | 49673 | 55976 | 43505 |
| nC16 | 53454 | 41787 | 29683 | 45898 | 45937 | 53132 | 39257 |
| IP18 | 17984 | 12500 | 4220 | 38640 | 26823 | 35474 | 32985 |
| nC17 | 49732 | 39112 | 26607 | 47824 | 45825 | 54421 | 41910 |
| Pristane | 18861 | 12675 | 4990 | 48256 | 30264 | 40970 | 41927 |
| nC18 | 44093 | 33861 | 20379 | 39946 | 39340 | 47957 | 35325 |
| Phytane | 9925 | 6602 | 2612 | 24166 | 15769 | 21983 | 21467 |
| nC19 | 42725 | 31598 | 17647 | 39741 | 38492 | 47913 | 34304 |
| Total Identified | 2959957 | 2977271 | 2965920 | 2468276 | 2622351 | 2961117 | 2350318 |

| | Ex. 12 | Ex. 10 | Ex. 7 | Ex. 16 | Ex. 11 | Ex. 17 | Ex. 14 |
|---|---|---|---|---|---|---|---|
| Compound | 375/200/1000 | 375/50/400 | 375/200/0 | 393/200/400 | 393/200/1000 | 393/50/400 | 393/200/0 |
| nC3 | 7269 | 5661 | 1236 | 6699 | 11669 | 7397 | 13908 |
| iC4 | 15739 | 12807 | 5387 | 17390 | 30066 | 16581 | 25595 |
| nC4 | 48363 | 37939 | 20264 | 65885 | 119228 | 63253 | 85782 |
| iC5 | 90064 | 72666 | 55106 | 97547 | 156244 | 89843 | 121034 |
| nC5 | 137002 | 110901 | 88871 | 169827 | 286914 | 160735 | 194479 |
| 2MC5 | 115526 | 101998 | 85673 | 93272 | 108420 | 84693 | 120618 |
| 3MC5 | 31677 | 26342 | 21791 | 28694 | 40690 | 26860 | 33718 |
| nC6 | 118146 | 100618 | 86522 | 129334 | 184005 | 126052 | 142388 |
| MCyC5 | 37084 | 28565 | 22003 | 47374 | 109121 | 46573 | 42081 |
| Bz | 7853 | 5046 | 1462 | 13841 | 32635 | 13298 | 5281 |
| CyC6 | 7216 | 6215 | 4677 | 7782 | 16185 | 7796 | 6702 |
| 2MC6 | 15916 | 13707 | 11498 | 14158 | 18493 | 13526 | 15983 |
| 2_3DMC5 | 10200 | 10045 | 8700 | 5872 | 5213 | 5221 | 8009 |
| 3MC6 | 36012 | 32032 | 27592 | 26590 | 27008 | 24198 | 34632 |
| c1_3DMCyC5 | 8745 | 6723 | 5653 | 10077 | 20876 | 10166 | 10196 |
| t1_3DMCyC5 | 7738 | 5919 | 4696 | 9288 | 20375 | 9410 | 8996 |
| t1_2DMCyC5 | 14871 | 12111 | 9093 | 14076 | 25486 | 13945 | 14016 |
| nC7 | 88478 | 79883 | 71376 | 84301 | 100677 | 83967 | 93851 |
| MCyC6 | 21185 | 16612 | 13077 | 25416 | 52122 | 26941 | 22527 |
| ECyC5 | 9321 | 7481 | 5938 | 10785 | 20809 | 11141 | 10494 |
| ctc1_2_4TMCyC5 | 5416 | 4342 | 3333 | 4973 | 8491 | 4876 | 5099 |
| 2_3_3TMC5 | 7701 | 6078 | 2721 | 8282 | 14107 | 8462 | 5820 |
| Tol | 16417 | 12571 | 7936 | 28390 | 81614 | 29969 | 15289 |
| 2MC7 | 64097 | 60547 | 53781 | 43620 | 34674 | 40804 | 56500 |
| 4MC7 | 15831 | 14584 | 12924 | 9493 | 7239 | 8556 | 13741 |
| 3MC7 + c1_3DMCyC6 | 16911 | 13585 | 10461 | 15740 | 25479 | 16746 | 15554 |
| 1_1DMCyC6 | 5142 | 4322 | 3549 | 4621 | 7524 | 4699 | 4489 |
| t1_2DMCyC6 | 5651 | 4462 | 3155 | 6174 | 11333 | 6599 | 5786 |
| nC8 | 72018 | 66566 | 61076 | 64505 | 72259 | 65997 | 72525 |
| ECyC6 | 7000 | 5801 | 5109 | 8070 | 13555 | 8580 | 8331 |
| IP9 | 30352 | 30154 | 29713 | 19489 | 10212 | 17783 | 28533 |
| 1_1_3TMCyC6 | 68812 | 65524 | 56646 | 46985 | 54500 | 46693 | 52535 |
| EBz | 5488 | 4430 | 3043 | 7868 | 22842 | 8534 | 5561 |
| mXyl | 45134 | 39173 | 27725 | 49533 | 89552 | 51545 | 39081 |
| 2_3DMC7 | 11746 | 12891 | 10928 | 17600 | 45058 | 18722 | 7594 |
| 3MC8 | 29983 | 28501 | 25005 | 18399 | 12310 | 17096 | 24731 |
| oXyl | 13177 | 10318 | 6919 | 18339 | 46078 | 19916 | 12553 |
| nC9 | 79626 | 75732 | 70146 | 66190 | 62650 | 66077 | 78141 |
| Cumene | 7177 | 7111 | 6494 | 3178 | 5533 | 4313 | 5740 |
| IP10 | 52477 | 52795 | 48281 | 27855 | 13405 | 24950 | 40164 |
| 1E3MBz | 13066 | 10278 | 7349 | 17552 | 40995 | 18896 | 13343 |
| 1E4MBz | 20286 | 18035 | 13450 | 19875 | 37841 | 20789 | 17314 |
| 1_3_5TMBz | 40192 | 40307 | 29445 | 18792 | 37623 | 20422 | 23307 |
| 4MC9 | 27153 | 26459 | 23814 | 14129 | 7667 | 13118 | 20482 |
| 2MC9 | 13381 | 12026 | 10378 | 12857 | 20387 | 13440 | 13690 |
| 3MC9 | 16690 | 19055 | 18064 | 4841 | 4289 | 16173 | 17458 |
| 1_2_4TMBz | 34267 | 29903 | 21548 | 35775 | 64065 | 37666 | 29801 |
| nC10 | 83296 | 80118 | 75273 | 64751 | 56048 | 65746 | 80593 |
| 1_2_3TMBz | 46723 | 43386 | 33967 | 37772 | 59761 | 39883 | 38341 |
| IP11 | 56917 | 58868 | 55471 | 26255 | 10528 | 23780 | 40754 |
| 1E3_5DMBz | 14220 | 13458 | 11588 | 10256 | 15328 | 10406 | 11157 |
| Transdecaline | 4627 | 4483 | 4161 | 4121 | 6263 | 4554 | 4332 |
| 5MC10 | 5910 | 7492 | 6898 | 2872 | 3790 | 3039 | 3820 |
| 2MC10 | 15854 | 14055 | 11002 | 9448 | 20313 | 10543 | 7992 |
| 1E2_3DMBz | 6445 | 6158 | 5036 | 5563 | 9942 | 6136 | 5707 |
| nC11 | 62410 | 62409 | 59850 | 47595 | 37001 | 48821 | 61647 |
| 2_4DMC10 | 5084 | 5554 | 4997 | 3855 | 6788 | 4178 | 3670 |
| Tetralin | 3859 | 3862 | 3175 | 3551 | 6009 | 3703 | 3485 |
| 5MC11 | 9507 | 9301 | 8384 | 5169 | 3236 | 4919 | 7054 |
| 2MC11 | 6420 | 6212 | 5903 | 2939 | 7434 | 4831 | 5815 |
| Naph | 4045 | 5115 | 3471 | 1421 | 1335 | 3192 | 2146 |
| nC12 | 47162 | 45929 | 45161 | 32755 | 22642 | 32990 | 43792 |
| IP13 | 36808 | 41276 | 38086 | 15279 | 7229 | 13961 | 24531 |
| HexylCyC6 | 6735 | 7805 | 7201 | 4063 | 10738 | 5909 | 5997 |
| 5MC12 | 6348 | 5805 | 5747 | 5449 | 8791 | 5702 | 5849 |
| 2MC12 | 6351 | 6715 | 6599 | 2403 | 2676 | 2476 | 3389 |
| 3MC12 | 7651 | 6800 | 6234 | 6489 | 9827 | 6687 | 7687 |
| IP14 | 32862 | 38407 | 35926 | 10800 | 3824 | 9345 | 21461 |
| 2MNaph | 11378 | 9218 | 8139 | 14756 | 30837 | 15289 | 11673 |
| 1MNaph | 7379 | 6890 | 5949 | 7823 | 18060 | 8439 | 7329 |
| nC13 | 49637 | 49013 | 49484 | 32927 | 21837 | 33203 | 46055 |
| IP15 | 34553 | 40638 | 39565 | 10930 | 6636 | 9395 | 20846 |
| DMNaph | 8910 | 7767 | 7155 | 9999 | 19469 | 10234 | 9255 |
| nC14 | 46665 | 45946 | 49294 | 32498 | 20702 | 31519 | 44152 |
| 1_3&1_7DMNaph | 11972 | 12768 | 13155 | 10766 | 20536 | 10877 | 9706 |
| 1_6DMNaph | 30203 | 28309 | 25197 | 27911 | 44268 | 28732 | 29189 |
| IP16 | 35572 | 44986 | 44159 | 9430 | 1828 | 7803 | 20599 |
| nC15 | 45224 | 45232 | 48033 | 29479 | 18676 | 29044 | 42973 |
| nC16 | 40865 | 41749 | 46405 | 26623 | 13755 | 25733 | 38544 |
| IP18 | 24431 | 31626 | 32339 | 6256 | 2357 | 5234 | 13352 |
| nC17 | 41281 | 42514 | 48243 | 23254 | 11287 | 24080 | 37090 |
| Pristane | 27360 | 39776 | 41674 | 5703 | 4003 | 5775 | 13853 |
| nC18 | 34948 | 36327 | 43770 | 20198 | 7554 | 19693 | 31365 |
| Phytane | 14044 | 20341 | 21713 | 3351 | 2448 | 3020 | 7617 |
| nC19 | 34009 | 35906 | 45087 | 19100 | 8779 | 19319 | 30772 |
| Total Identified | 2521261 | 2381033 | 2127100 | 2065143 | 2800050 | 2051204 | 2447041 |

Analyses by liquid chromatography (LC) followed by gas chromatography/mass spectrometry (GC/MS) were performed on liquid samples from Examples 6-20 (as indicated in the Examples). The LC procedure separated the saturate and aromatic hydrocarbon fractions from the liquid or sediment sample produced in each Example. The saturate and aromatic fractions were then analyzed separately by GC/MS. The procedures for these analyses are described in the following sections.

### LC Method

The LC procedure utilized silica gel as the separation medium and a selection of solvents in order to produce the saturate and aromatic extracts.

The LC procedure utilized the following laboratory equipment and chemicals:
1. Zymark Turbovap® Evaporator
2. Muffle furnace capable of operating at 400° C.
3. Centrifuge (1500 rpm capability such as Fisher Centrific®)
4. Glass chromatography columns (10 mm. ID×50 cm. length)
5. Other standard lab equipment as required—e.g., exhaust hood, analytical balance, hot plate (40° C.) and refrigerator
6. Pentane (Fisher Optima grade or equivalent)
7. Hexane (Fisher Optima grade or equivalent)
8. Methylene chloride (Fisher Optima grade or equivalent)
9. Methanol (Fisher Optima grade or equivalent)
10. Silica gel (Fisher grade 923 @ 100-200 mesh)
11. Internal standards mixture as described below

The internal standards mixture was made in hexane with the compounds and concentrations as shown Table 19:

TABLE 19

| Compound Name | Concentration (ng/μl) |
|---|---|
| 5β Cholane | 40.4 |
| C13 Benzene | 40.8 |
| d-Tetralin | 42.0 |
| 2-Fluoro Biphenyl | 31.2 |
| d8 Naphthalene | 30.0 |
| o-Terphenyl | 21.2 |
| d10 Fluoranthrene | 20.3 |

The amount of the internal standards mixture to be added (as described in the procedure to follow) was determined according to Table 20.

TABLE 20

| Pentane | Amount of Internal |
|---|---|
| Soluble (mg) | Std. Needed (μl) |
| 65-55 | 45 |
| 55-50 | 40 |
| 50-45 | 35 |
| 45-40 | 30 |
| 40-30 | 25 |
| 30-25 | 20 |
| 25-20 | 15 |
| 20-10 | 10 |
| 10-5 | 5 |

The internal standards were used to enable calculation of concentrations and as known markers to facilitate compound identifications.

This LC method was used because it easily separates the crude oil or sediment extract sample into 4 compound classes including saturates, aromatics, NSO's (nitrogen, sulfur and oxygenate compounds), and asphaltenes.

The first separations in this LC procedure were the removal of asphaltenes and the collection of the pentane soluble fraction as described in Table 21:

TABLE 21

| Step | Action |
|---|---|
| 1 | Label sample vials or centrifuge tubes with sample number and type (PN, ASPH, |
| | SAT, ARO, and NSO). |
| 2 | Weigh all vials and centrifuge tubes. |
| 3 | Weigh about 50 mgs of sample in a labeled centrifuge tube. |
| 4 | Add 10 mls of pentane to centrifuge tube to precipitate the asphaltenes. |
| 5 | Leave sample at room temperature for 4 hours, and then centrifuge for 30 minutes |
| | at 1500 rpm (setting of 12 on a Fisher Centrific ® centrifuge). |
| 6 | Decant solvent in centrifuge tube into a 20-ml pre-weighed vial and blow dry |
| | with nitrogen at 40° C. |
| | Note: This is the pentane soluble fraction (PN). |
| 7 | Weigh the PN fraction. |
| 8 | Transfer the residue in the centrifuge tube to a 20-ml pre-weighed vial using |
| | methylene chloride and blow dry with nitrogen at 40° C. |
| | Note: This is the asphaltene fraction (ASPH). |

Table 22 describes the liquid chromatographic separation procedure.

TABLE 22

| Step | Action |
|---|---|
| 1 | Activate the silica gel at 400° C. for 24 hours, then store in a desiccator. |
| 2 | Add the internal standards and approximately 2 mls of pentane to the PN fraction |
| | and mix. |
| | Note: Amount of internal standards is determined from Table 20. |
| 3 | Pour 14 gms of activated silica gel to a liquid chromatography column and tap to |
| | pack. |
| 4 | Add 5 mls of hexane to column as a pre-wet. As pre-wet enters gel, add contents of |
| | vial from step 2 to column followed by 5 mls of additional hexane and collect in a |
| | 50 ml evaporator tube (SAT fraction). |
| | Note: If the saturate fraction develops some color, the column was overloaded. |
| | Repeat the LC separation using less sample. |
| 5 | Wash PN vial with two 10 mls aliquots of hexane (20 mls total), adding to LC |
| | column as previous addition enters the gel. |
| 6 | Switch to solvent of methylene chloride, continue adding 5 mls of solvent to the |
| | column |
| 7 | As the 5 mls of methylene chloride enters the gel, remove the evaporation tube |
| | containing the saturate fraction from the base of the column and replace with |
| | another evaporation tube to collect the methylene chloride soluble fraction (ARO |
| | fraction). |
| 8 | Add two 10 mls aliquots of methylene chloride (20 mls total) to the column in the |
| | same manner as the hexane additions in step 5. |
| 9 | Switch to solvent of methylene chloride/methanol (50/50 mix), continue adding 5 mls |
| | of solvent to column. |
| 10 | As the 5 mls of methylene chloride/methanol enters the gel, remove the |
| | evaporation tube containing the aromatic fraction from the base of the column and |
| | replace with a20 mls pre-weighed vial to collect the methylene chloride/methanol |
| | soluble fraction (NSO fraction). |
| 11 | Add two 10 mls aliquots of methylene chloride/methanol to the column (20 mls |
| | total) in the same manner as the hexane additions in step 5. Collect until column is |
| | dry. |
| 12 | Using nitrogen, dry the evaporator tubes containing the hexane and methylene |
| | chloride fractions to a volume of 4 mls, using a Turbovap ® evaporator at 40° C. |
| 13 | Transfer the 4 mls to pre-weighed sample vials and dry to constant weight with |
| | nitrogen at 40° C. |
| 14 | Dry the methylene chloride/methanol vial to constant weight with nitrogen at |
| | 40° C. |
| 15 | Weigh the ASPH, SAT, ARO, and NSO fractions. |
| | Note: The sum of the weights of the saturate, aromatic and NSO fractions should |
| | be equal or less than the weight of the PN soluble (SAT + ARO + NSO <= PN). If the |
| | sum was greater after the fractions have been dried to constant weight, then the |
| | NSO fraction may have contained silica gel and a note was placed in the |
| | laboratory journal. Such an occurrence is of no consequence for the SAT and |
| | ARO fractions and their analyses, which are the focus of this application. |

Quality control for this LC procedure was maintained via the testing of a standard oil sample (North Sea), which was run with the samples of interest. Percentages of the SAT, ARO, NSO and ASPH fractions (based on the whole sample) were calculated and QC charts maintained.

GC/MS Method: The GC/MS was performed using an HP 6890 gas chromatograph (GC) connected to an HP 5973 MSD mass spectrometer (MS) equipped with a mass selective detector. In addition, an autosampler (equipped with a 10 μl syringe) and Agilent ChemStation software (version G1710DA for Microsoft XP®) were used. The mass spectrometric method used was SIM (selected ion monitoring) for analysis of the saturated and aromatic components, which were collected as described in the previous section.

Materials Used:

Hexane (Fisher Optima grade or equivalent)

GC column—J&W DB-5 fused silica (Agilent)

UHP Helium (Air Liquide)

Methylene chloride (Fisher Optima grade or equivalent)

Sample Preparation: Each liquid sample to be analyzed was transferred to a sample vial suitable for use in the GC/MS equipment, the concentration adjusted to 25 μg/μl by the addition of hexane. The vial was sealed and placed in the autosampler.

GC conditions: The GC employed a J&W DB-5 fused silica capillary column, which was 30 meters in length, 0.25 mm inner diameter and had 0.25 μm film thickness. GC settings for the saturate fractions were those shown in Table 23.

TABLE 23

| Parameter | Setting |
|---|---|
| Injection mode | Split |
| Inlet Temp | 310° C. |
| Pressure | 9 psi |
| Split ratio | 5:1 |
| Total flow | 8.0 mL/min |
| Temperature program | 75 - 200 @ 5° C./min - 315 @ |
| | 3° C./min (hold for 15 mins) |

GC settings for the aromatic fractions were as indicated in Table 24.

TABLE 24

| Parameter | Setting |
|---|---|
| Injection mode | Split with split ratio = 5:1 |
| Inlet Temp | 310° C. |
| Pressure | 9.38 psi |
| Total flow | 7.0 mL/min |
| Temperature program | 80 - 200 @ 5° C./min - 315 @ |
| | 6° C./min, hold for 20 mins |

MS conditions: Methylene chloride was used to clean components of the MS as needed. MS source and quad temperatures were set as shown in Table 25.

TABLE 25

| Parameter | Setting |
|---|---|
| MS Quad | 150° C., maximum 200° C. |
| MS Source | 230° C., maximum 250° C. |

MS SIM parameters for the saturate and aromatic fractions were set as shown in Table 26.

TABLE 26

| Parameter | Setting |
|---|---|
| Solvent delay | 20 min |
| Acquisition mode | SIM |
| Monitoring ions | See list below |
| Dwell time | 100 ms |

Additionally the following list of saturates and aromatics was used to facilitate compound identification.

### Saturates:

m/z 123: terpenoids
m/z 125: beta-carotane
m/z 177: C29 terpanes, 25-norhopanes

m/z 183: isoprenoids

m/z 191: terpanes
m/z 205: methyl hopanes
m/z 217: steranes
m/z 218: abb-steranes
m/z 231: steranes
m/z 259: diasteranes

### Aromatics:

Naphthalenes: m/z 128 (C0), m/z 142 (C1), m/z 156 (C2), m/z 170 (C3), m/z 184 (C4 & dibenzothiophene)

### Phenanthrenes: m/z 178 (C0), m/z 192 (C1), m/z 206 (C2), m/z 220 (C3)

Dibenzothiophenes: m/z 184 (C0 & C4 naphthalenes), m/z 198 (C1), m/z 212 (C2)
Aromatic Steroids: m/z 231 (and C1 benzocarbozoles), m/z 245 (and C2 benzocarbozoles), m/z 253

### Chrysenes: m/z 228 (C0), m/z 242 (C1), m/z 256 (C2), m/z 270 (C3)

### Carbozoles: m/z 167 (C0), m/z 181 (C1), m/z 195 (C2)

Benzocarbozoles: m/z 217 (C0), m/z 231 (C1 and triaromatic steroids), m/z 245 (C2 and triaromatic dinosteroids)

### Benzohopanes: m/z 191

### Biphenyls: m/z 154 (C0), m/z 168 (C1), m/z 182 (C2)

### Fluorenes: m/z 166 (C0), m/z 180 (C1), m/z 194 (C2)

MS instrument settings were adjusted to achieve the detection limits listed in Table 27 over a range of mass values:

TABLE 27

| Tune target | Limit |
|---|---|
| Mass 50 | 1 |
| Mass 131 | 40 |
| Mass 219 | 60 |
| Mass 414 | 10 |
| Mass 502 | 5 |
| Peak width | 0.55 |
| Abundance | 400,000 |

Analysis and reporting of the data: Peak assignments in the GC/MS chromatograms were made utilizing a known oil reference standard (North Sea, Jurassic sourced oil from the Kimmeridge) and an internal standard (5β-Cholane for the saturates and o-Terphenyl, C13 Benzene for the aromatics). The geochemical patterns were confirmed by both retention time and diagnostic mass to charge ratios since most of these geochemical compounds are not commercially available. In addition, use was made of spectral patterns of typical compounds found in oil samples 221- and studied extensively in the literature such as those described in the following reference: The Biomarker Guide, by Kenneth E. Peters and J. Michael Moldowan, Prentice Hall Publishing, 1993.

The spectral quality and reproducibility of the GC/MS data were maintained by frequent runs of a previously known crude oil that was separated into fractions by liquid chromatography (as described in this method). Ratios from this standard (i.e., the North Sea oil mentioned above) were used for quality control purposes. A mass calibrant (perfluorotributylamine) was used for the MS prior to instrumental analysis. These data were used to ensure accurate peak positions and to ensure reproducibility of peak area ratios through time.

The concentrations of molecular species found in the chromatograms generated by the GC/MS analysis were determined by manually measuring peak heights for each compound of interest. The baseline for each measurement was taken as the mean baseline for the peak extrapolated from the low background to the high background. When nearby peaks caused the immediately adjacent background to be too high the background was estimated by linear fit to average background for compounds eluting within 2 minutes of the molecule of interest. For each sample, all peak heights were measured in the same chromatogram or scaled to a single chromatogram in instances where very large peak heights introduced graphical error in measuring smaller peaks.

The above-described processes may be of merit in connection with the recovery of hydrocarbons in the Piceance Basin of Colorado. Some have estimated that in some oil shale deposits of the Western United States, up to 1 million barrels of oil may be recoverable per surface acre. One study has estimated the oil shale resource within the nahcolite-bearing portions of the oil shale formations of the Piceance Basin to be 400 billion barrels of shale oil in place. Overall, up to 1 trillion barrels of shale oil may exist in the Piceance Basin alone.

Certain features of the present invention are described in terms of a set of numerical upper limits and a set of numerical lower limits. It should be appreciated that ranges formed by any combination of these limits are within the scope of the invention unless otherwise indicated. Although some of the dependent claims have single dependencies in accordance with U.S. practice, each of the features in any of such dependent claims can be combined with each of the features of one or more of the other dependent claims dependent upon the same independent claim or claims.

While it will be apparent that the invention herein described is well calculated to achieve the benefits and advantages set forth above, it will be appreciated that the invention is susceptible to modification, variation and change without departing from the spirit thereof.

What is claimed is:

1. A hydrocarbon fluid produced through in situ pyrolysis of oil shale within an oil shale formation, the hydrocarbon fluid comprising a condensable hydrocarbon portion, the condensable hydrocarbon portion having:
a) one or more of a n-C6 to benzene weight ratio less than 35.0, a n-C7 to toluene weight ratio less than 7.0, a n-C8 to ethylbenzene weight ratio less than 16.0, a n-C8 to ortho-xylene weight ratio less than 7.0, a n-C8 to meta-xylene weight ratio less than 1.9, a n-C9 to 1-ethyl-3-methylbenzene weight ratio less than 8.2, a n-C9 to 1-ethyl-4-methylbenzene weight ratio less than 4.4, a n-C9 to 1,2,4-trimethylbenzene weight ratio less than 2.7, a n-C10 to 1-ethyl-2,3-dimethylbenzene weight ratio less than 13.5, a n-C10 to tetralin weight ratio less than 25.0, a n-C12 to 2-methylnaphthalene weight ratio less than 4.9, and a n-C12 to 1-methylnaphthalene weight ratio less than 6.9;
b) one or more of a n-C9 to IP-9 weight ratio greater than 2.4, a n-C10 to IP-10 weight ratio greater than 1.4, a n-C11 to IP-11 weight ratio greater than 1.0, a n-C13 to IP-13 weight ratio greater than 1.1, a n-C14 to IP-14 weight ratio greater than 1.1, a n-C15 to IP-15 weight ratio greater than 1.0, a n-C16 to IP-16 weight ratio greater than 0.8, a n-C18 to IP-18 weight ratio greater than 1.0, and a n-C19 to pristane weight ratio greater than 1.6;
c) one or more of a n-C7 to cis 1,3-dimethyl cyclopentane weight ratio less than 13.1, a n-C7 to trans 1,3-dimethyl cyclopentane weight ratio less than 14.9, a n-C7 to trans 1,2-dimethyl cyclopentane weight ratio less than 7.0, a n-C7 to methyl cyclohexane weight ratio less than 5.2, a n-C7 to ethyl cyclopentane weight ratio less than 11.3, a n-C8 to 1,1-dimethyl cyclohexane weight ratio less than 16.0, a n-C8 to trans 1,2-dimethyl cyclohexane weight ratio less than 17.5, and a n-C8 to ethyl cyclohexane weight ratio less than 12.3;
d) one or more of a total C9 to total C20 weight ratio between 2.5 and 6.0, a total C10 to total C20 weight ratio between 2.8 and 7.3, a total C11 to total C20 weight ratio between 2.6 and 6.5, a total C12 to total C20 weight ratio between 2.6 and 6.4 and a total C13 to total C20 weight ratio between 3.2 and 8.0;
e) one or more of a total C10 to total C25 weight ratio between 7.1 and 24.5, a total C11 to total C25 weight ratio between 6.5 and 22.0, a total C12 to total C25 weight ratio between 6.5 and 22.0, and a total C13 to total C25 weight ratio between 8.0 and 27.0;
f) one or more of a total C10 to total C29 weight ratio between 15.0 and 60.0, a total C11 to total C29 weight ratio between 13.0 and 54.0, a total C12 to total C29 weight ratio between 12.5 and 53.0, and a total C13 to total C29 weight ratio between 16.0 and 65.0;
g) one or more of a normal-C7 to normal-C20 weight ratio greater than 0.9, a normal-C8 to normal-C20 weight ratio greater than 2.0, a normal-C9 to normal-C20 weight ratio greater than 1.9, a normal-C10 to normal-C20 weight ratio greater than 2.2, a normal-C11 to normal-C20 weight ratio greater than 1.9, a normal-C12 to normal-C20 weight ratio greater than 1.9, a normal-C13 to normal-C20 weight ratio greater than 2.3, a normal-C14 to normal-C20 weight ratio greater than 1.8, a normal-C15 to normal-C20 weight ratio greater than 1.8, and normal-C16 to normal-C20 weight ratio greater than 1.3; 1
h) one or more of a normal-C7 to normal-C25 weight ratio greater than 1.9, a normal-C8 to normal-C25 weight ratio greater than 3.9, a normal-C9 to normal-C25 weight ratio greater than 3.7, a normal-C10 to normal-C25 weight ratio greater than 4.4, a normal-C11 to normal-C25 weight ratio greater than 3.8, a normal-C12 to normal-C25 weight ratio greater than 3.7, a normal-C13 to normal-C25 weight ratio greater than 4.7, a normal-C14 to normal-C25 weight ratio greater than 3.7, a normal-C15 to normal-C25 weight ratio greater than 3.7, a normal-C16 to normal-C25 weight ratio greater than 2.5, a normal-C17 to normal-C25 weight ratio greater than 3.0, and a normal-C18 to normal-C25 weight ratio greater than 3.4;
i) one or more of a normal-C7 to normal-C29 weight ratio greater than 18.0, a normal-C8 to normal-C29 weight ratio greater than 16.0, a normal-C9 to normal-C29 weight ratio greater than 14.0, a normal-C10 to normal-C29 weight ratio greater than 14.0, a normal-C11 to normal-C29 weight ratio greater than 13.0, a normal-C12 to normal-C29 weight ratio greater than 11.0, a normal-C13 to normal-C29 weight ratio greater than 10.0, a normal-C14 to normal-C29 weight ratio greater than 9.0, a normal-C15 to normal-C29 weight ratio greater than 8.0, a normal-C16 to normal-C29 weight ratio greater than 8.0, a normal-C17 to normal-C29 weight ratio greater than 6.0, a normal-C18 to normal-C29 weight ratio greater than 6.0, a normal-C19 to normal-C29 weight ratio greater than 5.0, a normal-C20 to normal-C29 weight ratio greater than 4.0, a normal-C21 to normal-C29 weight ratio greater than 3.6, and a normal-C22 to normal-C29 weight ratio greater than 2.8; or
j) one or more of a normal-C10 to total C10 weight ratio less than 0.31, a normal-C11 to total C11 weight ratio less than 0.32, a normal-C12 to total C12 weight ratio less than 0.29, a normal-C13 to total C13 weight ratio less than 0.28, a normal-C14 to total C14 weight ratio less than 0.31, a normal-C15 to total C15 weight ratio less than 0.27, a normal-C16 to total C16 weight ratio less than 0.31, a normal-C17 to total C17 weight ratio less than 0.31, a normal-C18 to total C18 weight ratio less than 0.37, normal-C19 to total C19 weight ratio less than 0.37, a normal-C20 to total C20 weight ratio less than 0.37, a normal-C21 to total C21 weight ratio less than 0.37, a normal-C22 to total C22 weight ratio less than 0.38, normal-C23 to total C23 weight ratio less than 0.43, a normal-C24 to total C24 weight ratio less than 0.48, and a normal-C25 to total C25 weight ratio less than 0.53.

2. The hydrocarbon fluid of claim 1, wherein the condensable hydrocarbon portion has one or more of a (trisnorhopane maturable) to (trisnorhopane maturable+trisnorhopane stable) weight ratio greater than 0.7, a [C-29 17α(H), 21β(H) hopane] to [C-29 17α(H), 21β(H) hopane+C-29 17β(H), 21β(H) hopane] weight ratio less than 0.9, a [C-30 17α(H), 21β(H) hopane] to [C-30 17α(H), 21β(H) hopane+C-30 17β(H), 21β(H) hopane] weight ratio less than 0.9, a [C-31 17αC(H), 21β(H), 22S homohopane] to [C-31 17α(H), 21β(H), 22S homohopane+C-31 17αC(H), 21β(H), 22R homohopane] weight ratio less than 0.6, a [C-29 5 α, 14 α, 17 α (H) 20R steranes] to [C-29 5 α, 14 α, 17 α (H) 20R steranes+C-29 5 α, 14 α, 17 α (H) 20S steranes] weight ratio less than 0.7, a [C-29 5 α, 14 α, 17 β (H) 20S+C-29 5 α, 14 β, 17 β (H) 20R steranes] to [C-29 5 α, 14 β, 17 β (H) 20S+C-29 5 α, 14 β, 17 β (H) 20R steranes+C-29 5 α, 14 α, 17 α (H) 20S+C-29 5 α, 14 α, 17 α (H) 20R steranes] weight ratio less than 0.7, and a [3-methyl phenanthrene (3-MP)+2-methyl phenanthrene (2-MP)] to [1-methyl phenanthrene (1-MP)+9-methyl phenanthrene (9-MP)] weight ratio greater than 0.5.

3. The hydrocarbon fluid of claim 2, wherein the condensable hydrocarbon portion has two or more of a n-C6 to benzene weight ratio less than 24.0, a n-C7 to toluene weight ratio less than 6.6, a n-C8 to ethylbenzene weight ratio less than 15.0, a n-C8 to ortho-xylene weight ratio less than 6.6, a n-C8 to meta-xylene weight ratio less than 1.8, a n-C9 to 1-ethyl-3-methylbenzene weight ratio less than 7.9, a n-C9 to 1-ethyl-4-methylbenzene weight ratio less than 4.3, a n-C9 to 1,2,4-trimethylbenzene weight ratio less than 2.6, a n-C10 to 1-ethyl-2,3-dimethylbenzene weight ratio less than 13.1, a n-C10 to tetralin weight ratio less than 23.7, a n-C12 to 2-methylnaphthalene weight ratio less than 5.0, and a n-C12 to 1-methylnaphthalene weight ratio less than 6.8.

4. The hydrocarbon fluid of claim 2, wherein the condensable hydrocarbon portion has two or more of a n-C9 to IP-9 weight ratio greater than 2.5, a n-C10 to IP-10 weight ratio greater than 1.5, a n-C11 to IP-11 weight ratio greater than 1.1, a n-C13 to IP-13 weight ratio greater than 1.2, a n-C14 to IP-14 weight ratio greater than 1.2, a n-C15 to IP-15 weight ratio greater than 1.1, a n-C16 to IP-16 weight ratio greater than 0.9, a n-C18 to IP-18 weight ratio greater than 1.1, and a n-C19 to pristane weight ratio greater than 1.8.

5. The hydrocarbon fluid of claim 2, wherein the condensable hydrocarbon portion has two or more of a n-C7 to cis 1,3-dimethyl cyclopentane weight ratio less than 12.7, a n-C7 to trans 1,3-dimethyl cyclopentane weight ratio less than 14.7, a n-C7 to trans 1,2-dimethyl cyclopentane weight ratio less than 6.6, a n-C7 to methyl cyclohexane weight ratio less than 5.0, a n-C7 to ethyl cyclopentane weight ratio less than 10.9, a n-C8 to 1,1-dimethyl cyclohexane weight ratio less than 15.4, a n-C8 to trans 1,2-dimethyl cyclohexane weight ratio less than 16.5, and a n-C8 to ethyl cyclohexane weight ratio less than 12.0.

6. The hydrocarbon fluid of claim 2, wherein the condensable hydrocarbon portion has two or more of a total C9 to total C20 weight ratio between 3.0 and 5.5, a total C10 to total C20 weight ratio between 3.2 and 7.0, a total C11 to total C20 weight ratio between 3.0 and 6.0, a total C12 to total C20 weight ratio between 3.0 and 6.0, and a total C13 to total C20 weight ratio between 3.3 and 7.0.

7. The hydrocarbon fluid of claim 2, wherein the condensable hydrocarbon portion has two or more of a total C10 to total C25 weight ratio between 10.0 and 24.0, a total C11 to total C25 weight ratio between 10.0 and 21.5, a total C12 to total C25 weight ratio between 10.0 and 21.5, and a total C13 to total C25 weight ratio between 9.0 and 25.0.

8. The hydrocarbon fluid of claim 2, wherein the condensable hydrocarbon portion has two or more of a total C10 to total C29 weight ratio between 17.0 and 58.0, a total C11 to total C29 weight ratio between 15.0 and 52.0, a total C12 to total C29 weight ratio between 14.0 and 50.0, and a total C13 to total C29 weight ratio between 17.0 and 60.0.

9. The hydrocarbon fluid of claim 2, wherein the condensable hydrocarbon portion has two or more of a normal-C7 to normal-C20 weight ratio greater than 4.4, a normal-C8 to normal-C20 weight ratio greater than 3.7, a normal-C9 to normal-C20 weight ratio greater than 3.5, a normal-C10 to normal-C20 weight ratio greater than 3.4, a normal-C11 to normal-C20 weight ratio greater than 3.0, and a normal-C12 to normal-C20 weight ratio greater than 2.7.

10. The hydrocarbon fluid of claim 2, wherein the condensable hydrocarbon portion has two or more of a normal-C7 to normal-C25 weight ratio greater than 10, a normal-C8 to normal-C25 weight ratio greater than 8.0, a normal-C9 to normal-C25 weight ratio greater than 7.0, a normal-C10 to normal-C25 weight ratio greater than 7.0, a normal-C11 to normal-C25 weight ratio greater than 7.0, and a normal-C12 to normal-C25 weight ratio greater than 6.0.

11. The hydrocarbon fluid of claim 2, wherein the condensable hydrocarbon portion has two or more of a normal-C7 to normal-C29 weight ratio greater than 20.0, a normal-C8 to normal-C29 weight ratio greater than 18.0, a normal-C9 to normal-C29 weight ratio greater than 17.0, a normal-C10 to normal-C29 weight ratio greater than 16.0, a normal-C11 to normal-C29 weight ratio greater than 15.0, a normal-C12 to normal-C29 weight ratio greater than 12.5, a normal-C13 to normal-C29 weight ratio greater than 11.0, a normal-C14 to normal-C29 weight ratio greater than 10.0, a normal-C15 to normal-C29 weight ratio greater than 8.0, a normal-C16 to normal-C29 weight ratio greater than 8.0, a normal-C17 to normal-C29 weight ratio greater than 7.0, a normal-C18 to normal-C29 weight ratio greater than 6.5, a normal-C19 to normal-C29 weight ratio greater than 5.5, a normal-C20 to normal-C29 weight ratio greater than 4.5, and a normal-C21 to normal-C29 weight ratio greater than 4.0.

12. The hydrocarbon fluid of claim 2, wherein the condensable hydrocarbon portion has two or more of a normal-C11 to total C11 weight ratio less than 0.30, a normal-C12 to total C12 weight ratio less than 0.27, a normal-C13 to total C13 weight ratio less than 0.26, a normal-C14 to total C14 weight ratio less than 0.29, a normal-C15 to total C15 weight ratio less than 0.24, a normal-C16 to total C16 weight ratio less than 0.25, a normal-C17 to total C17 weight ratio less than 0.29, a normal-C18 to total C18 weight ratio less than 0.31, normal-C19 to total C19 weight ratio less than 0.35, a normal-C20 to total C20 weight ratio less than 0.33, a normal-C21 to total C21 weight ratio less than 0.33, a normal-C22 to total C22 weight ratio less than 0.35, normal-C23 to total C23 weight ratio less than 0.40, a normal-C24 to total C24 weight ratio less than 0.45, and a normal-C25 to total C25 weight ratio less than 0.49.

13. The hydrocarbon fluid of claim 2, wherein the condensable hydrocarbon portion has three or more of a n-C6 to benzene weight ratio less than 24.0, a n-C7 to toluene weight ratio less than 6.6, a n-C8 to ethylbenzene weight ratio less than 15.0, a n-C8 to ortho-xylene weight ratio less than 6.6, a n-C8 to meta-xylene weight ratio less than 1.8, a n-C9 to 1-ethyl-3-methylbenzene weight ratio less than 7.9, a n-C9 to 1-ethyl-4-methylbenzene weight ratio less than 4.3, a n-C9 to 1,2,4-trimethylbenzene weight ratio less than 2.6, a n-C10 to 1-ethyl-2,3-dimethylbenzene weight ratio less than 13.1, a n-C10 to tetralin weight ratio less than 23.7, a n-C12 to 2-methylnaphthalene weight ratio less than 5.0, and a n-C12 to 1-methylnaphthalene weight ratio less than 6.8.

14. The hydrocarbon fluid of claim 2, wherein the condensable hydrocarbon portion has three or more of a n-C9 to IP-9 weight ratio greater than 2.5, a n-C10 to IP-10 weight ratio greater than 1.5, a n-C11 to IP-11 weight ratio greater than 1.1, a n-C13 to IP-13 weight ratio greater than 1.2, a n-C14 to IP-14 weight ratio greater than 1.2, a n-C15 to IP-15 weight ratio greater than 1.1, a n-C16 to IP-16 weight ratio greater than 0.9, a n-C18 to IP-18 weight ratio greater than 1.1, and a n-C19 to pristane weight ratio greater than 1.8.

15. The hydrocarbon fluid of claim 2, wherein the condensable hydrocarbon portion has three or more of a n-C7 to cis 1,3-dimethyl cyclopentane weight ratio less than 12.7, a n-C7 to trans 1,3-dimethyl cyclopentane weight ratio less than 14.7, a n-C7 to trans 1,2-dimethyl cyclopentane weight ratio less than 6.6, a n-C7 to methyl cyclohexane weight ratio less than 5.0, a n-C7 to ethyl cyclopentane weight ratio less than 10.9, a n-C8 to 1,1-dimethyl cyclohexane weight ratio less than 15.4, a n-C8 to trans 1,2-dimethyl cyclohexane weight ratio less than 16.5, and a n-C8 to ethyl cyclohexane weight ratio less than 12.0.

16. The hydrocarbon fluid of claim 2, wherein the condensable hydrocarbon portion has three or more of a total C9 to total C20 weight ratio between 3.0 and 5.5, a total C10 to total C20 weight ratio between 3.2 and 7.0, a total C11 to total C20 weight ratio between 3.0 and 6.0, a total C12 to total C20 weight ratio between 3.0 and 6.0, and a total C13 to total C20 weight ratio between 3.3 and 7.0.

17. The hydrocarbon fluid of claim 2, wherein the condensable hydrocarbon portion has three or more of a total C10 to total C25 weight ratio between 10.0 and 24.0, a total C11 to total C25 weight ratio between 10.0 and 21.5, a total C12 to total C25 weight ratio between 10.0 and 21.5, and a total C13 to total C25 weight ratio between 9.0 and 25.0.

18. The hydrocarbon fluid of claim 2, wherein the condensable hydrocarbon portion has three or more of a total C10 to total C29 weight ratio between 17.0 and 58.0, a total C11 to total C29 weight ratio between 15.0 and 52.0, a total C12 to total C29 weight ratio between 14.0 and 50.0, and a total C13 to total C29 weight ratio between 17.0 and 60.0.

19. The hydrocarbon fluid of claim 2, wherein the condensable hydrocarbon portion has three or more of a normal-C7 to normal-C20 weight ratio greater than 4.4, a normal-C8 to normal-C20 weight ratio greater than 3.7, a normal-C9 to normal-C20 weight ratio greater than 3.5, a normal-C10 to normal-C20 weight ratio greater than 3.4, a normal-C11 to normal-C20 weight ratio greater than 3.0, and a normal-C12 to normal-C20 weight ratio greater than 2.7.

20. The hydrocarbon fluid of claim 2, wherein the condensable hydrocarbon portion has three or more of a normal-C7 to normal-C25 weight ratio greater than 10, a normal-C8 to normal-C25 weight ratio greater than 8.0, a normal-C9 to normal-C25 weight ratio greater than 7.0, a normal-C10 to normal-C25 weight ratio greater than 7.0, a normal-C11 to normal-C25 weight ratio greater than 7.0, and a normal-C12 to normal-C25 weight ratio greater than 6.0.

21. The hydrocarbon fluid of claim 2, wherein the condensable hydrocarbon portion has three or more of a normal-C7 to normal-C29 weight ratio greater than 20.0, a normal-C8 to normal-C29 weight ratio greater than 18.0, a normal-C9 to normal-C29 weight ratio greater than 17.0, a normal-C10 to normal-C29 weight ratio greater than 16.0, a normal-C11 to normal-C29 weight ratio greater than 15.0, a normal-C12 to normal-C29 weight ratio greater than 12.5, a normal-C13 to normal-C29 weight ratio greater than 11.0, a normal-C14 to normal-C29 weight ratio greater than 10.0, a normal-C15 to normal-C29 weight ratio greater than 8.0, a normal-C16 to normal-C29 weight ratio greater than 8.0, a normal-C17 to normal-C29 weight ratio greater than 7.0, a normal-C18 to normal-C29 weight ratio greater than 6.5, a normal-C19 to normal-C29 weight ratio greater than 5.5, a normal-C20 to normal-C29 weight ratio greater than 4.5, and a normal-C21 to normal-C29 weight ratio greater than 4.0.

22. The hydrocarbon fluid of claim 2, wherein the condensable hydrocarbon portion has three or more of a normal-C11 to total C11 weight ratio less than 0.30, a normal-C12 to total C12 weight ratio less than 0.27, a normal-C13 to total C13 weight ratio less than 0.26, a normal-C14 to total C14 weight ratio less than 0.29, a normal-C15 to total C15 weight ratio less than 0.24, a normal-C16 to total C16 weight ratio less than 0.25, a normal-C17 to total C17 weight ratio less than 0.29, a normal-C18 to total C18 weight ratio less than 0.31, normal-C19 to total C19 weight ratio less than 0.35, a normal-C20 to total C20 weight ratio less than 0.33, a normal-C21 to total C21 weight ratio less than 0.33, a normal-C22 to total C22 weight ratio less than 0.35, normal-C23 to total C23 weight ratio less than 0.40, a normal-C24 to total C24 weight ratio less than 0.45, and a normal-C25 to total C25 weight ratio less than 0.49.

23. The hydrocarbon fluid of claim 2, wherein the condensable hydrocarbon portion is a fluid present within a production well that is in fluid communication with the organic-rich rock formation, a fluid present within processing equipment adapted to process hydrocarbon fluids produced from an organic-rich rock formation, a fluid present within a fluid storage vessel, or a fluid present within a fluid transportation pipeline.

24. An in situ method of producing hydrocarbon fluids from an organic-rich rock formation, comprising:
a) heating in situ a section of an organic-rich rock formation containing formation hydrocarbons, the section of the organic-rich rock formation having a lithostatic stress greater than 200 psi;
b) pyrolyzing at least a portion of the formation hydrocarbons thereby forming a hydrocarbon fluid; and
c) producing the hydrocarbon fluid from the organic-rich rock formation, the produced hydrocarbon fluid comprising a condensable hydrocarbon portion, the condensable hydrocarbon portion having:
i) one or more of a n-C6 to benzene weight ratio less than 35.0, a n-C7 to toluene weight ratio less than 7.0, a n-C8 to ethylbenzene weight ratio less than 16.0, a n-C8 to ortho-xylene weight ratio less than 7.0, a n-C8 to meta-xylene weight ratio less than 1.9, a n-C9 to 1-ethyl-3-methylbenzene weight ratio less than 8.2, a n-C9 to 1-ethyl-4-methylbenzene weight ratio less than 4.4, a n-C9 to 1,2,4-trimethylbenzene weight ratio less than 2.7, a n-C10 to 1-ethyl-2,3-dimethylbenzene weight ratio less than 13.5, a n-C10 to tetralin weight ratio less than 25.0, a n-C12 to 2-methylnaphthalene weight ratio less than 4.9, and a n-C12 to 1-methylnaphthalene weight ratio less than 6.9;
ii) one or more of a n-C9 to IP-9 weight ratio greater than 2.4, a n-C10 to IP-10 weight ratio greater than 1.4, a n-C11 to IP-11 weight ratio greater than 1.0, a n-C13 to IP-13 weight ratio greater than 1.1, a n-C14 to IP-14 weight ratio greater than 1.1, a n-C15 to IP-15 weight ratio greater than 1.0, a n-C16 to IP-16 weight ratio greater than 0.8, a n-C18 to IP-18 weight ratio greater than 1.0, and a n-C19 to pristane weight ratio greater than 1.6;
iii) one or more of a n-C7 to cis 1,3-dimethyl cyclopentane weight ratio less than 13.1, a n-C7 to trans 1,3-dimethyl cyclopentane weight ratio less than 14.9, a n-C7 to trans 1,2-dimethyl cyclopentane weight ratio less than 7.0, a n-C7 to methyl cyclohexane weight ratio less than 5.2, a n-C7 to ethyl cyclopentane weight ratio less than 11.3, a n-C8 to 1,1-dimethyl cyclohexane weight ratio less than 16.0, a n-C8 to trans 1,2-dimethyl cyclohexane weight ratio less than 17.5, and a n-C8 to ethyl cyclohexane weight ratio less than 12.3;
iv) one or more of a total C10 to total C20 weight ratio greater than 2.8, a total C11 to total C20 weight ratio greater than 2.3, a total C12 to total C20 weight ratio greater than 2.3, a total C13 to total C20 weight ratio greater than 2.9, a total C14 to total C20 weight ratio greater than 2.2, a total C15 to total C20 weight ratio greater than 2.2, and a total C16 to total C20 weight ratio greater than 1.6;
v) one or more of a total C10 to total C25 weight ratio greater than 7.5, a total C11 to total C25 weight ratio greater than 6.5, a total C12 to total C25 weight ratio greater than 6.5, a total C13 to total C25 weight ratio greater than 8.0, a total C14 to total C25 weight ratio greater than 6.0, a total C15 to total C25 weight ratio greater than 6.0, a total C16 to total C25 weight ratio greater than 4.5, a total C17 to total C25 weight ratio greater than 4.8, and a total C18 to total C25 weight ratio greater than 4.5;
vi) one or more of a total C7 to total C29 weight ratio greater than 3.5, a total C8 to total C29 weight ratio greater than 9.0, a total C9 to total C29 weight ratio greater than 12.0, a total C10 to total C29 weight ratio greater than 15.0, a total C11 to total C29 weight ratio greater than 13.0, a total C12 to total C29 weight ratio greater than 12.5, a total C13 to total C29 weight ratio greater than 16.0, a total C14 to total C29 weight ratio greater than 12.0, a total C15 to total C29 weight ratio greater than 12.0, a total C16 to total C29 weight ratio greater than 9.0, a total C17 to total C29 weight ratio greater than 10.0, a total C18 to total C29 weight ratio greater than 8.8, a total C19 to total C29 weight ratio greater than 7.0, a total C20 to total C29 weight ratio greater than 6.0, a total C21 to total C29 weight ratio greater than 5.5, and a total C22 to total C29 weight ratio greater than 4.2;
vii) one or more of a normal-C7 to normal-C20 weight ratio greater than 0.9, a normal-C8 to normal-C20 weight ratio greater than 2.0, a normal-C9 to normal-C20 weight ratio greater than 1.9, a normal-C10 to normal-C20 weight ratio greater than 2.2, a normal-C11 to normal-C20 weight ratio greater than 1.9, a normal-C12 to normal-C20 weight ratio greater than 1.9, a normal-C13 to normal-C20 weight ratio greater than 2.3, a normal-C14 to normal-C20 weight ratio greater than 1.8, a normal-C15 to normal-C20 weight ratio greater than 1.8, and normal-C16 to normal-C20 weight ratio greater than 1.3;
viii) one or more of a normal-C10 to normal-C25 weight ratio greater than 4.4, a normal-C11 to normal-C25 weight ratio greater than 3.8, a normal-C12 to normal-C25 weight ratio greater than 3.7, a normal-C13 to normal-C25 weight ratio greater than 4.7, a normal-C14 to normal-C25 weight ratio greater than 3.7, a normal-C15 to normal-C25 weight ratio greater than 3.7, a normal-C16 to normal-C25 weight ratio greater than 2.5, a normal-C17 to normal-C25 weight ratio greater than 3.0, and a normal-C18 to normal-C25 weight ratio greater than 3.4;
ix) one or more of a normal-C7 to normal-C29 weight ratio greater than 18.0, a normal-C8 to normal-C29 weight ratio greater than 16.0, a normal-C9 to normal-C29 weight ratio greater than 14.0, a normal-C10 to normal-C29 weight ratio greater than 14.0, a normal-C11 to normal-C29 weight ratio greater than 13.0, a normal-C12 to normal-C29 weight ratio greater than 11.0, a normal-C13 to normal-C29 weight ratio greater than 10.0, a normal-C14 to normal-C29 weight ratio greater than 9.0, a normal-C15 to normal-C29 weight ratio greater than 8.0, a normal-C16 to normal-C29 weight ratio greater than 8.0, a normal-C17 to normal-C29 weight ratio greater than 6.0, a normal-C18 to normal-C29 weight ratio greater than 6.0, a normal-C19 to normal-C29 weight ratio greater than 5.0, a normal-C20 to normal-C29 weight ratio greater than 4.0, a normal-C21 to normal-C29 weight ratio greater than 3.6, and a normal-C22 to normal-C29 weight ratio greater than 2.8; or
x) one or more of a normal-C10 to total C10 weight ratio less than 0.31, a normal-C11 to total C11 weight ratio less than 0.32, a normal-C12 to total C12 weight ratio less than 0.29, a normal-C13 to total C13 weight ratio less than 0.28, a normal-C14 to total C14 weight ratio less than 0.31, a normal-C15 to total C15 weight ratio less than 0.27, a normal-C16 to total C16 weight ratio less than 0.31, a normal-C17 to total C17 weight ratio less than 0.31, a normal-C18 to total C18 weight ratio less than 0.37, normal-C19 to total C19 weight ratio less than 0.37, a normal-C20 to total C20 weight ratio less than 0.37, a normal-C21 to total C21 weight ratio less than 0.37, a normal-C22 to total C22 weight ratio less than 0.38, normal-C23 to total C23 weight ratio less than 0.43, a normal-C24 to total C24 weight ratio less than 0.48, and a normal-C25 to total C25 weight ratio less than 0.53.

25. The method of claim 24, wherein the organic-rich rock formation is an oil shale formation.
